(19) United States
(12) Patent Application Publication
(10) Pub. No.: US 2002/0142303 A1
(43) Pub. Date: Oct. 3, 2002

(54) Proteins, genes and their use for diagnosis and treatment of Schizophrenia

(76) Inventors: Rajesh Bhikhu Parekh, Near Wendlebury (GB); Herath Mudiyanselage Athula Chandrasiri Herath, Abingdon (GB); Christn Rohlff, Oxford (GB)

Correspondence Address: PENNIE AND EDMONDS, 1155 AVENUE OF THE AMERICAS, NEW YORK, NY, 100362711

(21) Appl. No.: 09/791,378

(22) Filed: Feb. 23, 2001

Related U.S. Application Data
(63) Continuation-in-part of application No. 09/750,395, filed on Dec. 28, 2000.

(30) Foreign Application Priority Data
Feb. 24, 2000 (GB) 00044156.6

(51) Int. Cl. C12Q 1/68; C07H 21/04

(52) U.S. Cl. 435/6; 536/23.2; 536/24.3

(57) ABSTRACT

The present invention provides methods and compositions for screening, diagnosis and prognosis of Schizophrenia, for monitoring the effectiveness of Schizophrenia treatment, identifying patients most likely to respond to a particular therapeutic treatment and for drug development. Schizophrenia-Associated Features (SFs), detectable by two-dimensional electrophoresis of cerebrospinal fluid, serum or plasma are described. The invention further provides Schizophrenia-Associated Protein Isoforms (SPIs) detectable in cerebrospinal fluid, serum or plasma, preparations comprising isolated SPIs, antibodies immunospecific for SPIs, and kits comprising the aforesaid.

### 1. INTRODUCTION

The present invention relates to the identification of proteins and protein isoforms that are associated with Schizophrenia and its onset and development, and of genes encoding the same, and to their use for e.g., clinical screening, diagnosis, prognosis, therapy and prophylaxis, as well as for drug screening and drug development.

### 2. BACKGROUND OF THE INVENTION

In the majority of psychiatric disorders, little is known about a link between changes at a cellular and/or molecular level and nervous system structure and function. The paucity of detectable neuralgic defects distinguishes neuropsychiatric disorders such as Schizophrenia from neurological disorders, where manifestations of anatomical and biochemical changes have been identified in many cases. Consequently the identification and characterization of cellular and/or molecular causative defects and neuropathologies are necessary for improved treatment of neuropsychiatric disorders.

Schizophrenia is characterized by episodes of positive symptoms such as delusions, hallucinations, paranoia and psychosis and/or negative symptoms such as flattened affect, impaired attention, social withdrawal, and cognitive impairments (Ban et al, Psychiatr. Dev. (1984) 2:179-199). It afflicts 1.1% of the U.S. population and imposes a disproportionately large economic burden due to long-term expenditures for hospitalisation, treatment and rehabilitation, and lost productivity. Cost-of-illness studies have estimated that in 1990 the total economic burden of Schizophrenia in the US was $32.5 billion. (Rice J Clin Psychiatry (1999) 60 Suppl 1 4-6). In the UK, the total discounted cost to society attributable to an annual cohort of newly-diagnosed patients with Schizophrenia over the first 5 years following diagnosis has been estimated at £-862 million (Guest and Cookson Pharmacoeconomics (1999) 15:597-610). Co-morbid substance abuse disorders have emerged as one of the greatest obstacles to the effective treatment of persons with Schizophrenia. Estimates of the prevalence of such co-morbidity vary, but as many as half of persons with Schizophrenia may suffer from a co-morbid drug or alcohol disorder (Dixon Schizophr Res (1999) 35 Suppl:S 93-100). Effective treatments used early in the course of Schizophrenia can help reduce the costs associated with this illness.

The relative contribution of genetic and environmental factors to the disease etiology remain uncertain, although an increased prevalence of Schizophrenia has been demonstrated in family and twin studies (Kendler Am. J. Psychiatry (1983) 140:1413-1425) and resulted in the identification of candidate chromosomes including chromosome 6 and 22 and several candidate genes, such as the dopamine D3 receptor gene (Murphy et al, J Mol Neurosci (1996) 7:147-57). However, volumetric losses in the cerebral hemisphere and as well as changes in physiologic and neuropsychological performance deficits such as a decreased prefrontal regional cerebral blood flow in the same twin studies suggest a significant contribution of nonheritable factors to the pathogenesis of Schizophrenia (Goldberg Psychiatry Res. (1994) 55:51-61).

Although genetics and genotyping may help to define the heritable risk for Schizophrenia, the utility for diagnosis, prognosis and treatment of Schizophrenia may be considerably less. Furthermore, no CNS tissue necessary for any gene expression analysis can be obtained for a living patient under normal circumstances. Proteomic approaches appear most suitable for a molecular dissection of such disease phenotypes in the central nervous system (CNS). The entire CNS is largely inaccessible to meaningful mRNA expression-based analyses of primary human material, since post mortem delays in primary human brain tissue affects mRNAs more readily than proteins (Edgar et al, Molecular Psychiatry (1999) 4:173-17). Given that the CSF bathes the brain, changes in its protein composition may reveal alterations in CNS protein expression pattern causatively or diagnostically linked to the disease. Reasonable amounts of disease associated proteins (DAPs) are secreted or released into body fluids by diseased tissue in the living patient at the onset and/or during progression of the disease. In many cases these alterations will be independent of the genetic makeup of the individual and rather directly related to a set of molecular and cellular alterations contribution to the pathogenic phenotype (Carpenter J Psychiatr Res (1998) 32:191-5).

Currently diagnosis of Schizophrenia remains clinically based on the presence of certain types of hallucinations, delusions and thought disorders (Andreasen Lancet (1995) 346:477-481). It is made on the basis of a careful clinical interview and mental status examination according to international established manuals, in particular the DSM-IV or ICD 10. The core clinical symptoms comprise formal thought disorders, delusions, hallucinations (also summarized as positive symptoms), and negative symptoms such as lack of drive and affect flattening. Neuroimaging techniques such as magnetic resonance imaging or positron emission tomography show subtle changes of the frontal and temporal lobes and the basal ganglia (Buchsbaum, Schiz. Bull. (1990) 16:379-389) in the majority of patients. Since these alterations are of little value for the diagnosis, treatment, or prognosis of the disorder in individual patients the role of the neuroimaging techniques mentioned above is by and large restricted to the exclusion of other conditions which may be accompanied by schizophrenic symptoms such as brain tumors, hemorrhages, or—in combination with chemical parameters obtained in CSF-samples—infections of the central nervous system.

Neuroleptic agents are essential for the treatment of Schizophrenia. While typical neuroleptics effect primarily the dopaminergic system, newer atypical neuroleptics also afflict serotonergic synapses. In general, the latter have greater effects on negative symptoms and cause less extrapyramidal side effects than typical neuroleptic compounds. It is generally accepted that early and continuous neuroleptic treatment may improve the outcome of the disorder. Nevertheless, regardless of the particular drug used, neuroleptic treatment is still considered to be solely symptomatic and does not inhibit the causes of the disorder.

Currently Schizophrenia has no objective biochemical markers useful for diagnosis and prognosis in living patients. Many CNS pathologies involve increased neuronal loss and such neuronal loss or impaired synaptogenesis may result in disease associated alterations of neuronal and CSF proteins. Synaptic pathologies have been implicated in Schizophrenia (Heinonen et al, Neuroscience (1995) 64:375-384; Benes Schiz. Bull. (1993) 19:537-549.). Consequently, it is not surprising that changes in synaptic proteins such as SNAP 25 (Thompson et al, Neuropsychopharmacology (1999) 21:717-22), neurotensin (Sharma et al, Am J Psychiatry (1997) 154:1019-21) and N-CAM (Vawter et al, Schizophr Res (1998) 34:123-31) have been detected in CSF of Schizophrenia patients. N-CAM levels are altered in affected twins and not in healthy siblings (Poltorak et al, Brain Res (1997) 751:152-4) suggesting they may be directly linked to the pathogenesis of Schizophrenia. Such DAPs may provide important insights into disease pathology and opportunities for better diagnosis and treatment strategies However, these changes may also occur in other diseases, such as the elevation of −2 haptoglobin in Schizophrenia and Alzheimer's disease (Johnson et al, Applied and Theoretical Electrophoresis (1992) 3:47-53) and elevated SNAP-25 levels in Schizophrenia and bipolar patients (Thompson op. cit). Therefore, the specificity and the sensitivity of distinguishing individual neurological disorders as well as acute and chronic CNS disease may require the selection of a repertoire of DAPs rather than an individual protein.

Due to the high rates at which other disorders co-occur with Schizophrenia, the time consuming nature of existing, largely inadequate, tests and their expense it would be highly desirable to measure a substance or substances in samples of cerebrospinal fluid (CSF), blood or urine that would lead to a positive diagnosis of Schizophrenia or that would help to exclude Schizophrenia from the differential diagnosis.

Therefore, a need exists to identify DAPs as sensitive and specific biomarkers for the diagnosis of Schizophrenia in living subjects. Additionally, there is a clear need for new therapeutic agents for Schizophrenia that work quickly, potently, specifically, and with fewer side effects.

### 3. SUMMARY OF THE INVENTION

The present invention provides methods and compositions for clinical screening, diagnosis, prognosis, therapy and prophylaxis of Schizophrenia, for monitoring the effectiveness of Schizophrenia treatment, for selecting participants in clinical trials, for identifying patients most likely to respond to a particular therapeutic treatment and for screening and development of drugs for treatment of Schizophrenia. A first aspect of the invention provides methods for diagnosis of Schizophrenia that comprise analyzing a sample of cerebrospinal fluid (CSF) by two-dimensional electrophoresis to detect the presence or level of at least one Schizophrenia-Associated Feature (SF), e.g., one or more of the SFs disclosed herein or any combination thereof. These methods are also suitable for clinical screening, prognosis, monitoring the results of therapy, identifying patients most likely to respond to a particular therapeutic treatment, for drug screening and development, and identification of new targets for drug treatment.

A second aspect of the invention provides methods for diagnosis of Schizophrenia that comprise detecting in a sample of CSF the presence or level of at least one Schizophrenia-Associated Protein Isoform (SPI), e.g., one or more of the SPIs disclosed herein or any combination thereof. These methods are also suitable for clinical screening, prognosis, monitoring the results of therapy, identifying patients most likely to respond to a particular therapeutic treatment, drug screening and development, and identification of new targets for drug treatment.

A third aspect of the invention provides antibodies, e.g. monoclonal and polyclonal antibodies capable of immunospecific binding to an SPI, e.g., an SPI disclosed herein.

A fourth aspect of the invention provides a preparation comprising an isolated SPI, i.e., an SPI free from proteins or protein isoforms having a significantly different isoelectric point or a significantly different apparent molecular weight from the SPI.

A fifth aspect of the invention provides methods of treating Schizophrenia, comprising administering to a subject a therapeutically effective amount of an agent that modulates (e.g., upregulates or downregulates) the expression or activity (e.g. enzymatic or binding activity), or both, of an SPI in subjects having Schizophrenia, in order to prevent or delay the onset or development of Schizophrenia, to prevent or delay the progression of Schizophrenia, or to ameliorate the symptoms of Schizophrenia.

A sixth aspect of the invention provides methods of screening for agents that modulate (e.g., upregulate or downregulate) a characteristic of, e.g., the expression or the enzymatic or binding activity, of an SPI, an SPI analog, or an SPI-related polypeptide.

3.1. Definitions

The term “SPI analog” as used herein refers to a polypeptide that possesses a similar or identical function as an SPI but need not necessarily comprise an amino acid sequence that is similar or identical to the amino acid sequence of the SPI, or possess a structure that is similar or identical to that of the SPI. As used herein, an amino acid sequence of a polypeptide is “similar” to that of an SPI if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the SPI; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the SPI; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the SPI. As used herein, a polypeptide with “similar structure” to that of an SPI refers to a polypeptide that has a similar secondary, tertiary or quarternary structure as that of the SPI. The structure of a polypeptide can determined by methods known to those skilled in the art, including but not limited to, X-ray crystallography, nuclear magnetic resonance, and crystallographic electron microscopy.

The term “SPI fusion protein” as used herein refers to a polypeptide that comprises (i) an amino acid sequence of an SPI, an SPI fragment, an SPI-related polypeptide or a fragment of an SPI-related polypeptide and (ii) an amino acid sequence of a heterologous polypeptide (i.e., a non-SPI, non-SPI fragment or non-SPI-related polypeptide).

The term “SPI homolog” as used herein refers to a polypeptide that comprises an amino acid sequence similar to that of an SPI but does not necessarily possess a similar or identical function as the SPI.

The term “SPI ortholog” as used herein refers to a non-human polypeptide that (i) comprises an amino acid sequence similar to that of an SPI and (ii) possesses a similar or identical function to that of the SPI.

The term “SPI-related polypeptide” as used herein refers to an SPI homolog, an SPI analog, an isoform of SPI, an SPI ortholog, or any combination thereof.

The term “derivative” as used herein refers to a polypeptide that comprises an amino acid sequence of a second polypeptide which has been altered by the introduction of amino acid residue substitutions, deletions or additions. The derivative polypeptide possess a similar or identical function as the second polypeptide.

The term “fragment” as used herein refers to a peptide or polypeptide comprising an amino acid sequence of at least 5 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, at least 150 amino acid residues, at least 175 amino acid residues, at least 200 amino acid residues, or at least 250 amino acid residues) of the amino acid sequence of a second polypeptide. The fragment of an SPI may or may not possess a functional activity of the second polypeptide.

The term “fold change” includes “fold increase” and “fold decrease” and refers to the relative increase or decrease in abundance of an SF or the relative increase or decrease in expression or activity of a polypeptide (e.g. an SPI) in a first sample or sample set compared to a second sample (or sample set). An SF or polypeptide fold change may be measured by any technique known to those of skill in the art, however the observed increase or decrease will vary depending upon the technique used. Preferably, fold change is determined herein as described in the Examples infra.

The term “isoform” as used herein refers to variants of a polypeptide that are encoded by the same gene, but that differ in their pI or MW, or both. Such isoforms can differ in their amino acid composition (e.g. as a result of alternative splicing or limited proteolysis) and in addition, or in the alternative, may arise from differential post-translational modification (e.g., glycosylation, acylation, phosphorylation). As used herein, the term “isoform” also refers to a protein that exists in only a single form, i.e., it is not expressed as several variants.

The term “modulate” when used herein in reference to expression or activity of an SPI or an SPI-related polypeptide refers to the upregulation or downregulation of the expression or activity of the SPI or an SPI-related polypeptide. Based on the present disclosure, such modulation can be determined by assays known to those of skill in the art or described herein.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=# of identical positions/total # of positions×100).

The determination of percent identity between two sequences can be accomplished using a mathematical algorithm known to those of skill in the art. An example of a mathematical algorithm for comparing two sequences is the algorithm of Karlin and Altschul sequence of FIG. 2A, and the amino acid sequences determined by mass spectrometry are highlighted.

FIG. 5 shows tissue distribution of SPI-238 and SPI-240 mRNA. Levels of mRNA in samples of normal tissue were quantified by real time RT-PCR. The mRNA levels are expressed as the number of copies per nanogram cDNA. Note the 25 times difference in scale between the graph containing brain-related samples, and the graph containing body samples.

### 5. DETAILED DESCRIPTION OF THE INVENTION

The invention described in detail below provides methods and compositions for clinical screening, diagnosis and prognosis of Schizophrenia in a mammalian subject for identifying patients most likely to respond to a particular therapeutic treatment, for monitoring the results of Schizophrenia therapy, for drug screening and drug development. The invention also encompasses the administration of therapeutic compositions to a mammalian subject to treat or prevent Schizophrenia. The mammalian subject may be a non-human mammal, but is preferably human, more preferably a human adult, i.e. a human subject at least 21 (more preferably at least 35, at least 50, at least 60, at least 70, or at least 80) years old. For clarity of disclosure, and not by way of limitation, the invention will be described with respect to the analysis of CSF samples. However, as one skilled in the art will appreciate, the assays and techniques described below can be applied to other types of samples, including a body fluid (e.g. blood, serum, plasma, saliva or urine), a tissue sample from a subject at risk of having or developing Schizophrenia (e.g. a biopsy such as a brain biopsy) or homogenate thereof. The methods and compositions of the present invention are useful for screening, diagnosis and prognosis of a living subject, but may also be used for postmortem diagnosis in a subject, for example, to identify family members of the subject who are at risk of developing the same disease.

As used herein, cerebrospinal fluid (CSF) refers to the fluid that surrounds the bulk of the central nervous system, as described in Physiological Basis of Medical Practice (J. B. West, ed., Williams and Wilkins, Baltimore, Md. 1985). CSF includes ventricular CSF and lumbar CSF. As used herein, the term “serum” refers to the supernatant fluid produced by clotting and centrifugal sedimentation of a blood sample. As used herein, the term “plasma” refers to the supernatant fluid produced by inhibition of clotting (for example, by citrate or EDTA) and centrifugal sedimentation of a blood sample. The term “blood” as used herein includes serum and plasma.

5.1 Schizophrenia-Associated Features (SFs)

In one aspect of the invention, two-dimensional electrophoresis is used to analyze CSF from a subject, preferably a living subject, in order to detect or quantify the expression Proc. Natl. Acad. Sci. USA (1990) 87:2264-2268, modified as in Karlin and Altschul (1993) Proc. Natl. Acad. Sci. USA 90:5873-5877. The NBLAST and XBLAST programs of Altschul et al, J. Mol. Biol. (1990) 215:403-410 have incorporated such an algorithm. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al, Nucleic Acids Res. (1997) 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See htt://www.ncbi.nlm.nih.gov.

Another example of a mathematical algorithm utilized for the comparison of sequences is the algorithm of Myers and Miller, CABIOS (1989). The ALIGN program (version 2.0) which is part of the GCG sequence alignment software package has incorporated such an algorithm. Other algorithms for sequence analysis known in the art include ADVANCE and ADAM as described in Torellis and Robotti Comput. Appl. Biosci. (1994) 10:3-5; and FASTA described in Pearson and Lipman Proc. Natl. Acad. Sci. USA (1988) 85:2444-8. Within FASTA, ktup is a control option that sets the sensitivity and speed of the search.

### 4. BRIEF DESCRIPTION OF THE FIGURES

FIG. 1 is an image obtained from 2-dimensional electrophoresis of human CSF, which has been annotated to identify twelve landmark features, designated CSF1 to CSF12.

FIG. 2 comprises amino acid sequences of SPI-206 (FIG. 2A) and the nucleic acid sequence encoding the amino acid sequence of FIG. 2A (FIG. 2B). Peptides of SPI-206 identified by mass spectrometry are underlined in the sequence of FIG. 2A, and the amino acid sequences determined by mass spectrometry are highlighted.

FIG. 3 shows tissue distribution of SPI-206 mRNA. Levels of mRNA in samples of normal tissue were quantified by real time RT-PCR. The mRNA levels are expressed as the number of copies per nanogram cDNA. Note the 25 times difference in scale between the graph containing brain-related samples, and the graph containing body samples.

FIG. 4 comprises amino acid sequences of SPI-238 and SPI-240 (FIG. 2A) and the nucleic acid sequence encoding the amino acid sequence of FIG. 2A (FIG. 2B). Peptides of SPI-238 and SPI-240 identified by mass spectrometry are underlined in the of one or more Schizophrenia-Associated Features (SFs) for screening, prevention or diagnosis of Schizophrenia, to determine the prognosis of a subject having Schizophrenia, to monitor progression of Schizophrenia, to monitor the effectiveness of Schizophrenia therapy, for identifying patients most likely to respond to a particular therapeutic treatment, or for drug development. As used herein, “two-dimensional electrophoresis” (2D-electrophoresis) means a technique comprising isoelectric focusing, followed by denaturing electrophoresis; this generates a two- dimensional gel (2D-gel) containing a plurality of separated proteins. Preferably, the step of denaturing electrophoresis uses polyacrylamide electrophoresis in the presence of sodium dodecyl sulfate (SDS-PAGE). Especially preferred are the highly accurate and automatable methods and apparatus (“the Preferred Technology”) described in International Application No. 97GB3307 (published as WO 98/23950) and in U.S. application Ser. No. 08/980,574, both filed Dec. 1, 1997, each of which is incorporated herein by reference in its entirety with particular reference to the protocol at pages 23-35. Briefly, the Preferred Technology provides efficient, computer-assisted methods and apparatus for identifying, selecting and characterizing biomolecules (e.g. proteins, including glycoproteins) in a biological sample. A two-dimensional array is generated by separating biomolecules on a two-dimensional gel according to their electrophoretic mobility and isoelectric point. A computer-generated digital profile of the array is generated, representing the identity, apparent molecular weight, isoelectric point, and relative abundance of a plurality of biomolecules detected in the two-dimensional array, thereby permitting computer-mediated comparison of profiles from multiple biological samples, as well as computer aided excision of separated proteins of interest.

A preferred scanner for detecting fluorescently labelled proteins is described in WO 96/36882 and in the Ph.D. thesis of David A. Basiji, entitled “Development of a High-throughput Fluorescence Scanner Employing Internal Reflection Optics and Phase-sensitive Detection (Total Internal Reflection, Electrophoresis)”, University of Washington (1997), Volume 58/12-B of Dissertation Abstracts International, page 6686, the contents of each of which are incorporated herein by reference. These documents describe an image scanner designed specifically for automated, integrated operation at high speeds. The scanner can image gels that have been stained with fluorescent dyes or silver stains, as well as storage phosphor screens. The Basiji thesis provides a phase-sensitive detection system for discriminating modulated fluorescence from baseline noise due to laser scatter or homogeneous fluorescence, but the scanner can also be operated in a non-phase-sensitive mode. This phase-sensitive detection capability would increase the sensitivity of the instrument by an order of magnitude or more compared to conventional fluorescence imaging systems. The increased sensitivity would reduce the sample-preparation load on the upstream instruments while the enhanced image quality simplifies image analysis downstream in the process.

A more highly preferred scanner is the Apollo 2 scanner (Oxford Glycosciences, Oxford, UK), which is a modified version of the above described scanner. In the Apollo 2 scanner, the gel is transported through the scanner on a precision lead-screw drive system. This is preferable to laying the glass plate on the belt-driven system that is described in the Basiji thesis, as it provides a reproducible means of accurately transporting the gel past the imaging optics.

In the Apollo 2 scanner, the gel is secured against three alignment stops that rigidly hold the glass plate in a known position. By doing this in conjunction with the above precision transport system, the absolute position of the gel can be predicted and recorded. This ensures that co-ordinates of each feature on the gel can be determined more accurately and communicated, if desired, to a cutting robot for excision of the feature. In the Apollo 2 scanner, the carrier that holds the gel has four integral fluorescent markers for use to correct the image geometry. These markers are a quality control feature that confirms that the scanning has been performed correctly.

In comparison to the scanner described in the Basiji thesis, the optical components of the Apollo 2 scanner have been inverted. In the Apollo 2 scanner, the laser, mirror, waveguide and other optical components are above the glass plate being scanned. The scanner described in the Basiji thesis has these components underneath. In the Apollo 2 scanner, the glass plate is mounted onto the scanner gel side down, so that the optical path remains through the glass plate. By doing this, any particles of gel that may break away from the glass plate will fall onto the base of the instrument rather than into the optics. This does not affect the functionality of the system, but increases its reliability.

Still more preferred is the Apollo 3 scanner, in which the signal output is digitized to the full 16-bit data without any peak saturation or without square root encoding of the signal. A compensation algorithm has also been applied to correct for any variation in detection sensitivity along the path of the scanning beam. This variation is due to anomalies in the optics and differences in collection efficiency across the waveguide. A calibration is performed using a perspex plate with an even fluorescence throughout. The data received from a scan of this plate are used to determine the multiplication factors needed to increase the signal from each pixel level to a target level. These factors are then used in subsequent scans of gels to remove any internal optical variations.

As used herein, the term “feature” refers to a spot detected in a 2D gel, and the term “Schizophrenia-Associated Feature” (SF) refers to a feature that is differentially present in a sample (e.g. a sample of CSF) from a subject having Schizophrenia compared with a sample (e.g. a sample of CSF) from a subject free from Schizophrenia. As used herein, a feature (or a protein isoform of SPI, as defined infra) is “differentially present” in a first sample with respect to a second sample when a method for detecting the feature, isoform or SPI (e.g., 2D electrophoresis or an immunoassay) gives a different signal when applied to the first and second samples. A feature, isoform or SPI is “increased” in the first sample with respect to the second if the method of detection indicates that the feature, isoform or SPI is more abundant in the first sample than in the second sample, or if the feature, isoform or SPI is detectable in the first sample and undetectable in the second sample. Conversely, a feature, isoform or SPI is “decreased” in the first sample with respect to the second if the method of detection indicates that the feature, isoform or SPI is less abundant in the first sample than in the second sample or if the feature, isoform or SPI is undetectable in the first sample and detectable in the second sample.

Preferably, the relative abundance of a feature in two samples is determined in two steps. First, the signal obtained upon detecting the feature in a sample is normalized by reference to a suitable background parameter, e.g., (a) to the total protein in the sample being analyzed (e.g., total protein loaded onto a gel); (b) to an Expression Reference Feature (ERF) i.e., a feature whose abundance is invariant, within the limits of variability of the Preferred Technology, in the population of subjects being examined, e.g. the ERFs disclosed below, or (c) more preferably to the total signal detected from all proteins in the sample.

Secondly, the normalized signal for the feature in one sample or sample set is compared with the normalized signal for the same feature in another sample or sample set in order to identify features that are “differentially present” in the first sample (or sample set) with respect to the second.

The SFs disclosed herein have been identified by comparing CSF samples from subjects having Schizophrenia against CSF samples from subjects free from Schizophrenia. Subjects free from Schizophrenia include subjects with no known disease or condition (normal subjects) and subjects with diseases (including neurological and neurodegenerative diseases) other than Schizophrenia.

Two groups of SFs have been identified through the methods and apparatus of the Preferred Technology. The first group consists of SFs that are decreased in the CSF of subjects having Schizophrenia as compared with the CSF of subjects free from Schizophrenia. These SFs can be described by apparent molecular weight (MW) and isoelectric point (pI) as provided in Table I.

TABLE I
SFs Decreased in CSF of Subjects Having Schizophrenia

| | | | Fold | Rank Sum P- |
|---|---|---|---|---|
| SF# | pl | MW (Da) | Decrease | Value |
| SF-14 | 6.24 | 102603 | 44.24 | |
| SF-16 | 4.73 | 28954 | 12.99 | |
| SF-17 | 4.89 | 18534 | 8.71 | |
| SF-18 | 6.04 | 43920 | 6.38 | |
| SF-19 | 8.99 | 21801 | 7.11 | |
| SF-20 | 4.25 | 64918 | 7.51 | |
| SF-21 | 7.10 | 10885 | 6.07 | |
| SF-22 | 9.58 | 20268 | 6.32 | |
| SF-23 | 9.81 | 14171 | 6.52 | |
| SF-24 | 4.81 | 12637 | 6.62 | 0.03689 |
| SF-25 | 4.19 | 71670 | 5.78 | |
| SF-26 | 7.17 | 47823 | 4.79 | |
| SF-27 | 8.14 | 13783 | 4.90 | |
| SF-28 | 9.25 | 12001 | 3.99 | |
| SF-29 | 8.89 | 11749 | 3.71 | |
| SF-30 | 4.52 | 109372 | 4.19 | |
| SF-31 | 5.43 | 112518 | 4.07 | |
| SF-32 | 5.43 | 48238 | 3.27 | 0.01219 |
| SF-33 | 4.25 | 106909 | 3.85 | |
| SF-34 | 9.83 | 10120 | 3.29 | |
| SF-35 | 5.03 | 36795 | 3.48 | |
| SF-36 | 9.58 | 21021 | 3.44 | |
| SF-37 | 6.08 | 93159 | 2.94 | 0.03671 |
| SF-38 | 5.67 | 48092 | 2.37 | 0.01219 |
| SF-39 | 4.67 | 14570 | 2.94 | 0.01996 |
| SF-40 | 6.93 | 27331 | 2.11 | 0.01219 |
| SF-41 | 5.19 | 50178 | 2.23 | 0.01219 |
| SF-42 | 5.98 | 90092 | 2.04 | 0.03671 |
| SF-43 | 5.43 | 49573 | 2.13 | 0.03577 |
| SF-44 | 8.16 | 24182 | 1.88 | 0.01219 |
| SF-45 | 5.30 | 49423 | 1.90 | 0.01219 |
| SF-46 | 7.39 | 68161 | 1.73 | 0.03615 |
| SF-47 | 4.86 | 38741 | 1.94 | 0.03671 |
| SF-48 | 5.11 | 35613 | 1.74 | 0.01219 |
| SF-49 | 5.90 | 23795 | 1.56 | 0.02157 |
| SF-51 | 7.10 | 23117 | 1.43 | 0.02157 |
| SF-52 | 6.00 | 49723 | 1.78 | 0.02157 |
| SF-53 | 4.72 | 20882 | 1.60 | 0.01193 |
| SF-55 | 4.94 | 59286 | 1.69 | 0.03671 |
| SF-56 | 5.04 | 57690 | 1.57 | 0.01219 |
| SF-57 | 5.36 | 20134 | 1.30 | 0.02118 |
| SF-58 | 7.20 | 19285 | 1.44 | 0.01945 |
| SF-368 | 6.18 | 105482 | 31.19 | |
| SF-369 | 4.39 | 62654 | 26.06 | |
| SF-370 | 7.71 | 57865 | 14.56 | |
| SF-371 | 7.27 | 26663 | 11.95 | |
| SF-372 | 6.58 | 14769 | 11.27 | |
| SF-373 | 5.96 | 99056 | 10.95 | |
| SF-374 | 5.00 | 161367 | 8.83 | |
| SF-375 | 7.38 | 38741 | 7.65 | |
| SF-376 | 5.42 | 18290 | 7.30 | |
| SF-377 | 6.18 | 187641 | 7.18 | |
| SF-378 | 6.45 | 60068 | 6.40 | |
| SF-379 | 5.12 | 15174 | 6.21 | |
| SF-380 | 9.83 | 39766 | 5.95 | |
| SF-381 | 4.70 | 19478 | 5.65 | |
| SF-382 | 8.54 | 54625 | 5.05 | |
| SF-383 | 7.49 | 52637 | 4.74 | |
| SF-384 | 6.27 | 186027 | 4.29 | |
| SF-385 | 5.99 | 147226 | 4.20 | |
| SF-386 | 5.94 | 70146 | 4.02 | |
| SF-387 | 6.58 | 93680 | 3.79 | |
| SF-388 | 5.89 | 102725 | 3.53 | |
| SF-389 | 5.19 | 25665 | 3.45 | |
| SF-390 | 6.30 | 186832 | 3.44 | |
| SF-391 | 4.53 | 35202 | 3.32 | |
| SF-392 | 4.99 | 21951 | 3.28 | |
| SF-393 | 8.79 | 24182 | 3.20 | |
| SF-394 | 6.45 | 16614 | 3.18 | |
| SF-395 | 4.51 | 12420 | 3.11 | |
| SF-396 | 5.56 | 23599 | 3.04 | |
| SF-397 | 9.39 | 11427 | 2.99 | |
| SF-398 | 6.32 | 22090 | 2.97 | |
| SF-399 | 8.17 | 12814 | 2.93 | |
| SF-400 | 7.50 | 20201 | 2.92 | |
| SF-401 | 5.09 | 11621 | 2.89 | |
| SF-402 | 6.03 | 13175 | 2.88 | |
| SF-403 | 4.80 | 49063 | 2.15 | |
| SF-404 | 4.50 | 32266 | 2.12 | 0.03734 |
| SF-405 | 5.89 | 60151 | 1.90 | |
| SF-406 | 4.91 | 38741 | 1.82 | 0.03671 |
| SF-407 | 9.35 | 13879 | 1.78 | |
| SF-408 | 7.20 | 37524 | 1.74 | 0.02157 |
| SF-409 | 4.78 | 136566 | 1.69 | 0.01219 |
| SF-410 | 5.13 | 65925 | 1.61 | 0.03655 |
| SF-411 | 9.55 | 18969 | 1.54 | |
| SF-412 | 4.62 | 36556 | 1.50 | |
| SF-413 | 4.98 | 182153 | 1.41 | |
| SF-414 | 5.03 | 65526 | 1.38 | 0.02157 |
| SF-415 | 5.12 | 121995 | 1.33 | |
| SF-416 | 4.99 | 58394 | 1.32 | 0.03671 |
| SF-417 | 6.01 | 21999 | 1.27 | |
| SF-418 | 9.54 | 26377 | 1.19 | |
| SF-419 | 4.85 | 52993 | 1.18 | |
| SF-420 | 4.63 | 27331 | 1.18 | |
| SF-421 | 4.86 | 153822 | 1.16 | |
| SF-422 | 5.84 | 55594 | 1.08 | |
| SF-423 | 5.43 | 143548 | 1.04 | |

Where p values are given in Table I, the statistical technique used was the Wilcoxon Rank-Sum test as described in method (a) of Section 6.1.13, Statistical Analysis of the Profiles. Where no p value is reported, the method used to select these features was on the basis of a significant fold change or qualitative presence or absence alone as described in methods (b) and (c) of Section 6.1.13 Statistical Analysis of the Profiles.

The second group consists of SFs that are increased in the CSF of subjects having Schizophrenia as compared with the CSF of subjects free from Schizophrenia. These SFs can be described by MW and pI as follows:

TABLE II
SFs Increased in CSF of Subjects Having Schizophrenia

| | | | Fold | Rank Sum P- |
|---|---|---|---|---|
| SF# | pl | MW (Da) | Increase | Value |
| SF-80 | 4.65 | 10120 | 50.81 | |
| SF-81 | 5.39 | 28439 | 30.63 | |
| SF-82 | 9.74 | 56994 | 26.22 | |
| SF-83 | 7.65 | 61670 | 22.83 | |
| SF-84 | 6.54 | 13783 | 22.09 | |
| SF-85 | 6.60 | 14652 | 20.33 | |
| SF-86 | 7.01 | 44664 | 19.00 | |
| SF-87 | 5.37 | 29604 | 18.85 | |
| SF-88 | 6.60 | 57865 | 18.41 | |
| SF-89 | 4.78 | 14319 | 16.20 | |
| SF-90 | 6.16 | 52513 | 15.76 | |
| SF-91 | 5.64 | 14171 | 14.88 | |
| SF-92 | 7.65 | 46181 | 12.52 | |
| SF-93 | 6.61 | 11467 | 10.16 | |
| SF-94 | 4.26 | 113057 | 19.74 | |
| SF-95 | 5.84 | 39743 | 13.92 | |
| SF-96 | 6.05 | 63583 | 14.25 | |
| SF-97 | 7.21 | 50321 | 13.81 | |
| SF-98 | 5.87 | 45227 | 10.44 | |
| SF-99 | 5.65 | 12549 | 11.10 | |
| SF-100 | 6.39 | 14220 | 8.00 | |
| SF-101 | 9.28 | 51958 | 10.65 | |
| SF-102 | 5.56 | 14133 | 13.04 | |
| SF-103 | 5.81 | 87306 | 7.41 | |
| SF-104 | 7.68 | 130651 | 7.24 | |
| SF-105 | 4.99 | 12018 | 10.36 | |
| SF-106 | 6.81 | 12593 | 8.39 | |
| SF-107 | 5.72 | 26522 | 8.58 | |
| SF-108 | 6.12 | 15430 | 9.33 | |
| SF-109 | 9.01 | 14280 | 7.99 | |
| SF-110 | 5.74 | 111425 | 8.18 | |
| SF-111 | 7.92 | 12182 | 10.87 | |
| SF-112 | 5.79 | 14576 | 12.88 | |
| SF-113 | 7.64 | 130785 | 5.91 | |
| SF-114 | 4.47 | 13640 | 10.76 | |
| SF-115 | 4.28 | 39080 | 9.55 | |
| SF-116 | 6.85 | 30358 | 6.43 | |
| SF-117 | 9.61 | 49898 | 7.09 | |
| SF-118 | 6.86 | 50636 | 7.01 | |
| SF-119 | 9.09 | 55966 | 10.76 | |
| SF-120 | 6.26 | 12963 | 7.51 | |
| SF-123 | 5.56 | 28440 | 6.57 | 0.01996 |
| SF-124 | 6.69 | 19285 | 5.78 | |
| SF-125 | 6.48 | 68087 | 7.59 | |
| SF-126 | 6.19 | 46232 | 6.83 | |
| SF-130 | 5.72 | 18040 | 3.94 | |
| SF-131 | 7.97 | 64150 | 6.89 | 0.03577 |
| SF-132 | 4.82 | 104557 | 7.57 | |
| SF-135 | 7.23 | 31838 | 9.87 | |
| SF-136 | 5.79 | 113516 | 6.37 | |
| SF-137 | 5.83 | 13847 | 8.77 | |
| SF-139 | 4.75 | 11568 | 6.66 | |
| SF-141 | 6.45 | 137832 | 6.74 | |
| SF-142 | 4.81 | 120492 | 5.94 | |
| SF-143 | 7.46 | 43043 | 6.51 | 0.03689 |
| SF-144 | 7.26 | 12594 | 7.16 | |
| SF-147 | 7.38 | 125122 | 8.01 | |
| SF-148 | 6.01 | 41015 | 5.74 | |
| SF-150 | 5.66 | 11503 | 6.00 | |
| SF-151 | 6.28 | 30170 | 5.44 | 0.01996 |
| SF-153 | 6.95 | 14369 | 5.11 | 0.03689 |
| SF-154 | 6.39 | 12122 | 6.10 | 0.01996 |
| SF-155 | 10.04 | 14024 | 6.39 | |
| SF-157 | 4.65 | 26063 | 6.90 | |
| SF-158 | 4.73 | 11509 | 6.46 | |
| SF-159 | 5.03 | 16103 | 6.78 | |
| SF-160 | 6.35 | 32266 | 4.30 | 0.03577 |
| SF-161 | 6.55 | 12903 | 6.74 | 0.03689 |
| SF-162 | 4.40 | 19285 | 5.61 | 0.03038 |
| SF-163 | 5.26 | 14319 | 7.15 | 0.03689 |
| SF-164 | 6.98 | 59466 | 6.46 | 0.03689 |
| SF-165 | 6.45 | 20882 | 5.07 | 0.01219 |
| SF-166 | 5.78 | 33716 | 6.36 | 0.03038 |
| SF-167 | 5.17 | 15486 | 5.33 | 0.01996 |
| SF-168 | 6.07 | 31433 | 3.53 | 0.03689 |
| SF-169 | 5.76 | 29267 | 5.94 | 0.01219 |
| SF-170 | 7.50 | 14319 | 4.06 | 0.03689 |
| SF-171 | 6.69 | 24664 | 5.41 | 0.01996 |
| SF-172 | 5.68 | 39422 | 5.09 | 0.01945 |
| SF-173 | 6.39 | 44664 | 2.98 | 0.01996 |
| SF-174 | 5.19 | 12080 | 3.27 | 0.03689 |
| SF-175 | 5.79 | 179707 | 3.50 | 0.03689 |
| SF-176 | 6.37 | 34096 | 3.96 | 0.01996 |
| SF-177 | 4.46 | 48679 | 3.35 | 0.01996 |
| SF-178 | 7.68 | 64540 | 3.59 | 0.01945 |
| SF-179 | 6.05 | 30643 | 3.18 | 0.03689 |
| SF-180 | 6.21 | 67544 | 4.12 | 0.01996 |
| SF-181 | 6.29 | 80131 | 3.60 | 0.01996 |
| SF-182 | 4.95 | 14570 | 4.65 | 0.03689 |
| SF-183 | 6.67 | 38376 | 2.13 | 0.02940 |
| SF-184 | 6.52 | 60192 | 3.21 | 0.03038 |
| SF-186 | 7.48 | 59646 | 4.70 | 0.01996 |
| SF-187 | 7.27 | 59466 | 3.57 | 0.01996 |
| SF-188 | 7.01 | 40510 | 13.04 | 0.01219 |
| SF-189 | 6.01 | 53953 | 2.58 | 0.03038 |
| SF-190 | 4.91 | 70663 | 2.23 | 0.03734 |
| SF-191 | 6.74 | 54791 | 4.40 | 0.02157 |
| SF-194 | 7.03 | 55966 | 3.73 | 0.01996 |
| SF-195 | 6.53 | 66326 | 2.79 | 0.03038 |
| SF-196 | 5.52 | 178161 | 3.04 | 0.01945 |
| SF-197 | 5.32 | 15381 | 4.88 | 0.01219 |
| SF-198 | 7.73 | 15277 | 3.62 | 0.01219 |
| SF-199 | 6.28 | 67135 | 3.75 | 0.01219 |
| SF-200 | 6.03 | 135312 | 2.23 | 0.03038 |
| SF-201 | 6.10 | 57515 | 2.41 | 0.02940 |
| SF-202 | 5.13 | 42039 | 2.44 | 0.03689 |
| SF-203 | 5.67 | 178932 | 2.78 | 0.03734 |
| SF-204 | 5.49 | 38854 | 2.38 | 0.01996 |
| SF-208 | 6.37 | 63376 | 2.48 | 0.01996 |
| SF-209 | 6.53 | 10226 | 3.34 | 0.01996 |
| SF-211 | 6.53 | 25861 | 2.94 | 0.03689 |
| SF-212 | 5.48 | 179707 | 2.55 | 0.03671 |
| SF-213 | 4.87 | 45882 | 2.47 | 0.02157 |
| SF-215 | 5.55 | 178161 | 3.28 | 0.01219 |
| SF-216 | 6.59 | 60374 | 2.38 | 0.03038 |
| SF-217 | 5.03 | 17230 | 2.22 | 0.01219 |
| SF-218 | 6.42 | 32454 | 1.93 | 0.03734 |
| SF-219 | 6.56 | 20744 | 2.77 | 0.03689 |
| SF-220 | 6.74 | 40716 | 1.79 | 0.01945 |
| SF-221 | 6.86 | 100168 | 2.85 | 0.01945 |
| SF-222 | 6.37 | 66932 | 3.08 | 0.01996 |
| SF-226 | 4.81 | 50178 | 2.44 | 0.03734 |
| SF-227 | 6.46 | 52673 | 2.13 | 0.01193 |
| SF-228 | 5.97 | 14520 | 3.68 | 0.01219 |
| SF-229 | 7.42 | 56136 | 3.58 | 0.01996 |
| SF-230 | 4.31 | 63376 | 2.86 | 0.03038 |
| SF-231 | 7.81 | 59828 | 2.82 | 0.01219 |
| SF-232 | 7.31 | 64759 | 2.79 | 0.03689 |
| SF-233 | 5.02 | 50026 | 2.47 | 0.03734 |
| SF-235 | 4.49 | 18350 | 2.31 | 0.01996 |
| SF-237 | 5.77 | 85533 | 1.83 | 0.03655 |
| SF-238 | 5.77 | 19330 | 1.85 | 0.03671 |
| SF-239 | 7.67 | 104514 | 2.52 | 0.03689 |
| SF-242 | 5.48 | 11872 | 2.37 | 0.03577 |
| SF-243 | 7.65 | 52513 | 2.07 | 0.01996 |
| SF-244 | 6.65 | 12463 | 1.80 | 0.03734 |
| SF-248 | 5.40 | 11996 | 2.14 | 0.03655 |
| SF-249 | 6.18 | 178932 | 2.23 | 0.03655 |
| SF-250 | 5.05 | 15381 | 3.03 | 0.01996 |
| SF-255 | 7.03 | 155828 | 2.25 | 0.01193 |
| SF-257 | 5.75 | 60558 | 2.60 | 0.01996 |
| SF-258 | 5.06 | 49723 | 2.03 | 0.03689 |
| SF-261 | 6.05 | 27854 | 1.69 | 0.03734 |
| SF-262 | 6.72 | 57865 | 2.53 | 0.01996 |
| SF-264 | 5.50 | 151186 | 2.30 | 0.01996 |
| SF-265 | 6.90 | 156503 | 2.34 | 0.03689 |
| SF-267 | 5.30 | 43920 | 2.02 | 0.03671 |
| SF-268 | 7.22 | 155156 | 2.24 | 0.02157 |
| SF-269 | 6.18 | 52038 | 1.98 | 0.03615 |
| SF-271 | 5.06 | 13452 | 2.76 | 0.03038 |
| SF-272 | 5.17 | 64933 | 1.57 | 0.01996 |
| SF-273 | 6.09 | 67749 | 2.05 | 0.03671 |
| SF-280 | 4.65 | 45728 | 1.82 | 0.03689 |
| SF-282 | 4.86 | 31780 | 1.46 | 0.01193 |
| SF-283 | 5.49 | 60558 | 2.17 | 0.03615 |
| SF-286 | 4.99 | 61670 | 1.73 | 0.03689 |
| SF-289 | 6.28 | 178161 | 2.02 | 0.02157 |
| SF-291 | 7.14 | 32549 | 2.34 | 0.01996 |
| SF-292 | 7.27 | 48975 | 1.89 | 0.01193 |
| SF-293 | 9.24 | 35821 | 1.87 | 0.01996 |
| SF-294 | 6.62 | 101661 | 2.20 | 0.01219 |
| SF-296 | 6.52 | 175109 | 1.82 | 0.03615 |
| SF-300 | 7.39 | 153822 | 1.81 | 0.02157 |
| SF-301 | 7.14 | 95262 | 1.86 | 0.03577 |
| SF-302 | 5.41 | 44664 | 1.70 | 0.02157 |
| SF-303 | 6.88 | 40613 | 1.68 | 0.01167 |
| SF-304 | 7.25 | 67622 | 1.81 | 0.02940 |
| SF-306 | 5.72 | 100168 | 2.41 | 0.02000 |
| SF-307 | 6.43 | 50636 | 1.83 | 0.01219 |
| SF-309 | 5.28 | 72474 | 1.94 | 0.01219 |
| SF-312 | 6.57 | 122917 | 1.59 | 0.02940 |
| SF-317 | 5.59 | 43773 | 1.25 | 0.01945 |
| SF-320 | 6.26 | 21818 | 1.84 | 0.01219 |
| SF-321 | 6.72 | 101661 | 1.70 | 0.01193 |
| SF-322 | 5.99 | 26797 | 1.52 | 0.01996 |
| SF-324 | 5.20 | 43920 | 2.03 | 0.02157 |
| SF-326 | 4.96 | 74524 | 1.82 | 0.02157 |
| SF-327 | 4.40 | 16835 | 1.50 | 0.01219 |
| SF-332 | 9.05 | 72071 | 2.19 | 0.03689 |
| SF-333 | 4.50 | 47610 | 1.60 | 0.03671 |
| SF-336 | 7.03 | 107446 | 1.97 | 0.01996 |
| SF-340 | 5.03 | 46659 | 1.46 | 0.03671 |
| SF-348 | 6.30 | 50790 | 1.75 | 0.01219 |
| SF-349 | 7.16 | 39536 | 1.34 | 0.03615 |
| SF-352 | 7.09 | 19543 | 1.62 | 0.03689 |
| SF-358 | 5.67 | 68161 | 1.52 | 0.03038 |
| SF-424 | 6.07 | 177393 | 1.02 | |
| SF-425 | 8.99 | 61111 | 1.05 | |
| SF-426 | 5.61 | 113933 | 1.05 | |
| SF-427 | 5.53 | 91613 | 1.21 | |
| SF-428 | 5.95 | 178932 | 1.22 | |
| SF-429 | 5.56 | 34240 | 1.34 | |
| SF-430 | 5.53 | 12527 | 1.37 | |
| SF-431 | 6.87 | 62896 | 1.43 | |
| SF-432 | 7.42 | 89485 | 1.44 | |
| SF-433 | 4.81 | 32909 | 1.46 | |
| SF-434 | 4.41 | 24762 | 1.49 | |
| SF-435 | 9.92 | 57167 | 1.58 | |
| SF-436 | 6.22 | 87429 | 1.60 | |
| SF-437 | 7.49 | 118924 | 1.60 | |
| SF-438 | 5.87 | 101769 | 1.61 | |
| SF-439 | 7.15 | 11799 | 1.75 | |
| SF-440 | 7.31 | 64933 | 1.77 | |
| SF-441 | 6.86 | 42595 | 1.80 | |
| SF-442 | 7.19 | 43883 | 1.81 | |
| SF-443 | 4.24 | 39855 | 1.88 | |
| SF-444 | 5.56 | 27604 | 2.00 | |
| SF-445 | 6.65 | 40716 | 2.02 | |
| SF-446 | 6.14 | 47484 | 2.04 | |
| SF-447 | 7.85 | 45269 | 2.12 | |
| SF-448 | 4.34 | 10961 | 2.12 | |
| SF-449 | 5.32 | 16835 | 2.36 | |
| SF-450 | 7.05 | 12377 | 2.44 | |
| SF-451 | 9.26 | 17225 | 2.49 | |
| SF-452 | 7.47 | 12814 | 2.61 | |
| SF-453 | 6.07 | 11167 | 3.16 | |
| SF-454 | 7.42 | 46424 | 3.18 | |
| SF-455 | 5.73 | 15824 | 3.24 | |
| SF-456 | 5.71 | 45728 | 3.32 | |
| SF-457 | 5.76 | 45728 | 3.47 | |
| SF-458 | 6.65 | 13831 | 3.50 | |
| SF-459 | 9.61 | 29902 | 3.78 | |
| SF-460 | 6.78 | 11955 | 3.87 | |
| SF-461 | 6.29 | 18044 | 3.99 | |
| SF-462 | 5.00 | 31104 | 4.10 | |
| SF-463 | 5.95 | 29529 | 4.11 | |
| SF-464 | 8.19 | 27009 | 4.27 | |
| SF-465 | 6.48 | 21974 | 4.31 | |
| SF-466 | 6.28 | 48238 | 4.85 | |
| SF-467 | 5.66 | 12221 | 5.17 | |
| SF-468 | 5.58 | 73872 | 5.25 | |
| SF-469 | 4.44 | 100168 | 5.62 | |
| SF-470 | 5.70 | 79242 | 5.92 | |
| SF-471 | 4.88 | 15911 | 5.99 | |
| SF-472 | 7.51 | 24762 | 6.05 | |
| SF-473 | 6.99 | 64834 | 6.31 | |
| SF-474 | 6.22 | 17863 | 6.43 | |
| SF-475 | 5.68 | 73979 | 7.33 | |
| SF-476 | 4.24 | 65625 | 7.33 | |
| SF-477 | 8.04 | 55531 | 8.58 | |
| SF-478 | 6.80 | 32080 | 9.79 | |
| SF-479 | 5.19 | 39007 | 9.94 | |
| SF-480 | 6.06 | 34648 | 10.13 | |
| SF-481 | 8.44 | 41146 | 10.31 | |
| SF-482 | 9.70 | 57692 | 10.68 | |
| SF-483 | 6.56 | 44727 | 10.68 | |
| SF-484 | 5.62 | 38698 | 11.97 | |
| SF-485 | 7.09 | 42796 | 12.15 | |
| SF-486 | 5.31 | 39135 | 12.15 | |
| SF-487 | 7.28 | 34494 | 12.33 | |
| SF-488 | 6.23 | 167704 | 12.70 | |
| SF-489 | 6.35 | 31104 | 13.07 | |
| SF-490 | 7.08 | 84191 | 13.99 | |
| SF-491 | 6.31 | 43188 | 14.36 | |
| SF-492 | 6.28 | 41481 | 15.10 | |
| SF-493 | 5.27 | 11955 | 15.28 | |
| SF-494 | 6.69 | 39193 | 16.75 | |
| SF-495 | 5.75 | 11240 | 17.67 | |
| SF-496 | 6.92 | 109447 | 17.86 | |
| SF-497 | 9.60 | 51912 | 18.04 | |
| SF-498 | 10.65 | 11457 | 24.12 | |
| SF-499 | 7.68 | 12761 | 27.98 | |
| SF-500 | 5.64 | 13658 | 28.35 | |
| SF-501 | 7.86 | 24230 | 34.24 | |
| SF-502 | 4.23 | 39766 | 35.16 | |

Where p values are given in Table II, the statistical technique used was the Wilcoxon Rank-Sum test as described in method (a) of Section 6.1.13 Statistical Analysis of the Profiles. Where no p value is reported, the method used to select these features was on the basis of fold change or qualitative presence or absence alone as described in methods (b) and (c) of Section 6.1.13 Statistical Analysis of the Profiles.

For any given SF, the signal obtained upon analyzing CSF from subjects having Schizophrenia relative to the signal obtained upon analyzing CSF from subjects free from Schizophrenia will depend upon the particular analytical protocol and detection technique that is used. Accordingly, the present invention contemplates that each laboratory will, based on the present description, establish a reference range for each SF in subjects free from Schizophrenia according to the analytical protocol and detection technique in use, as is conventional in the diagnostic art. Preferably, at least one control positive CSF sample from a subject known to have Schizophrenia or at least one control negative CSF sample from a subject known to be free from Schizophrenia (and more preferably both positive and negative control samples) are included in each batch of test samples analyzed. In one embodiment, the level of expression of a feature is determined relative to a background value, which is defined as the level of signal obtained from a proximal region of the image that (a) is equivalent in area to the particular feature in question; and (b) contains no discernable protein feature. The reference range, depending upon the method of detection used and the conditions under which detection is carried out, can include no feature or isoform present, or non-detectable levels of feature or isoform present. Proteins described by pI and MW provided in Tables I and II can be identified by searching 2D-PAGE databases with those pI and MW values. Examples of such databases are provided on the ExPASy Molecular Biology Server (http://www.expasy.ch) under the “SWISS-2DPAGE” section, and other databases are further referenced on this server. Such databases typically provide interactive 2D gels images for a given set of sample and preparation protocol, and the skilled artisan can obtain information relevant to a given feature by pointing and clicking the appropriate section of the image.

In a preferred embodiment, the signal associated with an SF in the CSF of a subject (e.g., a subject suspected of having or known to have Schizophrenia) is normalized with reference to one or more ERFs detected in the same 2D gel. As will be apparent to one of ordinary skill in the art, such ERFs may readily be determined by comparing different samples using the Preferred Technology. Suitable ERFs include (but are not limited to) that described in the following table.

TABLE III
Expression Reference Features

| ERF# | pl | MW (Da) |
|---|---|---|
| ERF-1 | 6.28 | 48238 |
| ERF-2 | 4.28 | 26797 |

As those of skill in the art will readily appreciate, the measured MW and pI of a given feature or protein isoform will vary to some extent depending on the precise protocol used for each step of the 2D electrophoresis and for landmark matching. As used herein, the terms “MW” and “pI” are defined, respectively, to mean the apparent molecular weight and the apparent isoelectric point of a feature or protein isoform as measured in exact accordance with the Reference Protocol identified in Section 6 below. When the Reference Protocol is followed and when samples are run in duplicate or a higher number of replicates, variation in the measured mean pI of an SF or SPI is typically less than 3% and variation in the measured mean MW of an SF or SPI is typically less than 5%. Where the skilled artisan wishes to deviate from the Reference Protocol, calibration experiments should be performed to compare the MW and pI for each SF or protein isoform as detected (a) by the Reference Protocol and (b) by the deviant protocol.

SFs can be used for detection, prognosis, diagnosis, or monitoring of Schizophrenia, or for identifying patients most likely to respond to a specific therapeutic treatment, or for drug development. In one embodiment of the invention, CSF from a subject (e.g., a subject suspected of having Schizophrenia) is analyzed by 2D electrophoresis for quantitative detection of one or more of the following SFs: SF-14, SF-16, SF-17, SF-18, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SF-43, SF-44, SF-45, SF-46, SF-47, SF-48, SF-49, SF-51, SF-52, SF-53, SF-55, SF-56, SF-57, SF-58, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF-400, SF-401, SF-402, SF-403, SF-404, SF-405, SF-406, SF-407, SF-408, SF-409, SF-410, SF-411, SF-412, SF-413, SF-414, SF-415, SF-416, SF-417, SF-418, SF-419, SF-420, SF-421, SF-422, SF-423, A decreased abundance of said one or more SFs in the CSF from the subject relative to CSF from a subject or subjects free from Schizophrenia (e.g., a control sample or a previously determined reference range) indicates the presence of Schizophrenia.

In another embodiment of the invention, CSF from a subject is analyzed by 2D electrophoresis for quantitative detection of one or more of the following SFs: SF-80, SF-81, SF-82, SF-83, SF-84, SF-85, SF-86, SF-87, SF-88, SF-89, SF-90, SF-91, SF-92, SF-93, SF-94, SF-95, SF-96, SF-97, SF-98, SF-99, SF-100, SF-101, SF-102, SF-103, SF-104, SF-105, SF-106, SF-107, SF-108, SF-109, SF-110, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-211, SF-212, SF-213, SF-215, SF-216, SF-217, SF-218, SF-219, SF-220, SF-221, SF-222, SF-226, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-424, SF-425, SF-426, SF-427, SF-428, SF-429, SF-430, SF-431, SF-432, SF-433, SF-434, SF-435, SF-436, SF-437, SF-438, SF-439, SF-440, SF-441, SF-442, SF-443, SF-444, SF-445, SF-446, SF-447, SF-448, SF-449, SF-450, SF-451, SF-452, SF-453, SF-454, SF-455, SF-456, SF-457, SF-458, SF-459, SF-460, SF-461, SF-462, SF-463, SF-464, SF-465, SF-466, SF-467, SF-468, SF-469, SF-470, SF-471, SF-472, SF-473, SF-474, SF-475, SF-476, SF-477, SF-478, SF-479, SF-480, SF-481, SF-482, SF-483, SF-484, SF-485, SF-486, SF-487, SF-488, SF-489, SF-490, SF-491, SF-492, SF-493, SF-494, SF-495, SF-496, SF-497, SF-498, SF-499, SF-500, SF-501, SF-502. An increased abundance of said one or more SFs in the CSF from the subject relative to CSF from a subject or subjects free from Schizophrenia (e.g., a control sample or a previously determined reference range) indicates the presence of Schizophrenia.

In yet another embodiment, CSF from a subject is analyzed by 2D electrophoresis for quantitative detection of (a) one or more SFs or any combination of them, whose decreased abundance indicates the presence of Schizophrenia, i.e., SF-14, SF-16, SF-17, SF-18, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SF-43, SF-44, SF-45, SF-46, SF-47, SF-48, SF-49, SF-51, SF-52, SF-53, SF-55, SF-56, SF-57, SF-58, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF-400, SF-401, SF-402, SF-403, SF-404, SF-405, SF-406, SF-407, SF-408, SF-409, SF-410, SF-411, SF-412, SF-413, SF-414, SF-415, SF-416, SF-417, SF-418, SF-419, SF-420, SF-421, SF-422, SF-423; and (b) one or more SFs or any combination of them, whose increased abundance indicates the presence of Schizophrenia i.e., SF-80, SF-81, SF-82, SF-83, SF-84, SF-85, SF-86, SF-87, SF-88, SF-89, SF-90, SF-91, SF-92, SF-93, SF-94, SF-95, SF-96, SF-98, SF-99, SF-100, SF-101, SF-102, SF-103, SF-104, SF-105, SF-106, SF-107, SF-108, SF-109, SF-110, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-211, SF-212, SF-213, SF-215, SF-216, SF-217, SF-218, SF-219, SF-220, SF-221, SF-222, SF-226, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-424, SF-425, SF-426, SF-427, SF-428, SF-429, SF-430, SF-431, SF-432, SF-433, SF-434, SF-435, SF-436, SF-437, SF-438, SF-439, SF-440, SF-441, SF-442, SF-443, SF-444, SF-445, SF-446, SF-447, SF-448, SF-449, SF-450, SF-451, SF-452, SF-453, SF-454, SF-455, SF-456, SF-457, SF-458, SF-459, SF-460, SF-461, SF-462, SF-463, SF-464, SF-465, SF-466, SF-467, SF-468, SF-469, SF-470, SF-471, SF-472, SF-473, SF-474, SF-475, SF-476, SF-477, SF-478, SF-479, SF-480, SF-481, SF-482, SF-483, SF-484, SF-485, SF-486, SF-487, SF-488, SF-489, SF-490, SF-491, SF-492, SF-493, SF-494, SF-495, SF-496, SF-497, SF-498, SF-499, SF-500, SF-501, SF-502.

In yet another embodiment of the invention, CSF from a subject is analyzed by 2D electrophoresis for quantitative detection of one or more of the following SFs: SF-14, SF-16, SF-17, SF-18, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SF-43, SF-44, SF-45, SF-46, SF-47, SF-48, SF-49, SF-51, SF-52, SF-53, SF-55, SF-56, SF-57, SF-58, SF-80, SF-81, SF-82, SF-83, SF-84, SF-85, SF-86, SF-87, SF-88, SF-89, SF-90, SF-91, SF-92, SF-93, SF-94, SF-95, SF-96, SF-97, SF-98, SF-99, SF-100, SF-101, SF-102, SF-103, SF-104, SF-105, SF-106, SF-107, SF-108, SF-109, SF-110, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-211, SF-212, SF-213, SF-215, SF-216, SF-217, SF-218, SF-219, SF-220, SF-221, SF-222, SF-226, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF-400, SF-401, SF-402, SF-403, SF-404, SF-405, SF-406, SF-407, SF-408, SF-409, SF-410, SF-411, SF-412, SF-413, SF-414, SF-415, SF-416, SF-417, SF-418, SF-419, SF-420, SF-421, SF-422, SF-423, SF-424, SF-425, SF-426, SF-427, SF-428, SF-429, SF-430, SF-431, SF-432, SF-433, SF-434, SF-435, SF-436, SF-437, SF-438, SF-439, SF-440, SF-441, SF-442, SF-443, SF-444, SF-445, SF-446, SF-447, SF-448, SF-449, SF-450, SF-451, SF-452, SF-453, SF-454, SF-455, SF-456, SF-457, SF-458, SF-459, SF-460, SF-461, SF-462, SF-463, SF-464, SF-465, SF-466, SF-467, SF-468, SF-469, SF-470, SF-471, SF-472, SF-473, SF-474, SF-475, SF-476, SF-477, SF-478, SF-479, SF-480, SF-481, SF-482, SF-483, SF-484, SF-485, SF-486, SF-487, SF-488, SF-489, SF-490, SF-491, SF-492, SF-493, SF-494, SF-495, SF-496, SF-497, SF-498, SF-499, SF-500, SF-501, SF-502 wherein the ratio of the one or more SFs relative to an Expression Reference Feature (ERF) indicates whether Schizophrenia is present. In a specific embodiment, a decrease in one or more SF/ERF ratios in a test sample relative to the SF/ERF ratios in a control sample or a reference range indicates the presence of Schizophrenia; SF-14, SF-16, SF-17, SF-18, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SF-43, SF-44, SF-45, SF-46, SF-47, SF-48, SF-49, SF-51, SF-52, SF-53, SF-55, SF-56, SF-57, SF-58, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF-400, SF-401, SF-402, SF-403, SF-404, SF-405, SF-406, SF-407, SF-408, SF-409, SF410, SF-411, SF-412, SF-413, SF-414, SF-415, SF-416, SF-417, SF-418, SF-419, SF-420, SF-421, SF-422, SF-423 are suitable SFs for this purpose. In another specific embodiment, an increase in one or more SF/ERF ratios in a test sample relative to the SF/ERF ratios in a control sample or a reference range indicates the presence of Schizophrenia; SF-80, SF-81, SF-82, SF-83, SF-84, SF-85, SF-86, SF-87, SF-88, SF-89, SF-90, SF-91, SF-92, SF-93, SF-94, SF-95, SF-96, SF-97, SF-98, SF-99, SF-100, SF-101, SF-102, SF-103, SF-104, SF-105, SF-106, SF-107, SF-108, SF-109, SF-110, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-211, SF-212, SF-213, SF-215, SF-216, SF-217, SF-218, SF-219, SF-220, SF-221, SF-222, SF-226, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-424, SF-425, SF-426, SF-427, SF-428, SF-429, SF-430, SF-431, SF-432, SF-433, SF-434, SF-435, SF-436, SF-437, SF-438, SF-439, SF-440, SF-441, SF-442, SF-443, SF-444, SF-445, SF-446, SF-447, SF-448, SF-449, SF-450, SF-451, SF-452, SF-453, SF-454, SF-455, SF-456, SF-457, SF-458, SF-459, SF-460, SF-461, SF-462, SF-463, SF-464, SF-465, SF-466, SF-467, SF-468, SF-469, SF-470, SF-471, SF-472, SF-473, SF-474, SF-475, SF-476, SF-477, SF-478, SF-479, SF-480, SF-481, SF-482, SF-483, SF-484, SF-485, SF-486, SF-487, SF-488, SF-489, SF-490, SF-491, SF-492, SF-493, SF-494, SF-495, SF-496, SF-497, SF-498, SF-499, SF-500, SF-501, SF-502 are suitable SFs for this purpose.

In a further embodiment of the invention, CSF from a subject is analyzed by 2D electrophoresis for quantitative detection of (a) one or more SFs, or any combination of them, whose decreased SF/ERF ratio(s) in a test sample relative to the SF/ERF ratio(s) in a control sample indicates the presence of Schizophrenia, i.e., SF-14, SF-16, SF-1 7, SF-1 8, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SF-43, SF-44, SF-45, SF-46, SF-47, SF-48, SF-49, SF-51, SF-52, SF-53, SF-55, SF-56, SF-57, SF-58, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF-400, SF-401, SF-402, SF-403, SF-404, SF-405, SF-406, SF-407, SF-408, SF-409, SF-410, SF-411, SF-412, SF-413, SF-414, SF-415, SF-416, SF-417, SF-418, SF-419, SF-420, SF-421, SF-422, SF-423; (b) one or more SFs, or any combination of them, whose increased SF/ERF ratio(s) in a test sample relative to the SF/ERF ratio(s) in a control sample indicates the presence of Schizophrenia, i.e., SF-80, SF-81, SF-82, SF-83, SF-84, SF-85, SF-86, SF-87, SF-88, SF-89, SF-90, SF-91, SF-92, SF-93, SF-94, SF-95, SF-96, SF-97, SF-98, SF-99, SF-100, SF-101, SF-102, SF-103, SF-104, SF-105, SF-106, SF-107, SF-108, SF-109, SF-101, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-211, SF-212, SF-213, SF-215, SF-216, SF-217, SF-218, SF-219, SF-220, SF-221, SF-222, SF-226, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-424, SF-425, SF-426, SF-427, SF-428, SF-429, SF-430, SF-431, SF-432, SF-433, SF-434, SF-435, SF-436, SF-437, SF-438, SF-439, SF-440, SF-441, SF-442, SF-443, SF-444, SF-445, SF-446, SF-447, SF-448, SF-449, SF-450, SF-451, SF-452, SF-453, SF-454, SF-455, SF-456, SF-457, SF-458, SF-459, SF-460, SF-461, SF-462, SF-463, SF-464, SF-465, SF-466, SF-467, SF-468, SF-469, SF-470, SF-471, SF-472, SF-473, SF-474, SF-475, SF-476, SF-477, SF-478, SF-479, SF-480, SF-481, SF-482, SF-483, SF-484, SF-485, SF-486, SF-487, SF-488, SF-489, SF-490, SF-491, SF-492, SF-493, SF-494, SF-495, SF-496, SF-497, SF-498, SF-499, SF-500, SF-501, SF-502.

In a preferred embodiment, CSF from a subject is analyzed for quantitative detection of a plurality of SFs.

5.2 Schizophrenia-Associated Protein Isoforms (SPIs)

In another aspect of the invention, CSF from a subject, preferably a living subject, is analyzed for quantitative detection of one or more Schizophrenia-Associated Protein Isoforms (SPIs) for screening or diagnosis of Schizophrenia, to determine the prognosis of a subject having Schizophrenia, to monitor the effectiveness of Schizophrenia therapy, for identifying patients most likely to respond to a particular therapeutic treatment or for drug development. As is well known in the art, a given protein may be expressed as variants (isoforms) that differ in their amino acid composition (e.g. as a result of alternative mRNA or premRNA processing, e.g. alternative splicing or limited proteolysis) or as a result of differential post-translational modification (e.g., glycosylation, phosphorylation, acylation), or both, so that proteins of identical amino acid sequence can differ in their pI, MW, or both. It follows that differential presence of a protein isoform does not require differential expression of the gene encoding the protein in question. As used herein, the term “Schizophrenia-Associated Protein Isoform” refers to a protein isoform that is differentially present in CSF from a subject having Schizophrenia compared with CSF from a subject free from Schizophrenia. As used herein, the term “isoform” also refers to a protein that exists in only a single form, i.e., it is not expressed as several variants.

Two groups of SPIs have been identified by amino acid sequencing of SFs. SPIs were isolated, subjected to proteolysis, and analyzed by mass spectrometry using the methods and apparatus of the Preferred Technology. One skilled in the art can identify sequence information from proteins analyzed by mass spectrometry and/or tandem mass spectrometry using various spectral interpretation methods and database searching tools. Examples of some of these methods and tools can be found at the Swiss Institute of Bioinformatics web site at http://www.expasy.ch/, and the European Molecular Biology Laboratory web site at www.mann.embl-heidelberg.de/Services/PeptideSearch/. Identification of SPIs was performed primarily using the SEQUEST search program (Eng et al, J. Am. Soc. Mass Spectrom. (1994) 5:976-989) with raw, uninterpreted tandem mass spectra of tryptic digest peptides as described in the Examples, infra. The first group consists of SPIs that are decreased in the CSF of subjects having Schizophrenia as compared with the CSF of subjects free from Schizophrenia, where the differential presence is significant. The amino acid sequences of tryptic digest peptides of these SPIs identified by tandem mass spectrometry and database searching as described in the Examples, infra are listed in Table IV in addition to the pIs and MWs of these SPIs. For SPI-238 and SPI-240, the partial sequence information for these SPIs derived from tandem mass spectrometry was not found to be described in any known public database. These SPIs are listed as ‘NOVEL’ in Table IV, and further described below.

TABLE IV
SPIs Decreased in CSF of Subjects Having Schizophrenia

| | | | | Amino Acid Sequences of |
|---|---|---|---|---|
| SF# | SPI# | pl | MW (Da) | Tryptic Digest Peptides |
| SF-14 | SPI-6 | 6.24 | 102603 | AASGTQNNVLR, |
| | | | | EQTMSECEAGALR |
| SF-16 | SPI-231 | 4.73 | 28954 | IPTTFENGR |
| SF-19 | SPI-312 | 8.99 | 21801 | AQGFTEDTIVFLPQTDK |
| SF-20 | SPI-352 | 4.25 | 64918 | DQDGEILLPR, |
| | | | | SAVEEMEAEEAAAK, |
| | | | | QELEDLER |
| SF-21 | SPI-232 | 7.10 | 10885 | QNLEPLFEQYINNLR |
| SF-22 | SPI-7 | 9.58 | 20268 | TMLLQPAGSLGSYSYR |
| SF-24 | SPI-353 | 4.81 | 12637 | LVGGPMDASVEEEGVR |
| SF-24 | SPI-354 | 4.81 | 12637 | SGFIEEDELGFILK |
| SF-27 | SPI-233 | 8.14 | 13783 | LVGGPMDASVEEEGVR, |
| | | | | ALDFAVGEYNK |
| SF-28 | SPI-8 | 9.25 | 12001 | LVGGPMDASVEEEGVR, |
| | | | | ALDFAVGEYNK |
| SF-29 | SPI-9 | 8.89 | 11749 | LVGGPMDASVEEEGVR, |
| | | | | ALDFAVGEYNK |
| SF-30 | SPI-10 | 4.52 | 109372 | VESLEQEAANER, |
| | | | | QQLVETHMAR |
| SF-31 | SPI-11 | 5.43 | 112518 | YLELESSGHR, |
| | | | | TCPTCNDFHGLVQK, |
| | | | | AFLFQDTPR, NNAHGYFK, |
| | | | | TYFEGER, LDQCYCER, |
| | | | | HNGQIWVLENDR, |
| | | | | CVTDPCQADTIR |
| SF-32 | SPI-13 | 5.43 | 48238 | DTDTGALLFIGK, |
| | | | | TVQAVLTVPK, |
| | | | | LSYEGEVTK, |
| | | | | LAAAVSNFGYDLYR, |
| | | | | SSFVAPLEK, |
| | | | | TSLEDFYLDEER |
| SF-32 | SPI-234 | 5.43 | 48238 | VELEDWNGR |
| SF-33 | SPI-355 | 4.25 | 106909 | VESLEQEAANER |
| SF-35 | SPI-15 | 5.03 | 36795 | SWFEPLVEDMQR, |
| | | | | LGPLVEQGR, |
| | | | | GEVQAMLGQSTEELR, |
| | | | | LEEQAQQIR, |
| | | | | SELEEQLTPVAEETR |
| SF-35 | SPI-16 | 5.03 | 36795 | ELDESLQVAER, |
| | | | | ASSIIDELFQDR, |
| | | | | TLLSNLEEAK |
| SF-36 | SPI-17 | 9.58 | 21021 | TMLLQPAGSLGSYSYR |
| SF-37 | SPI-18 | 6.08 | 93159 | EPGLQIWR, |
| | | | | HVVPNEVVVQR |
| SF-38 | SPI-235 | 5.67 | 48092 | LCTVATLR |
| SF-38 | SPI-236 | 5.67 | 48092 | SEDTGLDSVATR |
| SF-38 | SPI-19 | 5.67 | 48092 | DTDTGALLFIGK, |
| | | | | KTSLEDFYLDEER, |
| | | | | ELLDTVTAPQK, |
| | | | | LSYEGEVTK, |
| | | | | LAAAVSNFGYDLYR, |
| | | | | SSFVAPLEK |
| SF-39 | SPI-357 | 4.67 | 14570 | GLEEELQFSLGSK |
| SF-40 | SPI-20 | 6.93 | 27331 | KPNLQVFLGK, |
| | | | | LSELIQPLPLER, |
| | | | | GLVSWGNIPCGSK, |
| | | | | LVHGGPCDK, |
| | | | | EKPGVYTNVCR, |
| | | | | ESSQEQSSVVR, YTNWIQK |
| SF-41 | SPI-21 | 5.19 | 50178 | TVQAVLTVPK, |
| | | | | LSYEGEVTK, |
| | | | | LAAAVSNFGYDLYR, |
| | | | | SSFVAPLEK, |
| | | | | TSLEDFYLDEER |
| SF-42 | SPI-23 | 5.98 | 90092 | EPGLQIWR, |
| | | | | HVVPNEVVVQR, |
| | | | | YIETDPANR |
| SF-43 | SPI-26 | 5.43 | 49573 | TALASGGVLDASGDYR |
| SF-43 | SPI-24 | 5.43 | 49573 | LAAAVSNFGYDLYR, |
| | | | | TSLEDFYLDEER |
| SF-43 | SPI-25 | 5.43 | 49573 | LTIGEGQQHHLGGAK, |
| | | | | VELEDWNGR, |
| | | | | YLQEIYNSNNQK, |
| | | | | RLDGSVDFK |
| SF44 | SPI-28 | 8.16 | 24182 | TMLLQPAGSLGSYSYR, |
| | | | | AQGFTEDTIVFLPQTDK, |
| | | | | APEAQVSVQPNFQQDK |
| SF-45 | SPI-29 | 5.30 | 49423 | DTDTGALLFIGK |
| | | | | LSYEGEVTK |
| | | | | LAAAVSNFGYDLYR, |
| | | | | SSFVAPLEK, |
| | | | | TSLEDFYLDEER |
| SF-45 | SPI-30 | 5.30 | 49423 | EPGEFALLR, |
| | | | | TALASGGVLDASGDYR |
| SF-46 | SPI-32 | 7.39 | 68161 | FYYIYNEK, |
| | | | | SGIPIVTSPYQIHFTK, |
| | | | | LVAYYTLIGASGQR, |
| | | | | TIYTPGSTVLYR, |
| | | | | IPIEDGSGEVVLSR |
| SF-47 | SPI-33 | 4.86 | 38741 | DFDFVPPVVR, |
| | | | | DICEEQVNSLPGSITK, |
| | | | | GYTQQLAFR, RQGALELIK, |
| | | | | AGDFLEANYMNLQR, |
| | | | | KGYTQQLAFR |
| SF-48 | SPI-34 | 5.11 | 35613 | ELDESLQVAER, |
| | | | | ASSIIDELFQDR, |
| | | | | EILSVDCSTNNPSQAK |
| SF-48 | SPI-35 | 5.11 | 35613 | SWFEPLVEDMQR, |
| | | | | LGADMEDVCGR, |
| | | | | QWAGLVEK, LGPLVEQGR, |
| | | | | GEVQAMLGQSTEELR, |
| | | | | LEEQAQQIR, |
| | | | | SELEEQLTPVAEETR, |
| | | | | AATVGSLAGQPLQER |
| SF-49 | SPI-36 | 5.90 | 23795 | TMLLQPAGSLGSYSYR, |
| | | | | AQGFTEDTIVFLPQTDK, |
| | | | | APEAQVSVQPNFQQDK |
| SF-51 | SPI-38 | 7.10 | 23117 | TMLLQPAGSLGSYSYR, |
| | | | | AQGFTEDTIVFLPQTDK, |
| | | | | APEAQVSVQPNFQQDK |
| SF-52 | SPI-39 | 6.00 | 49723 | EPGEFALLR, |
| | | | | TALASGGVLDASGDYR, |
| | | | | YEAAVPDPR, |
| | | | | VAMHLVCPSR |
| SF-53 | SPI-237 | 4.72 | 20882 | THPHFVIPYR |
| SF-55 | SPI-238 | 4.94 | 59286 | NOVEL (cloned) |
| SF-55 | SPI-239 | 4.94 | 59286 | ALEFLQLHNGR |
| SF-55 | SPI-41 | 4.94 | 59286 | VLSALQAVQGLLVAQGR, |
| | | | | ALQDQLVLVAAK, |
| | | | | DPTFIPAPIQAK |
| SF-56 | SPI-240 | 5.04 | 57690 | NOVEL (cloned) |
| SF-56 | SPI-42 | 5.04 | 57690 | LPGIVAEGR, |
| | | | | DDLYVSDAFHK, |
| | | | | VAEGTQVLELPFK, |
| | | | | EVPLNTIIFMGR |
| SF-57 | SPI-43 | 5.36 | 20134 | CFLAFTQTK, |
| | | | | EQQALQTVCLK, |
| | | | | LDTLAQEVALLK, |
| | | | | TFHEASEDCISR, |
| | | | | NWETEITAQPDGGK |
| SF-58 | SPI-241 | 7.20 | 19285 | APEAQVSVQPNFQQDK |
| SF-58 | SPI-44 | 7.20 | 19285 | LYTLVLTDPDAPSR, |
| | | | | CDEPILSNR |
| SF-368 | SPI-401 | 6.18 | 105482 | GCPTEEGCGER, |
| | | | | AASGTQNNVLR |
| SF-368 | SPI-402 | 6.18 | 105482 | NAVGVSLPR |
| SF-369 | SPI-403 | 4.39 | 62654 | LPPNVVEESAR |
| SF-370 | SPI-404 | 7.71 | 57865 | TIYTPGSTVLYR, |
| | | | | IPIEDGSGEVVLSR |
| SF-372 | SPI-405 | 6.58 | 14769 | LVGGPMDASVEEEGVR, |
| | | | | ALDFAVGEYNK |
| SF-373 | SPI-406 | 5.96 | 99056 | VSYNVPLEAR |
| SF-373 | SPI-407 | 5.96 | 99056 | TGAQELLR |
| SF-376 | SPI-408 | 5.42 | 18290 | QSLEASLAETEGR |
| SF-376 | SPI-409 | 5.42 | 18290 | LEGEACGVYTPR |
| SF-379 | SPI-410 | 5.12 | 15174 | SELEEQLTPVAEETR, |
| | | | | GEVQAMLGQSTEELR |
| SF-380 | SPI-411 | 9.83 | 39766 | LVGGPMDASVEEEGVR, |
| | | | | ALDFAVGEYNK |
| SF-382 | SPI-412 | 8.54 | 54625 | TIYTPGSTVLYR, |
| | | | | IPIEDGSGEVVLSR |
| SF-389 | SPI-413 | 5.19 | 25665 | THLAPYSDELR |
| SF-391 | SPI-414 | 4.53 | 35202 | IPTTFENGR |
| SF-393 | SPI-415 | 8.79 | 24182 | TMLLQPAGSLGSYSYR, |
| | | | | APEAQVSVQPNFQQDK, |
| | | | | AQGFTEDTIVFLPQTDK |
| SF-396 | SPI-416 | 5.56 | 23599 | SELEEQLTPVAEETR, |
| SF-396 | SPI-417 | 5.56 | 23599 | TMLLQPAGSLGSYSYR, |
| | | | | AQGFTEDTIVFLPQTDK |
| SF-397 | SPI-418 | 9.39 | 11427 | LVGGPMDASVEEEGVR, |
| | | | | ALDFAVGEYNK |
| SF-398 | SPI-419 | 6.32 | 22090 | TMLLQPAGSLGSYSYR, |
| | | | | AQGFTEDTIVFLPQTDK |
| SF-399 | SPI-420 | 8.17 | 12814 | LVGGPMDASVEEEGVR |
| SF-402 | SPI-421 | 6.03 | 13175 | GSPAINVAVHVFR |
| SF-404 | SPI-422 | 4.50 | 32266 | IPTTFENGR |
| SF-405 | SPI-423 | 5.89 | 60151 | DASGVTFTWTPSSGK, |
| | | | | SAVQGPPER, |
| | | | | TFTCTAAYPESK, |
| | | | | WLQGSQELPR |
| SF-406 | SPI-424 | 4.91 | 38741 | KGYTQQLAFR, |
| | | | | DICEEQVNSLPGSITK, |
| | | | | AGDFLEANYMNLQR, |
| | | | | DFDFVPPVVR, |
| SF-406 | SPI-425 | 4.91 | 38741 | ASSIIDELFQDR, |
| | | | | ELDESLQVAER |
| SF-407 | SPI-426 | 9.35 | 13879 | LVGGPMDASVEEEGVR |
| SF-409 | SPI-427 | 4.78 | 136566 | FSSCGGGGGSFGAGGGF |
| | | | | GSR, NMQDMVEDYR |
| SF-409 | SPI-428 | 4.78 | 136566 | QYDSILR, |
| | | | | EGLDLQVLEDSGR, |
| | | | | QFPTPGIR |
| SF-410 | SPI-429 | 5.13 | 65925 | LCQDLGPGAFR, |
| | | | | FDPSLTQR |
| SF-410 | SPI-430 | 5.13 | 65925 | SIEVFGQFNGK, |
| | | | | DGNTLTYYR, |
| | | | | DVVLTTTFVDDIK, |
| | | | | AIEDYINEFSVR |
| SF-410 | SPI-431 | 5.13 | 65925 | WLQGSQELPR, |
| SF-411 | SPI-432 | 9.55 | 18969 | QLYGDTGVLGR, |
| | | | | SLPVSDSVLSGFEQR |
| SF-412 | SPI-433 | 4.62 | 36556 | ASSIIDELFQDR |
| SF-412 | SPI-434 | 4.62 | 36556 | ILEVVNQIQDEER |
| SF-414 | SPI-435 | 5.03 | 65526 | LCQDLGPGAFR |
| SF-416 | SPI-436 | 4.99 | 58394 | YTFELSR |
| SF-416 | SPI-437 | 4.99 | 58394 | EWVAIESDSVQPVPR, |
| | | | | MMAVAADTLQR, |
| | | | | GPVLAWINAVSAFR, |
| | | | | ALEQDLPVNIK, |
| | | | | AIHLDLEEYR, |
| | | | | EEILMHLWR, HLEDVFSK, |
| | | | | TVFGTEPDMIR, |
| | | | | MFQEIVHK, WNYIEGTK |
| SF-416 | SPI-438 | 4.99 | 58394 | DPTFIPAPIQAK, |
| | | | | ALQDQLVLVAAK, |
| | | | | LQAILGVPWK, |
| | | | | VLSALQAVQGLLVAQGR, |
| | | | | SLDFTELDVAAEK, |
| | | | | FMQAVTGWK |
| SF-416 | SPI-439 | 4.99 | 58394 | DTEEEDFHVDQATTVK, |
| | | | | VFSNGADLSGVTEEAPLK |
| SF-417 | SPI-440 | 6.01 | 21999 | APEAQVSVQPNFQQDK |
| SF-420 | SPI-441 | 4.63 | 27331 | IPTTFENGR |
| SF-421 | SPI-442 | 4.86 | 153822 | IIMLFTDGGEER, |
| | | | | FVVTDGGITR |
| SF-422 | SPI-443 | 5.84 | 55594 | DPTFIPAPIQAK, |
| | | | | ALQDQLVLVAAK, |
| | | | | SLDFTELDVAAEK |
| SF-423 | SPI-444 | 5.43 | 143548 | AETYEGVYQCTAR, |
| | | | | QPEYAVVQR, |

The second group comprises SPIs that are increased in the CSF of subjects having Schizophrenia as compared with the CSF of subjects free from Schizophrenia, where the differential presence is significant. The amino acid sequences of tryptic digest peptides of these SPIs identified by tandem mass spectrometry and database searching are listed in Table V in addition to the pIs and MWs of these SPIs. For SPI-206, the partial sequence information derived from tandem mass spectrometry was not found to be described in any known public database. This SPI is listed as ‘NOVEL’ in Table V, and further described below.

TABLE V
SPIs Increased in CSF of Subjects Having Schizophrenia

| | | | | Amino Acid Sequences of |
|---|---|---|---|---|
| SF# | SPI# | pl | MW (Da) | Tryptic Digest Peptides |
| SF-81 | SPI-321 | 5.39 | 28439 | EELVYELNPLDHR, |
| | | | | GSFEFPVGDAVSK |
| SF-81 | SPI-322 | 5.39 | 28439 | TMLLQPAGSLGSYSYR, |
| | | | | AQGFTEDTIVFLPQTDK |
| SF-82 | SPI-323 | 9.74 | 56994 | VGDTLNLNLR, |
| | | | | TTNIQGINLLFSSR |
| SF-83 | SPI-54 | 7.65 | 61670 | GGSTSYGTGSETESPR, |
| | | | | QFTSSTSYNR, |
| | | | | ESSSHHPGIAEFPSR |
| SF-84 | SPI-381 | 6.54 | 13783 | LEEQAQQIR |
| SF-85 | SPI-382 | 6.60 | 14652 | TMLLQPAGSLGSYSYR, |
| | | | | AQGFTEDTIVFLPQTDK |
| SF-86 | SPI-56 | 7.01 | 44664 | YLDGLTAER |
| SF-87 | SPI-383 | 5.37 | 29604 | TMLLQPAGSLGSYSYR, |
| | | | | AQGFTEDTIVFLPQTDK |
| SF-87 | SPI-384 | 5.37 | 29604 | TSLEDFYLDEER |
| SF-88 | SPI-57 | 6.60 | 57865 | LNMGITDLQGLR, |
| | | | | VGDTLNLNLR |
| SF-90 | SPI-324 | 6.16 | 52513 | TIYTPGSTVLYR, |
| | | | | TVMVNIENPEGIPVK |
| SF-91 | SPI-325 | 5.64 | 14171 | LEEQAQQIR, |
| | | | | LGPLVEQGR, |
| | | | | SWFEPLVEDMQR |
| SF-92 | SP-326 | 7.65 | 46181 | ELTTEIDNNIEQISSYK |
| SF-93 | SPI-359 | 6.61 | 11467 | EFTPPVQAAYQK, |
| | | | | LLVVYPWTQR, |
| | | | | VNVDAVGGEALGR |
| SF-93 | SPI-360 | 6.61 | 11467 | QMLNIPNQPK |
| SF-94 | SPI-58 | 4.26 | 113057 | LLDSLPSDTR, |
| | | | | FQPTLLTLPR |
| SF-96 | SPI-361 | 6.05 | 63583 | NFPSPVDAAFR, |
| | | | | GECQAEGVLFFQGDR |
| SF-97 | SPI-327 | 7.21 | 50321 | AVLYNYR, |
| | | | | SNLDEDIIAEENIVSR |
| SF-98 | SPI-362 | 5.87 | 45227 | FQNALLVR, |
| | | | | CCAAADPHECYAK, |
| | | | | VPQVSTPTLVEVSR |
| SF-99 | SPI-328 | 5.65 | 12549 | YGLVTYATYPK |
| SF-100 | SPI-242 | 6.39 | 14220 | LEEQAQQIR, LGPLVEQGR |
| SF-101 | SPI-329 | 9.28 | 51958 | IPIEDGSGEVVLSR |
| SF-102 | SPI-59 | 5.56 | 14133 | LAAAVSNFGYDLYR |
| SF-102 | SPI-60 | 5.56 | 14133 | LGPLVEQGR, LEEQAQQIR |
| SF-107 | SPI-243 | 5.72 | 26522 | AAPSVTLFPPSSEELQANK |
| SF-108 | SPI-244 | 6.12 | 15430 | GLQDEDGYR |
| SF-111 | SPI-62 | 7.92 | 12182 | LVGGPMDASVEEEGVR, |
| | | | | ALDFAVGEYNK |
| SF-112 | SPI-331 | 5.79 | 14576 | AQGFTEDTIVFLPQTDK, |
| | | | | TMLLQPAGSLGSYSYR |
| SF-114 | SPI-332 | 4.47 | 13640 | LAAAVSNFGYDLYR, |
| | | | | ELLDTVTAPQK |
| SF-115 | SPI-63 | 4.28 | 39080 | TYMLAFDVNDEK, |
| | | | | EQLGEFYEALDCLR, |
| | | | | SDVVYTDWK, TEDTIFLR |
| SF-116 | SPI-65 | 6.85 | 30358 | WLQGSQELPR |
| SF-118 | SPI-334 | 6.86 | 50636 | FQNALLVR |
| SF-123 | SPI-335 | 5.56 | 28440 | VWNYFQR |
| SF-124 | SPI-385 | 6.69 | 19285 | QPPFTDYR |
| SF-126 | SPI-245 | 6.19 | 46232 | TEAESWYQTK, |
| | | | | EYQELMNVK |
| SF-132 | SPI-336 | 4.82 | 104557 | FAFQAEVNR, |
| | | | | EEEAIQLDGLNASQIR |
| SF-135 | SPI-67 | 7.23 | 31838 | CSVFYGAPSK, |
| | | | | GLQDEDGYR, VEYGFQVK, |
| | | | | ITQVLHFTK |
| SF-143 | SPI-337 | 7.46 | 43043 | FEEILTR, |
| | | | | SFLVWVNEEDHLR |
| SF-144 | SPI-363 | 7.26 | 12594 | VLGAFSDGLAHLDNLK, |
| | | | | LLVVYPWTQR, |
| | | | | GTFATLSELHCDK, |
| | | | | EFTPPVQAAYQK |
| SF-151 | SPI-69 | 6.28 | 30170 | TSLEDFYLDEER, |
| | | | | SSFVAPLEK |
| SF-153 | SPI-338 | 6.95 | 14369 | LVVEWQLQDDK |
| SF-153 | SPI-339 | 6.95 | 14369 | EVVADSVWVDVK |
| SF-154 | SPI-365 | 6.39 | 12122 | EFTPPVQAAYQK, |
| | | | | VVAGVANALAHK, |
| | | | | VHLTPEEK |
| SF-157 | SPI-340 | 4.65 | 26063 | AQGFTEDTIVFLPQTDK |
| SF-158 | SPI-387 | 4.73 | 11509 | AQGFTEDTIVFLPQTDK |
| SF-158 | SPI-388 | 4.73 | 11509 | VETALEACSLPSSR |
| SF-159 | SPI-73 | 5.03 | 16103 | LGPLVEQGR, LEEQAQQIR |
| SF-160 | SPI-74 | 6.35 | 32266 | FACYYPR |
| SF-161 | SPI-75 | 6.55 | 12903 | QWAGLVEK, LGPLVEQGR, |
| | | | | LEEQAQQIR, |
| | | | | AATVGSLAGQPLQER |
| SF-163 | SPI-76 | 5.26 | 14319 | LGPLVEQGR, |
| | | | | LEEQAQQIR, |
| | | | | AATVGSLAGQPLQER |
| SF-164 | SPI-77 | 6.98 | 59466 | LNMGITDLQGLR, |
| | | | | AEFQDALEK, |
| | | | | VGDTLNLNLR |
| SF-165 | SPI-389 | 6.45 | 20882 | FSNTDYAVGYMLR, |
| | | | | LVMGIPTFGR |
| SF-166 | SPI-246 | 5.78 | 33716 | ELDESLQVAER, |
| | | | | EILSVDCSTNNPSQAK |
| SF-167 | SPI-78 | 5.17 | 15486 | QQTEWQSGQR, |
| | | | | VEQAVETEPEPELR, |
| | | | | GEVQAMLGQSTEELR, |
| | | | | SELEEQLTPVAEETR |
| SF-168 | SPI-390 | 6.07 | 31433 | SSFVAPLEK, |
| | | | | TSLEDFYLDEER |
| SF-169 | SR-391 | 5.76 | 29267 | VWNYFQR, WVEELMK, |
| | | | | SYPEILTLK |
| SF-170 | SPI-80 | 7.50 | 14319 | LGPLVEQGR, |
| | | | | LEEQAQQIR, |
| | | | | AATVGSLAGQPLQER |
| SF-171 | SPI-392 | 6.69 | 24664 | TMLLQPAGSLGSYSYR, |
| | | | | AQGFTEDTIVFLPQTDK |
| SF-172 | SPI-393 | 5.68 | 39422 | VWNYFQR, WVEELMK, |
| SF-173 | SPI-247 | 6.39 | 44664 | LVAEFDFR |
| SF-173 | SPI-81 | 6.39 | 44664 | IVQLIQDTR, SIPQVSPVR |
| SF-174 | SPI-82 | 5.19 | 12080 | GSPAINVAVHVFR |
| SF-176 | SPI-83 | 6.37 | 34096 | LQSLFDSPDFSK, |
| | | | | YGLDSDLSCK, |
| | | | | LSYEGEVTK, SSFVAPLEK, |
| | | | | TSLEDFYLDEER |
| SF-176 | SPI-248 | 6.37 | 34096 | ISYEEWAK |
| SF-176 | SPI-249 | 6.37 | 34096 | IVIEYVDR |
| SF-177 | SPI-85 | 4.46 | 48679 | ALGHLDLSGNR, |
| | | | | VAAGAFQGLR, YLFLNGNK |
| SF-178 | SPI-250 | 7.68 | 64540 | TIYTPGSTVLYR |
| SF-179 | SPI-87 | 6.05 | 30643 | TSLEDFYLDEER |
| SF-180 | SPI-251 | 6.21 | 67544 | DGFVQDEGTMFPVGK |
| SF-181 | SPI-88 | 6.29 | 80131 | VSVFVPPR |
| SF-182 | SPI-252 | 4.95 | 14570 | LGADMEDVCGR, |
| | | | | GEVQAMLGQSTEELR, |
| | | | | SELEEQLTPVAEETR |
| SF-184 | SPI-253 | 6.52 | 60192 | MLQWDDIICVR |
| SF-186 | SPI-254 | 7.48 | 59646 | LNMGITDLQGLR, |
| | | | | GQIVFMNR, |
| | | | | EMSGSPASGIPVK |
| SF-187 | SPI-255 | 7.27 | 59466 | LNMGITDLQGLR, |
| | | | | GQIVFMNR, |
| | | | | EMSGSPASGIPVK, |
| | | | | AEFQDALEK, |
| | | | | VGDTLNLNLR |
| SF-188 | SPI-394 | 7.01 | 40510 | LNDLEEALQQAK |
| SF-189 | SPI-91 | 6.01 | 53953 | GECQAEGVLFFQGDR, |
| | | | | DYFMPCPGR |
| SF-190 | SPI-257 | 4.91 | 70663 | TGYYFDGISR, |
| | | | | CLAFECPENYR, |
| | | | | IIEVEEEQEDPYLNDR |
| SF-190 | SPI-258 | 4.91 | 70663 | FDPSLTQR, |
| | | | | LCQDLGPGAFR |
| SF-191 | SPI-92 | 6.74 | 54791 | QELSEAEQATR, |
| | | | | TIYTPGSTVLYR, |
| | | | | IPIEDGSGEVVLSR |
| SF-191 | SPI-259 | 6.74 | 54791 | ATVVYQGER |
| SF-194 | SPI-261 | 7.03 | 55966 | IPIEDGSGEVVLSR |
| SF-196 | SPI-262 | 5.52 | 178161 | RPYFPVAVGK, |
| | | | | SCDIPVFMNAR |
| SF-197 | SPI-95 | 5.32 | 15381 | TMLLQPAGSLGSYSYR |
| SF-197 | SPI-93 | 5.32 | 15381 | LGADMEDVCGR, |
| | | | | VEQAVETEPEPELR, |
| | | | | GEVQAMLGQSTEELR, |
| | | | | SELEEQLTPVAEETR |
| SF-198 | SPI-96 | 7.73 | 15277 | EPGLQIWR, |
| | | | | HVVPNEVVVQR |
| SF-199 | SPI-97 | 6.28 | 67135 | EQTMSECEAGALR |
| SF-200 | SPI-99 | 6.03 | 135312 | TLNICEVGTIR, |
| | | | | QLEWGLER, |
| | | | | HEGSFIQGAEK |
| SF-201 | SPI-100 | 6.10 | 57515 | IAPANADFAFR |
| SF-202 | SPI-101 | 5.13 | 42039 | YVMLPVADQEK |
| SF-209 | SPI-105 | 6.53 | 10226 | SCDLALLETYCATPAK, |
| | | | | GIVEECCFR |
| SF-211 | SPI-367 | 6.53 | 25861 | GLVSWGNIPCGSK |
| SF-212 | SPI-263 | 5.48 | 179707 | TGESVEFVCK, |
| | | | | IDVHLVPDR |
| SF-213 | SPI-264 | 4.87 | 45882 | ELDESLQVAER |
| SF-213 | SPI-107 | 4.87 | 45882 | DHAVDLIQK, |
| | | | | TEQWSTLPPETK, |
| | | | | VLSLAQEQVGGSPEK, |
| | | | | QGSFQGGFR, |
| | | | | KADGSYAAWLSR, |
| | | | | AEMADQAAAWLTR |
| SF-215 | SPI-341 | 5.55 | 178161 | EIMENYNIALR |
| SF-217 | SPI-113 | 5.03 | 17230 | GEVQAMLGQSTEELR, |
| | | | | KVEQAVETEPEPELR, |
| | | | | SELEEQLTPVAEETR |
| SF-219 | SPI-114 | 6.56 | 20744 | EVDSGNDIYGNPIK, |
| | | | | SDGSCAWYR |
| SF-221 | SPI-342 | 6.86 | 100168 | TGAQELLR |
| SF-222 | SPI-115 | 6.37 | 66932 | AASGTQNNVLR, |
| | | | | EQTMSECEAGALR |
| SF-222 | SPI-265 | 6.37 | 66932 | YLYEIAR, CCTESLVNR |
| SF-223 | SPI-118 | 5.74 | 38251 | IETALTSLHQR, |
| | | | | LENLEQYSR |
| SF-226 | SPI-266 | 4.81 | 50178 | VEQATQAIPMER, |
| | | | | QMYPELQIAR |
| SF-226 | SPI-267 | 4.81 | 50178 | ATVNPSAPR, VLDLSCNR |
| SF-227 | SPI-268 | 6.46 | 52673 | VPPTLEVTQQPVR |
| SF-227 | SPI-269 | 6.46 | 52673 | IAPANADFAFR, |
| | | | | DFYVDENTTVR |
| SF-228 | SPI-270 | 5.97 | 14520 | IWDVVEK, |
| | | | | QPVPGQQMTLK |
| SF-229 | SPI-122 | 7.42 | 56136 | QELSEAEQATR, |
| | | | | GLEVTITAR, |
| | | | | TIYTPGSTVLYR, |
| | | | | IPIEDGSGEVVLSR |
| SF-230 | SPI-123 | 4.31 | 63376 | DQDGEILLPR, QELEDLER |
| SF-231 | SPI-124 | 7.81 | 59828 | IPGIFELGISSQSDR, |
| | | | | LPLEYSYGEYR |
| SF-232 | SPI-343 | 7.31 | 64759 | AVLYNYR |
| SF-233 | SPI-344 | 5.02 | 50026 | TALASGGVLDASGDYR |
| SF-233 | SPI-345 | 5.02 | 50026 | WLQGSQELPR |
| SF-235 | SPI-346 | 4.49 | 18350 | NFPSPVDAAFR |
| SF-239 | SPI-127 | 7.67 | 104514 | WELCDIPR, |
| | | | | HSIFTPETNPR, YEFLNGR |
| SF-242 | SPI-129 | 5.48 | 11872 | FSSCGGGGGSFGAGGGF |
| | | | | GSR |
| SF-243 | SPI-130 | 7.65 | 52513 | QDGSVDFGR, |
| | | | | LESDVSAQMEYCR, |
| | | | | EDGGGWWYNR, |
| | | | | QGFGNVATNTDGK |
| SF-243 | SPI-273 | 7.65 | 52513 | EDQYHYLLDR, |
| | | | | GFQQLLQELNQPR, |
| | | | | TLYLADTFPTNFR |
| SF-244 | SPI-369 | 6.65 | 12463 | VHLTPEEK, |
| | | | | GTFATLSELHCDK, |
| | | | | VLGAFSDGLAHLDNLK, |
| | | | | LLVVYPWTQR, |
| | | | | EFTPPVQAAYQK |
| SF-248 | SPI-370 | 5.40 | 11996 | LVGGPMDASVEEEGVR |
| SF-249 | SPI-274 | 6.18 | 178932 | TGDEITYQCR |
| SF-250 | SPI-133 | 5.05 | 15381 | RAKAELAKETDPLRR |
| SF-250 | SPI-132 | 5.05 | 15381 | VEQAVETEPEPELR, |
| | | | | GEVQAMLGQSTEELR, |
| | | | | LEEQAQQIR, |
| | | | | SELEEQLTPVAEETR |
| SF-255 | SPI-138 | 7.03 | 155828 | GPPGPPGGVVVR, |
| | | | | GGEILIPCQPR, |
| | | | | VEVLAGDLR, |
| | | | | FAQLNLAAEDTR |
| SF-257 | SPI-275 | 5.75 | 60558 | SAVQGPPER, |
| | | | | WLQGSQELPR, |
| | | | | TFTCTAAYPESK, |
| | | | | DASGVTFTWTPSSGK |
| SF-258 | SPI-139 | 5.06 | 49723 | LTVGAAQVPAQLLVGALR |
| SF-262 | SPI-397 | 6.72 | 57865 | EPGLQIWR, |
| | | | | EVQGFESATFLGYFK, |
| | | | | HVVPNEVVVQR, |
| | | | | QTQVSVLPEGGETPLFK |
| SF-264 | SPI-141 | 5.50 | 151186 | VQVTSQEYSAR |
| SF-265 | SPI-142 | 6.90 | 156503 | GPPGPPGGVVVR, |
| | | | | FAQLNLAAEDTR |
| SF-267 | SPI-143 | 5.30 | 43920 | SYELPDGQVITIGNER, |
| | | | | GYSFTTTAER, |
| | | | | QEYDESGPSIVHR |
| SF-268 | SPI-151 | 7.22 | 155156 | GPPGPPGGVVVR, |
| | | | | VISDTEADIGSNLR, |
| | | | | VTVTPDGTLIIR, |
| | | | | FAQLNLAAEDTR, |
| | | | | GGEILIPCQPR |
| SF-269 | SPI-152 | 6.18 | 52038 | GECQAEGVLFFQGDR, |
| | | | | RLWWLDLK, |
| | | | | DYFMPCPGR, |
| | | | | YYCFQGNQFLR |
| SF-271 | SPI-154 | 5.06 | 13452 | GSPAINVAVHVFR, |
| | | | | AADDTWEPFASGK |
| SF-272 | SPI-155 | 5.17 | 64933 | FDPSLTQR, |
| | | | | LCQDLGPGAFR |
| SF-273 | SPI-348 | 6.09 | 67749 | VLFYVDSEK |
| SF-280 | SPI-164 | 4.65 | 45728 | TEQWSTLPPETK, |
| | | | | VLSLAQEQVGGSPEK, |
| | | | | QGSFQGGFR, |
| | | | | AEMADQAAAWLTR |
| SF-282 | SPI-166 | 4.86 | 31780 | ESYNVQLQLPAR |
| SF-283 | SPI-167 | 5.49 | 60558 | SAVQGPPER, |
| | | | | WLQGSQELPR, |
| | | | | DASGVTFTWTPSSGK |
| SF-286 | SPI-169 | 4.99 | 61670 | YFIDFVAR, |
| | | | | YNSQNQSNNQFVLYR, |
| | | | | TVGSDTFYSFK |
| SF-286 | SPI-170 | 4.99 | 61670 | QEPSQGTTTFAVTSILR, |
| | | | | WLQGSQELPR |
| SF-289 | SPI-398 | 6.28 | 178161 | EIMENYNIALR |
| SF-291 | SPI-176 | 7.14 | 32549 | TSLEDFYLDEER |
| SF-291 | SPI-175 | 7.14 | 32549 | CSVFYGAPSK, |
| | | | | GLQDEDGYR, VEYGFQVK, |
| | | | | ITQVLHFTK, FACYYPR, |
| | | | | VHYTVCIWR |
| SF-292 | SPI-349 | 7.27 | 48975 | VVIGMDVAASEFFR |
| SF-293 | SPI-372 | 9.24 | 35821 | VPTANVSVVDLTCR, |
| | | | | LISWYDNEFGYSNR |
| SF-296 | SPI-278 | 6.52 | 175109 | IDVHLVPDR |
| SF-300 | SPI-179 | 7.39 | 153822 | GPPGPPGGVVVR, |
| | | | | FAQLNLAAEDTR |
| SF-300 | SPI-281 | 7.39 | 153822 | GPPGPVGPPGEK |
| SF-301 | SPI-375 | 7.14 | 95262 | TIYTPGSTVLYR, |
| | | | | IPIEDGSGEVVLSR |
| SF-302 | SPI-376 | 5.41 | 44664 | TIYTPGSTVLYR, |
| | | | | IPIEDGSGEVVLSR |
| SF-303 | SPI-181 | 6.88 | 40613 | ITWSNPPAQGAR, |
| | | | | VGGVQSLGGTGALR, |
| | | | | NFGLYNER, HIYLLPSGR |
| SF-304 | SPI-182 | 7.25 | 67622 | IPSETLNR, |
| | | | | QAGLGNHLSGSER, |
| | | | | ILGDPEALR |
| SF-306 | SPI-399 | 5.72 | 100168 | IEIFQTLPVR, |
| | | | | MLLELAPTSDNDFGR |
| SF-307 | SPI-183 | 6.43 | 50636 | GECQAEGVLFFQGDR, |
| | | | | DYFMPCPGR, |
| | | | | YYCFQGNQFLR |
| SF-309 | SPI-185 | 5.28 | 72474 | GECQAEGVLFFQGDR, |
| | | | | NFPSPVDAAFR, |
| | | | | VWVYPPEK |
| SF-309 | SPI-184 | 5.28 | 72474 | NGVAQEPVHLDSPAIK |
| | | | | ATWSGAVLAGR, |
| | | | | CLAPLEGAR, |
| | | | | HQFLLTGDTQGR |
| SF-317 | SPI-400 | 5.59 | 43773 | EGPVLILGR |
| SF-320 | SPI-189 | 6.26 | 21818 | TMLLQPAGSLGSYSYR, |
| | | | | APEAQVSVQPNFQQDK |
| SF-321 | SPI-379 | 6.72 | 101661 | YGLVTYATYPK |
| SF-322 | SPI-190 | 5.99 | 26797 | LQNNENNISCVER |
| SF-324 | SPI-193 | 5.20 | 43920 | ISASAEELR, LAPLAEDVR, |
| | | | | ALVQQMEQLR, |
| | | | | LEPYADQLR, |
| | | | | RVEPYGENFNK |
| SF-326 | SPI-285 | 4.96 | 74524 | NGVAQEPVHLDSPAIK, |
| | | | | HQFLLTGDTQGR, |
| | | | | ATWSGAVLAGR |
| SF-327 | SPI-195 | 4.40 | 16835 | TQSSLVPALTDFVR |
| SF-332 | SPI-289 | 9.05 | 72071 | LNMGITDLQGLR, |
| | | | | SCGLHQLLR, |
| | | | | VGDTLNLNLR |
| SF-333 | SPI-200 | 4.50 | 47610 | ALGHLDLSGNR, |
| | | | | VAAGAFQGLR |
| SF-336 | SPI-290 | 7.03 | 107446 | FVTWIEGVMR, YEFLNGR |
| SF-340 | SPI-205 | 5.03 | 46659 | VLSLAQEQVGGSPEK |
| | | | | QGSFQGGFR, |
| | | | | AEMADQAAAWLTR |
| SF-342 | SPI-206 | 5.08 | 29463 | NOVEL |
| SF-344 | SPI-296 | 4.76 | 23795 | EVAGLWIK, |
| | | | | TYGLPCHCPFK |
| SF-348 | SPI-211 | 6.30 | 50790 | GECQAEGVLFFQGDR, |
| | | | | VWVYPPEK, DYFMPCPGR, |
| | | | | YYCFQGNQFLR |
| SF-348 | SPI-302 | 6.30 | 50790 | SVLVAAGETATLR |
| SF-349 | SPI-303 | 7.16 | 39536 | ITWSNPPAQGAR, |
| | | | | VGGVQSLGGTGALR |
| SF-352 | SPI-213 | 7.09 | 19543 | LYTLVLTDPDAPSR |
| SF-352 | SPI-214 | 7.09 | 19543 | TMLLQPAGSLGSYSYR, |
| | | | | APEAQVSVQPNFQQDK |
| SF-354 | SPI-306 | 4.59 | 22458 | VVEQMCITQYER |
| SF-424 | SPI-445 | 6.07 | 177393 | TGDEITYQCR |
| SF-425 | SPI-446 | 8.99 | 61111 | LVGGPMDASVEEEGVR, |
| | | | | ALDFAVGEYNK |
| SF-434 | SPI-447 | 4.41 | 24762 | LPYTASSGLMAPR |
| SF-440 | SPI-448 | 7.31 | 64933 | GLIDEVNQDFTNR, |
| | | | | ESSSHHPGIAEFPSR, |
| SF-443 | SPI-449 | 4.24 | 39855 | WFYIASAFR, TEDTIFLR, |
| | | | | YVGGQEHFAHLLILR, |
| | | | | TYMLAFDVNDEK, |
| | | | | NWGLSVYADKPETTK, |
| | | | | EQLGEFYEALDCLR, |
| | | | | SDVVYTDWK |
| SF-446 | SPI-450 | 6.14 | 47484 | TALASGGVLDASGDYR, |
| | | | | EPGEFALLR |
| SF-448 | SPI-451 | 4.34 | 10961 | DQDGEILLPR |
| SF-451 | SPI-452 | 9.26 | 17225 | LVGGPMDASVEEEGVR |
| SF-459 | SPI-453 | 9.61 | 29902 | QSLEASLAETEGR |
| SF-462 | SPI-454 | 5.00 | 31104 | GSPAINVAVHVFR, |
| | | | | AADDTWEPFASGK |
| SF-462 | SPI-455 | 5.00 | 31104 | AEAIGYAYPTR |
| SF-464 | SPI-456 | 8.19 | 27009 | TMLLQPAGSLGSYSYR |
| SF-471 | SPI-457 | 4.88 | 15911 | LGPLVEQGR, |
| | | | | AATVGSLAGQPLQER, |
| | | | | LEEQAQQIR, |
| | | | | SWFEPLVEDMQR |
| SF-472 | SPI-458 | 7.51 | 24762 | EIVLTQSPATLSLSPGER, |
| | | | | FSGSGSGTDFTLTISR, |
| | | | | VYACEVTHQGLSSPVTK |
| SF-472 | SPI-459 | 7.51 | 24762 | TMLLQPAGSLGSYSYR, |
| | | | | AQGFTEDTIVFLPQTDK |
| SF-475 | SPI-460 | 5.68 | 73979 | WELLQQVDTTTR |
| SF-477 | SPI-461 | 8.04 | 55531 | QELSEAEQATR |
| SF-478 | SPI-462 | 6.80 | 32080 | VVEEQESR, VHYTVCIWR, |
| | | | | CSVFYGAPSK, FACYYPR, |
| | | | | VEYGFQVK, ITQVLHFTK, |
| | | | | GLQDEDGYR |
| SF-487 | SPI-463 | 7.28 | 34494 | TELLPGDR, DNLAIQTR |
| SF-494 | SPI-464 | 6.69 | 39193 | INHGILYDEEK, |
| | | | | EIMENYNIALR |
| SF-496 | SPI-465 | 6.92 | 109447 | CEEDEEFTCR, WELCDIPR |
| SF-496 | SPI-466 | 6.92 | 109447 | CFELQEAGPPDCR, |
| SF-502 | SPI-467 | 4.23 | 39766 | WFYIASAFR, TEDTIFLR, |
| | | | | YVGGQEHFAHLLILR, |
| | | | | TYMLAFDVNDEK, |
| | | | | NWGLSVYADKPETTK, |
| | | | | EQLGEFYEALDCLR, |
| | | | | SDVVYTDWK |

As will be evident to one of skill in the art, based upon the present description, a given SPI can be described according to the data provided for that SPI in Table IV or V. The SPI is a protein comprising a peptide sequence described for that SPI (preferably comprising a plurality of, more preferably all of, the peptide sequences described for that SPI) and has a pI of about the value stated for that SPI (preferably within 10%, more preferably within 5% still more preferably within 1% of the stated value) and has a MW of about the value stated for that SPI (preferably within 10%, more preferably within 5%, still more preferably within 1% of the stated value). Proteins comprising the peptide sequences provided in Table IV and V can be identified by searching sequence databases with those peptides using search tools known to those skilled in the art. Examples of search algorithm tools that can be used to identify proteins from peptide sequences include:

BLAST (Basic Local Alignment Search Tool): BLAST is maintained at the National Center for Biotechnology Information (NCBI) (http://www.ncbi.nlm.nih.gov) and is based on a statistical theory developed by Samuel Karlin and Steven Altschul (Proc. Natl Acad. Sci. USA (1990) 87:2284-2268), later modified as in Karlin and Altschul (Proc. Natl Acad. Sci. (1993) 90:5873). BLASTP can be used to search a protein sequence against a protein database. TBLASTN can be used to search a Protein Sequence against a Nucleotide Database, by translating each database Nucleotide sequence in all 6 reading frames.

FASTA as described in Pearson and Lipman (1988) Proc. Natl. Acad. Sci. 85:2444-8. See also Pearson Methods Enzymol. (1990) 183:63-98 and Pearson Genomics (1991) 11(3):635-50.

Examples of available protein sequence databases include:

The nr protein database maintained at the National Center for Biotechnology Information (NCBI) (http://www.ncbi.nlm.nih.gov). The nr protein database is compiled of entries from various sources including SwissProt, SwissProt updates, PIR, and PDB. The BLAST resource is available for sequence searching.

SwissProt and TrEMBL databases developed by the Swiss Bioinformatics Institute (SIB) and the European can be found at http://www.expasy.ch. BLASTP resources are available for sequence searching.

The PIR-International Protein Sequence Database maintained by the Protein Information Resource (PIR), in collaboration with the Munich Information Center for Protein Sequences (MIPS) and the Japanese International Protein Sequence Database (JIPID). The Protein Identification Resource (PIR) is a division of the National Biomedical Research Foundation (NBRF) which is affiliated with Georgetown University Medical Center and can be found at http://www nbrf.georgetown.edu/pir/searchdb.html. The database can be searched using BLAST and FASTA search algorithm tools.

The Protein Data Bank, maintained by Brookhaven National Laboratory (Long Island, N.Y., USA) which can be found at http://www.rcsb.org/pdb/. The FASTA resource is available at this website for sequence searching.

In one embodiment, CSF from a subject is analyzed for quantitative detection of one or more of the following SPIs: SPI-6, SPI-7, SPI-8, SPI-9, SPI-10, SPI-1, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-231, SPI-232, SPI-233, SPI-234, SPI-235, SPI-236, SPI-237, SPI-238, SPI-239, SPI-240, SPI-241, SPI-312, SPI-352, SPI-353, SPI-354, SPI-355, SPI-357, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, or any combination of them, wherein a decreased abundance of the SPI or SPIs (or any combination of them) in the CSF from the subject relative to CSF from a subject or subjects free from Schizophrenia (e.g., a control sample or a previously determined reference range) indicates the presence of Schizophrenia.

In another embodiment of the invention, CSF from a subject is analyzed for quantitative detection of one or more of the following SPIs: SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-211, SPI-213, SPI-214, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-359, SPI-360, SPI-361, SF-362, SF-363,SF-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, SPI-467, or any combination of them, wherein an increased abundance of the SPI or SPIs (or any combination of them) in CSF from the subject relative to CSF from a subject or subjects free from Schizophrenia (e.g., a control sample or a previously determined reference range) indicates the presence of Schizophrenia.

In a further embodiment, CSF from a subject is analyzed for quantitative detection of (a) one or more SPIs, or any combination of them, whose decreased abundance indicates the presence of Schizophrenia, i.e., SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-231, SPI-232, SPI-233, SPI-234, SPI-235, SPI-236, SPI-237, SPI-238, SPI-239, SPI-240, SPI-241, SPI-312, SPI-352, SPI-353, SPI-354, SPI-355, SPI-357, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444; and (b) one or more SPIs, or any combination of them, whose increased abundance indicates the presence of Schizophrenia, i.e., SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, SPI-467.

In yet a further embodiment, CSF from a subject is analyzed for quantitative detection of one or more SPIs and one or more previously known biomarkers of Schizophrenia (e.g., candidate markers such as hypersensitive platelet glutamate receptors (Berk et al, Int Clin Psychopharmacol (1999) 14:199-122)). In accordance with this embodiment, the abundance of each SPI and known biomarker relative to a control or reference range indicates whether a subject has Schizophrenia.

Preferably, the abundance of an SPI is normalized to an Expression Reference Protein Isoform (ERPI). ERPIs can be identified by partial amino acid sequencing of ERFs, which are described above, using the methods and apparatus of the Preferred Technology. The partial amino acid sequences of an ERPI, and the known proteins to which it is homologous is presented in Table VI.

TABLE VI
Expression Reference Protein Isoforms

| | | Amino Acid Sequences of |
|---|---|---|
| ERF# | ERPI# | Tryptic Digest Peptides |
| ERF-2 | ERPI-1 | TGAQELLR |
| ERF-2 | ERPI-2 | TMLLQPAGSLGSYSYR, |
| | | AQGFTEDTIVFLPQTDK |

As shown above, the SPIs described herein include previously unknown proteins, as well as isoforms of known proteins where the isoforms were not previously known to be associated with Schizophrenia. For each SPI, the present invention additionally provides: (a) a preparation comprising the isolated SPI; (b) a preparation comprising one or more fragments of the SPI; and (c) antibodies that bind to said SPI, to said fragments, or both to said SPI and to said fragments. As used herein, an SPI is “isolated” when it is present in a preparation that is substantially free of contaminating proteins, i.e., a preparation in which less than 10% (preferably less than 5%, more preferably less than 1%) of the total protein present is contaminating protein(s). A contaminating protein is a protein or protein isoform having a significantly different pI or MW from those of the isolated SPI, as determined by 2D electrophoresis. As used herein, a “significantly different” pI or MW is one that permits the contaminating protein to be resolved from the SPI on 2D electrophoresis, performed according to the Reference Protocol.

In one embodiment, an isolated protein is provided, said protein comprising a peptide with the amino acid sequence identified in Table IV or V for an SPI, said protein having a pI and MW within 10% (preferably within 5%, more preferably within 1%) of the values identified in Table IV or V for that SPI.

The SPIs of the invention can be qualitatively or quantitatively detected by any method known to those skilled in the art, including but not limited to the Preferred Technology described herein, kinase assays, enzyme assays, binding assays and other functional assays, immunoassays, and western blotting. In one embodiment, the SPIs are separated on a 2-D gel by virtue of their MWs and pIs and visualized by staining the gel. In one embodiment, the SPIs are stained with a fluorescent dye and imaged with a fluorescence scanner. Sypro Red (Molecular Probes, Inc., Eugene, Oregon) is a suitable dye for this purpose. A preferred fluorescent dye is Pyridinium, 4-[2-[4- (dipentylamino)-2-trifluoromethylphenyl] ethenyl]-1-(sulfobutyl)-, inner salt. See U.S. application Ser. No. 09/412,168, filed on Oct. 5, 1999, which is incorporated herein by reference in its entirety.

Alternatively, SPIs can be detected in an immunoassay. In one embodiment, an immunoassay is performed by contacting a sample from a subject to be tested with an anti-SPI antibody under conditions such that immunospecific binding can occur if the SPI is present, and detecting or measuring the amount of any immunospecific binding by the antibody. Anti-SPI antibodies can be produced by the methods and techniques taught herein; examples of such antibodies known in the art are set forth in Table VII. These antibodies shown in Table VII are already known to bind to the protein of which the SPI is itself a family member. Preferably, the anti-SPI antibody preferentially binds to the SPI rather than to other isoforms of the same protein. In a preferred embodiment, the anti-SPI antibody binds to the SPI with at least 2-fold greater affinity, more preferably at least 5-fold greater affinity, still more preferably at least 10-fold greater affinity, than to said other isoforms of the same protein.

SPIs can be transferred from the gel to a suitable membrane (e.g. a PVDF membrane) and subsequently probed in suitable assays that include, without limitation, competitive and non-competitive assay systems using techniques such as western blots and “sandwich” immunoassays using anti-SPI antibodies as described herein, e.g., the antibodies identified in Table VII, or others raised against the SPIs of interest. The immunoblots can be used to identify those anti-SPI antibodies displaying the selectivity required to immuno-specifically differentiate an SPI from other isoforms encoded by the same gene.

TABLE VII
Known Antibodies That Recognize SPIs or SPI-Related Polypeptides

| SPI# | Antibody | Manufacturer | Catalogue No. |
|---|---|---|---|
| SPI-6 | C7 Complement, Goat anti- | ACCURATE CHEMICAL & | BMD-G34 |
| | Human | SCIENTIFIC CORPORATION | |
| SPI-8 | Cystatin C, Rabbit anti-Human | ACCURATE CHEMICAL & | AXL-574 |
| | | SCIENTIFIC CORPORATION | |
| SPI-9 | Cystatin C, Rabbit anti-Human | ACCURATE CHEMICAL & | AXL-574 |
| | | SCIENTIFIC CORPORATION | |
| SPI-10 | Anti-Alzheimer precursor protein | RDI RESEARCH | RDI-ALZHPA4abm |
| | A4 | DIAGNOSTICS, INC | |
| SPI-15 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-16 | Goat anti-Clusterin (human) | RDI RESEARCH | RDI-CLUSTRCabG |
| | | DIAGNOSTICS, INC | |
| SPI-18 | Gelsolin, plasma + cytoplasmic, | ACCURATE CHEMICAL & | YBG-4628-6210 |
| | Sheep anti- | SCIENTIFIC CORPORATION | |
| SPI-23 | Gelsolin, plasma + cytoplasmic, | ACCURATE CHEMICAL & | YBG-4628-6210 |
| | Sheep anti- | SCIENTIFIC CORPORATION | |
| SPI-32 | C3 Complement, Chicken anti- | ACCURATE CHEMICAL & | IMS-01-001-02 |
| | Human | SCIENTIFIC CORPORATION | |
| SPI-33 | C3 Complement, Chicken anti- | ACCURATE CHEMICAL & | IMS-01-001-02 |
| | Human | SCIENTIFIC CORPORATION | |
| SPI-34 | Goat anti-Clusterin (human) | RDI RESEARCH | RDI-CLUSTRCabG |
| | | DIAGNOSTICS, INC | |
| SPI-35 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-41 | AT1 (306) | SANTA CRUZ | sc-579 |
| | | BIOTECHNOLOGY, INC- | |
| | | RESEARCH ANTIBODIES | |
| | | 98/99 | |
| SPI-42 | Antithrombin III, Clone: BL- | ACCURATE CHEMICAL & | BYA-9009-1 |
| | ATIII/3, Mab anti-Human | SCIENTIFIC CORPORATION | |
| SPI-43 | Tetranectin, Rabbit anti-Human | ACCURATE CHEMICAL & | AXL-494 |
| | | SCIENTIFIC CORPORATION | |
| SPI-54 | Monoclonal anti-human | BIODESIGN INTERNATIONAL | N77190M |
| | Fibrinogen | | |
| SPI-57 | C4 Complement, Chicken anti- | ACCURATE CHEMICAL & | IMS-01-032-02 |
| | Human | SCIENTIFIC CORPORATION | |
| SPI-60 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-62 | Cystatin C, Rabbit anti-Human | ACCURATE CHEMICAL & | AXL-574 |
| | | SCIENTIFIC CORPORATION | |
| SPI-63 | Alpha-1-Acid Glycoprotein, | ACCURATE CHEMICAL & | BYA-6189-1 |
| | Clone: AGP-47, Mab anti- | SCIENTIFIC CORPORATION | |
| | Human | | |
| SPI-67 | C4 Complement, Chicken anti- | ACCURATE CHEMICAL & | IMS-01-032-02 |
| | Human | SCIENTIFIC CORPORATION | |
| SPI-73 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-74 | C4 Complement, Chicken anti- | ACCURATE CHEMICAL & | IMS-01-032-02 |
| | Human | SCIENTIFIC CORPORATION | |
| SPI-75 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-76 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-77 | C4 Complement, Chicken anti- | ACCURATE CHEMICAL & | IMS-01-032-02 |
| | Human | SCIENTIFIC CORPORATION | |
| SPI-78 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-80 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-82 | Transthyretin, Prealbuminm, | ACCURATE CHEMICAL & | MED-CLA 193 |
| | 55kD, Rabbit anti-Human | SCIENTIFIC CORPORATION | |
| SPI-91 | Hemopexin, Beta-1, Rabbit anti- | ACCURATE CHEMICAL & | YN-RHHPX |
| | Human, precipitating | SCIENTIFIC CORPORATION | |
| SPI-92 | C3 Complement, Chicken anti- | ACCURATE CHEMICAL & | IMS-01-001-02 |
| | Human | SCIENTIFIC CORPORATION | |
| SPI-93 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-96 | Gelsolin, plasma + cytoplasmic, | ACCURATE CHEMICAL & | YBG-4628-6210 |
| | Sheep anti- | SCIENTIFIC CORPORATION | |
| SPI-97 | C7 Complement, Goat anti- | ACCURATE CHEMICAL & | BMD-G34 |
| | Human | SCIENTIFIC CORPORATION | |
| SPI-99 | C6 Complement, Goat anti- | ACCURATE CHEMICAL & | BMD-G33 |
| | Human | SCIENTIFIC CORPORATION | |
| SPI-100 | Monoclonal anti-Prekallikrein | BIODESIGN INTERNATIONAL | N55199M |
| | Heavy Chain | | |
| SPI-101 | Goat anti-Haptoglobin | BIODESIGN INTERNATIONAL | L15320G |
| SPI-105 | Insulin Like Growth Factor II | ACCURATE CHEMICAL & | MAS-976p |
| | (IGF-II), Clone: W2H1, Mab | SCIENTIFIC CORPORATION | |
| | anti-, frozen, IH/ELISA/RIA | | |
| SPI-107 | C4 Complement, Chicken anti- | ACCURATE CHEMICAL & | IMS-01-032-02 |
| | Human | SCIENTIFIC CORPORATION | |
| SPI-113 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-114 | Tissue Inhibitor of Matrix | ACCURATE CHEMICAL & | MED-CLA498 |
| | Metalloproteinase 2 (TIMP2) | SCIENTIFIC CORPORATION | |
| | (NO X w/TIMP1), Clone: 3A4, | | |
| | Mab anti-Human, paraffin, IH | | |
| SPI-115 | C7 Complement, Goat anti- | ACCURATE CHEMICAL & | BMD-G34 |
| | Human | SCIENTIFIC CORPORATION | |
| SPI-122 | C3 Complement, Chicken anti- | ACCURATE CHEMICAL & | IMS-01-001-02 |
| | Human | SCIENTIFIC CORPORATION | |
| SPI-124 | C8 Complement, Goat anti- | ACCURATE CHEMICAL & | BMD-G35 |
| | Human | SCIENTIFIC CORPORATION | |
| SPI-127 | Monoclonal mouse anti-human | RDI RESEARCH | RDI-TRK4P11-4D2 |
| | plasminogen | DIAGNOSTICS, INC | |
| SPI-129 | Polylconal Rabbit anti-Human | RDI RESEARCH | RDI-CYTOK1abr |
| | Cytokeratin 1 (Keratin 1) | DIAGNOSTICS, INC | |
| SPI-130 | Fibrinogen, Fibrin I, B-beta | ACCURATE CHEMICAL & | NYB-18C6 |
| | chain (Bβ 1-42), Clone: 18C6, | SCIENTIFIC CORPORATION | |
| | Mab anti-Human | | |
| SPI-132 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-143 | Actin, beta, Clone: AC-74, Mab | ACCURATE CHEMICAL & | BYA-6553-1 |
| | anti- | SCIENTIFIC CORPORATION | |
| SPI-152 | Hemopexin, Beta-1, Rabbit anti- | ACCURATE CHEMICAL & | YN-RHHPX |
| | Human, precipitating | SCIENTIFIC CORPORATION | |
| SPI-154 | Transthyretin, Prealbuminm, | ACCURATE CHEMICAL & | MED-CLA 193 |
| | 55kD, Rabbit anti-Human | SCIENTIFIC CORPORATION | |
| SPI-155 | Sheep anti-Alpha 2 Antiplasmin | BIODESIGN INTERNATIONAL | K90038C |
| SPI-164 | C4 Complement, Chicken anti- | ACCURATE CHEMICAL & | IMS-01-032-02 |
| | Human | SCIENTIFIC CORPORATION | |
| SPI-167 | Monoclonal mouse anti-human | RDI RESEARCH | RDI-TRK1A2-2B5 |
| | IgA1 | DIAGNOSTICS, INC | |
| SPI-170 | Monoclonal mouse anti-human | RDI RESEARCH | RDI-TRK1A2-2B5 |
| | IgA1 | DIAGNOSTICS, INC | |
| SPI-175 | C4 Complement, Chicken anti- | ACCURATE CHEMICAL & | IMS-01-032-02 |
| | Human | SCIENTIFIC CORPORATION | |
| SPI-183 | Hemopexin, Beta-1, Rabbit anti- | ACCURATE CHEMICAL & | YN-RHHPX |
| | Human, precipitating | SCIENTIFIC CORPORATION | |
| SPI-184 | Alpha-1-Acid Glycoprotein, | ACCURATE CHEMICAL & | BYA-6189-1 |
| | Clone: AGP-47, Mab anti- | SCIENTIFIC CORPORATION | |
| | Human | | |
| SPI-185 | Hemopexin, Beta-1, Rabbit anti- | ACCURATE CHEMICAL & | YN-RHHPX |
| | Human, precipitating | SCIENTIFIC CORPORATION | |
| SPI-190 | Factor H (Complement), | ACCURATE CHEMICAL & | IMS-01-066-02 |
| | Chicken anti-Human | SCIENTIFIC CORPORATION | |
| SPI-193 | Apolipoprotein A (HDL), Sheep | ACCURATE CHEMICAL & | ACL-20075AP |
| | anti-Human | SCIENTIFIC CORPORATION | |
| SPI-205 | C4 Complement, Chicken anti- | ACCURATE CHEMICAL & | IMS-01-032-02 |
| | Human | SCIENTIFIC CORPORATION | |
| SPI-211 | Hemopexin, Beta-1, Rabbit anti- | ACCURATE CHEMICAL & | YN-RHHPX |
| | Human, precipitating | SCIENTIFIC CORPORATION | |
| SPI-231 | Apolipoprotein D, Clone: 36C6, | ACCURATE CHEMICAL & | MED-CLA457 |
| | Mab anti-Human, paraffin, | SCIENTIFIC CORPORATION | |
| | IH/WB | | |
| SPI-233 | Cystatin C, Rabbit anti-Human | ACCURATE CHEMICAL & | AXL-574 |
| | | SCIENTIFIC CORPORATION | |
| SPI-237 | Anti-Alzheimer precursor protein | RDI RESEARCH | RDI-ALZHPA4abm |
| | A4 | DIAGNOSTICS, INC | |
| SPI-242 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-244 | C4 Complement, Chicken anti- | ACCURATE CHEMICAL & | IMS-01-032-02 |
| | Human | SCIENTIFIC CORPORATION | |
| SPI-246 | Goat anti-Clusterin (human) | RDI RESEARCH | RDI-CLUSTRCabG |
| | | DIAGNOSTICS, INC | |
| SPI-252 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-254 | C4 Complement, Chicken anti- | ACCURATE CHEMICAL & | IMS-01-032-02 |
| | Human | SCIENTIFIC CORPORATION | |
| SPI-255 | C4 Complement, Chicken anti- | ACCURATE CHEMICAL & | IMS-01-032-02 |
| | Human | SCIENTIFIC CORPORATION | |
| SPI-258 | Sheep anti-Alpha 2 Antiplasmin | BIODESIGN INTERNATIONAL | K90038C |
| SPI-261 | C3 Complement, Chicken anti- | ACCURATE CHEMICAL & | IMS-01-001-02 |
| | Human | SCIENTIFIC CORPORATION | |
| SPI-264 | Goat anti-Clusterin (human) | RDI RESEARCH | RDI-CLUSTRCabG |
| | | DIAGNOSTICS, INC | |
| SPI-265 | Albumin, Human, Chicken anti- | ACCURATE CHEMICAL & | IMS-01-026-02 |
| | | SCIENTIFIC CORPORATION | |
| SPI-269 | Monoclonal anti-Prekallikrein | BIODESIGN INTERNATIONAL | N55199M |
| | Heavy Chain | | |
| SPI-275 | Monoclonal mouse anti-human | RDI RESEARCH | RDI-TRK1A2-2B5 |
| | IgA1 | DIAGNOSTICS, INC | |
| SPI-285 | Alpha-1-Acid Glycoprotein, | ACCURATE CHEMICAL & | BYA-6189-1 |
| | Clone: AGP-47, Mab anti- | SCIENTIFIC CORPORATION | |
| | Human | | |
| SPI-289 | C4 Complement, Chicken anti- | ACCURATE CHEMICAL & | IMS-01-032-02 |
| | Human | SCIENTIFIC CORPORATION | |
| SPI-290 | Monoclonal mouse anti-human | RDI RESEARCH | RDI-TRK4P11-4D2 |
| | plasminogen | DIAGNOSTICS, INC | |
| SPI-321 | C4 Complement, Chicken anti- | ACCURATE CHEMICAL & | IMS-01-032-02 |
| | Human | SCIENTIFIC CORPORATION | |
| SPI-323 | C4 Complement, Chicken anti- | ACCURATE CHEMICAL & | IMS-01-032-02 |
| | Human | SCIENTIFIC CORPORATION | |
| SPI-325 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-326 | ANTI-CYTOKERATIN TYPE 10 | RDI RESEARCH | RDI-CBL196 |
| | | DIAGNOSTICS, INC | |
| SPI-327 | C3 Complement, Chicken anti- | ACCURATE CHEMICAL & | IMS-01-001-02 |
| | Human | SCIENTIFIC CORPORATION | |
| SPI-328 | Complement Factor B, 03 | ACCURATE CHEMICAL & | AXL-466/2 |
| | proactivator, Rabbit anti-Human | SCIENTIFIC CORPORATION | |
| SPI-329 | C3 Complement, Chicken anti- | ACCURATE CHEMICAL & | IMS-01-001-02 |
| | Human | SCIENTIFIC CORPORATION | |
| SPI-334 | Albumin, Human, Chicken anti- | ACCURATE CHEMICAL & | IMS-01-026-02 |
| | | SCIENTIFIC CORPORATION | |
| SPI-339 | C3 Complement, Chicken anti- | ACCURATE CHEMICAL & | IMS-01-001-02 |
| | Human | SCIENTIFIC CORPORATION | |
| SPI-342 | Gelsolin, plasma + cytoplasmic, | ACCURATE CHEMICAL & | YBG-4628-6210 |
| | Sheep anti- | SCIENTIFIC CORPORATION | |
| SPI-343 | C3 Complement, Chicken anti- | ACCURATE CHEMICAL & | IMS-01-001-02 |
| | Human | SCIENTIFIC CORPORATION | |
| SPI-345 | Monoclonal mouse anti-human | RDI RESEARCH | RDI-TRK1A2-2B5 |
| | IgA1 | DIAGNOSTICS, INC | |
| SPI-346 | Hemopexin, Beta-1, Rabbit anti- | ACCURATE CHEMICAL & | YN-RHHPX |
| | Human, precipitating | SCIENTIFIC CORPORATION | |
| SPI-347 | ANTI-CYTOKERATIN TYPE 10 | RDI RESEARCH | RDI-CBL196 |
| | | DIAGNOSTICS, INC | |
| SPI-348 | C7 Complement, Goat anti- | ACCURATE CHEMICAL & | BMD-G34 |
| | Human | SCIENTIFIC CORPORATION | |
| SPI-349 | Monoclonal anti-Neuron | BIODESIGN INTERNATIONAL | M37403M |
| | Specific Enolase | | |
| SPI-353 | Cystatin C, Rabbit anti-Human | ACCURATE CHEMICAL & | AXL-574 |
| | | SCIENTIFIC CORPORATION | |
| SPI-355 | Anti-Alzheimer precursor protein | RDI RESEARCH | RDI-ALZHPA4abm |
| | A4 | DIAGNOSTICS, INC | |
| SPI-357 | C4 Complement, Chicken anti- | ACCURATE CHEMICAL & | IMS-01-032-02 |
| | Human | SCIENTIFIC CORPORATION | |
| SPI-361 | Hemopexin, Beta-1, Rabbit anti- | ACCURATE CHEMICAL & | YN-RHHPX |
| | Human, precipitating | SCIENTIFIC CORPORATION | |
| SPI-362 | Albumin, Human, Chicken anti- | ACCURATE CHEMICAL & | IMS-01-026-02 |
| | | SCIENTIFIC CORPORATION | |
| SPI-370 | Cystatin C, Rabbit anti-Human | ACCURATE CHEMICAL & | AXL-574 |
| | | SCIENTIFIC CORPORATION | |
| SPI-372 | Glyceraldehyde-3-Phosphate | BIODESIGN INTERNATIONAL | H86504M |
| | Dehydrogenase | | |
| SPI-375 | C3 Complement, Chicken anti- | ACCURATE CHEMICAL & | IMS-01-001-02 |
| | Human | SCIENTIFIC CORPORATION | |
| SPI-376 | C3 Complement, Chicken anti- | ACCURATE CHEMICAL & | IMS-01-001-02 |
| | Human | SCIENTIFIC CORPORATION | |
| SPI-379 | Complement Factor B, C3 | ACCURATE CHEMICAL & | AXL-466/2 |
| | proactivator, Rabbit anti-Human | SCIENTIFIC CORPORATION | |
| SPI-381 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-397 | Gelsolin, plasma + cytoplasmic, | ACCURATE CHEMICAL & | YBG-4628-6210 |
| | Sheep anti- | SCIENTIFIC CORPORATION | |
| SPI-402 | Gelsolin, plasma +cytoplasmic, | ACCURATE CHEMICAL & | YBG-4628-6210 |
| | Sheep anti- | SCIENTIFIC CORPORATION | |
| SPI-404 | C3 Complement, Chicken anti- | ACCURATE CHEMICAL & | IMS-01-001-02 |
| | Human | SCIENTIFIC CORPORATION | |
| SPI-405 | Cystatin C, Rabbit anti-Human | ACCURATE CHEMICAL & | AXL-574 |
| | | SCIENTIFIC CORPORATION | |
| SPI-407 | Gelsolin, plasma + cytoplasmic, | ACCURATE CHEMICAL & | YBG-4628-6210 |
| | Sheep anti- | SCIENTIFIC CORPORATION | |
| SPI-408 | ANTI-CYTOKERATIN TYPE 10 | RDI RESEARCH | RDI-CBL196 |
| | | DIAGNOSTICS, INC | |
| SPI-409 | RABBIT anti-human INSULIN | RDI RESEARCH | RDI-IGFBP2abr |
| | GROWTH FACTOR BINDING | DIAGNOSTICS, INC | |
| | PROTEIN 2 | | |
| SPI-410 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-411 | Cystatin C, Rabbit anti-Human | ACCURATE CHEMICAL & | AXL-574 |
| | | SCIENTIFIC CORPORATION | |
| SPI-412 | C3 Complement, Chicken anti- | ACCURATE CHEMICAL & | IMS-01-001-02 |
| | Human | SCIENTIFIC CORPORATION | |
| SPI-413 | Apolipoprotein A (HDL), Sheep | ACCURATE CHEMICAL & | ACL-20075AP |
| | anti-Human | SCIENTIFIC CORPORATION | |
| SPI-414 | Apolipoprotein D, Clone: 36C6, | ACCURATE CHEMICAL & | MED-CLA457 |
| | Mab anti-Human, paraffin, | SCIENTIFIC CORPORATION | |
| | IH/WB | | |
| SPI-416 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-418 | Cystatin C, Rabbit anti-Human | ACCURATE CHEMICAL & | AXL-574 |
| | | SCIENTIFIC CORPORATION | |
| SPI-420 | Cystatin C, Rabbit anti-Human | ACCURATE CHEMICAL & AXL-574 | |
| | | SCIENTIFIC CORPORATION | |
| SPI-421 | Transthyretin, Prealbuminm, | ACCURATE CHEMICAL & | MED-CLA 193 |
| | 55kD, Rabbit anti-Human | SCIENTIFIC CORPORATION | |
| SPI-422 | Apolipoprotein D, Clone: 36C6, | ACCURATE CHEMICAL & | MED-CLA457 |
| | Mab anti-Human, paraffin, | SCIENTIFIC CORPORATION | |
| | IH/WB | | |
| SPI-423 | Monoclonal mouse anti-human | RDI RESEARCH | RDI-TRKIA2-2B5 |
| | IgA1 | DIAGNOSTICS, INC | |
| SPI-424 | C3 Complement, Chicken anti- | ACCURATE CHEMICAL & | IMS-01-001-02 |
| | Human | SCIENTIFIC CORPORATION | |
| SPI-425 | Goat anti-Clusterin (human) | RDI RESEARCH | RDI-CLUSTRCabG |
| | | DIAGNOSTICS, INC | |
| SPI-426 | Cystatin C, Rabbit anti-Human | ACCURATE CHEMICAL & | AXL-574 |
| | | SCIENTIFIC CORPORATION | |
| SPI-427 | Polylconal Rabbit anti-Human | RDI RESEARCH | RDI-CYTOK1abr |
| | Cytokeratin 1 (Keratin 1) | DIAGNOSTICS, INC | |
| SPI-429 | Sheep anti-Alpha 2 Antiplasmin | BIODESIGN INTERNATIONAL | K90038C |
| SPI-431 | Monoclonal mouse anti-human | RDI RESEARCH | RDI-TRK1A2-2B5 |
| | IgA1 | DIAGNOSTICS, INC | |
| SPI-432 | C8 Complement, Goat anti- | ACCURATE CHEMICAL & | |
| | Human | SCIENTIFIC CORPORATION | |
| SPI-433 | Goat anti-Clusterin (human) | RDI RESEARCH | RDI-CLUSTRCabG |
| | | DIAGNOSTICS, INC | |
| SPI-435 | Sheep anti-Alpha 2 Antiplasmin | BIODESIGN INTERNATIONAL | K90038C |
| SPI-438 | AT1 (306) | SANTA CRUZ | sc-579 |
| | | BIOTECHNOLOGY, INC- | |
| | | RESEARCH ANTIBODIES | |
| | | 98/99 | |
| SPI-441 | Apolipoprotein D, Clone: 36C6, | ACCURATE CHEMICAL & | MED-CLA457 |
| | Mab anti-Human, paraffin, | SCIENTIFIC CORPORATION | |
| | IH/WB | | |
| SPI-443 | AT1 (306) | SANTA CRUZ | sc-579 |
| | | BIOTECHNOLOGY, INC- | |
| | | RESEARCH ANTIBODIES | |
| | | 98/99 | |
| SPI-446 | Cystatin C, Rabbit anti-Human | ACCURATE CHEMICAL & | AXL-574 |
| | | SCIENTIFIC CORPORATION | |
| SPI-448 | Monoclonal anti-human | BIODESIGN INTERNATIONAL | N77190M |
| | Fibrinogen | | |
| SPI-449 | Alpha-1-Acid Glycoprotein, | ACCURATE CHEMICAL & | BYA-6189-1 |
| | Clone: AGP-47, Mab anti- | SCIENTIFIC CORPORATION | |
| | Human | | |
| SPI-452 | Cystatin C, Rabbit anti-Human | ACCURATE CHEMICAL & | AXL-574 |
| | | SCIENTIFIC CORPORATION | |
| SPI-453 | ANTI-CYTOKERATIN TYPE 10 | RDI RESEARCH | RDI-CBL196 |
| | | DIAGNOSTICS, INC | |
| SPI-454 | Transthyretin, Prealbuminm, | ACCURATE CHEMICAL & | MED-CLA 193 |
| | 55kD, Rabbit anti-Human | SCIENTIFIC CORPORATION | |
| SPI-457 | Apolipoprotein E, LDL, VLDL, | ACCURATE CHEMICAL & | YM-5029 |
| | Clone: 3D12, Mab anti-Human, | SCIENTIFIC CORPORATION | |
| | frozen/paraffin | | |
| SPI-461 | C3 Complement, Chicken anti- | ACCURATE CHEMICAL & | IMS-01-001-02 |
| | Human | SCIENTIFIC CORPORATION | |
| SPI-462 | C4 Complement, Chicken anti- | ACCURATE CHEMICAL & | IMS-01-032-02 |
| | Human | SCIENTIFIC CORPORATION | |
| SPI-464 | Factor H (Complement), | ACCURATE CHEMICAL & | IMS-01-066-02 |
| | Chicken anti-Human | SCIENTIFIC CORPORATION | |
| SPI-465 | Monoclonal mouse anti-human | RDI RESEARCH | RDI-TRK4P11-4D2 |
| | plasminogen | DIAGNOSTICS, INC | |
| SPI-467 | Alpha-1-Acid Glycoprotein, | ACCURATE CHEMICAL & | BYA-6189-1 |
| | Clone: AGP-47, Mab anti- | SCIENTIFIC CORPORATION | |
| | Human | | |

In one embodiment, binding of antibody in tissue sections can be used to detect aberrant SPI localization or an aberrant level of one or more SPIs. In a specific embodiment, antibody to an SPI can be used to assay a tissue sample (e.g., a brain biopsy) from a subject for the level of the SPI where an aberrant level of SPI is indicative of Schizophrenia. As used herein, an “aberrant level” means a level that is increased or decreased compared with the level in a subject free from Schizophrenia or a reference level. If desired, the comparison can be performed with a matched sample from the same subject, taken from a portion of the body not affected by Schizophrenia.

Any suitable immunoassay can be used, including, without limitation, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement- fixation assays, immunoradiometric assays, fluorescent immunoassays and protein A immunoassays.

For example, an SPI can be detected in a fluid sample (e.g., CSF, blood, urine, or tissue homogenate) by means of a two-step sandwich assay. In the first step, a capture reagent (e.g., an anti-SPI antibody) is used to capture the SPI. Examples of such antibodies known in the art are set forth in Table VII. The capture reagent can optionally be immobilized on a solid phase. In the second step, a directly or indirectly labelled detection reagent is used to detect the captured SPI. In one embodiment, the detection reagent is a lectin. Any lectin can be used for this purpose that preferentially binds to the SPI rather than to other isoforms that have the same core protein as the SPI or to other proteins that share the antigenic determinant recognized by the antibody. In a preferred embodiment, the chosen lectin binds to the SPI with at least 2-fold greater affinity, more preferably at least 5-fold greater affinity, still more preferably at least 10-fold greater affinity, than to said other isoforms that have the same core protein as the SPI or to said other proteins that share the antigenic determinant recognized by the antibody. Based on the present description, a lectin that is suitable for detecting a given SPI can readily be identified by methods well known in the art, for instance upon testing one or more lectins enumerated in Table I on pages 158-159 of Sumar et al, Lectins as Indicators of Disease-Associated Glycoforms, In: Gabius H-J & Gabius S (eds.), 1993, Lectins and Glycobiology, at pp. 158-174 (which is incorporated herein by reference in its entirety). Lectins with the desired oligosaccharide specificity can be identified, for example, by their ability to detect the SPI in a 2D gel, in a replica of a 2D gel following transfer to a suitable solid substrate such as a nitrocellulose membrane, or in a two-step assay following capture by an antibody. In an alternative embodiment, the detection reagent is an antibody, e.g., an antibody that immunospecifically detects other post-translational modifications, such as an antibody that immunospecifically binds to phosphorylated amino acids. Examples of such antibodies include those that bind to phosphotyrosine (BD Transduction Laboratories, catalog nos.: P11230-050/P11230-150; P11120; P38820; P39020), those that bind to phosphoserine (Zymed Laboratories Inc., South San Francisco, Calif., catalog no. 61-8100) and those that bind to phosphothreonine (Zymed Laboratories Inc., South San Francisco, Calif., catalog nos. 71-8200, 13-9200).

If desired, a gene encoding an SPI, a related gene, or related nucleic acid sequences or subsequences, including complementary sequences, can also be used in hybridization assays. A nucleotide encoding an SPI, or subsequences thereof comprising at least 8 nucleotides, preferably at least 12 nucleotides, and most preferably at least 15 nucleotides can be used as a hybridization probe. Hybridization assays can be used for detection, prognosis, diagnosis, or monitoring of conditions, disorders, or disease states, associated with aberrant expression of genes encoding SPIs, or for differential diagnosis of subjects with signs or symptoms suggestive of Schizophrenia. In particular, such a hybridization assay can be carried out by a method comprising contacting a subject's sample containing nucleic acid with a nucleic acid probe capable of hybridizing to a DNA or RNA that encodes an SPI, under conditions such that hybridization can occur, and detecting or measuring any resulting hybridization. Nucleotides can be used for therapy of subjects having Schizophrenia, as described below.

The methods and compositions for clinical screening, diagnosis and prognosis of Schizophrenia in a mammalian subject may be diagnostic of Schizophrenia or indicative of Schizophrenia.

Diagnostic methods and compositions are based on Schizophrenia-Associated. Features (SFs) and Schizophrenia-Associated Protein Isoforms (SPIs) which are specifically and particularly associated with Schizophrenia and are generally not associated with other diseases or conditions. Such diagnostic SFs or SPis, which are specifically associated with Schizophrenia, are useful in screening, diagnosis and prognosis as indicators of Schizophrenia. The administration of therapeutic compositions which are directed against or lead to modulation of diagnostic markers may have therapeutic value particularly in Schizophrenia.

Indicative methods and compositions are based on Schizophrenia-Associated Features (SFs) and Schizophrenia-Associated Protein Isoforms (SPIs) which are associated with Schizophrenia but may not be specific only for Schizophrenia, and may be associated with one or more other diseases or conditions. Such indicative SFs or SPis, which are associated with Schizophrenia, but not only with Schizophrenia, are useful in screening, diagnosis and prognosis as indicators of Schizophrenia. Indicative methods and compositions are particularly useful in the initial or general screening, diagnosis and prognosis of an individual subject, whereby a first indication of a subset of conditions or diseases, including Schizophrenia, is thereby provided. Additional assessment utilizing diagnostic or particular Schizophrenia SFs or SPIs may then be undertaken to provide specific, diagnostic screening, diagnosis and prognosis of the individual subject. The administration of therapeutic compositions which are directed against or lead to modulation of indicative markers may have therapeutic value in Schizophrenia and other disorders as well, or may be useful therapeutically in more than one disease or condition

Thus, a diagnostic marker changes (increases, decreases or otherwise alters form or character) significantly in only a single disease or condition or in only a small number of conditions, particularly in related conditions. One such diagnostic marker, SF-306, is provided below in Table VIII.

TABLE VIII
Example of a diagnostic marker for Schizophrenia:

| Feature # | Isoform # | Fold Change | pI | MW (Da) |
|---|---|---|---|---|
| SF-306 | SPI-399 | 2.41 | 5.72 | 100168 |

An indicative marker changes (increases, decreases or otherwise alters form or character) significantly in more than one condition, particularly in Schizophrenia and one or more other distinct diseases or conditions. One such indicative marker, SF-255, is found to increase in Schizophrenia and is provided in Table IX. This same marker, identified or characterised by the same pI and MW, is noted as DF-155 as similarly found to be increased in Bipolar Affective Disorder (BAD) and Unipolar Depression. The SF-255/DF-155 marker is therefore indicative of Schizophrenia and/or Depression.

Table IX: Example of an indicative marker for Schizophrenia:

TABLE IX
Example of an indicative marker for Schizophrenia:

| Feature # | Isoform # | Disease | Fold Change | pI | MW (Da) |
|---|---|---|---|---|---|
| SF-255 | SPI-138 | Schizophrenia | 2.25 | 7.03 | 155828 |
| DF-155 | DPI-93 | Depression | 1.92 | 7.03 | 155828 |

The invention also provides diagnostic kits, comprising an anti-SPI antibody. In addition, such a kit may optionally comprise one or more of the following: (1) instructions for using the anti-SPI antibody for diagnosis, prognosis, therapeutic monitoring or any combination of these applications; (2) a labelled binding partner to the antibody; (3) a solid phase (such as a reagent strip) upon which the anti-SPI antibody is immobilized; and (4) a label or insert indicating regulatory approval for diagnostic, prognostic or therapeutic use or any combination thereof. If no labelled binding partner to the antibody is provided, the anti-SPI antibody itself can be labelled with a detectable marker, e.g., a chemiluminescent, enzymatic, fluorescent, or radioactive moiety.

The invention also provides a kit comprising a nucleic acid probe capable of hybridizing to RNA encoding an SPI. In a specific embodiment, a kit comprises in one or more containers a pair of primers (e.g., each in the size range of 6-30 nucleotides, more preferably 10-30 nucleotides and still more preferably 10-20 nucleotides) that under appropriate reaction conditions can prime amplification of at least a portion of a nucleic acid encoding an SPI, such as by polymerase chain reaction (see, e.g., Innis et al, 1990, PCR Protocols, Academic Press, Inc., San Diego, Calif.), ligase chain reaction (see EP 320,308) use of Q replicase, cyclic probe reaction, or other methods known in the art.

Kits are also provided which allow for the detection of a plurality of SPIs or a plurality of nucleic acids each encoding an SPI. A kit can optionally further comprise a predetermined amount of an isolated SPI protein or a nucleic acid encoding an SPI, e.g., for use as a standard or control.

5.3 Statistical Techniques for Identifying SPIs and SPI Clusters

The uni-variate differential analysis tools, such as fold changes, wilcoxon rank sum test and t-test, are useful in identifying individual SFs or SPIs that are diagnostically associated with Schizophrenia or in identifying individual SPIs that regulate the disease process. In most cases, however, those skilled in the art appreciate that the disease process is associated with a combination of SFs or SPIs (and to be regulated by a combination of SPIs), rather than individual SFs and SPIs in isolation. The strategies for discovering such combinations of SFs and SPIs differ from those for discovering individual SFs and SPIs. In such cases, each individual SF and SPI can be regarded as one variable and the disease can be regarded as a joint, multi-variate effect caused by interaction of these variables.

The following steps can be used to identify markers from data produced by the Preferred Technology.

The first step is to identify a collection of SFs or SPIs that individually show significant association with Schizophrenia. The association between the identified SFs or SPIs and Schizophrenia need not be as highly significant as is desirable when an individual SF or SPI is used as a diagnostic. Any of the tests discussed above (fold changes, wilcoxon rank sum test, etc.) can be used at this stage. Once a suitable collection of SFs or SPIs has been identified, a sophisticated multi-variate analysis capable of identifying clusters can then be used to estimate the significant multivariate associations with Schizophrenia.

Linear Discriminant Analysis (LDA) is one such procedure, which can be used to detect significant association between a cluster of variables (i.e., SFs or SPIs) and Schizophrenia. In performing LDA, a set of weights is associated with each variable (i.e., SF or SPI) so that the linear combination of weights and the measured values of the variables can identify the disease state by discriminating between subjects having Schizophrenia and subjects free from Schizophrenia. Enhancements to the LDA allow stepwise inclusion (or removal) of variables to optimize the discriminant power of the model. The result of the LDA is therefore a cluster of SFs or SPIs which can be used, without limitation, for diagnosis, prognosis, therapy or drug development. Other enhanced variations of LDA, such as Flexible Discriminant Analysis permit the use of non-linear combinations of variables to discriminate a disease state from a normal state. The results of the discriminant analysis can be verified by post-hoc tests and also by repeating the analysis using alternative techniques such as classification trees.

A further category of SFs or SPIs can be identified by qualitative measures by comparing the percentage feature presence of an SF or SPI of one group of samples (e.g., samples from diseased subjects) with the percentage feature presence of an SF or SPI in another group of samples (e.g., samples from control subjects). The “percentage feature presence” of an SF or SPI is the percentage of samples in a group of samples in which the SF or SPI is detectable by the detection method of choice. For example, if an SF is detectable in 95 percent of samples from diseased subjects, the percentage feature presence of that SF in that sample group is 95 percent. If only 5 percent of samples from non-diseased subjects have detectable levels of the same SF, detection of that SF in the sample of a subject would suggest that it is likely that the subject suffers from Schizophrenia.

5.4 Use in Clinical Studies

The diagnostic methods and compositions of the present invention can assist in monitoring a clinical study, e.g. to evaluate drugs for therapy of Schizophrenia. In one embodiment, candidate molecules are tested for their ability to restore SF or SPI levels in a subject having Schizophrenia to levels found in subjects free from Schizophrenia or, in a treated subject (e.g. after treatment with Haloperidol, Pirenzepine, Perazine, Risperdal, Famotidine, Zyperexa, Clozaril, Mesoridazine, Quetiapine, atypical anti-psychotic medications of Risperidone, Olanzapine and Clozapine and any other Dibenzothiazepines), to preserve SF or SPI levels at or near non-Schizophrenia values. The levels of one or more SFs or SPIs can be assayed.

In another embodiment, the methods and compositions of the present invention are used to screen candidates for a clinical study to identify individuals having Schizophrenia; such individuals can then be either excluded from or included in the study or can be placed in a separate cohort for treatment or analysis. If desired, the candidates can concurrently be screened to identify individuals with Schizophrenia; procedures for these screens are well known in the art.

5.5 Purification of SPIs

In particular aspects, the invention provides isolated mammalian SPIs, preferably human SPIs, and fragments thereof which comprise an antigenic determinant (i.e., can be recognized by an antibody) or which are otherwise functionally active, as well as nucleic acid sequences encoding the foregoing. “Functionally active” as used herein refers to material displaying one or more functional activities associated with a full-length (wild-type) SPI, e.g., binding to an SPI substrate or SPI binding partner, antigenicity (binding to an anti-SPI antibody), immunogenicity, enzymatic activity and the like.

In specific embodiments, the invention provides fragments of an SPI comprising at least 5 amino acids, at least 10 amino acids, at least 50 amino acids, or at least 75 amino acids. Fragments lacking some or all of the regions of an SPI are also provided, as are proteins (e.g., fusion proteins) comprising such fragments. Nucleic acids encoding the foregoing are provided.

Once a recombinant nucleic acid which encodes the SPI, a portion of the SPI, or a precursor of the SPI is identified, the gene product can be analyzed. This is achieved by assays based on the physical or functional properties of the product, including radioactive labeling of the product followed by analysis by gel electrophoresis, immunoassay, etc.

The SPIs identified herein can be isolated and purified by standard methods including chromatography (e.g., ion exchange, affinity, and sizing column chromatography), centrifugation, differential solubility, or by any other standard technique for the purification of proteins.

Alternatively, once a recombinant nucleic acid that encodes the SPI is identified, the entire amino acid sequence of the SPI can be deduced from the nucleotide sequence of the gene coding region contained in the recombinant nucleic acid. As a result, the protein can be synthesized by standard chemical methods known in the art (e.g., see Hunkapiller et al, 1984, Nature 310:105-111).

In another alternative embodiment, native SPIs can be purified from natural sources, by standard methods such as those described above (e.g., immunoaffinity purification).

In a preferred embodiment, SPIs are isolated by the Preferred Technology described supra. For preparative-scale runs, a narrow-range “zoom gel” having a pH range of 2 pH units or less is preferred for the isoelectric step, according to the method described in Westermeier, 1993, Electrophoresis in Practice (VCH, Weinheim, Germany), pp. 197-209 (which is incorporated herein by reference in its entirety); this modification permits a larger quantity of a target protein to be loaded onto the gel, and thereby increases the quantity of isolated SPI that can be recovered from the gel. When used in this way for preparative-scale runs, the Preferred Technology typically provides up to 100 ng, and can provide up to 1000 ng, of an isolated SPI in a single run. Those of skill in the art will appreciate that a zoom gel can be used in any separation strategy that employs gel isoelectric focusing.

The invention thus provides an isolated SPI, an isolated SPI-related polypeptide, and an isolated derivative or fragment of an SPI or an SPI-related polypeptide; any of the foregoing can be produced by recombinant DNA techniques or by chemical synthetic methods.

5.6 Isolation of DNA Encoding an SPI

Specific embodiments for the cloning of a gene encoding an SPI, are presented below by way of example and not of limitation.

The nucleotide sequences of the present invention, including DNA and RNA, and comprising a sequence encoding an SPI or a fragment thereof, or an SPI-related polypeptide, may be synthesized using methods known in the art, such as using conventional chemical approaches or polymerase chain reaction (PCR) amplification. The nucleotide sequences of the present invention also permit the identification and cloning of the gene encoding an SPI homolog or SPI ortholog including, for example, by screening cDNA libraries, genomic libraries or expression libraries.

For example, to clone a gene encoding an SPI by PCR techniques, anchored degenerate oligonucleotides (or a set of most likely oligonucleotides) can be designed for all SPI peptide fragments identified as part of the same protein. PCR reactions under a variety of conditions can be performed with relevant cDNA and genomic DNAs (e.g., from brain tissue or from cells of the immune system) from one or more species. Also vectorette reactions can be performed on any available cDNA and genomic DNA using the oligonucleotides (which preferably are nested) as above. Vectorette PCR is a method that enables the amplification of specific DNA fragments in situations where the sequence of only one primer is known. Thus, it extends the application of PCR to stretches of DNA where the sequence information is only available at one end. (Arnold C, PCR Methods Appl (1991) l(1):39-42; Dyer K. D, Biotechniques, (1995) 19(4):550-2). Vectorette PCR may pe performed with probes that are, for example, anchored degenerate oligonucleotides (or most likely oligonucleotides) coding for SPI peptide fragments, using as a template a genomic library or cDNA library pools.

Anchored degenerate oligonucleotides (and most likely oligonucleotides) can be designed for all SPI peptide fragments. These oligonucleotides may be labelled and hybridized to filters containing cDNA and genomic DNA libraries. Oligonucleotides to different peptides from the same protein will often identify the same members of the library. The cDNA and genomic DNA libraries may be obtained from any suitable or desired mammalian species, for example from humans.

Nucleotide sequences comprising a nucleotide sequence encoding an SPI or SPI fragment of the present invention are useful for their ability to hybridize selectively with complementary stretches of genes encoding other proteins. Depending on the application, a variety of hybridization conditions may be employed to obtain nucleotide sequences at least 30%, 35%, 40%, 45%, 50%, 55%, 60%, 65%, 70%, 75%, 80%, 85%, 90%, 95%, or 99% identical, or 100% identical, to the sequence of a nucleotide encoding an SPI.

For a high degree of selectivity, relatively stringent conditions are used to form the duplexes, such as low salt or high temperature conditions. As used herein, “highly stringent conditions” means hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F. M. et al, eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2×SSC/0.1% SDS at 42° C. (Ausubel et al, 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding an SPI, 37° C. for 90 to 95% identity and 32° C. for 70 to 90% identity.

In the preparation of genomic libraries, DNA fragments are generated, some of which will encode parts or the whole of an SPI. Any suitable method for preparing DNA fragments may be used in the present invention. For example, the DNA may be cleaved at specific sites using various restriction enzymes. Alternatively, one may use DNAse in the presence of manganese to fragment the DNA, or the DNA can be physically sheared, as for example, by sonication. The DNA fragments can then be separated according to size by standard techniques, including but not limited to agarose and polyacrylamide gel electrophoresis, column chromatography and sucrose gradient centrifugation. The DNA fragments can then be inserted into suitable vectors, including but not limited to plasmids, cosmids, bacteriophages lambda or T4, and yeast artificial chromosome (YAC). (See, e.g., Sambrook et al, 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II; Ausubel F. M. et al, eds., 1989, Current Protocols in Molecular Biology, Vol. I, Green Publishing Associates, Inc., and John Wiley & sons, Inc., New York). The genomic library may be screened by nucleic acid hybridization to labelled probe (Benton and Davis, Science (1977) 196:180; Grunstein and Hogness, Proc. Nat. Acad. Sci. USA (1975) 72:3961).

Based on the present description, the genomic libraries may be screened with labelled degenerate oligonucleotide probes corresponding to the amino acid sequence of any peptide of the SPI using optimal approaches well known in the art. Any probe used is at least 10 nucleotides, at least 15 nucleotides, at least 20 nucleotides, at least 25 nucleotides, at least 30 nucleotides, at least 40 nucleotides, at least 50 nucleotides, at least 60 nucleotides, at least 70 nucleotides, at least 80 nucleotides, or at least 100 nucleotides. Preferably a probe is 10 nucleotides or longer, and more preferably 15 nucleotides or longer.

In Tables IV and V above, some SPIs disclosed herein were found to correspond to isoforms of previously identified proteins encoded by genes whose sequences are publicly known. (Sequence analysis and protein identification of SPIs was carried out using the methods described in Section 6.1.14). To screen such a gene, any probe may be used that is complementary to the gene or its complement; preferably the probe is 10 nucleotides or longer, more preferably 15 nucleotides or longer. The SWISS-PROT and trEMBL databases (held by the Swiss Institute of Bioinformatics (SIB) and the European Bioinformatics Institute (EBI) which are available at http://www.expasy.ch/) and the GenBank database (held by the National Institute of Health (NIH) which is available at http://www.ncbi.nlm.nih.gov/GenBank/) provide protein sequences for the SPIs listed in Tables IV and V under the following accession numbers and each sequence is incorporated herein by reference:

TABLE X
Nucleotide sequences encoding SPIs,
SPI Related Proteins or ERPIs

| | | Accession Numbers of |
|---|---|---|
| SF# | SPI# | Identified Sequences |
| SF-14 | SPI-6 | P10643 |
| SF-16 | SPI-231 | P05090 |
| SF-19 | SPI-312 | P41222 |
| SF-20 | SPI-352 | 6049608 (gb) |
| SF-21 | SPI-232 | P48668 |
| SF-22 | SPI-7 | P41222 |
| SF-24 | SPI-353 | P01034 |
| SF-24 | SPI-354 | P20472 |
| SF-27 | SPI-233 | P01034 |
| SF-28 | SPI-8 | P01034 |
| SF-29 | SPI-9 | P01034 |
| SF-30 | SPI-10 | P05067 |
| SF-31 | SPI-11 | Q99435 |
| SF-32 | SPI-13 | P36955 |
| SF-32 | SPI-234 | P02679 |
| SF-33 | SPI-355 | P05067 |
| SF-33 | SPI-356 | P05155 |
| SF-35 | SPI-15 | P02649 |
| SF-35 | SPI-16 | P10909 |
| SF-36 | SPI-17 | P41222 |
| SF-37 | SPI-18 | P06396 |
| SF-38 | SPI-19 | P36955 |
| SF-38 | SPI-235 | P35747 |
| SF-38 | SPI-236 | 4240271 (gb) |
| SF-39 | SPI-357 | P01028 |
| SF-40 | SPI-20 | Q92876 |
| SF-41 | SPI-21 | P36955 |
| SF-42 | SPI-23 | P06396 |
| SF-43 | SPI-26 | 5802984 (gb) |
| SF-43 | SPI-24 | P36955 |
| SF-43 | SPI-25 | P04469 |
| SF-44 | SPI-28 | P41222 |
| SF-45 | SPI-29 | P36955 |
| SF-45 | SPI-30 | 5802984 (gb) |
| SF-46 | SPI-32 | P01024 |
| SF-47 | SPI-33 | P01024 |
| SF-48 | SPI-34 | P10909 |
| SF-48 | SPI-35 | P02649 |
| SF-49 | SPI-36 | P41222 |
| SF-51 | SPI-38 | P41222 |
| SF-52 | SPI-39 | 5802984 (gb) |
| SF-53 | SPI-237 | P05067 |
| SF-55 | SPI-238 | NOVEL |
| SF-55 | SPI-239 | P80108 |
| SF-55 | SPI-41 | P01019 |
| SF-55 | SPI-240 | NOVEL |
| SF-56 | SPI-42 | P01008 |
| SF-57 | SPI-43 | P05452 |
| SF-58 | SPI-241 | P41222 |
| SF-58 | SPI-44 | P30086 |
| SF-81 | SPI-321 | P01028 |
| SF-81 | SPI-322 | P41222 |
| SF-82 | SPI-323 | P01028 |
| SF-83 | SPI-54 | P02671 |
| SF-84 | SPI-381 | P02649 |
| SF-85 | SPI-382 | P41222 |
| SF-86 | SPI-56 | P35908 |
| SF-87 | SPI-383 | P41222 |
| SF-87 | SPI-384 | P36955 |
| SF-88 | SPI-57 | P01028 |
| SF-90 | SPI-324 | P01027 |
| SF-91 | SPI-325 | P02649 |
| SF-92 | SPI-326 | P13645 |
| SF-93 | SPI-359 | P02024 |
| SF-93 | SPI-360 | 1095700.4 (gb) |
| SF-94 | SPI-58 | P05155 |
| SF-96 | SPI-361 | P02790 |
| SF-97 | SPI-327 | P01024 |
| SF-98 | SPI-362 | P02768 |
| SF-99 | SPI-328 | P00751 |
| SF-100 | SPI-242 | P02649 |
| SF-101 | SPI-329 | P01024 |
| SF-102 | SPI-59 | P36955 |
| SF-102 | SPI-60 | P02649 |
| SF-107 | SPI-243 | 106655 (gb) |
| SF-107 | SPI-330 | 106655 (gb) |
| SF-108 | SPI-244 | P01028 |
| SF-111 | SPI-62 | P01034 |
| SF-112 | SPI-331 | P41222 |
| SF-114 | SPI-332 | P36955 |
| SF-115 | SPI-63 | P02763 |
| SF-116 | SPI-65 | P01877 |
| SF-117 | SPI-333 | 1361979 (gb) |
| SF-118 | SPI-334 | P02768 |
| SF-123 | SPI-335 | 662290 (gb) |
| SF-124 | SPI-385 | 2647262 (gb) |
| SF-126 | SPI-245 | P02538 |
| SF-132 | SPI-336 | P14625 |
| SF-135 | SPI-67 | P01028 |
| SF-143 | SPI-337 | P06732 |
| SF-144 | SPI-363 | P02023 |
| SF-151 | SPI-69 | P36955 |
| SF-153 | SPI-338 | Q15668 |
| SF-153 | SPI-339 | P01024 |
| SF-154 | SPI-365 | P02023 |
| SF-157 | SPI-340 | P41222 |
| SF-158 | SPI-387 | P41222 |
| SF-158 | SPI-388 | 9368450 (gb) |
| SF-159 | SPI-73 | P02649 |
| SF-160 | SPI-74 | P01028 |
| SF-161 | SPI-75 | P02649 |
| SF-163 | SPI-76 | P02649 |
| SF-164 | SPI-77 | P01028 |
| SF-165 | SPI-389 | P36222 |
| SF-166 | SPI-246 | P10909 |
| SF-167 | SPI-78 | P02649 |
| SF-168 | SPI-390 | P36955 |
| SF-169 | SPI-391 | Q13827 |
| SF-170 | SPI-80 | P02649 |
| SF-171 | SPI-392 | P41222 |
| SF-172 | SPI-393 | Q13827 |
| SF-173 | SPI-247 | 401767 (gb) |
| SF-173 | SPI-81 | P15169 |
| SF-174 | SPI-82 | P02766 |
| SF-176 | SPI-83 | P36955 |
| SF-176 | SPI-248 | 7435109 (gb) |
| SF-176 | SPI-249 | P05156 |
| SF-177 | SPI-85 | P02750 |
| SF-178 | SPI-250 | P01027 |
| SF-179 | SPI-87 | P36955 |
| SF-180 | SPI-251 | 899271 (gb) |
| SF-181 | SPI-88 | P04220 |
| SF-182 | SPI-252 | 1942472 (gb) |
| SF-184 | SPI-253 | 1402890 (gb) |
| SF-186 | SPI-254 | P01028 |
| SF-187 | SPI-255 | P01028 |
| SF-188 | SPI-394 | P35908 |
| SF-189 | SPI-91 | P02790 |
| SF-190 | SPI-257 | P23144 |
| SF-190 | SPI-258 | P08697 |
| SF-191 | SPI-92 | P01024 |
| SF-191 | SPI-259 | P02749 |
| SF-194 | SPI-261 | P01024 |
| SF-196 | SPI-262 | P08603 |
| SF-197 | SPI-95 | P41222 |
| SF-197 | SPI-93 | 1942471 (gb) |
| SF-198 | SPI-96 | P06396 |
| SF-199 | SPI-97 | P10643 |
| SF-200 | SPI-99 | P13671 |
| SF-201 | SPI-100 | P29622 |
| SF-202 | SPI-101 | P06866 |
| SF-209 | SPI-105 | P01344 |
| SF-211 | SPI-367 | Q92876 |
| SF-212 | SPI-263 | P08603 |
| SF-213 | SPI-264 | P10909 |
| SF-213 | SPI-107 | P01028 |
| SF-215 | SPI-341 | P08603 |
| SF-217 | SPI-113 | 1942472 (gb) |
| SF-219 | SPI-114 | P16035 |
| SF-221 | SPI-342 | P06396 |
| SF-222 | SPI-115 | P10643 |
| SF-222 | SPI-265 | P02768 |
| SF-223 | SPI-118 | 015818 |
| SF-226 | SPI-266 | P51693 |
| SF-226 | SPI-267 | P08571 |
| SF-227 | SPI-268 | 4758978 (gb) |
| SF-227 | SPI-269 | P29622 |
| SF-228 | SPI-270 | P01027 |
| SF-229 | SPI-122 | P01024 |
| SF-230 | SPI-123 | 7019363 (gb) |
| SF-231 | SPI-124 | P07358 |
| SF-232 | SPI-343 | P01024 |
| SF-233 | SPI-344 | 2745741 (gb) |
| SF-233 | SPI-345 | P01876 |
| SF-235 | SPI-346 | P02790 |
| SF-239 | SPI-127 | P00747 |
| SF-242 | SPI-129 | P04264 |
| SF-243 | SPI-130 | P02675 |
| SF-243 | SPI-273 | P05154 |
| SF-244 | SPI-369 | P02023 |
| SF-248 | SPI-370 | P01034 |
| SF-249 | SPI-274 | P08603 |
| SF-250 | SPI-133 | P28340 |
| SF-250 | SPI-132 | P02649 |
| SF-255 | SPI-138 | Q02246 |
| SF-257 | SPI-275 | P01876 |
| SF-258 | SPI-139 | P08571 |
| SF-262 | SPI-397 | P06396 |
| SF-264 | SPI-141 | Q12860 |
| SF-265 | SPI-142 | Q02246 |
| SF-267 | SPI-143 | P02571 |
| SF-268 | SPI-151 | Q02246 |
| SF-269 | SPI-152 | P02790 |
| SF-270 | SPI-153 | P01028 |
| SF-271 | SPI-154 | P02766 |
| SF-272 | SPI-155 | P08697 |
| SF-273 | SPI-348 | P10643 |
| SF-280 | SPI-164 | P01028 |
| SF-282 | SPI-166 | Q14112 |
| SF-283 | SPI-167 | P01876 |
| SF-286 | SPI-169 | P01043 |
| SF-286 | SPI-170 | P01876 |
| SF-289 | SPI-398 | P08603 |
| SF-291 | SPI-176 | P36955 |
| SF-291 | SPI-175 | P01028 |
| SF-292 | SPI-349 | P06733 |
| SF-293 | SPI-372 | P04406 |
| SF-296 | SPI-278 | P08603 |
| SF-300 | SPI-179 | Q02246 |
| SF-300 | SPI-281 | 6753222 (gb) |
| SF-301 | SPI-375 | P01024 |
| SF-302 | SPI-376 | P01024 |
| SF-303 | SPI-181 | P17174 |
| SF-304 | SPI-182 | Q15582 |
| SF-306 | SPI-399 | O15394 |
| SF-307 | SPI-183 | P02790 |
| SF-309 | SPI-185 | P02790 |
| SF-309 | SPI-184 | P04217 |
| SF-317 | SPI-400 | P19021 |
| SF-320 | SPI-189 | P41222 |
| SF-321 | SPI-379 | P00751 |
| SF-322 | SPI-190 | Q03591 |
| SF-324 | SPI-193 | P06727 |
| SF-326 | SPI-285 | P04217 |
| SF-327 | SPI-195 | 7341255 (gb) |
| SF-332 | SPI-289 | P01028 |
| SF-333 | SPI-200 | P02750 |
| SF-336 | SPI-290 | P00747 |
| SF-340 | SPI-205 | P01028 |
| SF-342 | SPI-206 | NOVEL (AL008583) |
| SF-344 | SPI-296 | P17900 |
| SF-344 | SPI-297 | P41222 |
| SF-348 | SPI-211 | P02790 |
| SF-348 | SPI-302 | 6518913 (gb) |
| SF-349 | SPI-303 | P05201 |
| SF-352 | SPI-213 | P30086 |
| SF-352 | SPI-214 | P41222 |
| SF-354 | SPI-306 | P40252 |
| SF-368 | SPI-401 | P10643 |
| SF-368 | SPI-402 | 4507721 (gb) |
| SF-369 | SPI-403 | P01023 |
| SF-370 | SPI-404 | P01024 |
| SF-372 | SPI-405 | P01034 |
| SF-373 | SPI-406 | 1743885 (gb) |
| SF-373 | SPI-407 | P06396 |
| SF-376 | SPI-408 | P13645 |
| SF-376 | SPI-409 | P18065 |
| SF-379 | SPI-410 | P02649 |
| SF-380 | SPI-411 | P01034 |
| SF-382 | SPI-412 | P01024 |
| SF-389 | SPI-413 | P02647 |
| SF-391 | SPI-414 | P05090 |
| SF-393 | SPI-415 | P41222 |
| SF-396 | SPI-416 | P02649 |
| SF-396 | SPI-417 | P41222 |
| SF-397 | SPI-418 | P01034 |
| SF-398 | SPI-419 | P41222 |
| SF-399 | SPI-420 | P01034 |
| SF-402 | SPI-421 | P02766 |
| SF-404 | SPI-422 | P05090 |
| SF-405 | SPI-423 | P01876 |
| SF-406 | SPI-424 | P01024 |
| SF-406 | SPI-425 | P10909 |
| SF-407 | SPI-426 | P01034 |
| SF-409 | SPI-427 | P04264 |
| SF-409 | SPI-428 | 7662374 |
| SF-410 | SPI-429 | P08697 |
| SF-410 | SPI-430 | P02748 |
| SF-410 | SPI-431 | P01876 |
| SF-411 | SPI-432 | P07360 |
| SF-412 | SPI-433 | P10909 |
| SF-412 | SPI-434 | 4240149 (gb) |
| SF-414 | SPI-435 | P08697 |
| SF-416 | SPI-436 | P02774 |
| SF-416 | SPI-437 | 436857.2 |
| SF-416 | SPI-438 | P01019 |
| SF-416 | SPI-439 | 177836 (gb) |
| SF-417 | SPI-440 | 410564 |
| SF-420 | SPI-441 | P05090 |
| SF-421 | SPI-442 | P54289 |
| SF-422 | SPI-443 | P01019 |
| SF-423 | SPI-444 | 6651381 |
| SF-424 | SPI-445 | P08603 |
| SF-425 | SPI-446 | P01034 |
| SF-434 | SPI-447 | AK026519.1 |
| SF-440 | SPI-448 | P02671 |
| SF-443 | SPI-449 | P02763 |
| SF-446 | SPI-450 | 2745741 |
| SF-448 | SPI-451 | 8918224 |
| SF-451 | SPI-452 | P01034 |
| SF-459 | SPI-453 | P13645 |
| SF-462 | SPI-454 | P02766 |
| SF-462 | SPI-455 | 237026.3 |
| SF-464 | SPI-456 | P41222 |
| SF-471 | SPI-457 | P02649 |
| SF-472 | SPI-458 | 10835792 |
| SF-472 | SPI-459 | P41222 |
| SF-475 | SPI-460 | P04104 |
| SF-477 | SPI-461 | P01024 |
| SF-478 | SPI-462 | P01028 |
| SF-487 | SPI-463 | 1096891 (gb) |
| SF-494 | SPI-464 | Q03591 |
| SF-496 | SPI-465 | P00747 |
| SF-496 | SPI-466 | 1160616 |
| SF-502 | SPI-467 | P02763 |
| ERF-2 | ERPI-1 | P41222 |
| ERF-2 | ERPI-2 | P06396 |

For each of the following SPIs: SPI-206, SPI-238 and SPI-240, the partial sequence information derived from tandem mass spectrometry was not found to be described as a transcribed protein in any known public database. SPI-206, SPI-238 and SPI-240 are therefore listed as ‘NOVEL’ in Table X. SPI-206, SPI-238 and SPI-240 have been cloned, and are further described below. For any SPI, degenerate probes, or probes taken from the sequences described above by accession number may be used for screening. In the case of degenerate probes, they can be constructed from the partial amino sequence information obtained from tandem mass spectra of tryptic digest peptides of the SPI. To screen such a gene, any probe may be used that is complementary to the gene or its complement; preferably the probe is 10 nucleotides or longer, more preferably 15 nucleotides or longer. When a library is screened, clones with insert DNA encoding the SPI or a fragment thereof will hybridize to one or more members of the corresponding set of degenerate oligonucleotide probes (or their complement). Hybridization of such oligonucleotide probes to genomic libraries is carried out using methods known in the art. For example, hybridization with one of the above-mentioned degenerate sets of oligonucleotide probes, or their complement (or with any member of such a set, or its complement) can be performed under highly stringent or moderately stringent conditions as defined above, or can be carried out in 2×SSC, 1.0% SDS at 50° C. and washed using the washing conditions described supra for highly stringent or moderately stringent hybridization.

In yet another aspect of the invention, clones containing nucleotide sequences encoding the entire SPI, a fragment of an SPI, an SPI-related polypeptide, or a fragment of an SPI-related polypeptide any of the foregoing may also be obtained by screening expression libraries. For example, DNA from the relevant source is isolated and random fragments are prepared and ligated into an expression vector (e.g., a bacteriophage, plasmid, phagemid or cosmid) such that the inserted sequence in the vector is capable of being expressed by the host cell into which the vector is then introduced. Various screening assays can then be used to select for the expressed SPI or SPI-related polypeptides. In one embodiment, the various anti-SPI antibodies of the invention can be used to identify the desired clones using methods known in the art. See, for example, Harlow and Lane, 1988 Antibodies: A Laboratory Manual, Cold Spring Harbor Laboratory Press, Cold Spring Harbor, N.Y., Appendix IV. Colonies or plaques from the library are brought into contact with the antibodies to identify those clones that bind antibody.

In an embodiment, colonies or plaques containing DNA that encodes an SPI, a fragment of an SPI, an SPI-related polypeptide, or a fragment of an SPI-related polypeptide can be detected using DYNA Beads according to Olsvick et al, 29th ICAAC, Houston, Tex. 1989, incorporated herein by reference. Anti-SPI antibodies are crosslinked to tosylated DYNA Beads M280, and these antibody-containing beads are then contacted with colonies or plaques expressing recombinant polypeptides. Colonies or plaques expressing an SPI or SPI-related polypeptides are identified as any of those that bind the beads.

Alternatively, the anti-SPI antibodies can be nonspecifically immobilized to a suitable support, such as silica or Celite® resin. This material is then used to adsorb to bacterial colonies expressing the SPI protein or SPI-related polypeptide as described herein.

In another aspect, PCR amplification may be used to isolate from genomic DNA a substanially pure DNA (i.e., a DNA substanially free of contaminating nucleic acids) encoding the entire SPI or a part thereof. Preferably such a DNA is at least 95% pure, more preferably at least 99% pure. Oligonucleotide sequences, degenerate or otherwise, that correspond to peptide sequences of SPIs disclosed herein can be used as printers.

PCR can be carried out, e.g., by use of a Perkin-Elmer Cetus thermal cycler and Taq polymerase (Gene Amp® or AmpliTaq DNA polymerase). One can choose to synthesize several different degenerate primers, for use in the PCR reactions. It is also possible to vary the stringency of hybridization conditions used in priming the PCR reactions, to allow for greater or lesser degrees of nucleotide sequence similarity between the degenerate primers and the corresponding sequences in the DNA. After successful amplification of a segment of the sequence encoding an SPI, that segment may be molecularly cloned and sequenced, and utilized as a probe to isolate a complete genomic clone. This, in turn, will permit the determination of the gene's complete nucleotide sequence, the analysis of its expression, and the production of its protein product for functional analysis, as described infra.

The gene encoding an SPI can also be identified by mRNA selection by nucleic acid hybridization followed by in vitro translation. In this procedure, fragments are used to isolate complementary mRNAs by hybridization. Such DNA fragments may represent available, purified DNA encoding an SPI of another species (e.g., mouse, human). Immunoprecipitation analysis or functional assays (e.g., aggregation ability in vitro; binding to receptor) of the in vitro translation products of the isolated products of the isolated mRNAs identifies the mRNA and, therefore, the complementary DNA fragments that contain the desired sequences. In addition, specific mRNAs may be selected by adsorption of polysomes isolated from cells to immobilized antibodies that specifically recognize an SPI. A radiolabelled cDNA encoding an SPI can be synthesized using the selected mRNA (from the adsorbed polysomes) as a template. The radiolabelled mRNA or cDNA may then be used as a probe to identify the DNA fragments encoding an SPI from among other genomic DNA fragments.

Alternatives to isolating genomic DNA encoding an SPI include, but are not limited to, chemically synthesizing the gene sequence itself from a known sequence or making cDNA to the mRNA that encodes the SPI. For example, RNA for cDNA cloning of the gene encoding an SPI can be isolated from cells that express the SPI. Those skilled in the art will understand from the present description that other methods may be used and are within the scope of the invention.

Any suitable eukaryotic cell can serve as the nucleic acid source for the molecular cloning of the gene encoding an SPI. The nucleic acid sequences encoding the SPI can be isolated from vertebrate, mammalian, primate, human, porcine, bovine, feline, avian, equine, canine or murine sources. The DNA may be obtained by standard procedures known in the art from cloned DNA (e.g., a DNA “library”), by chemical synthesis, by cDNA cloning, or by the cloning of genomic DNA, or fragments thereof, purified from the desired cell. (See, e.g., Sambrook et al, 1989, Molecular Cloning, A Laboratory Manual, 2d Ed., Cold Spring Harbor Laboratory Press, Cold Spring Harbor, N.Y.; Glover, D. M. (ed.), 1985, DNA Cloning: A Practical Approach, MRL Press, Ltd., Oxford, U.K. Vol. I, II.) Clones derived from genomic DNA may contain regulatory and intron DNA regions in addition to coding regions; clones derived from cDNA will contain only exon sequences.

The identified and isolated gene or cDNA can then be inserted into any suitable cloning vector. A large number of vector-host systems known in the art may be used. As those skilled in the art will appreciate, the only limitation is that the vector system chosen be compatible with the host cell used. Such vectors include, but are not limited to, bacteriophages such as lambda derivatives, plasmids such as PBR322 or pUC plasmid derivatives or the Bluescript vector (Stratagene) or modified viruses such as adenoviruses, adeno-associated viruses or retroviruses. The insertion into a cloning vector can be accomplished, for example, by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. In an alternative method, the cleaved vector and the gene encoding an SPI may be modified by homopolymeric tailing. Recombinant molecules can be introduced into host cells via transformation, transfection, infection, electroporation, etc., so that many copies of the gene sequence are generated.

In specific embodiments, transformation of host cells with recombinant DNA molecules that incorporate the isolated gene encoding the SPI, cDNA, or synthesized DNA sequence enables generation of multiple copies of the gene. Thus, the gene may be obtained in large quantities by growing transformants, isolating the recombinant DNA molecules from the transformants and, when necessary, retrieving the inserted gene from the isolated recombinant DNA.

The nucleotide sequences of the present invention include nucleotide sequences encoding amino acid sequences with substantially the same amino acid sequences as native SPIs, nucleotide sequences encoding amino acid sequences with functionally equivalent amino acids, nucleotide sequences encoding SPIs, a fragments of SPIs, SPI-related polypeptides, or fragments of SPI-related polypeptides.

In a specific embodiment, an isolated nucleic acid molecule encoding an SPI-related polypeptide can be created by introducing one or more nucleotide substitutions, additions or deletions into the nucleotide sequence of an SPI such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

5.6.1 Cloning and Characterization of SPI-206

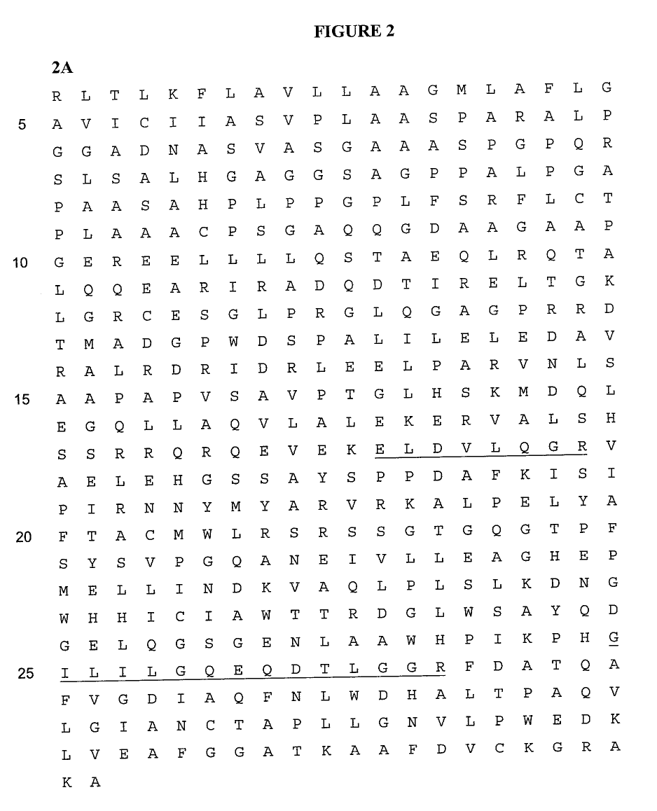

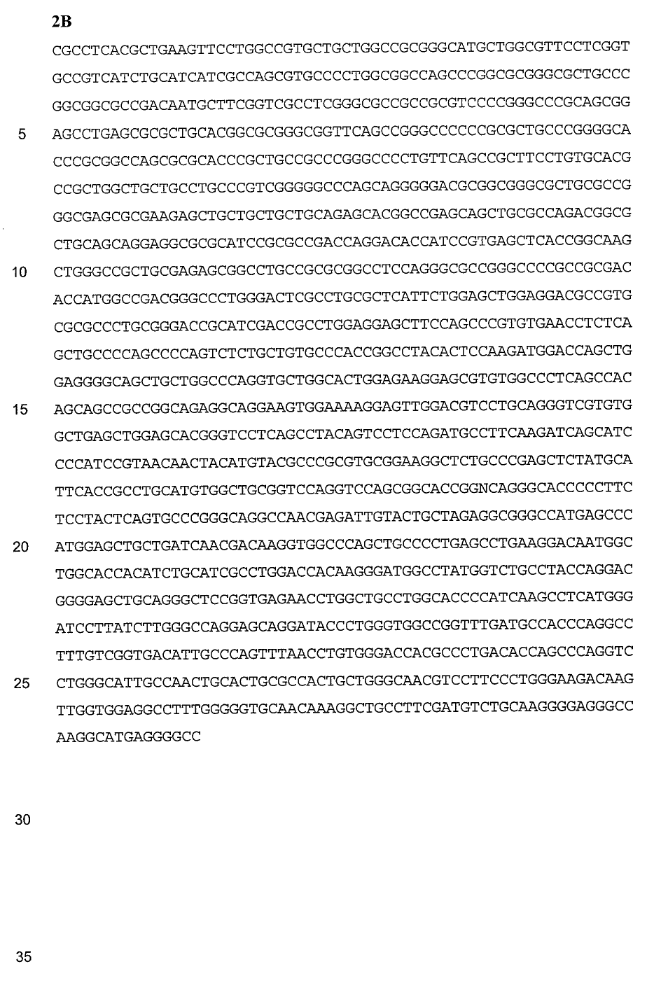

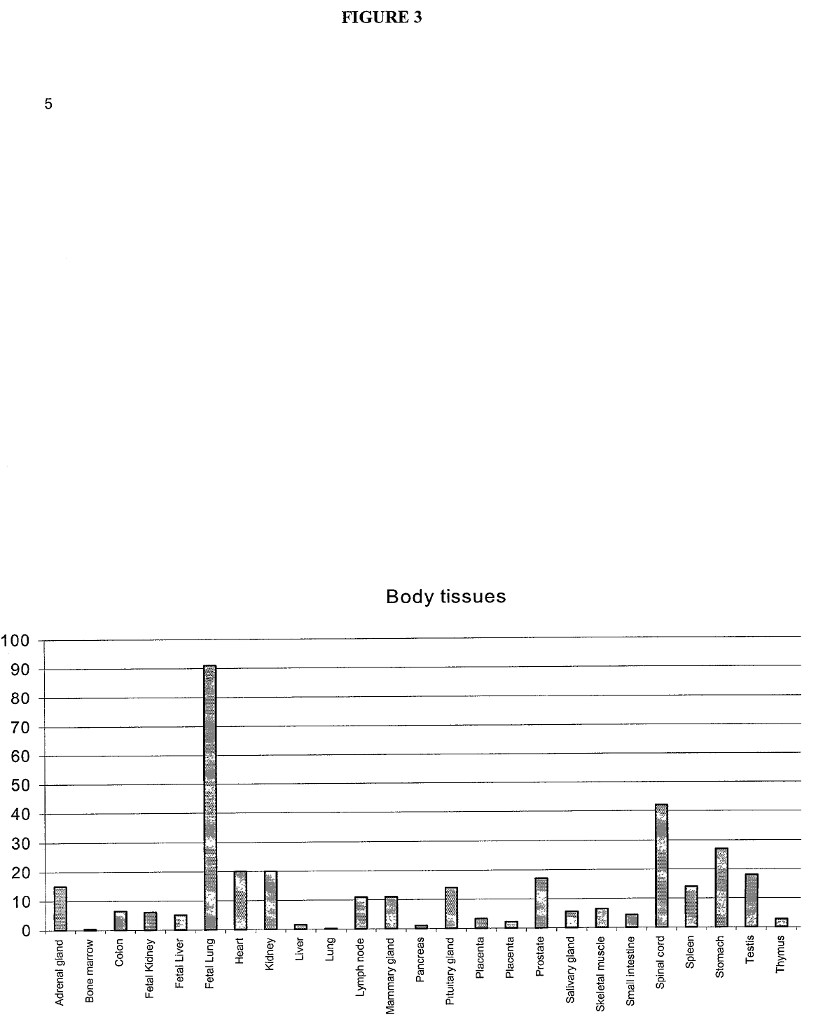

SPI-206 was isolated, subjected to proteolysis, and analyzed by mass spectrometry using the methods and apparatus of the Preferred Technology. Using the SEQUEST search program as described in the Examples, infra, uninterpreted tandem mass spectra of tryptic digest peptides were searched against a database of public domain proteins constructed of protein entries in the non-redundant database held by the National Centre for Biotechnology Information (NCBI) which is accessible at http://www.ncbi.nlm.nih.gov/. As a result of database searching, the following amino acid sequence of a tryptic digest peptide of SPI-206 was determined from a match to a tryptic digest peptide found in a translation of a human DNA sequence (protein ID CAA15430.1, accessible at http://www.ncbi.nlm.nih.gov/entrez/): ELDVLQGR (shown in FIG. 2).

In cases where no amino acid sequences could be determined through searching using the SEQUEST program, tandem mass spectra of the peptides were interpreted manually, using methods known in the art as described in the Examples, infra. In the method of tandem mass spectrometry used for sequencing peptides in the present invention, the following pairs of amino acids could not be distinguished from each other: leucine and isoleucine; and, under certain circumstances, glutamine and lysine, and phenylalanine and oxidized methionine. As used herein, an amino acid sequence “as determined by mass spectrometry” refers to the set of amino acid sequences containing at the indicated positions, one or other member of the designated pairs of amino acids. For example, the amino acid sequence P[L/I]A indicates the amino acid sequences PLA and PIA. As will be obvious to one of skill in the art, a sequence containing n designated pairs indicates 2ⁿ amino acid sequences. In Table XI, the possible amino acid sequence is listed for a single sequence determined by mass spectrometry.

TABLE XI
Partial Amino Acid Sequences of SPI-206

| | | Mass of peptide | | | | | |
|---|---|---|---|---|---|---|---|
| | | analyzed by mass | Partial amino acid | Mass to N- | Mass to C- | | |
| SF# | SPI# | spectrometry* | sequences determined | terminus | terminus | pl | MW |
| SF-342 | SPI-206 | 1569.9 | [I/L][I/L]GQ | 283.27 | 875.35 | 5.08 | 29463 |

*In Table XI, the masses determined by mass spectrometry have an error of mass measurement of 100 parts-per-million (ppm) or less. For a given measured mass, M, having an error of mass measurement of z ppm, the error of mass measurement can be calculated as (M x z ÷1000000).

As used herein, the “mass of the peptide analyzed by mass spectrometry” is the mass of the singly protonated peptide measured by mass spectrometry, and corresponds to the total mass of the constituent amino acid residues of the peptide with the addition of a water molecule (H₂O) and a single proton (H⁺). As used herein, the “mass to the N-terminus” corresponds to the total mass of the constituent amino acid residues extending from the start of the partial sequence to the N-terminus of the peptide. As used herein, the “mass to the C-terminus” corresponds to the total mass of the constituent amino acid residues extending from the end of the partial sequence to the C-terminus of the peptide with the addition of a water molecular (H₂O), and a single proton (H⁺).

The partial amino acid sequence and masses listed in Table XI were found to correspond to a peptide within the same putative human protein identified using SEQUEST, (i.e. protein ID CAA15430.1, accessible at http://www.ncbi.nlm.nih.gov/entrez/). The full amino acid sequence of the peptide listed in Table XI as a result of the match was found to be :GILILGQEQDTLGGR (shown in FIG. 2).

The DNA sequences encoding the two identified peptides are as follows:

E L D V L Q G R
gag ttg gac gtc ctg cag ggt cgt
G I L I L G Q E Q D T
ggg atc ctt atc ttg ggc cag gag cag gat acc ctg
L G G R
ggt ggc cgg

The human protein of database accession number CAA15430.1, the neuronal pentraxin receptor, whose gene is located on chromosome 22q12.3-13.2 (accession ID AL008583), is an ortholog of rat neuronal pentraxin receptor (Kirkpatrick LL, Matzuk MM, Dodds DC, Perin MS. Biochemical interactions of the neuronal pentraxins. Neuronal pentraxin (NP) receptor binds to taipoxin and taipoxin-associated calcium-binding protein 49 via NP1 and NP2. J Biol Chem. (2000) Jun 9;275(23):17786-92, Dodds D C, Omeis I A, Cushman S J, Helms J A, Perin M S. Neuronal pentraxin receptor (NPR), a novel putative integral membrane pentraxin that interacts with neuronal pentraxin 1 and 2 and taipoxin-associated calcium-binding protein 49. J. Biol Chem. (1997) Aug 22;272(34):21488-94).

A nucleotide sequence (FIG. 2B) encoding a peptide (FIG. 2A) comprising the above two peptides can be cloned by using the following primers:

5 cgcctcacgctgaagttcctg 3
5 ctggatgaggtggcccctcatgc 3

Primers useful for determining the sequence of the nucleotide sequence are:

5 tgttcagccgcttcctgtgcac 3
5 tctagcagtacaatctcgttgg 3

The peptide of FIG. 2A has 90% homology with the rat polypeptide (AAB62885). The rat protein, a putative integral membrane pentraxin, has 49 and 48% identity to neuronal pentraxin 1 (NP1) and neuronal pentraxin 2 (NP2), respectively (Dodds DC et al, supra; Hsu, et al, 1995, Genomics 28:2, 220-227). Theses proteins are suggested to be constituents of a pathway involved in the clearance of synaptic debris. Addition of NP1 to glial cultures renders them susceptible to a neurotoxin toxicity (Dodds DC et al, supra). NPR is expressed on the cell membrane and can form heteropentamers with NP1 and NP2 that can be released from cell membranes (Kirkpatrick et al, supra).

NP1 has homology to previously identified pentraxins, such as serum amyloid P protein (Dodds DC et al, supra). Serum amyloid P protein has been widely described for its role in amyloid (Botto M, et al, Nat Med. 1997 Aug;3(8):855-9; International Patent Publication WO9505394), and in particular its implication in Alzheimer disease progression (Tennent GA, Lovat LB, Pepys MB. Serum amyloid P component prevents proteolysis of the amyloid fibrils of Alzheimer disease and systemic amyloidosis. Proc Natl Acad Sci U.S.A. 1995 May 9;92(10):4299-303) and diagnostic (Hawkins PN, Rossor MN, Gallimore JR, Miller B, Moore EG, Pepys MB. Concentration of serum amyloid P component in the CSF as a possible marker of cerebral amyloid deposits in Alzheimer's disease. Biochem Biophys Res Commun. 1994 Jun 15;201(2):722-6) as well as other degenerative disorders (Kalaria RN, Galloway PG, Perry G. Widespread serum amyloid P immunoreactivity in cortical amyloid deposits and the neurofibrillary pathology of Alzheimer's disease and other degenerative disorders. Neuropathol Appl Neurobiol. 1991 Jun;17(3):189-201), and cerebral cell death (Urbanyi Z, Lakics V, Erdo SL. Serum amyloid P component-induced cell death in primary cultures of rat cerebral cortex. Eur J Pharmacol. 1994 Aug 3;270(4):375-8).

The peptide of FIG. 2A also exhibits homology to neuronal-activity-regulated pentraxin (NARP). See International Patent Publication WO9739133.

Therefore the peptide of FIG. 2A, which has 48% homology with serum amyloid P, and which is increased by 1.92 fold in Schizophrenia-affected patients, can be used for screening diagnosis, prognosis of Schizophrenia according to the methods of the invention. Any of the activities described in the above references concerning amyloid P, NARP, NPR, NP1 and NP2 can be used as the basis for functional assays for compounds capable of inhibiting or stimulating the relevant activity of the peptide of FIG. 2A. In addition, compounds capable of modulating the activities of amyloid P, NARP, NPR, NP1 and NP2 are candidate compounds for the treatment of Alzheimer's disease. Thus, assays for such compounds may also be performed, by, for example, recombinantly expressing amyloid P, NARP, NPR, NP1 or NP2 and testing for compounds capable of modulating the activity of the expressed protein.

The peptide of FIG. 2A may be recombinantly expressed by constructing an expression vector comprising the nucleic acid sequence of FIG. 2B, or portions thereof. The expressed recombinant protein may be used in assays for compounds capable of modulating the activity of the recombinant protein. In addition, assays for compounds capable of inhibiting or stimulating the cleavage of the peptide of FIG. 2A may be performed. In a particular embodiment, only a portion of the peptide is recombinantly produced. In a preferred embodiment, the portion of the peptide produced comprises a cleavage site. In a preferred embodiment, the portion of the peptide comprises amino acid residues 26 to 46, or residues 237 to 247. In another preferred embodiment, a truncated peptide comprising or consisting of the carboxyl terminus is produced, e.g., from amino acid residue 28, 36, 237, 243, 264 or 327 to the carboxyl terminus of the peptide.

5.6.2 Cloning and Characterization of SPI-238 and SPI-240

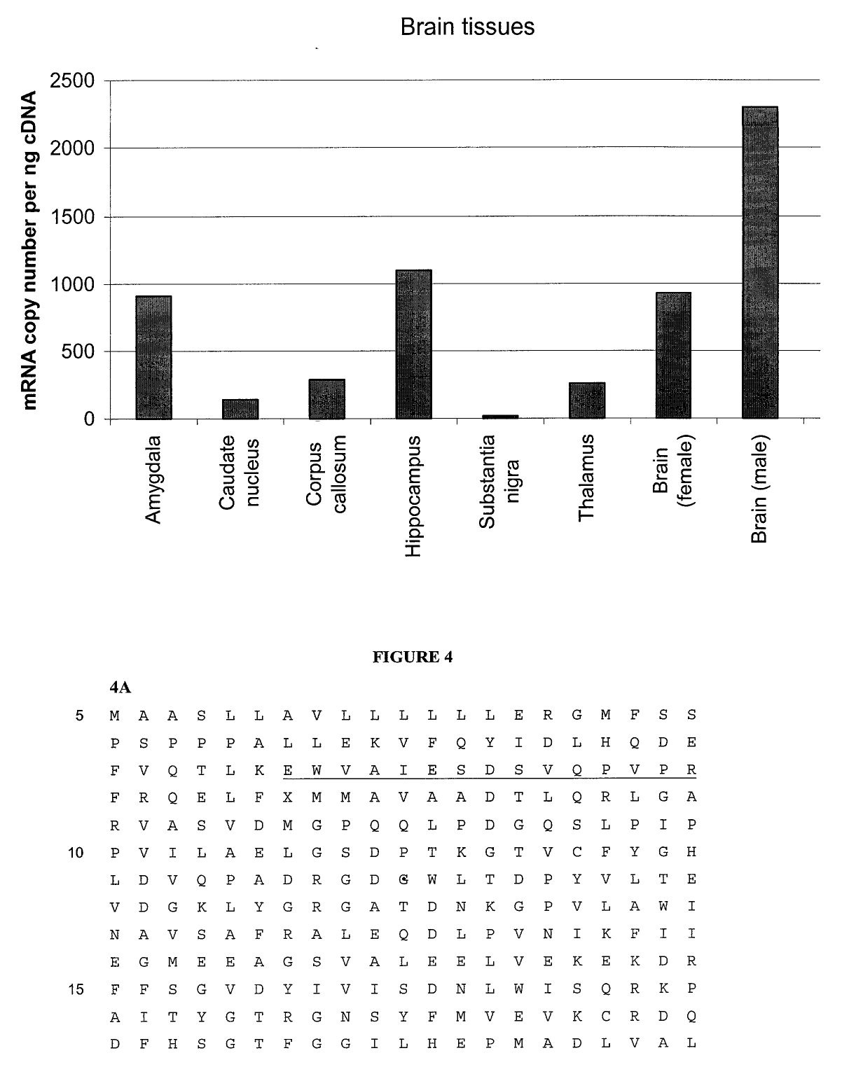

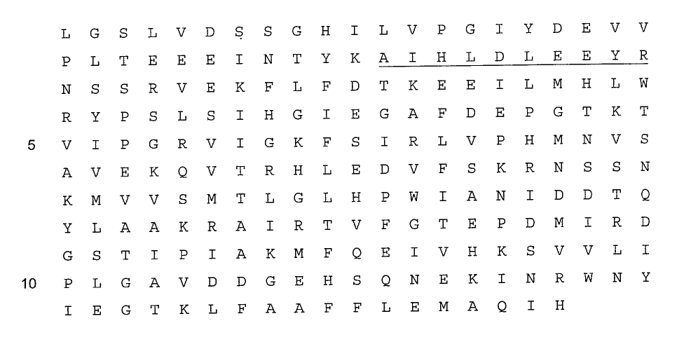

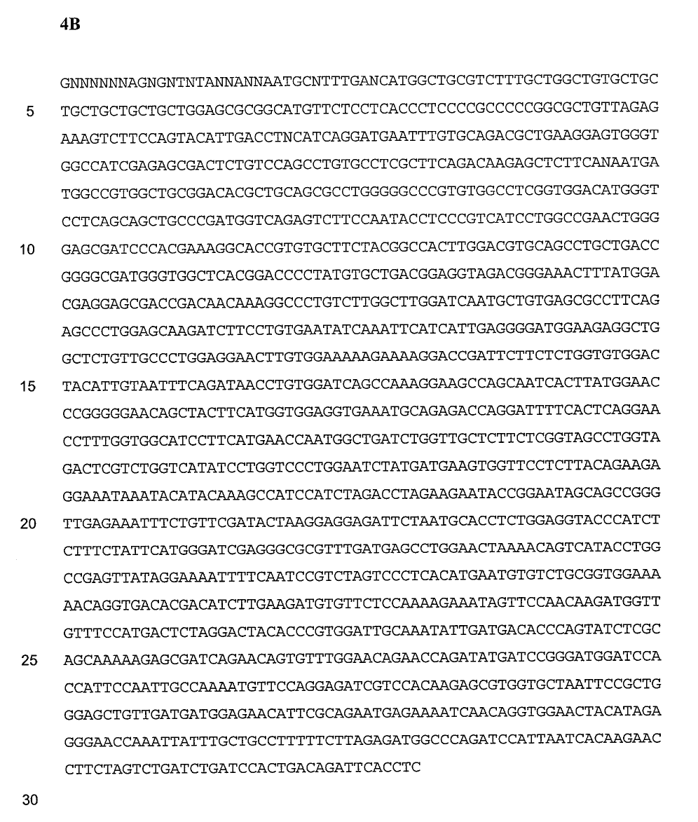

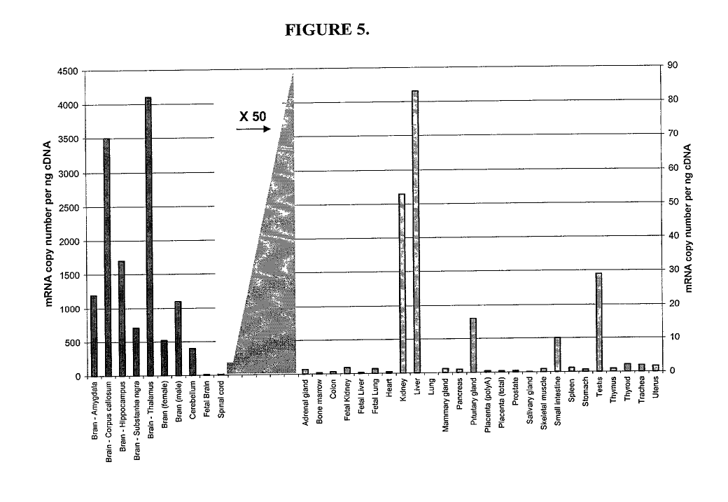

SPI-238 and SPI-240 were each isolated, subjected to proteolysis, and analyzed by mass spectrometry using the methods and apparatus of the Preferred Technology. Using the SEQUEST search program as described in the Examples, infra, uninterpreted tandem mass spectra of tryptic digest peptides were searched against a database of public domain proteins constructed of protein entries in the non-redundant database held by the National Centre for Biotechnology Information (NCBI) which is accessible at http://www.ncbi.nlm.nih.gov/and also constructed of Expressed Sequence Tags entries (http://www.ncbi.nlm.nih.gov/dbEST/index.html). As a result of database searching, the following amino acid sequence of a tryptic digest peptide of both SPI-238 and SPI-240 were determined from a match to a tryptic digest peptide in a conceptual translation of EST AA326679: EWVAIESDSVQPVPR (shown in FIG. 4B).

In cases where no amino acid sequences could be determined through searching using the SEQUEST program, tandem mass spectra of the peptides were interpreted manually, using methods known in the art as described in the Examples, infra. In the method of tandem mass spectrometry used for sequencing peptides in the present invention, the following pairs of amino acids could not be distinguished from each other: leucine and isoleucine; and, under certain circumstances, glutamine and lysine, and phenylalanine and oxidized methionine. As used herein, an amino acid sequence “as determined by mass spectrometry” refers to the set of amino acid sequences containing at the indicated positions, one or other member of the designated pairs of amino acids. For example, the amino acid sequence P[L/I]A indicates the amino acid sequences PLA and PIA. As will be obvious to one of skill in the art, a sequence containing n designated pairs indicates 2ⁿ amino acid sequences. For both SPI-238 and SPI-240 the same partial sequence was determined by mass spectrometry and is listed in Table XII.

TABLE XII
Partial Amino Acid Sequences of SPI-238 and
SPI-240 as Determined by Mass Spectrometry

| | | Mass of peptide | | | | | |
|---|---|---|---|---|---|---|---|
| | | analyzed by mass | Partial amino | Mass to | Mass to | | |
| SF# | SPI# | spectrometry* | acid sequences | N-terminus | C-terminus | pI | MW |
| SF-55 | SPI-238 | 1258.65 | H[L/I]D[L/I]EEYR | 184.07 | 0.00 | 4.94 | 59286 |
| SF-56 | SPI-240 | 1258.65 | H[L/I]D[L/I]EEYR | 184.07 | 0.00 | 5.04 | 57690 |

*The masses determined by mass spectrometry have an error of mass measurement of 100 parts-per-million (ppm) or less. For a given measured mass, M, having an error of mass measurement of z ppm, the error of mass measurement can be calculated as (M × z ÷ 1000000).

As used herein, the “mass of the peptide analyzed by mass spectrometry” is the mass of the singly protonated peptide measured by mass spectrometry, and corresponds to the total mass of the constituent amino acid residues of the peptide with the addition of a water molecule (H₂O) and a single proton (H⁺). As used herein, the “mass to N-terminus” corresponds to the total mass of the constituent amino acid residues extending from the start of the partial sequence to the N-terminus of the peptide. As used herein, the “mass to C-terminus” corresponds to the total mass of the constituent amino acid residues extending from the end of the partial sequence to the C-terminus of the peptide with the addition of a water molecular (H₂O), and a single proton (H⁺).

The partial amino acid sequence and masses listed in Table XII were not found to match to any sequences in the database used.

EST AA326679 shows 44% amino acid identity with a putative human protein derived from a conceptual translation of the cDNA CAB07646.1 (available at http://www.ncbi.nlm.nih.gov/entrez/). The C terminus of this protein sequence (CAB07646.1) shows a similar level of homology with a further brain tissue derived EST (AI589129) (TBlastN, BLAST, Altschul, Stephen F., Gish, Warren, Miller, Webb, Myers, Eugene W., and Lipman, David J. (1990). Basic local alignment search tool. J. Mol. Biol.215; 403-410.). This EST sequence does not overlap with EST AA326679 so that the possibility remained that the partial amino acid sequence and masses listed in Table XII could be encoded by the no-overlapping region of these 2 ESTs.

Opposing PCR primers (1 & 2 from Table XIII) from EST AA326679 and EST AI589129 were used in a PCR reaction (1 ml of Advantage 2 cDNA polymerase mix (Clontech) in a buffer containing 50 mM KCl, 10 mM Tris-HCl, 1.5 mM MgCl2, pH8.3; 0.2 mM each of dATP, dCTP, dGTP, dTTP and 10 pmoles of oligonucleotide primers. Reactions were routinely made to a final volume of 50 ml and amplification carried out in a PE GeneAmpSystems 9700 PCR machine with the following cycling conditions: initial denaturation of 94° C. for 1 minute followed by 30 cycles of 94° C. for 30 seconds, 55° C. for 30 seconds and 72° C. for 2 minutes. Reaction products were resolved by standard agarose gel electrophoresis and stained with Ethidium Bromide) on 10 ng of whole brain cDNA (Clontech, USA). The resulting 1.6 kb fragment was purified from primers and buffers (Qiagen, UK) and sequenced using the primers given in Table XIII (1,2, 3 & 4). This generated overlapping sequence for the entire product. Analysis of this DNA sequence (GCG, UK) shows a complete ORF now including the partial amino acid sequence and masses listed in Table XIII. FIG. 4B shows the DNA sequence. FIG. 4A show the protein sequence of the open reading frame (ORF), demonstrating the presence of the two peptides identified by mass spectrometry.

TABLE XIII
Primer Sequences

| Primer | Name | Sequence (5′ --- 3′) |
|---|---|---|
| 1 | SPI 238/240 F1 | gcctaatggntcccaaactc |
| 2 | SPI 238/240 R1 | gaggtgaatctgtcagtggatc |
| 3 | SPI 238/240 SF | atqgaagaggctggctctgttg |
| 4 | SPI 238/240 SR | aagagatgggtacctccagagg |

The DNA sequences encoding the sequences of two identified peptides are as follows:

gag tgg gtg gcc atc gag agc gac tct gtc cag cct
gtg cct
Glu Trp Val Ala Ile Glu Ser Asp Ser Val Gln Pro
Val Pro
and
gcc atc cat cta gac cta gaa gaa tac cgg
Ala Ile His Leu Asp Leu Glu Glu Tyr Arg

A Blast search against High Throughput Genomic Sequencing data (http://www.ncbi.nlm.nih.gov/blast) localised the SPI-238 and SPI-240 sequence EWVAIESDSVQPVPR to chromosome 18-clone RP11-231E4,map 18 (AC009704).

In a parallel study on Bipolar Affective Disorder (BAD), the protein corresponding to SPI-238 and SPI-240 was also found to be differentially present in samples of CSF from subjects having BAD compared with samples of CSF from subjects free from BAD, being decreased 2.27 fold (in the copending US patent application No. 60/254830 which is incorporated herein by reference).

The patent WO99/58660 disclosed 97 human secreted proteins. These included a sequence, identified as Gene No: 21, which corresponds to SPI-238 and SPI-240 discussed herein. However, this disclosure did not provide any isolated protein, nor did it identify SPI 238/240 as being differentially present in samples of CSF from subjects having BAD compared with a sample of CSF subjects free from BAD and in samples of CSF from subjects having Schizophrenia compared with a sample of CSF subjects free from Schizophrenia.

5.7 Expression of DNA Encoding SPIs

The nucleotide sequence coding for an SPI, an SPI analog, an SPI-related peptide, or a fragment or other derivative of any of the foregoing, can be inserted into an appropriate expression vector, i.e., a vector which contains the necessary elements for the transcription and translation of the inserted protein-coding sequence. The necessary transcriptional and translational signals can also be supplied by the native gene encoding the SPI or its flanking regions, or the native gene encoding the SPI-related polypeptide or its flanking regions. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used. In specific embodiments, a nucleotide sequence encoding a human gene (or a nucleotide sequence encoding a functionally active portion of a human SPI) is expressed. In yet another embodiment, a fragment of an SPI comprising a domain of the SPI is expressed.

Any of the methods previously described for the insertion of DNA fragments into a vector may be used to construct expression vectors containing a chimeric gene consisting of appropriate transcriptional and translational control signals and the protein coding sequences. These methods may include in vitro recombinant DNA and synthetic techniques and in vivo recombinants (genetic recombination). Expression of nucleic acid sequence encoding an SPI or fragment thereof may be regulated by a second nucleic acid sequence so that the SPI or fragment is expressed in a host transformed with the recombinant DNA molecule. For example, expression of an SPI may be controlled by any promoter or enhancer element known in the art. Promoters which may be used to control the expression of the gene encoding an SPI or an SPI-related polypeptide include, but are not limited to, the SV40 early promoter region (Bernoist and Chambon Nature (1981) 290:304-310), the promoter contained in the 3′long terminal repeat of Rous sarcoma virus (Yamamoto, et al, Cell (1980) 22:787-797), the herpes thymidine kinase promoter (Wagner et al, Proc. Natl. Acad. Sci. USA (1981) 78:1441-1445), the regulatory sequences of the metallothionein gene (Brinster et al, Nature (1982) 296:39-42), the tetracycline (Tet) promoter (Gossen et al, Proc. Nat. Acad. Sci. USA (1995) 89:5547-5551); prokaryotic expression vectors such as the β-lactamase promoter (Villa-Kamaroff, et al, Proc. Natl. Acad. Sci. USA (1978) 75:3727-3731), or the tac promoter (DeBoer, et al, Proc. Natl. Acad. Sci. USA (1983) 80:21-25; see also “Useful proteins from recombinant bacteria” in Scientific American (1980) 242:74-94); plant expression vectors comprising the nopaline synthetase promoter region (Herrera-Estrella et al, Nature (1984) 310(5973):115-20) or the cauliflower mosaic virus 35S RNA promoter (Gardner, et al, Nucl. Acids Res. (1981) 9:2871), and the promoter of the photosynthetic enzyme ribulose biphosphate carboxylase (Herrera-Estrella et al, Nature (1984) 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al, Cell (1984) 38:639-646; Ornitz et al, Cold Spring Harbor Symp. Quant. Biol. (1986) 50:399-409; MacDonald, Hepatology (1987) 7:425-515); insulin gene control region which is active in pancreatic beta cells (Hanahan, Nature (1985) 315:115-122), immunoglobulin gene control region which is active in lymphoid cells (Grosschedl et al, Cell (1984) 38:647-658; Adames et al, 1985, Nature 318:533-538; Alexander et al, Mol. Cell. Biol. (1987) 7:1436-1444), mouse mammary tumor virus control region which is active in testicular, breast, lymphoid and mast cells (Leder et al, Cell (1986) 45:485-495), albumin gene control region which is active in liver (Pinkert et al, Genes and Devel. (1987) 1:268-276), alpha-fetoprotein gene control region which is active in liver (Krumlauf et al, Mol. Cell. Biol. (1985) 5:1639-1648; Hammer et al, Science (1987) 235:53-58; alpha 1-antitrypsin gene control region which is active in the liver (Kelsey et al, Genes and Devel. (1987) 1:161-171), beta-globin gene control region which is active in myeloid cells (Mogram et al, Nature (1985) 315:338-340; Kollias et al, Cell (1986) 46:89-94; myelin basic protein gene control region which is active in oligodendrocyte cells in the brain (Readhead et al, Cell (1987) 48:703-712); myosin light chain-2 gene control region which is active in skeletal muscle (Sani, Nature (1985) 314:283-286); neuronal-specific enolase (NSE) which is active in neuronal cells (Morelli et al, Gen. Virol. (1999) 80:571-83); brain-derived neurotrophic factor (BDNF) gene control region which is active in neuronal cells (Tabuchi et al, Biochem. Biophysic. Res. Com. (1998) 253:818-823); glial fibrillary acidic protein (GFAP) promoter which is active in astrocytes (Gomes et al, Braz J Med Biol Res (1999) 32(5):619-631; Morelli et al, Gen. Virol. (1999) 80:571-83) and gonadotropic releasing hormone gene control region which is active in the hypothalamus (Mason et al, Science (1986) 234:1372-1378).

In a specific embodiment, a vector is used that comprises a promoter operably linked to an SPI-encoding nucleic acid, one or more origins of replication, and, optionally, one or more selectable markers (e.g., an antibiotic resistance gene).

In a specific embodiment, an expression construct is made by subcloning an SPI or an SPI-related polypeptide coding sequence into the EcoRI restriction site of each of the three pGEX vectors (Glutathione S-Transferase expression vectors; Smith and Johnson, Gene (1988) 7:31-40). This allows for the expression of the SPI product or SPI-related polypeptide from the subclone in the correct reading frame.

In mammalian host cells, a number of viral-based expression systems may be utilized. In cases where an adenovirus is used as an expression vector, the SPI coding sequence or SPI-related polypeptide coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA (1984) 81:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al, Methods in Enzymol. (1987) 153:51-544).

Expression vectors containing inserts of a gene encoding an SPI or an SPI-related polypeptide can be identified by three general approaches: (a) nucleic acid hybridization, (b) presence or absence of “marker” gene functions, and (c) expression of inserted sequences. In the first approach, the presence of a gene encoding an SPI inserted in an expression vector can be detected by nucleic acid hybridization using probes comprising sequences that are homologous to an inserted gene encoding an SPI. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a gene encoding an SPI in the vector. For example, if the gene encoding the SPI is inserted within the marker gene sequence of the vector, recombinants containing the gene encoding the SPI insert can be identified by the absence of the marker gene function. In the third approach, recombinant expression vectors can be identified by assaying the gene product (i.e., SPI) expressed by the recombinant. Such assays can be based, for example, on the physical or functional properties of the SPI in in vitro assay systems, e.g., binding with anti-SPI antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered SPI or SPI-related polypeptide may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, W138, and in particular, neuronal cell lines such as, for example, SK-N-AS, SK-N-Fl, SK-N-DZ human neuroblastomas (Sugimoto et al, J. Natl. Cancer Inst. (1984) 73: 51-57), SK-N-SH human neuroblastoma (Biochim. Biophys. Acta, 1982, 704: 450-460), Daoy human cerebellar medulloblastoma (He et al, Cancer Res. (1992) 52: 1144-1148) DBTRG-05MG glioblastoma cells (Kruse et al, In vitro Cell. Dev. Biol. (1992) 28A: 609-614), IMR-32 human neuroblastoma (Cancer Res., (1970) 30:2110-2118), 1321N1 human astrocytoma (Proc. Natl Acad. Sci. USA (1977) 74:4816), MOG-G-CCM human astrocytoma (Br. J. Cancer, (1984) 49:269), U87MG human glioblastoma-astrocytoma (Acta Pathol. Microbiol. Scand., (1968) 74: 465-486), A172 human glioblastoma (Olopade et al, Cancer Res. (1992) 52:2523-2529), C6 rat glioma cells (Benda et al, Science (1968) 161:370-371), Neuro-2a mouse neuroblastoma (Proc. Natl. Acad. Sci. USA, (1970) 65: 129-136), NB41A3 mouse neuroblastoma (Proc. Natl. Acad. Sci. USA, (1962) 48:1184-1190), SCP sheep choroid plexus (Bolin et al, J. Virol. Methods (1994) 48: 211-221), G355-5, PG-4 Cat normal astrocyte (Haapala et al, J. Virol. (1985) 53:827-833), Mpf ferret brain (Trowbridge et al, In vitro (1982) 18:952-960), and normal cell lines such as, for example, CTX TNA2 rat normal cortex brain (Radany et al, Proc. Natl. Acad. Sci. USA (1992) 89:6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express the differentially expressed or pathway gene protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the differentially expressed or pathway gene protein. Such engineered cell lines may be particularly useful in screening and evaluation of compounds that affect the endogenous activity of the differentially expressed or pathway gene protein.

A number of selection systems may be used, including but not limited to the herpes simplex virus thymidine kinase (Wigler, et al, Cell (1977) 11:223), hypoxanthine-guanine phosphoribosyltransferase (Szybalska & Szybalski, Proc. Natl. Acad. Sci. USA (1962) 48:2026), and adenine phosphoribosyltransferase (Lowy, et al, Cell (1980) 22:817) genes can be employed in tk-, hgprt- or aprt-cells, respectively. Also, antimetabolite resistance can be used as the basis of selection for dhfr, which confers resistance to methotrexate (Wigler, et al, Proc. Natl. Acad. Sci. USA (1980) 77:3567; O'Hare, et al, Proc. Natl. Acad. Sci. USA (1981) 78:1527); gpt, which confers resistance to mycophenolic acid (Mulligan & Berg, Proc. Natl. Acad. Sci. USA (1981) 78:2072); neo, which confers resistance to the aminoglycoside G-418 (Colberre-Garapin, et al, J. Mol. Biol. (1981) 150:1); and hygro, which confers resistance to hygromycin (Santerre, et al, Gene (1984) 30:147) genes.

In other specific embodiments, the SPI, fragment, analog, or derivative may be expressed as a fusion, or chimeric protein product (comprising the protein, fragment, analog, or derivative joined via a peptide bond to a heterologous protein sequence). For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification, increase half-life in vivo, and enhance the delivery of an antigen across an epithelial barrier to the immune system. An increase in the half-life in vivo and facilitated purification has been shown for chimeric proteins consisting of the first two domains of the human CD4-polypeptide and various domains of the constant regions of the heavy or light chains of mammalian immunoglobulins. See, e.g., EP 394,827; Traunecker et al, Nature, (1988) 331:84-86 . Enhanced delivery of an antigen across the epithelial barrier to the immune system has been demonstrated for antigens (e.g., insulin) conjugated to an FcRn binding partner such as IgG or Fc fragments (see, e.g., PCT publications WO 96/22024 and WO 99/04813).

Nucleic acids encoding an SPI, a fragment of an SPI, an SPI-related polypeptide, or a fragment of an SPI-related polypeptide can be fused to an epitope tag (e.g., the hemagglutinin (“HA”) tag or flag tag) to aid in detection and purification of the expressed polypeptide. For example, a system described by Janknecht et al, allows for the ready purification of non-denatured fusion proteins expressed in human cell lines (Janknecht et al, Proc. Natl. Acad. Sci. USA (1991) 88:8972-897).

Fusion proteins can be made by ligating the appropriate nucleic acid sequences encoding the desired amino acid sequences to each other by methods known in the art, in the proper coding frame, and expressing the chimeric product by methods commonly known in the art. Alternatively, a fusion protein may be made by protein synthetic techniques, e.g., by use of a peptide synthesizer.

Both cDNA and genomic sequences can be cloned and expressed.

5.8 Domain Structure of SPIs

Domains of some SPIs are known in the art and have been described in the scientific literature. Moreover, domains of an SPI can be identified using techniques known to those of skill in the art. For example, one or more domains of an SPI can be identified by using one or more of the following programs: ProDom, TMpred, and SAPS. ProDom compares the amino acid sequence of a polypeptide to a database of compiled domains (see, e.g., http://www.toulouse.inra.fr/prodom.html; Corpet F., Gouzy J. & Kahn D., Nucleic Acids Res., (1999) 27:263-267). TMpred predicts membrane-spanning regions of a polypeptide and their orientation. This program uses an algorithm that is based on the statistical analysis of TMbase, a database of naturally occuring transmembrane proteins (see, e.g., http://www.ch.embnet.org/software/TMPRED_form.html; Hofmann & Stoffel. (1993) “TMbase—A database of membrane spanning proteins segments.” Biol. Chem. Hoppe-Seyler 347,166). The SAPS program analyzes polypeptides for statistically significant features like charge-clusters, repeats, hydrophobic regions, compositional domains (see, e.g., Brendel et al, Proc. Natl. Acad. Sci. USA (1992) 89: 2002-2006). Thus, based on the present description, the skilled artisan can identify domains of an SPI having enzymatic or binding activity, and further can identify nucleotide sequences encoding such domains. These nucleotide sequences can then be used for recombinant expression of an SPI fragment that retains the enzymatic or binding activity of the SPI.

Based on the present description, the skilled artisan can identify domains of an SPI having enzymatic or binding activity, and further can identify nucleotide sequences encoding such domains. These nucleotide sequences can then be used for recombinant expression of SPI fragments that retain the enzymatic or binding activity of the SPI.

In one embodiment, an SPI has an amino acid sequence sufficiently similar to an identified domain of a known polypeptide. As used herein, the term “sufficiently similar” refers to a first amino acid or nucleotide sequence which contains a sufficient number of identical or equivalent (e.g., with a similar side chain) amino acid residues or nucleotides to a second amino acid or nucleotide sequence such that the first and second amino acid or nucleotide sequences have or encode a common structural domain or common functional activity or both.

An SPI domain can be assessed for its function using techniques well known to those of skill in the art. For example, a domain can be assessed for its kinase activity or for its ability to bind to DNA using techniques known to the skilled artisan. Kinase activity can be assessed, for example, by measuring the ability of a polypeptide to phosphorylate a substrate. DNA binding activity can be assessed, for example, by measuring the ability of a polypeptide to bind to a DNA binding element in a electromobility shift assay. In a preferred embodiment, the function of a domain of an SPI is determined using an assay described in one or more of the references identified in Table XIV, infra.

5.9 Production of Antibodies to SPIs

According to the invention an SPI, SPI analog, SPI-related protein or a fragment or derivative of any of the foregoing may be used as an immunogen to generate antibodies which immunospecifically bind such an immunogen. Such immunogens can be isolated by any convenient means, including the methods described above. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule.

In one embodiment, antibodies that recognize gene products of genes encoding SPIs are publicly available. For example, antibodies that recognize these SPIs and/or their isoforms include antibodies recognizing: SPI-6, SPI-8, SPI-9, SPI-10, SPI-15, SPI-16, SPI-18, SPI-23, SPI-32, SPI-33, SPI-34, SPI-35, SPI-41, SPI-42, SPI-43, SPI-54, SPI-57, SPI-60, SPI-62, SPI-63, SPI-67, SPI-73, SPI-74, SPI-75, SPI-76, SPI-77, SPI-78, SPI-80, SPI-82, SPI-91, SPI-92, SPI-93, SPI-96, SPI-97, SPI-99, SPI-100, SPI-101, SPI-105, SPI-107, SPI-113, SPI-114, SPI-115, SPI-122, SPI-124, SPI-127, SPI-129, SPI-130, SPI-132, SPI-143, SPI-152, SPI-154, SPI-155, SPI-164, SPI-167, SPI-170, SPI-175, SPI-183, SPI-184, SPI-185, SPI-190, SPI-193, SPI-205, SPI-211, SPI-231, SPI-233, SPI-237, SPI-242, SPI-244, SPI-246, SPI-252, SPI-254, SPI-255, SPI-258, SPI-261, SPI-264, SPI-265, SPI-269, SPI-275, SPI-285, SPI-289, SPI-290, SPI-321, SPI-323, SPI-325, SPI-326, SPI-327, SPI-328, SPI-329, SPI-334, SPI-339, SPI-342, SPI-343, SPI-345, SPI-346, SPI-347, SPI-348, SPI-349, SPI-353, SPI-355, SPI-357, SPI-361, SPI-362, SPI-370, SPI-372, SPI-375, SPI-376, SPI-379, SPI-381, SPI-397, SPI-402, SPI-404, SPI-405, SPI-407, SPI-408, SPI-409, SPI-410, SPI-41 1, SPI-412, SPI-413, SPI-414, SPI-416, SPI-418, SPI-420, SPI-421, SPI-422, SPI-423, SPI-424, SPI-425, SPI-426, SPI-427, SPI-429, SPI-431, SPI-432, SPI-433, SPI-435, SPI-438, SPI-441, SPI-443, SPI-446, SPI-448, SPI-449, SPI-452, SPI-453, SPI-454, SPI-457, SPI-461, SPI-462, SPI-464, SPI-465, SPI-467, which antibodies can be purchased from commercial sources as shown in Table VII above. In another embodiment, methods known to those skilled in the art are used to produce antibodies that recognize an SPI, an SPI analog, an SPI-related polypeptide, or a derivative or fragment of any of the foregoing.

In one embodiment of the invention, antibodies to a specific domain of an SPI are produced. In a specific embodiment, hydrophilic fragments of an SPI are used as immunogens for antibody production.

In the production of antibodies, screening for the desired antibody can be accomplished by techniques known in the art, e.g. ELISA (enzyme-linked immunosorbent assay). For example, to select antibodies which recognize a specific domain of an SPI, one may assay generated hybridomas for a product which binds to an SPI fragment containing such domain. For selection of an antibody that specifically binds a first SPI homolog but which does not specifically bind to (or binds less avidly to) a second SPI homolog, one can select on the basis of positive binding to the first SPI homolog and a lack of binding to (or reduced binding to) the second SPI homolog. Similarly, for selection of an antibody that specifically binds an SPI but which does not specifically bind to (or binds less avidly to) a different isoform of the same protein (such as a different glycoform having the same core peptide as the SPI), one can select on the basis of positive binding to the SPI and a lack of binding to (or reduced binding to) the different isoform (e.g., a different glycoform). Thus, the present invention provides an antibody (preferably a monoclonal antibody) that binds with greater affinity (preferably at least 2-fold, more preferably at least 5-fold still more preferably at least 10-fold greater affinity) to an SPI than to a different isoform or isoforms (e.g., glycoforms) of the SPI.

Polyclonal antibodies that may be used in the methods of the invention are heterogeneous populations of antibody molecules derived from the sera of immunized animals. Unfractionated immune serum can also be used. Various procedures known in the art may be used for the production of polyclonal antibodies to an SPI, a fragment of an SPI, an SPI-related polypeptide, or a fragment of an SPI-related polypeptide. In a particular embodiment, rabbit polyclonal antibodies to an epitope of an SPI or an SPI-related polypeptide can be obtained. For example, for the production of polyclonal or monoclonal antibodies, various host animals can be immunized by injection with the native or a synthetic (e.g., recombinant) version of an SPI, a fragment of an SPI, an SPI-related polypeptide, or a fragment of an SPI-related polypeptide, including but not limited to rabbits, mice, rats, etc. The Preferred Technology described herein provides isolated SPIs suitable for such immunization. If the SPI is purified by gel electrophoresis, the SPI can be used for immunization with or without prior extraction from the polyacrylamide gel. Various adjuvants may be used to enhance the immunological response, depending on the host species, including, but not limited to, complete or incomplete Freund's adjuvant, a mineral gel such as aluminum hydroxide, surface active substance such as lysolecithin, pluronic polyol, a polyanion, a peptide, an oil emulsion, keyhole limpet hemocyanin, dinitrophenol, and an adjuvant such as BCG (bacille Calmette-Guerin) or corynebacterium parvum. Additional adjuvants are also well known in the art.

For preparation of monoclonal antibodies (mAbs) directed toward an SPI, a fragment of an SPI, an SPI-related polypeptide, or a fragment of an SPI-related polypeptide, any technique which provides for the production of antibody molecules by continuous cell lines in culture may be used. For example, the hybridoma technique originally developed by Kohler and Milstein (Nature (1975) 256:495-497), as well as the trioma technique, the human B-cell hybridoma technique (Kozbor et al, Immunology Today (1983) 4:72), and the EBV-hybridoma technique to produce human monoclonal antibodies (Cole et al, (1985) in Monoclonal Antibodies and Cancer Therapy, Alan R. Liss, Inc., pp. 77-96). Such antibodies may be of any immunoglobulin class including IgG, IgM, IgE, IgA, IgD and any subclass thereof. The hybridoma producing the mAbs of the invention may be cultivated in vitro or in vivo. In an additional embodiment of the invention, monoclonal antibodies can be produced in germ-free animals utilizing known technology (PCT/US90/02545, incorporated herein by reference).

The monoclonal antibodies include but are not limited to human monoclonal antibodies and chimeric monoclonal antibodies (e.g., human-mouse chimeras). A chimeric antibody is a molecule in which different portions are derived from different animal species, such as those having a human immunoglobulin constant region and a variable region derived from a murine mAb. (See, e.g., Cabilly et al, U.S. Pat. No. 4,816,567; and Boss et al, U.S. Pat. No. 4,816397, which are incorporated herein by reference in their entirety.) Humanized antibodies are antibody molecules from non- human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.)

Chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al, 1988, Science 240:1041-1043; Liu et al, Proc. Natl. Acad. Sci. USA (1987) 84:3439-3443; Liu et al, J. Immunol. (1987) 139:3521-3526; Sun et al, Proc. Natl. Acad. Sci. USA (1987) 84:214-218; Nishimura et al, Canc. Res. (1987) 47:999-1005; Wood et al, Nature (1985) 314:446-449; and Shaw et al, J. Natl. Cancer Inst. (1988) 80:1553-1559; Morrison, Science (1985) 229:1202-1207; Oi et al, Bio/Techniques (1986) 4:214; U.S. Pat. No. 5,225,539; Jones et al, Nature (1986) 321:552-525; Verhoeyan et al, Science (1988) 239:1534; and Beidler et al, J. Immunol. (1988) 141:4053-4060.

Completely human antibodies are particularly desirable for therapeutic treatment of human subjects. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of an SPI of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (Int. Rev. Immunol. (1995) 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al, Bio/technology (1994) 12:899-903).

The antibodies of the present invention can also be generated using various phage display methods known in the art. In phage display methods, functional antibody domains are displayed on the surface of phage particles that carry the polynucleotide sequences encoding them. In a particular, such phage can be utilized to display antigen binding domains expressed from a repertoire or combinatorial antibody library (e.g., human or murine). Phage expressing an antigen binding domain that binds the antigen of interest can be selected or identified with antigen, e.g., using labelled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Phage display methods that can be used to make the antibodies of the present invention include those disclosed in Brinkman et al, J. Immunol. Methods (1995) 182:41-50 ; Ames et al, J. Immunol. Methods (1995) 184:177-186 ; Kettleborough et al, Eur. J. Immunol. (1994) 24:952-958 ; Persic et al, Gene (1997) 187 9-18 ; Burton et al, Advances in Immunology (1994) 57:191-280; PCT Application No. PCT/GB91/01134; PCT Publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)2 fragments can also be employed using methods known in the art such as those disclosed in PCT publication WO 92/22324; Mullinax et al, BioTechniques 12(6):864-869 (1992); and Sawai et al, (1995) AJRI 34:26-34 ; and Better et al, Science (1988) 240:1041-1043 (said references incorporated by reference in their entireties).

Examples of techniques which can be used to produce single-chain Fvs and antibodies include those described in U.S. Pat. Nos. 4,946,778 and 5,258,498; Huston et al, Methods in Enzymology 203:46-88 (1991); Shu et al, Proc. Natl. Sci Acad. USA (1993) 90:7995-7999; and Skerra et al, Science (1988) 240:1038-1040 .

The invention further provides for the use of bispecific antibodies, which can be made by methods known in the art. Traditional production of full length bispecific antibodies is based on the coexpression of two immunoglobulin heavy chain-light chain pairs, where the two chains have different specificities (Milstein et al, Nature (1983) 305:537-539). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, published May 13, 1993, and in Traunecker et al, EMBO J. (1991) 10:3655-3659.

According to a different and more preferred approach, antibody variable domains with the desired binding specificities (antibody-antigen combining sites) are fused to immunoglobulin constant domain sequences. The fusion preferably is with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details for generating bispecific antibodies see, for example, Suresh et al, Methods in Enzymology (1986) 121:210.

The invention provides functionally active fragments, derivatives or analogs of the anti-SPI immunoglobulin molecules. Functionally active means that the fragment, derivative or analog is able to elicit anti-anti-idiotype antibodies (i.e., tertiary antibodies) that recognize the same antigen that is recognized by the antibody from which the fragment, derivative or analog is derived. Specifically, in a preferred embodiment the antigenicity of the idiotype of the immunoglobulin molecule may be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

The present invention provides antibody fragments such as, but not limited to, F(ab′)2 fragments and Fab fragments. Antibody fragments which recognize specific epitopes may be generated by known techniques. F(ab′)2 fragments consist of the variable region, the light chain constant region and the CH1 domain of the heavy chain and are generated by pepsin digestion of the antibody molecule. Fab fragments are generated by reducing the disulfide bridges of the F(ab′)2 fragments. The invention also provides heavy chain and light chain dimers of the antibodies of the invention, or any minimal fragment thereof such as Fvs or single chain antibodies (SCAs) (e.g., as described in U.S. Pat. No. 4,946,778; Bird, Science (1988) 242:423-42; Huston et al, Proc. Natl. Acad. Sci. USA (1988) 85:5879-5883; and Ward et al, Nature (1989) 334:544-54), or any other molecule with the same specificity as the antibody of the invention. Single chain antibodies are formed by linking the heavy and light chain fragments of the Fv region via an amino acid bridge, resulting in a single chain polypeptide. Techniques for the assembly of functional Fv fragments in E. coli may be used (Skerra et al, Science (1988) 242:1038-1041).

In other embodiments, the invention provides fusion proteins of the immunoglobulins of the invention (or functionally active fragments thereof), for example in which the immunoglobulin is fused via a covalent bond (e.g., a peptide bond), at either the N-terminus or the C-terminus to an amino acid sequence of another protein (or portion thereof, preferably at least 10, 20 or 50 amino acid portion of the protein) that is not the immunoglobulin. Preferably the immunoglobulin, or fragment thereof, is covalently linked to the other protein at the N-terminus of the constant domain. As stated above, such fusion proteins may facilitate purification, increase half-life in vivo, and enhance the delivery of an antigen across an epithelial barrier to the immune system.

The immunoglobulins of the invention include analogs and derivatives that are either modified, i.e., by the covalent attachment of any type of molecule as long as such covalent attachment that does not impair immunospecific binding. For example, but not by way of limitation, the derivatives and analogs of the immunoglobulins include those that have been further modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the analog or derivative may contain one or more non-classical amino acids.

The foregoing antibodies can be used in methods known in the art relating to the localization and activity of the SPIs of the invention, e.g., for imaging these proteins, measuring levels thereof in appropriate physiological samples, in diagnostic methods, etc.

5.10 Expression Of Antibodies

The antibodies of the invention can be produced by any method known in the art for the synthesis of antibodies, in particular, by chemical synthesis or by recombinant expression, and are preferably produced by recombinant expression techniques.

Recombinant expression of antibodies, or fragments, derivatives or analogs thereof, requires construction of a nucleic acid that encodes the antibody. If the nucleotide sequence of the antibody is known, a nucleic acid encoding the antibody may be assembled from chemically synthesized oligonucleotides (e.g., as described in Kutmeier et al, BioTechniques (1994) 17:242), which, briefly, involves the synthesis of overlapping oligonucleotides containing portions of the sequence encoding antibody, annealing and ligation of those oligonucleotides, and then amplification of the ligated oligonucleotides by PCR.

Alternatively, the nucleic acid encoding the antibody may be obtained by cloning the antibody. If a clone containing the nucleic acid encoding the particular antibody is not available, but the sequence of the antibody molecule is known, a nucleic acid encoding the antibody may be obtained from a suitable source (e.g., an antibody cDNA library, or cDNA library generated from any tissue or cells expressing the antibody) by PCR amplification using synthetic primers hybridizable to the 3 and 5 ends of the sequence or by cloning using an oligonucleotide probe specific for the particular gene sequence.

If an antibody molecule that specifically recognizes a particular antigen is not available (or a source for a cDNA library for cloning a nucleic acid encoding such an antibody), antibodies specific for a particular antigen may be generated by any method known in the art, for example, by immunizing an animal, such as a rabbit, to generate polyclonal antibodies or, more preferably, by generating monoclonal antibodies. Alternatively, a clone encoding at least the Fab portion of the antibody may be obtained by screening Fab expression libraries (e.g., as described in Huse et al, Science (1989) 246:1275-1281) for clones of Fab fragments that bind the specific antigen or by screening antibody libraries (See, e.g., Clackson et al, Nature (1991) 352:624; Hane et al, Proc. Natl. Acad. Sci. USA (1997) 94:4937).

Once a nucleic acid encoding at least the variable domain of the antibody molecule is obtained, it may be introduced into a vector containing the nucleotide sequence encoding the constant region of the antibody molecule (see, e.g., PCT Publication WO 86/05807; PCT Publication WO 89/01036; and U.S. Pat. No. 5,122,464). Vectors containing the complete light or heavy chain for co-expression with the nucleic acid to allow the expression of a complete antibody molecule are also available. Then, the nucleic acid encoding the antibody can be used to introduce the nucleotide substitution(s) or deletion(s) necessary to substitute (or delete) the one or more variable region cysteine residues participating in an intrachain disulfide bond with an amino acid residue that does not contain a sulfhydyl group. Such modifications can be carried out by any method known in the art for the introduction of specific mutations or deletions in a nucleotide sequence, for example, but not limited to, chemical mutagenesis, in vitro site directed mutagenesis (Hutchinson et al, J. Biol. Chem. (1978) 253:6551), PCT based methods, etc.

In addition, techniques developed for the production of “chimeric antibodies” (Morrison et al, Proc. Natl. Acad. Sci. (1984) 81:851-855; Neuberger et al, Nature (1984) 312:604-608; Takeda et al, Nature (1985) 314:452-454) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human antibody constant region, e.g., humanized antibodies.

Once a nucleic acid encoding an antibody molecule of the invention has been obtained, the vector for the production of the antibody molecule may be produced by recombinant DNA technology using techniques well known in the art. Thus, methods for preparing the protein of the invention by expressing nucleic acid containing the antibody molecule sequences are described herein. Methods which are well known to those skilled in the art can be used to construct expression vectors containing an antibody molecule coding sequences and appropriate transcriptional and translational control signals. These methods include, for example, in vitro recombinant DNA techniques, synthetic techniques, and in vivo genetic recombination. See, for example, the techniques described in Sambrook et al, (1990, Molecular Cloning, A Laboratory Manual, 2d Ed., Cold Spring Harbor Laboratory, Cold Spring Harbor, N.Y.) and Ausubel et al, (eds., 1998, Current Protocols in Molecular Biology, John Wiley & Sons, NY).

The expression vector is transferred to a host cell by conventional techniques and the transfected cells are then cultured by conventional techniques to produce an antibody of the invention.

The host cells used to express a recombinant antibody of the invention may be either bacterial cells such as Escherichia coli, or, preferably, eukaryotic cells, especially for the expression of whole recombinant antibody molecule. In particular, mammalian cells such as Chinese hamster ovary cells (CHO), in conjunction with a vector such as the major intermediate early gene promoter element from human cytomegalovirus is an effective expression system for antibodies (Foecking et al, Gene (1986) 45:101; Cockett et al, Bio/Technology (1990) 8:2).

A variety of host-expression vector systems may be utilized to express an antibody molecule of the invention. Such host-expression systems represent vehicles by which the coding sequences of interest may be produced and subsequently purified, but also represent cells which may, when transformed or transfected with the appropriate nucleotide coding sequences, express the antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antibody molecule being expressed. For example, when a large quantity of such a protein is to be produced, for the generation of pharmaceutical compositions comprising an antibody molecule, vectors which direct the expression of high levels of fusion protein products that are readily purified may be desirable. Such vectors include, but are not limited, to the E. coli expression vector pUR278 (Ruther et al, EMBO J. (1983) 2:1791), in which the antibody coding sequence may be ligated individually into the vector in frame with the lac Z coding region so that a fusion protein is produced; pIN vectors (Inouye & Inouye, Nucleic Acids Res. (1985) 13:3101-3109; Van Heeke & Schuster, J. Biol. Chem. (1989) 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter). In mammalian host cells, a number of viral-based expression systems (e.g., an adenovirus expression system) may be utilized.

As discussed above, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein.

For long-term, high-yield production of recombinant antibodies, stable expression is preferred. For example, cells lines that stably express an antibody of interest can be produced by transfecting the cells with an expression vector comprising the nucleotide sequence of the antibody and the nucleotide sequence of a selectable (e.g., neomycin or hygromycin), and selecting for expression of the selectable marker. Such engineered cell lines may be particularly useful in screening and evaluation of compounds that interact directly or indirectly with the antibody molecule.

The expression levels of the antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al, 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain should be placed before the heavy chain to avoid an excess of toxic free heavy chain (Proudfoot, Nature (1986) 322:52; Kohler, Proc. Natl. Acad. Sci. USA (1980) 77:2197). The coding sequences for the heavy and light chains may comprise cDNA or genomic DNA.

Once the antibody molecule of the invention has been recombinantly expressed, it may be purified by any method known in the art for purification of an antibody molecule, for example, by chromatography (e.g., ion exchange chromatography, affinity chromatography such as with protein A or specific antigen, and sizing column chromatography), centrifugation, differential solubility, or by any other standard technique for the purification of proteins.

Alternatively, any fusion protein may be readily purified by utilizing an antibody specific for the fusion protein being expressed. For example, a system described by Janknecht et al, allows for the ready purification of non-denatured fusion proteins expressed in human cell lines (Janknecht et al, Proc. Natl. Acad. Sci. USA (1991) 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with recombinant vaccinia virus are loaded onto Ni2+nitriloacetic acid-agarose columns and histidine-tagged proteins are selectively eluted with imidazole-containing buffers.

5.11 Conjugated Antibodies

In a preferred embodiment, anti-SPI antibodies or fragments thereof are conjugated to a diagnostic or therapeutic moiety. The antibodies can be used for diagnosis or to determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics according to the present invention. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

Anti-SPI antibodies or fragments thereof can be conjugated to a therapeutic agent or drug moiety to modify a given biological response. The therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, -interferon, -interferon, nerve growth factor, platelet derived growth factor, tissue plasminogen activator, a thrombotic agent or an anti-angiogenic agent, e.g., angiostatin or endostatin; or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Techniques for conjugating such therapeutic moiety to antibodies are well known, see, e.g., Arnon et al, “Monoclonal Antibodies For Immunotargeting Of Drugs In Cancer Therapy”, in Monoclonal Antibodies And Cancer Therapy, Reisfeld et al, (eds.), pp. 243-56 (Alan R. Liss, Inc. 1985); Hellstrom et al, “Antibodies For Drug Delivery”, in Controlled Drug Delivery (2nd Ed.), Robinson et al, (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al, (eds.), pp. 475-506 (1985); “Analysis, Results, And Future Prospective Of The Therapeutic Use Of Radiolabelled Antibody In Cancer Therapy”, in Monoclonal Antibodies For Cancer Detection And Therapy, Baldwin et al, (eds.), pp. 303-16 (Academic Press 1985), and Thorpe et al, “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980.

An antibody with or without a therapeutic moiety conjugated to it can be used as a therapeutic that is administered alone or in combination with cytotoxic factor(s) and/or cytokine(s).

5.12 Diagnosis of Schizophrenia

In accordance with the present invention, test samples of cerebrospinal fluid (CSF), serum, plasma or urine obtained from a subject suspected of having or known to have Schizophrenia can be used for diagnosis or monitoring. In one embodiment, a decreased abundance of one or more SFs or SPIs (or any combination of them) in a test sample relative to a control sample (from a subject or subjects free from Schizophrenia) or a previously determined reference range indicates the presence of Schizophrenia; SFs and SPIs suitable for this purpose are identified in Tables I and IV, respectively, as described in detail above. In another embodiment of the invention, an increased abundance of one or more SFs or SPIs (or any combination of them) in a test sample compared to a control sample or a previously determined reference range indicates the presence of Schizophrenia; SFs and SPIs suitable for this purpose are identified in Tables II and V, respectively, as described in detail above. In another embodiment, the relative abundance of one or more SFs or SPIs (or any combination of them) in a test sample compared to a control sample or a previously determined reference range indicates a subtype of Schizophrenia (e.g., familial or sporadic Schizophrenia). In yet another embodiment, the relative abundance of one or more SFs or SPIs (or any combination of them) in a test sample relative to a control sample or a previously determined reference range indicates the degree or severity of Schizophrenia. In any of the aforesaid methods, detection of one or more SPIs described herein may optionally be combined with detection of one or more additional biomarkers for Schizophrenia including, but not limited to. Any suitable method in the art can be employed to measure the level of SFs and SPIs, including but not limited to the Preferred Technology described herein, kinase assays, immunoassays to detect and/or visualize the SPI (e.g., Western blot, immunoprecipitation followed by sodium dodecyl sulfate polyacrylamide gel electrophoresis, immunocytochemistry, etc.). In cases where an SPI has a known function, an assay for that function may be used to measure SPI expression. In a further embodiment, a decreased abundance of mRNA encoding one or more SPIs identified in Table IV (or any combination of them) in a test sample relative to a control sample or a previously determined reference range indicates the presence of Schizophrenia. In yet a further embodiment, an increased abundance of mRNA encoding one or more SPIs identified in Table V (or any combination of them) in a test sample relative to a control sample or previously determined reference range indicates the presence of Schizophrenia. Any suitable hybridization assay can be used to detect SPI expression by detecting and/or visualizing mRNA encoding the SPI (e.g., Northern assays, dot blots, in situ hybridization, etc.).

In another embodiment of the invention, labelled antibodies, derivatives and analogs thereof, which specifically bind to an SPI can be used for diagnostic purposes to detect, diagnose, or monitor Schizophrenia. Preferably, Schizophrenia is detected in an animal, more preferably in a mammal and most preferably in a human.

5.13 Screening Assays

The invention provides methods for identifying agents (e.g., candidate compounds or test compounds) that bind to an SPI or have a stimulatory or inhibitory effect on the expression or activity of an SPI. The invention also provides methods of identifying agents, candidate compounds or test compounds that bind to an SPI-related polypeptide or an SPI fusion protein or have a stimulatory or inhibitory effect on the expression or activity of an SPI-related polypeptide or an SPI fusion protein. Examples of agents, candidate compounds or test compounds include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. (1997) 12:145; U.S. Pat. No. 5,738,996; and U.S. Pat. No.5,807,683, each of which is incorporated herein in its entirety by reference).

Examples of methods for the synthesis of molecular libraries can be found in the art, for example in: DeWitt et al, Proc. Natl. Acad. Sci. USA (1993) 90:6909; Erb et al, Proc. Natl. Acad. Sci. USA (1994) 91:11422; Zuckermann et al, J. Med. Chem. (1994) 37:2678; Cho et al, Science (1993) 261:1303; Carrell et al, Angew. Chem. Int. Ed. Engl. (1994) 33:2059;

Carell et al, Angew. Chem. Int. Ed. Engl. (1994) 33:2061; and Gallop et al, J. Med. Chem. (1994) 37:1233, each of which is incorporated herein in its entirety by reference. Libraries of compounds may be presented, e.g., presented in solution (e.g., Houghten, Bio/Techniques (1992) 13:412-421), or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al, Proc. Natl. Acad. Sci. USA (1992) 89:1865-1869) or phage (Scott and Smith, Science (1990) 249:386-390; Devlin, Science (1990) 249:404-406; Cwirla et al, Proc. Natl Acad. Sci. USA (1990) 87:6378-6382; and Felici, J. Mol. Biol. (1990) 222:301-310), each of which is incorporated herein in its entirety by reference.

In one embodiment, agents that interact with (i.e., bind to) an SPI, an SPI fragment (e.g. a functionally active fragment), an SPI-related polypeptide, a fragment of an SPI-related polypeptide, or an SPI fusion protein are identified in a cell-based assay system. In accordance with this embodiment, cells expressing an SPI, a fragment of an SPI, an SPI-related polypeptide, a fragment of an SPI-related polypeptide, or an SPI fusion protein are contacted with a candidate compound or a control compound and the ability of the candidate compound to interact with the SPI is determined. If desired, this assay may be used to screen a plurality (e.g. a library) of candidate compounds. The cell, for example, can be of prokaryotic origin (e.g., E. coli) or eukaryotic origin (e.g., yeast or mammalian). Further, the cells can express the SPI, fragment of the SPI, SPI-related polypeptide, a fragment of the SPI-related polypeptide, or an SPI fusion protein endogenously or be genetically engineered to express the SPI, fragment of the SPI, SPI-related polypeptide, a fragment of the SPI-related polypeptide, or an SPI fusion protein. In certain instances, the SPI, fragment of the SPI, SPI-related polypeptide, a fragment of the SPI-related polypeptide, or an SPI fusion protein or the candidate compound is labelled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between an SPI and a candidate compound. The ability of the candidate compound to interact directly or indirectly with an SPI, a fragment of an SPI, an SPI-related polypeptide, a fragment of an SPI-related polypeptide, or an SPI fusion protein can be determined by methods known to those of skill in the art. For example, the interaction between a candidate compound and an SPI, a fragment of an SPI, an SPI-related polypeptide, a fragment of an SPI-related polypeptide, or an SPI fusion protein can be determined by flow cytometry, a scintillation assay, immunoprecipitation or western blot analysis.

In another embodiment, agents that interact with (i.e., bind to) an SPI, an SPI fragment (e.g., a functionally active fragment) an SPI-related polypeptide, a fragment of an SPI-related polypeptide, or an SPI fusion protein are identified in a cell-free assay system. In accordance with this embodiment, a native or recombinant SPI or fragment thereof, or a native or recombinant SPI-related polypeptide or fragment thereof, or an SPI-fusion protein or fragment thereof, is contacted with a candidate compound or a control compound and the ability of the candidate compound to interact with the SPI or SPI-related polypeptide, or SPI fusion protein is determined. If desired, this assay may be used to screen a plurality (e.g. a library) of candidate compounds. Preferably, the SPI, SPI fragment, SPI-related polypeptide, a fragment of an SPI-related polypeptide, or an SPI-fusion protein is first immobilized, by, for example, contacting the SPI, SPI fragment, SPI-related polypeptide, a fragment of an SPI-related polypeptide, or an SPI fusion protein with an immobilized antibody which specifically recognizes and binds it, or by contacting a purified preparation of the SPI, SPI fragment, SPI-related polypeptide, fragment of an SPI-related polypeptide, or an SPI fusion protein with a surface designed to bind proteins. The SPI, SPI fragment, SPI-related polypeptide, a fragment of an SPI-related polypeptide, or an SPI fusion protein may be partially or completely purified (e.g., partially or completely free of other polypeptides) or part of a cell lysate. Further, the SPI, SPI fragment, SPI-related polypeptide, a fragment of an SPI-related polypeptide may be a fusion protein comprising the SPI or a biologically active portion thereof, or SPI-related polypeptide and a domain such as glutathionine-S-transferase. Alternatively, the SPI, SPI fragment, SPI-related polypeptide, fragment of an SPI-related polypeptide or SPI fusion protein can be biotinylated using techniques well known to those of skill in the art (e.g., biotinylation kit, Pierce Chemicals; Rockford, Ill.). The ability of the candidate compound to interact with an SPI, SPI fragment, SPI-related polypeptide, a fragment of an SPI-related polypeptide, or an SPI fusion protein can be can be determined by methods known to those of skill in the art.

In another embodiment, a cell-based assay system is used to identify agents that bind to or modulate the activity of a protein, such as an enzyme, or a biologically active portion thereof, which is responsible for the production or degradation of an SPI or is responsible for the post-translational modification of an SPI. In a primary screen, a plurality (e.g., a library) of compounds are contacted with cells that naturally or recombinantly express: (i) an SPI, an isoform of an SPI, an SPI homolog an SPI-related polypeptide, an SPI fusion protein, or a biologically active fragment of any of the foregoing; and (ii) a protein that is responsible for processing of the SPI, SPI isoform, SPI homolog, SPI-related polypeptide, SPI fusion protein, or fragment in order to identify compounds that modulate the production, degradation, or post-translational modification of the SPI, SPI isoform, SPI homolog, SPI-related polypeptide, SPI fusion protein or fragment. If desired, compounds identified in the primary screen can then be assayed in a secondary screen against cells naturally or recombinantly expressing the specific SPI of interest. The ability of the candidate compound to modulate the production, degradation or post-translational modification of an SPI, isoform, homolog, SPI-related polypeptide, or SPI fusion protein can be determined by methods known to those of skill in the art, including without limitation, flow cytometry, a scintillation assay, immunoprecipitation and western blot analysis.

In another embodiment, agents that competitively interact with (i.e., bind to) an SPI, SPI fragment, SPI-related polypeptide, a fragment of an SPI-related polypeptide, or an SPI fusion protein are identified in a competitive binding assay. In accordance with this embodiment, cells expressing an SPI, SPI fragment, SPI-related polypeptide, a fragment of an SPI-related polypeptide, or an SPI fusion protein are contacted with a candidate compound and a compound known to interact with the SPI, SPI fragment, SPI-related polypeptide, a fragment of an SPI-related polypeptide or an SPI fusion protein; the ability of the candidate compound to competitively interact with the SPI, SPI fragment, SPI-related polypeptide, fragment of an SPI-related polypeptide, or an SPI fusion protein is then determined. Alternatively, agents that competitively interact with (i.e., bind to) an SPI, SPI fragment, SPI-related polypeptide or fragment of an SPI-related polypeptide are identified in a cell-free assay system by contacting an SPI, SPI fragment, SPI-related polypeptide, fragment of an SPI-related polypeptide, or an SPI fusion protein with a candidate compound and a compound known to interact with the SPI, SPI-related polypeptide or SPI fusion protein. As stated above, the ability of the candidate compound to interact with an SPI, SPI fragment, SPI-related polypeptide, a fragment of an SPI-related polypeptide, or an SPI fusion protein can be determined by methods known to those of skill in the art. These assays, whether cell-based or cell-free, can be used to screen a plurality (e.g., a library) of candidate compounds.

In another embodiment, agents that modulate (i.e., upregulate or downregulate) the expression of an SPI, or an SPI-related polypeptide are identified by contacting cells (e.g., cells of prokaryotic origin or eukaryotic origin) expressing the SPI, or SPI-related polypeptide with a candidate compound or a control compound (e.g., phosphate buffered saline (PBS)) and determining the expression of the SPI, SPI-related polypeptide, or SPI fusion protein, mRNA encoding the SPI, or mRNA encoding the SPI-related polypeptide. The level of expression of a selected SPI, SPI-related polypeptide, mRNA encoding the SPI, or mRNA encoding the SPI-related polypeptide in the presence of the candidate compound is compared to the level of expression of the SPI, SPI-related polypeptide, mRNA encoding the SPI, or mRNA encoding the SPI-related polypeptide in the absence of the candidate compound (e.g., in the presence of a control compound). The candidate compound can then be identified as a modulator of the expression of the SPI, or an SPI-related polypeptide based on this comparison. For example, when expression of the SPI or mRNA is significantly greater in the presence of the candidate compound than in its absence, the candidate compound is identified as a stimulator of expression of the SPI or mRNA. Alternatively, when expression of the SPI or mRNA is significantly less in the presence of the candidate compound than in its absence, the candidate compound is identified as an inhibitor of the expression of the SPI or mRNA. The level of expression of an SPI or the mRNA that encodes it can be determined by methods known to those of skill in the art. For example, mRNA expression can be assessed by Northern blot analysis or RT-PCR, and protein levels can be assessed by western blot analysis.

In another embodiment, agents that modulate the activity of an SPI, or an SPI-related polypeptide are identified by contacting a preparation containing the SPI or SPI-related polypeptide, or cells (e.g., prokaryotic or eukaryotic cells) expressing the SPI or SPI-related polypeptide with a test compound or a control compound and determining the ability of the test compound to modulate (e.g., stimulate or inhibit) the activity of the SPI or SPI-related polypeptide. The activity of an SPI or an SPI-related polypeptide can be assessed by detecting induction of a cellular signal transduction pathway of the SPI or SPI-related polypeptide (e.g., intracellular Ca2+, diacylglycerol, IP3, etc.), detecting catalytic or enzymatic activity of the target on a suitable substrate, detecting the induction of a reporter gene ( e.g., a regulatory element that is responsive to an SPI or an SPI-related polypeptide and is operably linked to a nucleic acid encoding a detectable marker, e.g., luciferase), or detecting a cellular response, for example, cellular differentiation, or cell proliferation. Based on the present description, techniques known to those of skill in the art can be used for measuring these activities (see, e.g., U.S. Pat. No. 5,401,639, which is incorporated herein by reference). The candidate compound can then be identified as a modulator of the activity of an SPI or SPI-related polypeptide by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of an SPI or SPI-related polypeptide are identified in an animal model. Examples of suitable animals include, but are not limited to, mice, rats, rabbits, monkeys, guinea pigs, dogs and cats. Preferably, the animal used represent a model of Schizophrenia (e.g.,Phencyclidine treated rodents (Sams-Dodd Rev Neurosci (1999) 10, 59-90), an animal model of deficient sensorimotor gating (Swerdlow and Geyer Schizophr Bull (1998) 24:2 285-301), neonatal insult to the hippocampal region (Beauregard and Bachevalier Can J Psychiatry (1996) Sep 41:7 446-56), models based on neonatal excitotoxic hippocampal damage (Lillrank et al, Clin Neurosci (1995) 3:2 98-104), attention deficit models (Feldon et al, J Psychiatr Res 4, 345-66) and NMDA deficient rodent models (Mohn et al, Cell (1999) 98, 427-436). In accordance with this embodiment, the test compound or a control compound is administered (e.g., orally, rectally or parenterally such as intraperitoneally or intravenously) to a suitable animal and the effect on the expression, activity or both expression and activity of the SPI or SPI-related polypeptide is determined. Changes in the expression of an SPI or SPI-related polypeptide can be assessed by the methods outlined above.

In yet another embodiment, an SPI or SPI-related polypeptide is used as a “bait protein” in a two-hybrid assay or three hybrid assay to identify other proteins that bind to or interact with an SPI or SPI-related polypeptide (see, e.g., U.S. Pat. No. 5,283,317; Zervos et al, Cell (1993) 72:223-232; Madura et al, J. Biol. Chem. (1993) 268:12046-12054; Bartel et al, Bio/Techniques (1993) 14:920-924; Iwabuchi et al, Oncogene (1993) 8:1693-1696; and PCT Publication No. WO 94/10300). As those skilled in the art will appreciate, such binding proteins are also likely to be involved in the propagation of signals by the SPIs of the invention as, for example, upstream or downstream elements of a signaling pathway involving the SPIs of the invention.

Table XIV enumerates scientific publications describing suitable assays for detecting or quantifying enzymatic or binding activity of an SPI, an SPI analog, an SPI-related polypeptide, or a fragment of any of the foregoing. Each such reference is hereby incorporated in its entirety. In a preferred embodiment, as assay referenced in Table XIV is used in the screens and assays described herein, for example to screen for or identify a compound that modulates the activity of (or that modulates both the expression and activity of) an SPI, SPI analog, or SPI-related polypeptide, a fragment of any of the foregoing.

TABLE XIV

| SPI | References |
|---|---|
| SPI-82 | Structural Biology 2000 7: 312-321, |
| SPI-109 | J. Am. Chem. Soc. 2000 122: 2178-2192, |
| SPI-154 | |
| SPI-188 | |
| SPI-3 | Clin Chem 1993 Feb 39(2): 309-12 |
| SPI-32 | J Immunol Methods 1987 Aug 24 102:1 7-14 |
| SPI-33 | |
| SPI-45 | |
| SPI-92 | |
| SPI-122 | |
| SPI-165 | |
| SPI-57 | J Clin Lab Immunol 1986 Dec 21(4): 201-7 |
| SPI-67 | |
| SPI-74 | |
| SPI-77 | |
| SPI-107 | |
| SPI-153 | |
| SPI-162 | |
| SPI-164 | |
| SPI-175 | |
| SPI-186 | |
| SPI-205 | |
| SPI-216 | |
| SPI-41 | Neuroendocrinology 1992 Mar 55:3 308-16 |
| SPI-47 | J Chromatogr 1987 Dec 18 411: 498-501 |
| | Eisei Shikenjo Hokoku 1972 90: 89-92 |
| | Analyst 1990 Aug 115:8 1143-4 |
| SPI-194 | Biochem J 1997 Mar 1 322 (Pt 2): 455-60; |
| | Biochem Soc Trans 1997 Nov 25:4 S591; |
| | Biochim Biophys Acta 1986 Oct 10 888:3 325-31 |
| | http://www.promega.com |

This invention further provides novel agents identified by the above-described screening assays and uses thereof for treatments as described herein.

5.14 Therapeutic Uses of SPIs

The invention provides for treatment or prevention of various diseases and disorders by administration of a therapeutic compound. Such compounds include but are not limited to: SPIs, SPI analogs, SPI-related polypeptides and derivatives (including fragments) thereof; antibodies to the foregoing; nucleic acids encoding SPIs, SPI analogs, SPI-related polypeptides and fragments thereof; antisense nucleic acids to a gene encoding an SPI or SPI-related polypeptide; and modulator (e.g., agonists and antagonists) of a gene encoding an SPI or SPI-related polypeptide. An important feature of the present invention is the identification of genes encoding SPIs involved in Schizophrenia. Schizophrenia can be treated (e.g. to ameliorate symptoms or to retard onset or progression) or prevented by administration of a therapeutic compound that promotes function or expression of one or more SPIs that are decreased in the CSF of Schizophrenia subjects having Schizophrenia, or by administration of a therapeutic compound that reduces function or expression of one or more SPIs that are increased in the CSF of subjects having Schizophrenia.

In one embodiment, one or more antibodies each specifically binding to an SPI are administered alone or in combination with one or more additional therapeutic compounds or treatments. Examples of such therapeutic compounds or treatments include, but are not limited to, Sertindole, Haloperidol, Pirenzepine, Perazine, Risperdal, Famotidine, Clozaril, Mesoridazine, Quetiapine, atypical anti-psychotic medications of Risperidone, Zyperexa (Olanzapine) and Clozapine and any other Dibenzothiazepines. The compounds of the invention may be given in combination with any other compound, including Sertindole, Haloperidol, Pirenzepine, Perazine, Risperdal, Famotidine, Clozaril, Mesoridazine, Quetiapine, atypical anti-psychotic medications of Risperidone, Zyperexa (Olanzapine) and Clozapine and any other Dibenzothiazepines.

Preferably, a biological product such as an antibody is allogeneic to the subject to which it is administered. In a preferred embodiment, a human SPI or a human SPI-related polypeptide, a nucleotide sequence encoding a human SPI or a human SPI-related polypeptide, or an antibody to a human SPI or a human SPI-related polypeptide, is administered to a human subject for therapy (e.g. to ameliorate symptoms or to retard onset or progression) or prophylaxis.

5.14.1 Treatment And Prevention Of Schizophrenia

Schizophrenia is treated or prevented by administration to a subject suspected of having or known to have Schizophrenia or to be at risk of developing Schizophrenia of a compound that modulates (i.e., increases or decreases) the level or activity (i.e., function) of one or more SPIs or the level of one or more SFs that are differentially present in the CSF of subjects having Schizophrenia compared with CSF of subjects free from Schizophrenia. In one embodiment, Schizophrenia is treated or prevented by administering to a subject suspected of having or known to have Schizophrenia or to be at risk of developing Schizophrenia a compound that upregulates (i.e., increases) the level or activity (i.e., function) of one or more SPIs or the level of one or more SFs that are decreased in the CSF of subjects having Schizophrenia. In another embodiment, a compound is administered that downregulates the level or activity (i.e., function) of one or more SPIs or the level of one or more SFs that are increased in the CSF of subjects having Schizophrenia. Examples of such a compound include but are not limited to: SPIs, SPI fragments and SPI-related polypeptides; nucleic acids encoding an SPI, an SPI fragment and an SPI-related polypeptide (e.g., for use in gene therapy); and, for those SPIs or SPI-related polypeptides with enzymatic activity, compounds or molecules known to modulate that enzymatic activity. Other compounds that can be used, e.g., SPI agonists, can be identified using in vitro assays.

Schizophrenia is also treated or prevented by administration to a subject suspected of having or known to have Schizophrenia or to be at risk of developing Schizophrenia of a compound that downregulates the level or activity of one or more SPIs or the level of one or more SFs that are increased in the CSF of subjects having Schizophrenia. In another embodiment, a compound is administered that upregulates the level or activity of one or more SPIs or the level of one or more SFs that are decreased in the CSF of subjects having Schizophrenia. Examples of such a compound include, but are not limited to, SPI antisense oligonucleotides, ribozymes, antibodies directed against SPIs, and compounds that inhibit the enzymatic activity of an SPI. Other useful compounds e.g., SPI antagonists and small molecule SPI antagonists, can be identified using in vitro assays.

In a preferred embodiment, therapy or prophylaxis is tailored to the needs of an individual subject. Thus, in specific embodiments, compounds that promote the level or function of one or more SPIs, or the level of one or more SFs, are therapeutically or prophylactically administered to a subject suspected of having or known to have Schizophrenia, in whom the levels or functions of said one or more SPIs, or levels of said one or more SFs, are absent or are decreased relative to a control or normal reference range. In further embodiments, compounds that promote the level or function of one or more SPIs, or the level of one or more SFs, are therapeutically or prophylactically administered to a subject suspected of having or known to have Schizophrenia in whom the levels or functions of said one or more SPIs, or levels of said one or more SFs, are increased relative to a control or to a reference range. In further embodiments, compounds that decrease the level or function of one or more SPIs, or the level of one or more SFs, are therapeutically or prophylactically administered to a subject suspected of having or known to have Schizophrenia in whom the levels or functions of said one or more SPIs, or levels of said one or more SFs, are increased relative to a control or to a reference range. In further embodiments, compounds that decrease the level or function of one or more SPIs, or the level of one or more SFs, are therapeutically or prophylactically administered to a subject suspected of having or known to have Schizophrenia in whom the levels or functions of said one or more SPIs, or levels of said one or more SFs, are decreased relative to a control or to a reference range. The change in SPI function or level, or SF level, due to the administration of such compounds can be readily detected, e.g., by obtaining a sample (e.g., a sample of CSF, blood or urine or a tissue sample such as biopsy tissue) and assaying in vitro the levels of said SFs or the levels or activities of said SPIs, or the levels of mRNAs encoding said SPIs. or any combination of the foregoing. Such assays can be performed before and after the administration of the compound as described herein.

The compounds of the invention include but are not limited to any compound, e.g., a small organic molecule, protein, peptide, antibody, nucleic acid, etc. that restores the Schizophrenia SPI or SF profile towards normal with the proviso that such compounds do not include Haloperidol, Pirenzepine, Perazine, Risperdal, Famotidine, Clozaril, Mesoridazine, Quetiapine, atypical anti-psychotic medications of Risperidone, Zyperexa (Olanzapine) and Clozapine and any other Dibenzothiazepines.

5.14.2 Gene Therapy

In a specific embodiment, nucleic acids comprising a sequence encoding an SPI, an SPI fragment, SPI-related polypeptide or fragment of an SPI-related polypeptide, are administered to promote SPI function by way of gene therapy. Gene therapy refers to administration to a subject of an expressed or expressible nucleic acid. In this embodiment, the nucleic acid produces its encoded polypeptide that mediates a therapeutic effect by promoting SPI function.

Any of the methods for gene therapy available in the art can be used according to the present invention. Exemplary methods are described below.

For general reviews of the methods of gene therapy, see Goldspiel et al, Clinical Pharmacy (1993) 12:488-505; Wu and Wu, Biotherapy (1991) 3:87-95; Tolstoshev, Ann. Rev. Pharmacol. Toxicol. (1993) 32:573-596; Mulligan, Science (1993) 260:926-932; and Morgan and Anderson, Ann. Rev. Biochem. (1993) 62:191-217; May, 1993, TIBTECH 11(5):155-215. Methods commonly known in the art of recombinant DNA technology which can be used are described in Ausubel et al, (eds.), 1993, Current Protocols in Molecular Biology, John Wiley & Sons, NY; and Kriegler, 1990, Gene Transfer and Expression, A Laboratory Manual, Stockton Press, NY.

In a preferred aspect, the compound comprises a nucleic acid encoding an SPI or fragment or chimeric protein thereof, said nucleic acid being part of an expression vector that expresses an SPI or fragment or chimeric protein thereof in a suitable host. In particular, such a nucleic acid has a promoter operably linked to the SPI coding region, said promoter being inducible or constitutive (and, optionally, tissue-specific). In another particular embodiment, a nucleic acid molecule is used in which the SPI coding sequences and any other desired sequences are flanked by regions that promote homologous recombination at a desired site in the genome, thus providing for intrachromosomal expression of the SPI nucleic acid (Koller and Smithies, Proc. Natl. Acad. Sci. USA (1989) 86:8932-8935; Zijlstra et al, Nature (1989) 342:435-438).

Delivery of the nucleic acid into a subject may be direct, in which case the subject is directly exposed to the nucleic acid or nucleic acid-carrying vector; this approach is known as in vivo gene therapy. Alternatively, delivery of the nucleic acid into the subject may be indirect, in which case cells are first transformed with the nucleic acid in vitro and then transplanted into the subject; this approach is known as ex vivo gene therapy.

In a specific embodiment, the nucleic acid is directly administered in vivo, where it is expressed to produce the encoded product. This can be accomplished by any of numerous methods known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286); by direct injection of naked DNA; by use of microparticle bombardment (e.g., a gene gun; Biolistic, Dupont); by coating with lipids, cell-surface receptors or transfecting agents; by encapsulation in liposomes, microparticles or microcapsules; by administering it in linkage to a peptide which is known to enter the nucleus; or by administering it in linkage to a ligand subject to receptor-mediated endocytosis (see, e.g., Wu and Wu, J. Biol. Chem. (1987) 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992 (Wu et al,); WO 92/22635 dated Dec. 23, 1992 (Wilson et al,); WO92/20316 dated Nov. 26, 1992 (Findeis et al,); WO93/14188 dated Jul. 22, 1993 (Clarke et al,), WO 93/20221 dated Oct. 14, 1993 (Young)). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al, Nature (1989) 342:435-438).

In a specific embodiment, a viral vector that contains a nucleic acid encoding an SPI is used. For example, a retroviral vector can be used (see Miller et al, Meth. Enzymol. (1993) 217:581-599). These retroviral vectors have been modified to delete retroviral sequences that are not necessary for packaging of the viral genome and integration into host cell DNA. The nucleic acid encoding the SPI to be used in gene therapy is cloned into the vector, which facilitates delivery of the gene into a subject. More detail about retroviral vectors can be found in Boesen et al, Biotherapy (1994) 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al, J. Clin. Invest. (1994) 93:644-651; Kiem et al, Blood (1994) 83:1467-1473; Salmons and Gunzberg, Human Gene Therapy (1993) 4:129-141; and Grossman and Wilson, Curr. Opin. in Genetics and Devel. (1993) 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development (1993) 3:499-503 present a review of adenovirus-based gene therapy. Bout et al, Human Gene Therapy (1994) 5:3-10 demonstrated the use of adenovirus vectors to transfer genes to the respiratory epithelia of rhesus monkeys. Other instances of the use of adenoviruses in gene therapy can be found in Rosenfeld et al, Science (1991) 252:431-434; Rosenfeld et al, Cell (1992) 68:143-155; Mastrangeli et al, J. Clin. Invest. (1993) 91:225-234; PCT Publication W094/12649; and Wang, et al, Gene Therapy (1995) 2:775-783.

Adeno-associated virus (AAV) has also been proposed for use in gene therapy (Walsh et al, Proc. Soc. Exp. Biol. Med. (1993) 204:289-300; U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. (1993) 217:599-618; Cohen et al, Meth. Enzymol. (1993) 217:618-644; Cline, Pharmac. Ther. (1985) 29:69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. In a preferred embodiment, epithelial cells are injected, e.g., subcutaneously. In another embodiment, recombinant skin cells may be applied as a skin graft onto the subject. Recombinant blood cells (e.g., hematopoietic stem or progenitor cells) are preferably administered intravenously. The amount of cells envisioned for use depends on the desired effect, the condition of the subject, etc., and can be determined by one skilled in the art.

Cells into which a nucleic acid can be introduced for purposes of gene therapy encompass any desired, available cell type, and include but are not limited to neuronal cells, glial cells (e.g., oligodendrocytes or astrocytes), epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood or fetal liver.

In a preferred embodiment, the cell used for gene therapy is autologous to the subject that is treated.

In an embodiment in which recombinant cells are used in gene therapy, a nucleic acid encoding an SPI is introduced into the cells such that it is expressible by the cells or their progeny, and the recombinant cells are then administered in vivo for therapeutic effect. In a specific embodiment, stem or progenitor cells are used. Any stem or progenitor cells which can be isolated and maintained in vitro can be used in accordance with this embodiment of the present invention (see e.g. PCT Publication WO 94/08598, dated Apr. 28, 1994; Stemple and Anderson, 1992, Cell 71:973-985; Rheinwald, Meth. Cell Bio. (1980) 21A:229; and Pittelkow and Scott, Mayo Clinic Proc. (1986) 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

Direct injection of a DNA coding for an SPI may also be performed according to, for example, the techniques described in U.S. Pat. No. 5,589,466. These techniques involve the injection of “naked DNA”, i.e., isolated DNA molecules in the absence of liposomes, cells, or any other material besides a suitable carrier. The injection of DNA encoding a protein and operably linked to a suitable promoter results in the production of the protein in cells near the site of injection and the elicitation of an immune response in the subject to the protein encoded by the injected DNA. In a preferred embodiment, naked DNA comprising (a) DNA encoding an SPI and (b) a promoter are injected into a subject to elicit an immune response to the SPI.

5.14.3 Inhibition of SPIs to Treat Schizophrenia

In one embodiment of the invention, Schizophrenia is treated or prevented by administration of a compound that antagonizes (inhibits) the level(s) and/or function(s) of one or more SPIs which are elevated in the CSF of subjects having Schizophrenia as compared with CSF of subjects free from Schizophrenia. Compounds useful for this purpose include but are not limited to anti-SPI antibodies (and fragments and derivatives containing the binding region thereof), SPI antisense or ribozyme nucleic acids, and nucleic acids encoding dysfunctional SPIs that are used to “knockout” endogenous SPI function by homologous recombination (see, e.g., Capecchi, Science (1989) 244:1288-1292). Other compounds that inhibit SPI function can be identified by use of known in vitro assays, e.g., assays for the ability of a test compound to inhibit binding of an SPI to another protein or a binding partner, or to inhibit a known SPI function. Preferably such inhibition is assayed in vitro or in cell culture, but genetic assays may also be employed. The Preferred Technology can also be used to detect levels of the SPI before and after the administration of the compound. Preferably, suitable in vitro or in vivo assays are utilized to determine the effect of a specific compound and whether its administration is indicated for treatment of the affected tissue, as described in more detail below.

In a specific embodiment, a compound that inhibits an SPI function is administered therapeutically or prophylactically to a subject in whom an increased CSF level or functional activity of the SPI (e.g., greater than the normal level or desired level) is detected as compared with CSF of subjects free from Schizophrenia or a predetermined reference range. Methods standard in the art can be employed to measure the increase in an SPI level or function, as outlined above. Preferred SPI inhibitor compositions include small molecules, i.e., molecules of 1000 daltons or less. Such small molecules can be identified by the screening methods described herein.

5.14.4 Antisense Regulation of SPIs

In a specific embodiment, SPI expression is inhibited by use of SPI antisense nucleic acids. The present invention provides the therapeutic or prophylactic use of nucleic acids comprising at least six nucleotides that are antisense to a gene or cDNA encoding an SPI or a portion thereof. As used herein, an SPI “antisense” nucleic acid refers to a nucleic acid capable of hybridizing by virtue of some sequence complementarity to a portion of an RNA (preferably mRNA) encoding an SPI. The antisense nucleic acid may be complementary to a coding and/or noncoding region of an mRNA encoding an SPI. Such antisense nucleic acids have utility as compounds that inhibit SPI expression, and can be used in the treatment or prevention of Schizophrenia.

The antisense nucleic acids of the invention are double-stranded or single-stranded oligonucleotides, RNA or DNA or a modification or derivative thereof, and can be directly administered to a cell or produced intracellularly by transcription of exogenous, introduced sequences.

The invention further provides pharmaceutical compositions comprising an effective amount of the SPI antisense nucleic acids of the invention in a pharmaceutically acceptable carrier, as described infra.

In another embodiment, the invention provides methods for inhibiting the expression of an SPI nucleic acid sequence in a prokaryotic or eukaryotic cell comprising providing the cell with an effective amount of a composition comprising an SPI antisense nucleic acid of the invention.

SPI antisense nucleic acids and their uses are described in detail below.

5.14.5 SPI Antisense Nucleic Acids

The SPI antisense nucleic acids are of at least six nucleotides and are preferably oligonucleotides ranging from 6 to about 50 oligonucleotides. In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof and can be single- stranded or double-stranded. The oligonucleotide can be modified at the base moiety, sugar moiety, or phosphate backbone. The oligonucleotide may include other appended groups such as peptides; agents that facilitate transport across the cell membrane (see, e.g., Letsinger et al, Proc. Natl. Acad. Sci. USA (1989) 86:6553-6556; Lemaitre et al, Proc. Natl. Acad. Sci. USA (1987) 84:648-652; PCT Publication No. WO 88/09810, published Dec. 15, 1988) or blood-brain barrier (see, e.g., PCT Publication No. WO 89/10134, published Apr. 25, 1988); hybridization-triggered cleavage agents (see, e.g., Krol et al, BioTechniques (1988) 6:958-976) or intercalating agents (see, e.g., Zon, Pharm. Res. (1988) 5:539-549).

In a preferred aspect of the invention, an SPI antisense oligonucleotide is provided, preferably of single-stranded DNA. The oligonucleotide may be modified at any position on its structure with substituents generally known in the art.

The SPI antisense oligonucleotide may comprise at least one of the following modified base moieties: 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta- D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, and other base analogs.

In another embodiment, the oligonucleotide comprises at least one modified sugar moiety, e.g., one of the following sugar moieties: arabinose, 2-fluoroarabinose, xylulose, and hexose.

In yet another embodiment, the oligonucleotide comprises at least one of the following modified phosphate backbones: a phosphorothioate, a phosphorodithioate, a phosphoramidothioate, a phosphoramidate, a phosphordiamidate, a methylphosphonate, an alkyl phosphotriester, a formacetal, or an analog of formacetal.

In yet another embodiment, the oligonucleotide is an -anomeric oligonucleotide. An -anomeric oligonucleotide forms specific double-stranded hybrids with complementary RNA in which, contrary to the usual -units, the strands run parallel to each other (Gautier et al, 1987, Nucl. Acids Res. 15:6625-6641).

The oligonucleotide may be conjugated to another molecule, e.g., a peptide, hybridization triggered cross-linking agent, transport agent, or hybridization-triggered cleavage agent.

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al, (Nucl. Acids Res. (1988) 16:3209), and methylphosphonate oligonucleotides can be prepared by use of controlled pore glass polymer supports (Sarin et al, Proc. Natl. Acad. Sci. USA (1988) 85:7448-7451).

In a specific embodiment, the SPI antisense nucleic acid of the invention is produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the SPI antisense nucleic acid. Such a vector can remain episomal or become chromosomally integrated, as long as it can be transcribed to produce the desired antisense RNA. Such vectors can be constructed by recombinant DNA technology standard in the art. Vectors can be plasmid, viral, or others known in the art, used for replication and expression in mammalian cells. Expression of the sequence encoding the SPI antisense RNA can be by any promoter known in the art to act in mammalian, preferably human, cells. Such promoters can be inducible or constitutive. Examples of such promoters are outlined above.

The antisense nucleic acids of the invention comprise a sequence complementary to at least a portion of an RNA transcript of a gene encoding an SPI, preferably a human gene encoding an SPI. However, absolute complementarity, although preferred, is not required. A sequence “complementary to at least a portion of an RNA,” as referred to herein, means a sequence having sufficient complementarity to be able to hybridize under stringent conditions (e.g., highly stringent conditions comprising hybridization in 7% sodium dodecyl sulfate (SDS), 1 mM EDTA at 65° C. and washing in 0.1×SSC/0.1% SDS at 68° C., or moderately stringent conditions comprising washing in 0.2×SSC/0.1% SDS at 42° C.) with the RNA, forming a stable duplex; in the case of double-stranded SPI antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA encoding an SPI it may contain and still form a stable duplex (or triplex, as the case may be). One skilled in the art can ascertain a tolerable degree of mismatch by use of standard procedures to determine the melting point of the hybridized complex.

5.14.6 Therapeutic Use of SPI Antisense Nucleic Acids

The SPI antisense nucleic acids can be used to treat or prevent Schizophrenia when the target SPI is overexpressed in the CSF of subjects suspected of having or suffering from Schizophrenia. In a preferred embodiment, a single-stranded DNA antisense SPI oligonucleotide is used.

Cell types which express or overexpress RNA encoding an SPI can be identified by various methods known in the art. Such cell types include but are not limited to leukocytes (e.g., neutrophils, macrophages, monocytes) and resident cells (e.g., astrocytes, glial cells, neuronal cells, and ependymal cells). Such methods include, but are not limited to, hybridization with an SPI-specific nucleic acid (e.g., by Northern hybridization, dot blot hybridization, in situ hybridization), observing the ability of RNA from the cell type to be translated in vitro into an SPI, immunoassay, etc. In a preferred aspect, primary tissue from a subject can be assayed for SPI expression prior to treatment, e.g., by immunocytochemistry or in situ hybridization.

Pharmaceutical compositions of the invention, comprising an effective amount of an SPI antisense nucleic acid in a pharmaceutically acceptable carrier, can be administered to a subject having Schizophrenia.

The amount of SPI antisense nucleic acid which will be effective in the treatment of Schizophrenia can be determined by standard clinical techniques.

In a specific embodiment, pharmaceutical compositions comprising one or more SPI antisense nucleic acids are administered via liposomes, microparticles, or microcapsules. In various embodiments of the invention, such compositions may be used to achieve sustained release of the SPI antisense nucleic acids.

5.14.7 Inhibitory Ribozyme and Triple Helix Approaches

In another embodiment, symptoms of Schizophrenia may be ameliorated by decreasing the level of an SPI or SPI activity by using gene sequences encoding the SPI in conjunction with well-known gene “knock-out,” ribozyme or triple helix methods to decrease gene expression of an SPI. In this approach ribozyme or triple helix molecules are used to modulate the activity, expression or synthesis of the gene encoding the SPI, and thus to ameliorate the symptoms of Schizophrenia. Such molecules may be designed to reduce or inhibit expression of a mutant or non-mutant target gene. Techniques for the production and use of such molecules are well known to those of skill in the art.

Ribozyme molecules designed to catalytically cleave gene mRNA transcripts encoding an SPI can be used to prevent translation of target gene mRNA and, therefore, expression of the gene product. (See, e.g., PCT International Publication WO90/11364, published Oct. 4, 1990; Sarver et al, Science (1990) 247:1222-1225).

Ribozymes are enzymatic RNA molecules capable of catalyzing the specific cleavage of RNA. (For a review, see Rossi, Current Biology (1994) 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

While ribozymes that cleave mRNA at site specific recognition sequences can be used to destroy mRNAs encoding an SPI, the use of hammerhead ribozymes is preferred.

Hammerhead ribozymes cleave mRNAs at locations dictated by flanking regions that form complementary base pairs with the target mRNA. The sole requirement is that the target mRNA have the following sequence of two bases: 5-UG-3. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334, 585-591, each of which is incorporated herein by reference in its entirety.

Preferably the ribozyme is engineered so that the cleavage recognition site is located near the 5 end of the mRNA encoding the SPI, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug, et al, Science, (1984) 224:574-578; Zaug and Cech, Science, (1986) 231, 470-475; Zaug, et al, Nature, (1986) 324, 429-433; published International patent application No. WO 88/04300 by University Patents Inc.; Been and Cech, Cell, (1986) 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the gene encoding the SPI.

As in the antisense approach, the ribozymes can be composed of modified oligonucleotides (e.g., for improved stability, targeting, etc.) and should be delivered to cells that express the SPI in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the SPI and inhibit translation. Because ribozymes, unlike antisense molecules, are catalytic, a lower intracellular concentration is required for efficacy.

Endogenous SPI expression can also be reduced by inactivating or “knocking out” the gene encoding the SPI, or the promoter of such a gene, using targeted homologous recombination (e.g., see Smithies, et al, Nature (1985) 317:230-234; Thomas and Capecchi, Cell (1987) 51:503-512; Thompson et al, Cell (1989) 5:313-321; and Zijlstra et al, Nature (1989) 342:435-438, each of which is incorporated by reference herein in its entirety). For example, a mutant gene encoding a non-functional SPI (or a completely unrelated DNA sequence) flanked by DNA homologous to the endogenous gene (either the coding regions or regulatory regions of the gene encoding the SPI) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, the endogenous expression of a gene encoding an SPI can be reduced by targeting deoxyribonucleotide sequences complementary to the regulatory region of the gene (i.e., the gene promoter and/or enhancers) to form triple helical structures that prevent transcription of the gene encoding the SPI in target cells in the body. (See generally, Helene, 1991, Anticancer Drug Des., 6(6):569-584; Helene, et al, Ann. N.Y. Acad. Sci., (1992) 660:27-36; and Maher, Bioassays (1992) 14(12):807-815).

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5-3, 3-5 manner, such that they base pair with first one strand of a duplex and then the other, eliminating the necessity for a sizeable stretch of either purines or pyrimidines to be present on one strand of a duplex.

In instances wherein the antisense, ribozyme, or triple helix molecules described herein are utilized to inhibit mutant gene expression, it is possible that the technique may so efficiently reduce or inhibit the transcription (triple helix) or translation (antisense, ribozyme) of mRNA produced by normal gene alleles of an SPI that the situation may arise wherein the concentration of SPI present may be lower than is necessary for a normal phenotype. In such cases, to ensure that substantially normal levels of activity of a gene encoding an SPI are maintained, gene therapy may be used to introduce into cells nucleic acid molecules that encode and express the SPI that exhibit normal gene activity and that do not contain sequences susceptible to whatever antisense, ribozyme, or triple helix treatments are being utilized. Alternatively, in instances whereby the gene encodes an extracellular protein, normal SPI can be co-administered in order to maintain the requisite level of SPI activity.

Antisense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.15 Assays For Therapeutic Or Prophylactic Compounds

The present invention also provides assays for use in drug discovery in order to identify or verify the efficacy of compounds for treatment or prevention of Schizophrenia. Test compounds can be assayed for their ability to restore SF or SPI levels in a subject having Schizophrenia towards levels found in subjects free from Schizophrenia or to produce similar changes in experimental animal models of Schizophrenia. Compounds able to restore SF or SPI levels in a subject having Schizophrenia towards levels found in subjects free from Schizophrenia or to produce similar changes in experimental animal models of Schizophrenia can be used as lead compounds for further drug discovery, or used therapeutically. SF and SPI expression can be assayed by the Preferred Technology, immunoassays, gel electrophoresis followed by visualization, detection of SPI activity, or any other method taught herein or known to those skilled in the art. Such assays can be used to screen candidate drugs, in clinical monitoring or in drug development, where abundance of an SF or SPI can serve as a surrogate marker for clinical disease.

In various specific embodiments, in vitro assays can be carried out with cells representative of cell types involved in a subject's disorder, to determine if a compound has a desired effect upon such cell types.

Compounds for use in therapy can be tested in suitable animal model systems prior to testing in humans, including but not limited to rats, mice, chicken, cows, monkeys, rabbits, etc. For in vivo testing, prior to administration to humans, any animal model system known in the art may be used Examples of animal models of Schizophrenia include, but are not limited to, Phencyclidine treated rodents (Sams- Dodd Rev Neurosci (1999) 10:59-90), an animal model of deficient sensorimotor gating (Swerdlow and Geyer Schizophr Bull (1998) 24(2):285-301), neonatal insult to the hippocampal region (Beauregard and Bachevalier Can J Psychiatry (1996) Sep 41(7):446-56), models based on neonatal excitotoxic hippocampal damage (Lillrank et al, Clin Neurosci (1995) 3(2):98-104), attention deficit models (Feldon et al, J Psychiatr Res 4:345-66) and NMDA deficient rodent models (Mohn et al, Cell 1999, 98, 427-436), animals that show decreased expression of mRNAs for synaptophysin, GAP-43, cholecystokinin, and non-NMDA glutamate receptor subunits (GLU R 1 and 2), particularly in CA 3-4 associated with Schizophrenia (Weinberger Biol Psychiatry (1999) Feb 15 45:4 395-402) can be utilized to test compounds that modulate SF or SPI levels, since the neuropathology exhibited in these models is similar to that of Schizophrenia. It is also apparent to the skilled artisan that, based upon the present disclosure, transgenic animals can be produced with “knock-out” mutations of the gene or genes encoding one or more SPIs. A “knock-out” mutation of a gene is a mutation that causes the mutated gene to not be expressed, or expressed in an aberrant form or at a low level, such that the activity associated with the gene product is nearly or entirely absent. Preferably, the transgenic animal is a mammal, more preferably, the transgenic animal is a mouse.

In one embodiment, test compounds that modulate the expression of an SPI are identified in non-human animals (e.g., mice, rats, monkeys, rabbits, and guinea pigs), preferably non-human animal models for Schizophrenia, expressing the SPI. In accordance with this embodiment, a test compound or a control compound is administered to the animals, and the effect of the test compound on expression of one or more SPIs is determined. A test compound that alters the expression of an SPI (or a plurality of SPIs) can be identified by comparing the level of the selected SPI or SPIs (or mRNA(s) encoding the same) in an animal or group of animals treated with a test compound with the level of the SPI(s) or mRNA(s) in an animal or group of animals treated with a control compound. Techniques known to those of skill in the art can be used to determine the mRNA and protein levels, for example, in situ hybridization. The animals may or may not be sacrificed to assay the effects of a test compound.

In another embodiment, test compounds that modulate the activity of an SPI or a biologically active portion thereof are identified in non-human animals (e.g., mice, rats, monkeys, rabbits, and guinea pigs), preferably non-human animal models for Schizophrenia, expressing the SPI. In accordance with this embodiment, a test compound or a control compound is administered to the animals, and the effect of a test compound on the activity of an SPI is determined. A test compound that alters the activity of an SPI (or a plurality of SPIs) can be identified by assaying animals treated with a control compound and animals treated with the test compound. The activity of the SPI can be assessed by detecting induction of a cellular second messenger of the SPI (e.g., intracellular Ca2+, diacylglycerol, IP3, etc.), detecting catalytic or enzymatic activity of the SPI or binding partner thereof, detecting the induction of a reporter gene (e.g., a regulatory element that is responsive to an SPI of the invention operably linked to a nucleic acid encoding a detectable marker, such as luciferase or green fluorescent protein), or detecting a cellular response (e.g., cellular differentiation or cell proliferation). Techniques known to those of skill in the art can be utilized to detect changes in the activity of an SPI (see, e.g., U.S. Pat. No. 5,401,639, which is incorporated herein by reference).

In yet another embodiment, test compounds that modulate the level or expression of an SPI (or plurality of SPIs) are identified in human subjects having Schizophrenia, preferably those having Schizophrenia and most preferably those having severe Schizophrenia. In accordance with this embodiment, a test compound or a control compound is administered to the human subject, and the effect of a test compound on SPI expression is determined by analyzing the expression of the SPI or the mRNA encoding the same in a biological sample (e.g., CSF, serum, plasma, or urine). A test compound that alters the expression of an SPI can be identified by comparing the level of the SPI or mRNA encoding the same in a subject or group of subjects treated with a control compound to that in a subject or group of subjects treated with a test compound. Alternatively, alterations in the expression of an SPI can be identified by comparing the level of the SPI or mRNA encoding the same in a subject or group of subjects before and after the administration of a test compound. Techniques known to those of skill in the art can be used to obtain the biological sample and analyze the mRNA or protein expression. For example, the Preferred Technology described herein can be used to assess changes in the level of an SPI.

In another embodiment, test compounds that modulate the activity of an SPI (or plurality of SPIs) are identified in human subjects having Schizophrenia, preferably those having Schizophrenia and most preferably those with severe Schizophrenia. In this embodiment, a test compound or a control compound is administered to the human subject, and the effect of a test compound on the activity of an SPI is determined. A test compound that alters the activity of an SPI can be identified by comparing biological samples from subjects treated with a control compound to samples from subjects treated with the test compound. Alternatively, alterations in the activity of an SPI can be identified by comparing the activity of an SPI in a subject or group of subjects before and after the administration of a test compound. The activity of the SPI can be assessed by detecting in a biological sample (e.g., CSF, serum, plasma, or urine) induction of a cellular signal transduction pathway of the SPI (e.g., intracellular Ca2+, diacylglycerol, IP3, etc.), catalytic or enzymatic activity of the SPI or a binding partner thereof, or a cellular response, for example, cellular differentiation, or cell proliferation. Techniques known to those of skill in the art can be used to detect changes in the induction of a second messenger of an SPI or changes in a cellular response. For example, RT-PCR can be used to detect changes in the induction of a cellular second messenger.

In a preferred embodiment, a test compound that changes the level or expression of an SPI towards levels detected in control subjects (e.g., humans free from Schizophrenia) is selected for further testing or therapeutic use. In another preferred embodiment, a test compound that changes the activity of an SPI towards the activity found in control subjects (e.g., humans free from Schizophrenia) is selected for further testing or therapeutic use.

In another embodiment, test compounds that reduce the severity of one or more symptoms associated with Schizophrenia are identified in human subjects having Schizophrenia, preferably subjects having Schizophrenia and most preferably subjects with severe Schizophrenia. In accordance with this embodiment, a test compound or a control compound is administered to the subjects, and the effect of a test compound on one or more symptoms of Schizophrenia is determined. A test compound that reduces one or more symptoms can be identified by comparing the subjects treated with a control compound to the subjects treated with the test compound. Techniques known to physicians familiar with Schizophrenia can be used to determine whether a test compound reduces one or more symptoms associated with Schizophrenia. For example, a test compound that enhances memory or reduces confusion in a subject having Schizophrenia will be beneficial for treating subjects having Schizophrenia.

In a preferred embodiment, a test compound that reduces the severity of one or more symptoms associated with Schizophrenia in a human having Schizophrenia is selected for further testing or therapeutic use.

5.16 Therapeutic and Prophylactic Compositions and Their Use

The invention provides methods of treatment (and prophylaxis) comprising administering to a subject an effective amount of a compound of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In a specific embodiment, a non-human mammal is the subject.

Formulations and methods of administration that can be employed when the compound comprises a nucleic acid are described above; additional appropriate formulations and routes of administration are described below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. (1987) 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, or fibers. In one embodiment, administration can be by direct injection into CSF or at the site (or former site) of neurodegeneration or to CNS tissue.

In another embodiment, the compound can be delivered in a vesicle, in particular a liposome (see Langer, Science (1990) 249:1527-1533; Treat et al, in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton, 1987, CRC Crit. Ref. Biomed. Eng. 14:201; Buchwald et al, 1980, Surgery 88:507; Saudek et al, 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. (1983) 23:61; see also Levy et al, Science (1985) 228:190; During et al, Ann. Neurol. (1989) 25:351; Howard et al, J. Neurosurg. (1989) 71:105 ). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science (1990) 249:1527-1533).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al, Proc. Natl. Acad. Sci. USA (1991) 88:1864-1868), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment of Schizophrenia can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to-the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test systems.

Suppositories generally contain active ingredient in the range of 0.5% to 10% by weight; oral formulations preferably contain 10% to 95% active ingredient.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### 6. EXAMPLE

### Identification of Proteins Differentially Expressed in the CSE in Schizophrenia

Using the following procedure, proteins in CSF samples from 5 subjects having Schizophrenia and 5 control subjects were separated by isoelectric focusing followed by SDS-PAGE and analyzed. Parts 6.1.1 to 6.1.14 (inclusive) of the procedure set forth are hereby designated as the “Reference Protocol”

6.1 Materials and Methods

6.1.1 Sample Preparation

A protein assay (Pierce BCA Cat # 23225) was performed on each CSF sample as received. Prior to protein separation, each sample was processed for selective depletion of certain proteins, in order to enhance and simplify protein separation and facilitate analysis by removing proteins that may interfere with or limit analysis of proteins of interest. See International Patent Application No. PCT/GB99/01742, filed Jun. 1, 1999, which is incorporated by reference in its entirety, with particular reference to pages 3 and 6.

Removal of albumin, haptoglobin, transferrin and immunoglobin G (IgG) from CSF (“CSF depletion”) was achieved by an affinity chromatography purification step in which the sample was passed through a series of ‘Hi-Trap’ columns containing immobilized antibodies for selective removal of albumin, haptoglobin and transferrin, and protein G for selective removal of immunoglobin G. Two affinity columns in a tandem assembly were prepared by coupling antibodies to protein G-sepharose contained in Hi-Trap columns (Protein G-Sepharose Hi-Trap columns (1 ml) Pharmacia Cat. No. 17-0404-01). -This was done by circulating the following solutions sequentially through the columns: (1) Dulbecco's Phosphate Buffered Saline (Gibco BRL Cat. No. 14190-094); (2) concentrated antibody solution; (3) 200 mM sodium carbonate buffer, pH 8.35; (4) cross-linking solution (200 mM sodium carbonate buffer, pH 8.35, 20 mM dimethylpimelimidate); and (5) 500 mM ethanolamine, 500 mM NaCl. A third (un-derivatised) protein G Hi-Trap column was then attached to the lower end of the tandem column assembly.

The chromatographic procedure was automated using an Akta Fast Protein Liquid Chromatography (FPLC) System such that a series of up to seven runs could be performed sequentially. The samples were passed through the series of 3 Hi-Trap columns in which the affinity chromatography media selectively bind the above proteins thereby removing them from the sample. Fractions (typically 3 ml per tube) were collected of unbound material (“Flowthrough fractions”) that eluted through the column during column loading and washing stages and of bound proteins (“Bound/Eluted fractions”) that were eluted by step elution with Immunopure Gentle Ag/Ab Elution Buffer (Pierce Cat. No. 21013). The eluate containing unbound material was collected in fractions which were pooled, desalted/concentrated by centrifugal ultrafiltration and stored to await further analysis by 2D PAGE.

A volume of depleted CSF containing approximately 100-150 g of total protein was aliquoted and an equal volume of 10% (w/v) SDS (Fluka 71729), 2.3% (w/v) dithiothreitol (BDH 443852A) was added. The sample was heated at 95° C. for 5 mins, and then allowed to cool to 20° C. 125 1 of the following buffer was then added to the sample:

8M urea (BDH 452043w)

4% CHAPS (Sigma C3023)

65 mM dithiotheitol (DTT)

2% (v/v) Resolytes 3.5-10 (BDH 44338 2×) This mixture was vortexed, and centrifuged at 13000 rpm for 5 mins at 15° C., and the supernatant was analyzed by isoelectric focusing.

6.1.2 Isoelectric Focusing

Isoelectric focusing (IEF), was performed using the Immobiline® DryStrip Kit (Pharmacia BioTech), following the procedure described in the manufacturer's instructions, see Instructions for Immobiline® DryStrip Kit, Pharmacia, # 18-1038-63, Edition AB (incorporated herein by reference in its entirety). Immobilized pH Gradient (IPG) strips (18 cm, pH 3-10 non-linear strips; Pharmacia Cat. # 17-1235-01) were rehydrated overnight at 20° C. in a solution of 8M urea, 2% (w/v) CHAPS, 10 mM DTT, 2% (v/v) Resolytes 3.5-10, as described in the Immobiline DryStrip Users Manual. For IEF, 50 μl of supernatant (prepared as above) was loaded onto a strip, with the cup-loading units being placed at the basic. end of the strip. The loaded gels were then covered with mineral oil (Pharmacia 17-3335-01) and a voltage was immediately applied to the strips according to the following profile, using a Pharmacia EPS3500XL power supply (Cat 19-3500-01):

Initial voltage=300V for 2 hrs

Linear Ramp from 300V to 3500V over 3 hrs

Hold at 3500V for 19 hrs For all stages of the process, the current limit was set to

10 mA for 12 gels, and the wattage limit to 5 W. The temperature was held at 20° C. throughout the run.

6.1.3 Gel Equilibration and SDS-PAGE

After the final 19 hr step, the strips were immediately removed and immersed for 10 mins at 20° C. in a first solution of the following composition: 6M urea; 2% (w/v) DTT; 2% (w/v) SDS; 30% (v/v) glycerol (Fluka 49767); 0.05M Tris/HCl, pH 6.8 (Sigma Cat T-1503). The strips were removed from the first solution and immersed for 10 mins at 20° C. in a second solution of the following composition: 6M urea; 2% (w/v) iodoacetamide (Sigma I-6125); 2% (w/v) SDS; 30% (v/v) glycerol; 0.05M Tris/HCl, pH 6.8. After removal from the second solution, the strips were loaded onto supported gels for SDS-PAGE according to Hochstrasser et al, 1988, Analytical Biochemistry 173: 412-423 (incorporated herein by reference in its entirety), with modifications as specified below.

6.1.4 Preparation of supported gels

The gels were cast between two glass plates of the following dimensions: 23 cm wide×24 cm long (back plate); 23 cm wide×24 cm long with a 2 cm deep notch in the central 19 cm (front plate). To promote covalent attachment of SDS-PAGE gels, the back plate was treated with a 0.4% solution of γ-methacryl-oxypropyltrimethoxysilane in ethanol (BindSilane™; Pharmacia Cat. # 17-1330-01). The front plate was treated with (RepelSilane™ Pharmacia Cat. # 17-1332-01) to reduce adhesion of the gel. Excess reagent was removed by washing with water, and the plates were allowed to dry. At this stage, both as identification for the gel, and as a marker to identify the coated face of the plate, an adhesive bar-code was attached to the back plate in a position such that it would not come into contact with the gel matrix.

The dried plates were assembled into a casting box with a capacity of 13 gel sandwiches. The top and bottom plates of each sandwich were spaced by means of 1 mm thick spacers, 2.5 cm wide. The sandwiches were interleaved with acetate sheets to facilitate separation of the sandwiches after gel polymerization. Casting was then carried out according to Hochstrasser et al, op. cit.

A 9-16% linear polyacrylamide gradient was cast, extending up to a point 2 cm below the level of the notch in the front plate, using the Angelique gradient casting system (Large Scale Biology). Stock solutions were as follows. Acrylamide (40% in water) was from Serva (Cat. # 10677). The cross-linking agent was PDA (BioRad 161-0202), at a concentration of 2.6% (w/w) of the total starting monomer content. The gel buffer was 0.375M Tris/HCl, pH 8.8. The polymerization catalyst was 0.05% (v/v) TEMED (BioRad 161-0801), and the initiator was 0.1% (w/v) APS (BioRad 161-0700). No SDS was included in the gel and no stacking gel was used. The cast gels were allowed to polymerize at 20° C. overnight, and then stored at 4° C. in sealed polyethylene bags with 6 ml of gel buffer, and were used within 4 weeks.

6.1.5 SDS-PAGE

A solution of 0.5% (w/v) agarose (Fluka Cat 05075) was prepared in running buffer (0.025M Tris, 0.198M glycine (Fluka 50050), 1% (w/v) SDS, supplemented by a trace of bromophenol blue). The agarose suspension was heated to 70° C. with stirring, until the agarose had dissolved. The top of the supported 2nd D gel was filled with the agarose solution, and the equilibrated strip was placed into the agarose, and tapped gently with a palette knife until the gel was intimately in contact with the 2nd D gel. The gels were placed in the 2nd D running tank, as described by Amess et al, 1995, Electrophoresis 16:1255-1267 (incorporated herein by reference in its entirety). The tank was filled with running buffer (as above) until the level of the buffer was just higher than the top of the region of the 2nd D gels which contained polyacrylamide, so as to achieve efficient cooling of the active gel area. Running buffer was added to the top buffer compartments formed by the gels, and then voltage was applied immediately to the gels using a Consort E-833 power supply. For 1 hour, the gels were run at 20 mA/gel. The wattage limit was set to 150W for a tank containing 6 gels, and the voltage limit was set to 600V. After 1 hour, the gels were then run at 40 mA/gel, with the same voltage and wattage limits as before, until the bromophenol blue line was 0.5 cm from the bottom of the gel. The temperature of the buffer was held at 16° C. throughout the run. Gels were not run in duplicate.

6.1.6 Staining

Upon completion of the electrophoresis run, the gels were immediately removed from the tank for fixation. The top plate of the gel cassette was carefully removed, leaving the gel bonded to the bottom plate. The bottom plate with its attached gel was then placed into a staining apparatus, which can accommodate 12 gels. The gels were completely immersed in fixative solution of 40% (v/v) ethanol (BDH 28719), 10% (v/v) acetic acid (BDH 100016X), 50% (v/v) water (MilliQ-Millipore), which was continuously circulated over the gels. After an overnight incubation, the fixative was drained from the tank, and the gels were primed by immersion in 7.5% (v/v) acetic acid, 0.05% (w/v) SDS, 92.5% (v/v) water for 30 mins. The priming solution was then drained, and the gels were stained by complete immersion for 4 hours in a staining solution of Pyridinium, 4-[2-[4-(dipentylamino)-2-trifluoromethylphenyl] ethenyl]-1-(sulfobutyl)-, inner salt, prepared by diluting a stock solution of this dye (2 mg/ml in DMSO) in 7.5% (v/v) aqueous acetic acid to give a final concentration of 1.2 mg/l; the staining solution was vacuum filtered through a 0.4 m filter (Duropore) before use.

6.1.7 Imaging of the gel

A computer-readable output was produced by imaging the fluorescently stained gels with the Apollo 2 scanner (Oxford Glycosciences, Oxford, UK) described in section 5.1, supra. This scanner has a gel carrier with four integral fluorescent markers (Designated M1, M2, M3, M4) that are used to correct the image geometry and are a quality control feature to confirm that the scanning has been performed correctly.

For scanning, the gels were removed from the stain, rinsed with water and allowed to air dry briefly, and imaged on the Apollo 2. After imaging, the gels were sealed in polyethylene bags containing a small volume of staining solution, and then stored at 4° C.

6.1.8 Digital Analysis of the Data

The data were processed as described in U.S. application Ser. No. 08/980,574, (published as WO 98/23950) at Sections 5.4 and 5.5 (incorporated herein by reference), as set forth more particularly below.

The output from the scanner was first processed using the MELANIE® II 2D PAGE analysis program (Release 2.2, 1997, BioRad Laboratories, Hercules, Calif., Cat. # 170-7566) to autodetect the registration points, M1, M2, M3 and M4; to autocrop the images (i.e., to eliminate signals originating from areas of the scanned image lying outside the boundaries of the gel, e.g. the reference frame); to filter out artifacts due to dust; to detect and quantify features; and to create image files in GIF format. Features were detected using the following parameters:

Smooths=2

Laplacian threshold 50

Partials threshold 1

Saturation=100

Peakedness=0

Minimum Perimeter=10

6.1.9 Assignment of pI and MW Values

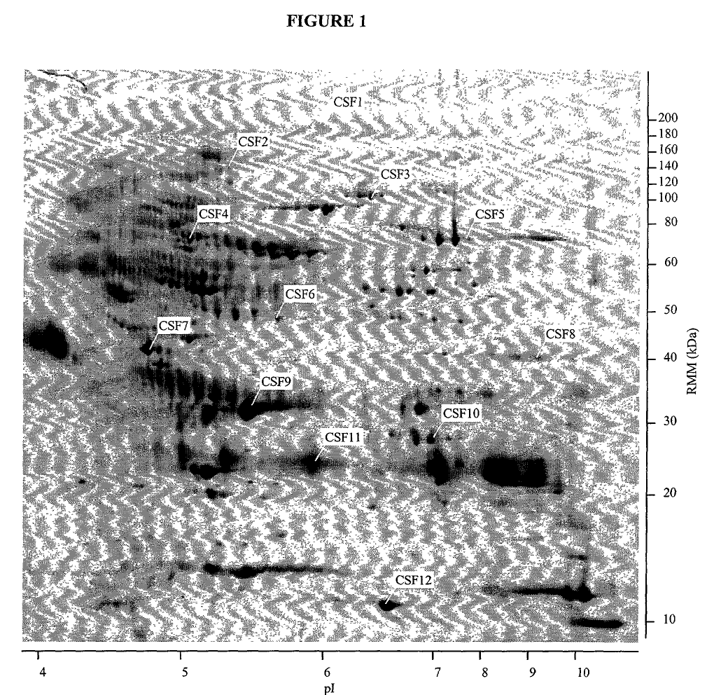

Landmark identification was used to determine the pI and MW of features detected in the images. Twelve landmark features, designated CSF1 to CSF12, were identified in a standard CSF image obtained from a pooled sample. These landmark features are identified in FIG. 1 and were assigned the pI and/or MW values identified in Table XV.

| | | MW | | | MW |
|---|---|---|---|---|---|
| Name | pl | (Da) | Name | pl | (Da) |
| CSF1 | 5.96 | 185230 | CSF7 | 4.78 | 41340 |
| CSF2 | 5.39 | 141700 | CSF8 | 9.2 | 40000 |
| CSF3 | 6.29 | 100730 | CSF9 | 5.5 | 31900 |
| CSF4 | 5.06 | 71270 | CSF10 | 6.94 | 27440 |
| CSF5 | 7.68 | 68370 | CSF11 | 5.9 | 23990 |
| CSF6 | 5.67 | 48090 | CSF12 | 6.43 | 10960 |

As many of these landmarks as possible were identified in each gel image of the dataset. Each feature in the study gels was then assigned a pI value by linear interpolation or extrapolation (using the MELANIE®-II software) to the two nearest landmarks, and was assigned a MW value by linear interpolation or extrapolation (using the MELANIE®-II software) to the two nearest landmarks.

6.1.10 Matching With Primary Master Image

Images were edited to remove gross artifacts such as dust, to reject images which had gross abnormalities such as smearing of protein features, or were of too low a loading or overall image intensity to allow identification of more than the most intense features, or were of too poor a resolution to allow accurate detection of features. Images were then compared by pairing with one common image from the whole sample set. This common image, the “primary master image”, was selected on the basis of protein load (maximum load consistent with maximum feature detection), a well resolved myoglobin region, (myoglobin was used as an internal standard), and general image quality. Additionally, the primary master image was chosen to be an image which appeared to be generally representative of all those to be included in the analysis. (This process by which a primary master gel was judged to be

We claim:

1. A method for screening, diagnosis or prognosis of Schizophrenia in a subject, for determining the stage or severity of Schizophrenia in a subject, for identifying a subject at risk of developing Schizophrenia, or for monitoring the effect of therapy administered to a subject having Schizophrenia, said method comprising:
(a) analyzing a test sample of body fluid from the subject by two dimensional electrophoresis to generate a two-dimensional array of features, said array comprising at least one chosen feature whose relative abundance correlates with the presence, absence, stage or severity of Schizophrenia or predicts the onset or course of Schizophrenia; and
(b) comparing the abundance of each chosen feature in the test sample with the abundance of that chosen feature in body fluid from one or more persons free from Schizophrenia, or with a previously determined reference range for that feature in subjects free from Schizophrenia, or with the abundance at least one Expression Reference Feature (ERF) in the test sample.

2. The method of claim 1, wherein the body fluid is cerebrospinal fluid (CSF).

3. The method of claim 1 or claim 2, wherein said method is for screening or diagnosis of Schizophrenia and the relative abundance of at least one chosen feature correlates with the presence or absence of Schizophrenia.

4. The method of claim 1 or claim 2, wherein said method is for monitoring the effect of therapy administered to a subject having Schizophrenia and the relative abundance of at least one chosen feature correlates with the severity of Schizophrenia.

5. The method of claim 2, wherein step (b) comprises comparing the abundance of each chosen feature in the sample with the abundance of that chosen feature in CSF from one or more persons free from Schizophrenia or with a previously determined reference range for that chosen feature in subjects free from Schizophrenia.

6. The method of claim 1 or claim 2, wherein step (b) comprises detecting one or more of the following Schizophrenia-Associated Features (SFs):
SF-14, SF-16, SF-17, SF-18, SF-19, SF-20, SF-21, SF-22, SF-23, SF-24, SF-25, SF-26, SF-27, SF-28, SF-29, SF-30, SF-31, SF-32, SF-33, SF-34, SF-35, SF-36, SF-37, SF-38, SF-39, SF-40, SF-41, SF-42, SPI-43, SF-44, SF-45, SF-46, SF-47, SF-48, SF-49, SF-51, SF-52, SF-53, SF-55, SF-56, SF-57, SF-58, SF-80, SF-81, SF-82, SF-83, SF-84, SF-85, SF-86, SF-87, SF-88, SF-89, SF-90, SF-91, SF-92, SF-93, SF-94, SF-95, SF-96, SF-97, SF-98, SF-99, SF-100, SF-101, SF-102, SF-103, SF-104, SF-105, SF-106, SF-107, SF-108, SF-109, SF-110, SF-111, SF-112, SF-113, SF-114, SF-115, SF-116, SF-117, SF-118, SF-119, SF-120, SF-123, SF-124, SF-125, SF-126, SF-130, SF-131, SF-132, SF-135, SF-136, SF-137, SF-139, SF-141, SF-142, SF-143, SF-144, SF-147, SF-148, SF-150, SF-151, SF-153, SF-154, SF-155, SF-157, SF-158, SF-159, SF-160, SF-161, SF-162, SF-163, SF-164, SF-165, SF-166, SF-167, SF-168, SF-169, SF-170, SF-171, SF-172, SF-173, SF-174, SF-175, SF-176, SF-177, SF-178, SF-179, SF-180, SF-181, SF-182, SF-183, SF-184, SF-186, SF-187, SF-188, SF-189, SF-190, SF-191, SF-194, SF-195, SF-196, SF-197, SF-198, SF-199, SF-200, SF-201, SF-202, SF-203, SF-204, SF-208, SF-209, SF-210, SF-212, SF-213, SF-215, SF-216, SF-217, SF-218, SF-219, SF-220, SF-221, SF-222, SF-223, SF-227, SF-228, SF-229, SF-230, SF-231, SF-232, SF-233, SF-235, SF-237, SF-238, SF-239, SF-242, SF-243, SF-244, SF-248, SF-249, SF-250, SF-255, SF-257, SF-258, SF-261, SF-262, SF-264, SF-265, SF-267, SF-268, SF-269, SF-271, SF-272, SF-273, SF-280, SF-282, SF-283, SF-286, SF-289, SF-291, SF-292, SF-293, SF-294, SF-296, SF-300, SF-301, SF-302, SF-303, SF-304, SF-306, SF-307, SF-309, SF-312, SF-317, SF-320, SF-321, SF-322, SF-324, SF-326, SF-327, SF-332, SF-333, SF-336, SF-340, SF-348, SF-349, SF-352, SF-358, SF-368, SF-369, SF-370, SF-371, SF-372, SF-373, SF-374, SF-375, SF-376, SF-377, SF-378, SF-379, SF-380, SF-381, SF-382, SF-383, SF-384, SF-385, SF-386, SF-387, SF-388, SF-389, SF-390, SF-391, SF-392, SF-393, SF-394, SF-395, SF-396, SF-397, SF-398, SF-399, SF400, SF401, SF402, SF403, SF-404, SF405, SF406, SF407, SF408, SF409, SF410, SF411, SF412, SF413, SF414, SF415, SF416, SF417, SF418, SF419, SF420, SF421, SF422, SF423, SF424, SF-425, SF-426, SF427, SF428, SF429, SF430, SF431, SF432, SF433, SF434, SF435, SF436, SF437, SF438, SF439, SF440, SF441, SF442, SF443, SF444, SF445, SF446, SF447, SF448, SF449, SF450, SF451, SF452, SF453, SF454, SF455, SF456, SF457, SF-458, SF-459, SF-460, SF461, SF462, SF463, SF464, SF465, SF466, SF467, SF468, SF469, SF-470, SF471, SF472, SF473, SF474, SF475, SF476, SF477, SF478, SF479, SF480, SF481, SF482, SF483, SF484, SF485, SF486, SF487, SF488, SF489, SF490, SF-491, SF492, SF493, SF494, SF495, SF496, SF497, SF-498, SF-499, SF-500, SF-501, or SF-502.

7. The method according to claim 1, 2, or 5, wherein step (a) comprises isoelectric focussing followed by sodium dodecyl sulfate polyacrylamide gel electrophoresis (SDS-PAGE).

8. A method for screening, diagnosis or prognosis of Schizophrenia in a subject, for determining the stage or severity of Schizophrenia in a subject, for identifying a subject at risk of developing Schizophrenia, or for monitoring the effect of therapy administered to a subject having Schizophrenia, said method comprising detecting, in a sample of cerebrospinal fluid from the subject, at least one of the following Schizophrenia-Associated Protein Isoforms (SPIs):
SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SP-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-43 1, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462 SPI-463, SPI-464, SPI-465, SPI-466, or SPI-467.

9. The method according to claim 8, wherein the step of detecting comprises testing at least one aliquot of the sample, said step of testing comprising:
(a) contacting the aliquot with an antibody that is immunospecific for a preselected SPI; and
(b) measuring any binding that has occurred between the antibody and at least one species in the aliquot.

10. The method according to claim 9, wherein the antibody is a monoclonal antibody.

11. The method according to claim 9, wherein the step of detecting comprises testing a plurality of aliquots with a plurality of antibodies for detection of a plurality of preselected SPIs.

12. The method according to claim 11, wherein the antibodies are monoclonal antibodies.

13. A preparation comprising one of the following isolated Schizophrenia-Associated Protein Isoforms (SPIs):
SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-99,
SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467.

14. A kit comprising the preparation of claim 13.

15. A kit comprising a plurality of distinct preparations of claim 14.

16. An antibody capable of immunospecific binding to one of the following Schizophrenia-Associated Protein Isoforms (SPIs):
SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-211, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-304, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467.

17. The antibody of claim 16, which is a monoclonal antibody.

18. The antibody of claim 16 or 17, which binds to the SPI with greater affinity than to another isoform of the SPI.

19. The antibody of claim 16, which binds to the SPI with greater affinity than to any other isoform of the SPI.

20. A kit comprising the antibody of claim 16.

21. A kit comprising a plurality of distinct antibodies of claim 16.

22. A pharmaceutical composition comprising a therapeutically effective amount of an antibody of claim 16 and a pharmaceutically acceptable carrier.

23. A pharmaceutical composition comprising: a therapeutically effective amount of a fragment or derivative of an antibody of claim 16, said fragment or derivative containing the binding domain of the antibody; and a pharmaceutically acceptable carrier.

24. A method of treating or preventing Schizophrenia comprising administering to a subject in need of such treatment or prevention a therapeutically effective amount of a nucleic acid encoding one of the following Schizophrenia- Associated Protein Isoforms (SPIs):
SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-41 1, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467.

25. A method of treating or preventing Schizophrenia comprising administering to a subject in need of such treatment or prevention a therapeutically effective amount of a nucleic acid that inhibits the function of one or more of the following Schizophrenia-Associated Protein Isoforms (SPIs):
SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-24, SPI-25, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467.

26. The method of claim 25, wherein the nucleic acid is an SPI antisense nucleic acid or ribozyme.

27. A method of screening for agents that interact with an SPI, an SPI fragment, or an SPI-related polypeptide, said method comprising:
(a) contacting an SPI, a biologically active portion of an SPI, or an SPI-related polypeptide with a candidate agent; and
(b) determining whether or not the candidate agent interacts with the SPI, the SPI fragment, or the SPI-related polypeptide.

28. The method of claim 27, wherein the SPI, the SPI fragment, or the SPI-related polypeptide is expressed by cells.

29. The method of claim 27, wherein the cells express a recombinant SPI, a recombinant SPI fragment, or a recombinant SPI-related polypeptide.

30. A method of screening for agents that modulate the expression or activity of an SPI or an SPI-related polypeptide comprising:
(a) contacting a first population of cells expressing an SPI or an SPI-related polypeptide with a candidate agent;
(b) contacting a second population of cells expressing said SPI or said SPI-related polypeptide with a control agent; and
(c) comparing the level of said SPI or said SPI-related polypeptide or mRNA encoding said SPI or said SPI-related polypeptide in the first and second populations of cells, or comparing the level of induction of a cellular second messenger in the first and second populations of cells.

31. The method of claim 30, wherein the level of said SPI or said SPI-related polypeptide, mRNA encoding said SPI or said SPI-related polypeptide, or said cellular second messenger is greater in the first population of cells than in the second population of cells.

32. The method of claim 30, wherein the level of said SPI or said SPI-related polypeptide, mRNA encoding said SPI or said SPI-related polypeptide, or said cellular second messenger is less in the first population of cells than in the second population of cells.

33. A method of screening for or identifying agents that modulate the expression or activity of an SPI or an SPI-related polypeptide comprising:
(a) administering a candidate agent to a first mammal or group of mammals;
(b) administering a control agent to a second mammal or group of mammals; and
(c) comparing the level of expression of the SPI or the SPI-related polypeptide or of mRNA encoding the SPI or the SPI-related polypeptide in the first and second groups, or comparing the level of induction of a cellular second messenger in the first and second groups.

34. The method of claim 33, wherein the mammals are animal models for Schizophrenia.

35. The method of claim 33 or 34, wherein the level of expression of said SPI or said SPI-related polypeptide, mRNA encoding said SPI or said SPI-related polypeptide, or of said cellular second messenger is greater in the first group than in the second group.

36. The method of claim 33 or 34, wherein the level of expression of said SPI or said SPI-related polypeptide, mRNA encoding said SPI or said SPI-related polypeptide, or of said cellular second messenger is less than in the first group than in the second group.

37. The method of claim 33, wherein the levels of said SPI or said SPI-related polypeptide, mRNA encoding said SPI or said SPI-related polypeptide, or of said cellular second messenger in the first and second groups are further compared to the level of said SPI or said SPI-related polypeptide or said mRNA encoding said SPI or said SPI-related polypeptide in normal control mammals.

38. The method of claim 33, wherein administration of said candidate agent modulates the level of said SPI or said SPI-related polypeptide, or said mRNA encoding said SPI or said SPI-related polypeptide, or said cellular second messenger in the first group towards the levels of said SPI or said SPI-related polypeptide or said mRNA or said cellular second messenger in the second group.

39. The method of claim 33, wherein said mammals are human subjects having Schizophrenia.

40. A method of screening for or identifying agents that interact with an SPI or an SPI-related polypeptide, comprising
(a) contacting a candidate agent with the SPI or the SPI-related polypeptide, and
(b) detecting binding, if any, between the agent and the SPI or the SPI-related polypeptide.

41. A method of screening for or identifying agents that modulate the activity of an SPI or an SPI-related polypeptide, comprising:
(a) in a first aliquot, contacting a candidate agent with the SPI or the SPI-related polypeptide, and
(b) comparing the activity of the SPI or the SPI-related polypeptide in the first aliquot after addition of the candidate agent with the activity of the SPI or the SPI-related polypeptide in a control aliquot, or with a previously determined reference range.

42. The method according to claim 40 or 41, wherein the SPI or the SPI-related polypeptide is recombinant protein.

43. The method according to claim 40 or 41, wherein the SPI or the SPI-related polypeptide is immobilized on a solid phase.

44. A method for screening, diagnosis or prognosis of Schizophrenia in a subject or for monitoring the effect of an anti-Schizophrenia drug or therapy administered to a subject, comprising:
(a) contacting at least one oligonucleotide probe comprising 10 or more consecutive nucleotides complementary to a nucleotide sequence encoding an SPI chosen from SPI-6, SPI-7, SPI-8, SPI-9, SPI-10, SPI-l1, SPI-13, SPI-15, SPI-16, SPI-17, SP-18, SPI-19, SPI-20, SPI-21, SPI-23, SPI-24, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-21 1, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-306, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467 with an RNA obtained from a biological sample from the subject or with cDNA copied from the RNA wherein said contacting occurs under conditions that permit hybridization of the probe to the nucleotide sequence if present;
(b) detecting hybridization, if any, between the probe and the nucleotide sequence; and
(c) comparing the hybridization, if any, detected in step (b) with the hybridization detected in a control sample, or with a previously determined reference range.

45. The method of claim 44, wherein step (a) comprises contacting a plurality of oligonucleotide probes comprising 10 or more consecutive nucleotides complementary to a nucleotide sequence encoding an SPI chosen from SPI-6, SPI-7, SPI-8, SPI-9, SPI-10, SPI-11, SPI-13, SPI-15, SPI-16, SPI-17, SPI-18, SPI-19, SPI-20, SPI-21, SPI-23, SPI-25, SPI-26, SPI-28, SPI-29, SPI-30, SPI-32, SPI-33, SPI-34, SPI-35, SPI-36, SPI-38, SPI-39, SPI-41, SPI-42, SPI-43, SPI-44, SPI-54, SPI-56, SPI-57, SPI-58, SPI-59, SPI-60, SPI-62, SPI-63, SPI-65, SPI-67, SPI-69, SPI-73, SPI-74, SPI-75, SPI-76, SPI-77, SPI-78, SPI-80, SPI-81, SPI-82, SPI-83, SPI-85, SPI-87, SPI-88, SPI-91, SPI-92, SPI-93, SPI-95, SPI-96, SPI-97, SPI-99, SPI-100, SPI-101, SPI-105, SPI-107, SPI-113, SPI-114, SPI-115, SPI-118, SPI-122, SPI-123, SPI-124, SPI-127, SPI-129, SPI-130, SPI-132, SPI-133, SPI-138, SPI-139, SPI-141, SPI-142, SPI-143, SPI-151, SPI-152, SPI-154, SPI-155, SPI-164, SPI-166, SPI-167, SPI-169, SPI-170, SPI-175, SPI-176, SPI-179, SPI-181, SPI-182, SPI-183, SPI-184, SPI-185, SPI-189, SPI-190, SPI-193, SPI-195, SPI-200, SPI-205, SPI-206, SPI-211, SPI-213, SPI-214, SPI-231, SPI-232, SPI-233, SPI-234, SPI-235, SPI-236, SPI-237, SPI-238, SPI-239, SPI-240, SPI-241, SPI-242, SPI-243, SPI-244, SPI-245, SPI-246, SPI-247, SPI-248, SPI-249, SPI-250, SPI-251, SPI-252, SPI-253, SPI-254, SPI-255, SPI-257, SPI-258, SPI-259, SPI-261, SPI-262, SPI-263, SPI-264, SPI-265, SPI-266, SPI-267, SPI-268, SPI-269, SPI-270, SPI-273, SPI-274, SPI-275, SPI-278, SPI-281, SPI-285, SPI-289, SPI-290, SPI-296, SPI-302, SPI-303, SPI-304, SPI-312, SPI-321, SPI-322, SPI-323, SPI-324, SPI-325, SPI-326, SPI-327, SPI-328, SPI-329, SPI-331, SPI-332, SPI-334, SPI-335, SPI-336, SPI-337, SPI-338, SPI-339, SPI-340, SPI-341, SPI-342, SPI-343, SPI-344, SPI-345, SPI-346, SPI-348, SPI-349, SPI-352, SPI-353, SPI-354, SPI-355, SPI-357, SPI-359, SPI-360, SPI-361, SPI-362, SPI-363, SPI-365, SPI-367, SPI-369, SPI-370, SPI-372, SPI-375, SPI-376, SPI-379, SPI-381, SPI-382, SPI-383, SPI-384, SPI-385, SPI-387, SPI-388, SPI-389, SPI-390, SPI-391, SPI-392, SPI-393, SPI-394, SPI-397, SPI-398, SPI-399, SPI-400, SPI-401, SPI-402, SPI-403, SPI-404, SPI-405, SPI-406, SPI-407, SPI-408, SPI-409, SPI-410, SPI-411, SPI-412, SPI-413, SPI-414, SPI-415, SPI-416, SPI-417, SPI-418, SPI-419, SPI-420, SPI-421, SPI-422, SPI-423, SPI-424, SPI-425, SPI-426, SPI-427, SPI-428, SPI-429, SPI-430, SPI-431, SPI-432, SPI-433, SPI-434, SPI-435, SPI-436, SPI-437, SPI-438, SPI-439, SPI-440, SPI-441, SPI-442, SPI-443, SPI-444, SPI-445, SPI-446, SPI-447, SPI-448, SPI-449, SPI-450, SPI-451, SPI-452, SPI-453, SPI-454, SPI-455, SPI-456, SPI-457, SPI-458, SPI-459, SPI-460, SPI-461, SPI-462, SPI-463, SPI-464, SPI-465, SPI-466, or SPI-467 with an RNA obtained from a biological sample from the subject or with cDNA copied from the RNA wherein said contacting occurs under conditions that permit hybridization of the probe to the nucleotide sequence if present.

46. The method of claim 44, wherein step (a) includes the step of hybridizing the nucleotide sequence to a DNA array, wherein one or more members of the array are the probes complementary to a plurality of nucleotide sequences encoding distinct SPIs.

47. A method of screening for agents effective for the treatment of Schizophrenia comprising:
(a) contacting NP-1 with a first population of cells expressing a receptor for NP-1 (hNPR) in the presence of a candidate agent;
(b) contacting NP-1 with a second population of cells expressing said receptor (hNPR) in the presence of a control agent;
(c) comparing the binding of said NP-1 to the first and second populations of cells, or comparing the level of induction of a cellular second messenger in the first and second populations of cells, or comparing the level of a NP-1 mediated activity in the first and second population of cells; and
(d) testing for the ability of agents able to modulate the activity of NP-1 to decrease clinical features of Schizophrenia in a Schizophrenia disease model system.

48. The method of claim 47 wherein the NP-1 or the receptor, or both, are isolated and recombinantly produced.

49. The method of claim 47, wherein the cells are glial cells and the NP-1 mediated activity is causing the glial cells to be susceptible to neurotoxin activity.

50. The method of claim 49, wherein the neurotoxin is taipoxin.

51. The method of claim 47, wherein the candidate agent comprises at least 10 consecutive amino acids of NP-1.

52. A method of screening for agents that modulate the binding of NP-1 to a binding partner comprising:
(a) contacting NP-1 with the NP-1 binding partner in the presence of a candidate agent;
(b) contacting NP-1 with a NP-1 binding partner in the presence of a control agent; and
(c) comparing the binding of said NP-1 to the binding partner in step (a) to the binding of said NP-1 to the binding partner in step (b).

53. The method of claim 51 wherein the binding partner is an isolated NP-1 receptor (hNPR).


SEQUENCE LISTING (677 sequences)

SEQ ID NO: 1 - protein, 11 aa, Homo sapiens
AASGTQNNVLR

SEQ ID NO: 2 - protein, 13 aa, Homo sapiens
EQTMSECEAGALR

SEQ ID NO: 3 - protein, 9 aa, Homo sapiens
IPTTFENGR

SEQ ID NO: 4 - protein, 17 aa, Homo sapiens
AQGFTEDTIVFLPQTDK

SEQ ID NO: 5 - protein, 10 aa, Homo sapiens
DQDGEILLPR

SEQ ID NO: 6 - protein, 14 aa, Homo sapiens
SAVEEMEAEEAAAK

SEQ ID NO: 7 - protein, 8 aa, Homo sapiens
QELEDLER

SEQ ID NO: 8 - protein, 15 aa, Homo sapiens
QNLEPLFEQYINNLR

SEQ ID NO: 9 - protein, 16 aa, Homo sapiens
TMLLQPAGSLGSYSYR

SEQ ID NO: 10 - protein, 16 aa, Homo sapiens
LVGGPMDASVEEEGVR

SEQ ID NO: 11 - protein, 14 aa, Homo sapiens
SGFIEEDELGFILK

SEQ ID NO: 12 - protein, 16 aa, Homo sapiens
LVGGPMDASVEEEGVR

SEQ ID NO: 13 - protein, 11 aa, Homo sapiens
ALDFAVGEYNK

SEQ ID NO: 14 - protein, 16 aa, Homo sapiens
LVGGPMDASVEEEGVR

SEQ ID NO: 15 - protein, 11 aa, Homo sapiens
ALDFAVGEYNK

SEQ ID NO: 16 - protein, 16 aa, Homo sapiens
LVGGPMDASVEEEGVR

SEQ ID NO: 17 - protein, 11 aa, Homo sapiens
ALDFAVGEYNK

SEQ ID NO: 18 - protein, 12 aa, Homo sapiens
VESLEQEAANER

SEQ ID NO: 19 - protein, 10 aa, Homo sapiens
QQLVETHMAR

SEQ ID NO: 20 - protein, 10 aa, Homo sapiens
YLELESSGHR

SEQ ID NO: 21 - protein, 14 aa, Homo sapiens
TCPTCNDFHGLVQK

SEQ ID NO: 22 - protein, 9 aa, Homo sapiens
AFLFQDTPR

SEQ ID NO: 23 - protein, 8 aa, Homo sapiens
NNAHGYFK

SEQ ID NO: 24 - protein, 7 aa, Homo sapiens
TYFEGER

SEQ ID NO: 25 - protein, 8 aa, Homo sapiens
LDQCYCER

SEQ ID NO: 26 - protein, 12 aa, Homo sapiens
HNGQIWVLENDR

SEQ ID NO: 27 - protein, 12 aa, Homo sapiens
CVTDPCQADTIR

SEQ ID NO: 28 - protein, 12 aa, Homo sapiens
DTDTGALLFIGK

SEQ ID NO: 29 - protein, 10 aa, Homo sapiens
TVQAVLTVPK

SEQ ID NO: 30 - protein, 9 aa, Homo sapiens
LSYEGEVTK

SEQ ID NO: 31 - protein, 14 aa, Homo sapiens
LAAAVSNFGYDLYR

SEQ ID NO: 32 - protein, 9 aa, Homo sapiens
SSFVAPLEK

SEQ ID NO: 33 - protein, 12 aa, Homo sapiens
TSLEDFYLDEER

SEQ ID NO: 34 - protein, 9 aa, Homo sapiens
VELEDWNGR

SEQ ID NO: 35 - protein, 12 aa, Homo sapiens
VESLEQEAANER

SEQ ID NO: 36 - protein, 12 aa, Homo sapiens
SWFEPLVEDMQR

SEQ ID NO: 37 - protein, 9 aa, Homo sapiens
LGPLVEQGR

SEQ ID NO: 38 - protein, 15 aa, Homo sapiens
GEVQAMLGQSTEELR

SEQ ID NO: 39 - protein, 9 aa, Homo sapiens
LEEQAQQIR

SEQ ID NO: 40 - protein, 15 aa, Homo sapiens
SELEEQLTPVAEETR

SEQ ID NO: 41 - protein, 11 aa, Homo sapiens
ELDESLQVAER

SEQ ID NO: 42 - protein, 12 aa, Homo sapiens
ASSIIDELFQDR

SEQ ID NO: 43 - protein, 10 aa, Homo sapiens
TLLSNLEEAK

SEQ ID NO: 44 - protein, 16 aa, Homo sapiens
TMLLQPAGSLGSYSYR

SEQ ID NO: 45 - protein, 8 aa, Homo sapiens
EPGLQIWR

SEQ ID NO: 46 - protein, 11 aa, Homo sapiens
HVVPNEVVVQR

SEQ ID NO: 47 - protein, 8 aa, Homo sapiens
LCTVATLR

SEQ ID NO: 48 - protein, 12 aa, Homo sapiens
SEDTGLDSVATR

SEQ ID NO: 49 - protein, 12 aa, Homo sapiens
DTDTGALLFIGK

SEQ ID NO: 50 - protein, 13 aa, Homo sapiens
KTSLEDFYLDEER

SEQ ID NO: 51 - protein, 11 aa, Homo sapiens
ELLDTVTAPQK

SEQ ID NO: 52 - protein, 9 aa, Homo sapiens
LSYEGEVTK

SEQ ID NO: 53 - protein, 14 aa, Homo sapiens
LAAAVSNFGYDLYR

SEQ ID NO: 54 - protein, 9 aa, Homo sapiens
SSFVAPLEK

SEQ ID NO: 55 - protein, 13 aa, Homo sapiens
GLEEELQFSLGSK

SEQ ID NO: 56 - protein, 10 aa, Homo sapiens
KPNLQVFLGK

SEQ ID NO: 57 - protein, 12 aa, Homo sapiens
LSELIQPLPLER

SEQ ID NO: 58 - protein, 13 aa, Homo sapiens
GLVSWGNIPCGSK

SEQ ID NO: 59 - protein, 9 aa, Homo sapiens
LVHGGPCDK

SEQ ID NO: 60 - protein, 11 aa, Homo sapiens
EKPGVYTNVCR

SEQ ID NO: 61 - protein, 11 aa, Homo sapiens
ESSQEQSSVVR

SEQ ID NO: 62 - protein, 7 aa, Homo sapiens
YTNWIQK

SEQ ID NO: 63 - protein, 10 aa, Homo sapiens
TVQAVLTVPK

SEQ ID NO: 64 - protein, 9 aa, Homo sapiens
LSYEGEVTK

SEQ ID NO: 65 - protein, 14 aa, Homo sapiens
LAAAVSNFGYDLYR

SEQ ID NO: 66 - protein, 9 aa, Homo sapiens
SSFVAPLEK

SEQ ID NO: 67 - protein, 12 aa, Homo sapiens
TSLEDFYLDEER

SEQ ID NO: 68 - protein, 8 aa, Homo sapiens
EPGLQIWR

SEQ ID NO: 69 - protein, 11 aa, Homo sapiens
HVVPNEVVVQR

SEQ ID NO: 70 - protein, 9 aa, Homo sapiens
YIETDPANR

SEQ ID NO: 71 - protein, 16 aa, Homo sapiens
TALASGGVLDASGDYR

SEQ ID NO: 72 - protein, 14 aa, Homo sapiens
LAAAVSNFGYDLYR

SEQ ID NO: 73 - protein, 12 aa, Homo sapiens
TSLEDFYLDEER

SEQ ID NO: 74 - protein, 15 aa, Homo sapiens
LTIGEGQQHHLGGAK

SEQ ID NO: 75 - protein, 9 aa, Homo sapiens
VELEDWNGR

SEQ ID NO: 76 - protein, 12 aa, Homo sapiens
YLQEIYNSNNQK

SEQ ID NO: 77 - protein, 9 aa, Homo sapiens
RLDGSVDFK

SEQ ID NO: 78 - protein, 16 aa, Homo sapiens
TMLLQPAGSLGSYSYR

SEQ ID NO: 79 - protein, 17 aa, Homo sapiens
AQGFTEDTIVFLPQTDK

SEQ ID NO: 80 - protein, 16 aa, Homo sapiens
APEAQVSVQPNFQQDK

SEQ ID NO: 81 - protein, 12 aa, Homo sapiens
DTDTGALLFIGK

SEQ ID NO: 82 - protein, 9 aa, Homo sapiens
LSYEGEVTK

SEQ ID NO: 83 - protein, 14 aa, Homo sapiens
LAAAVSNFGYDLYR

SEQ ID NO: 84 - protein, 9 aa, Homo sapiens
SSFVAPLEK

SEQ ID NO: 85 - protein, 12 aa, Homo sapiens
TSLEDFYLDEER

SEQ ID NO: 86 - protein, 9 aa, Homo sapiens
EPGEFALLR

SEQ ID NO: 87 - protein, 16 aa, Homo sapiens
TALASGGVLDASGDYR

SEQ ID NO: 88 - protein, 8 aa, Homo sapiens
FYYIYNEK

SEQ ID NO: 89 - protein, 16 aa, Homo sapiens
SGIPIVTSPYQIHFTK

SEQ ID NO: 90 - protein, 14 aa, Homo sapiens
LVAYYTLIGASGQR

SEQ ID NO: 91 - protein, 12 aa, Homo sapiens
TIYTPGSTVLYR

SEQ ID NO: 92 - protein, 14 aa, Homo sapiens
IPIEDGSGEVVLSR

SEQ ID NO: 93 - protein, 10 aa, Homo sapiens
DFDFVPPVVR

SEQ ID NO: 94 - protein, 16 aa, Homo sapiens
DICEEQVNSLPGSITK

SEQ ID NO: 95 - protein, 9 aa, Homo sapiens
GYTQQLAFR

SEQ ID NO: 96 - protein, 9 aa, Homo sapiens
RQGALELIK

SEQ ID NO: 97 - protein, 14 aa, Homo sapiens
AGDFLEANYMNLQR

SEQ ID NO: 98 - protein, 10 aa, Homo sapiens
KGYTQQLAFR

SEQ ID NO: 99 - protein, 11 aa, Homo sapiens
ELDESLQVAER

SEQ ID NO: 100 - protein, 12 aa, Homo sapiens
ASSIIDELFQDR

SEQ ID NO: 101 - protein, 16 aa, Homo sapiens
EILSVDCSTNNPSQAK

SEQ ID NO: 102 - protein, 12 aa, Homo sapiens
SWFEPLVEDMQR

SEQ ID NO: 103 - protein, 11 aa, Homo sapiens
LGADMEDVCGR

SEQ ID NO: 104 - protein, 8 aa, Homo sapiens
QWAGLVEK

SEQ ID NO: 105 - protein, 9 aa, Homo sapiens
LGPLVEQGR

SEQ ID NO: 106 - protein, 15 aa, Homo sapiens
GEVQAMLGQSTEELR

SEQ ID NO: 107 - protein, 9 aa, Homo sapiens
LEEQAQQIR

SEQ ID NO: 108 - protein, 15 aa, Homo sapiens
SELEEQLTPVAEETR

SEQ ID NO: 109 - protein, 15 aa, Homo sapiens
AATVGSLAGQPLQER

SEQ ID NO: 110 - protein, 16 aa, Homo sapiens
TMLLQPAGSLGSYSYR

SEQ ID NO: 111 - protein, 17 aa, Homo sapiens
AQGFTEDTIVFLPQTDK

SEQ ID NO: 112 - protein, 16 aa, Homo sapiens
APEAQVSVQPNFQQDK

SEQ ID NO: 113 - protein, 16 aa, Homo sapiens
TMLLQPAGSLGSYSYR

SEQ ID NO: 114 - protein, 17 aa, Homo sapiens
AQGFTEDTIVFLPQTDK

SEQ ID NO: 115 - protein, 16 aa, Homo sapiens
APEAQVSVQPNFQQDK

SEQ ID NO: 116 - protein, 9 aa, Homo sapiens
EPGEFALLR

SEQ ID NO: 117 - protein, 16 aa, Homo sapiens
TALASGGVLDASGDYR

SEQ ID NO: 118 - protein, 9 aa, Homo sapiens
YEAAVPDPR

SEQ ID NO: 119 - protein, 10 aa, Homo sapiens
VAMHLVCPSR

SEQ ID NO: 120 - protein, 10 aa, Homo sapiens
THPHFVIPYR

SEQ ID NO: 121 - protein, 11 aa, Homo sapiens
ALEFLQLHNGR

SEQ ID NO: 122 - protein, 17 aa, Homo sapiens
VLSALQAVQGLLVAQGR

SEQ ID NO: 123 - protein, 12 aa, Homo sapiens
ALQDQLVLVAAK

SEQ ID NO: 124 - protein, 12 aa, Homo sapiens
DPTFIPAPIQAK

SEQ ID NO: 125 - protein, 9 aa, Homo sapiens
LPGIVAEGR

SEQ ID NO: 126 - protein, 11 aa, Homo sapiens
DDLYVSDAFHK

SEQ ID NO: 127 - protein, 13 aa, Homo sapiens
VAEGTQVLELPFK

SEQ ID NO: 128 - protein, 12 aa, Homo sapiens
EVPLNTIIFMGR

SEQ ID NO: 129 - protein, 9 aa, Homo sapiens
CFLAFTQTK

SEQ ID NO: 130 - protein, 11 aa, Homo sapiens
EQQALQTVCLK

SEQ ID NO: 131 - protein, 12 aa, Homo sapiens
LDTLAQEVALLK

SEQ ID NO: 132 - protein, 12 aa, Homo sapiens
TFHEASEDCISR

SEQ ID NO: 133 - protein, 14 aa, Homo sapiens
NWETEITAQPDGGK

SEQ ID NO: 134 - protein, 16 aa, Homo sapiens
APEAQVSVQPNFQQDK

SEQ ID NO: 135 - protein, 14 aa, Homo sapiens
LYTLVLTDPDAPSR

SEQ ID NO: 136 - protein, 9 aa, Homo sapiens
CDEPILSNR

SEQ ID NO: 137 - protein, 11 aa, Homo sapiens
GCPTEEGCGER

SEQ ID NO: 138 - protein, 11 aa, Homo sapiens
AASGTQNNVLR

SEQ ID NO: 139 - protein, 9 aa, Homo sapiens
NAVGVSLPR

SEQ ID NO: 140 - protein, 11 aa, Homo sapiens
LPPNVVEESAR

SEQ ID NO: 141 - protein, 12 aa, Homo sapiens
TIYTPGSTVLYR

SEQ ID NO: 142 - protein, 14 aa, Homo sapiens
IPIEDGSGEVVLSR

SEQ ID NO: 143 - protein, 16 aa, Homo sapiens
LVGGPMDASVEEEGVR

SEQ ID NO: 144 - protein, 11 aa, Homo sapiens
ALDFAVGEYNK

SEQ ID NO: 145 - protein, 10 aa, Homo sapiens
VSYNVPLEAR

SEQ ID NO: 146 - protein, 8 aa, Homo sapiens
TGAQELLR

SEQ ID NO: 147 - protein, 13 aa, Homo sapiens
QSLEASLAETEGR

SEQ ID NO: 148 - protein, 12 aa, Homo sapiens
LEGEACGVYTPR

SEQ ID NO: 149 - protein, 15 aa, Homo sapiens
SELEEQLTPVAEETR

SEQ ID NO: 150 - protein, 15 aa, Homo sapiens
GEVQAMLGQSTEELR

SEQ ID NO: 151 - protein, 16 aa, Homo sapiens
LVGGPMDASVEEEGVR

SEQ ID NO: 152 - protein, 11 aa, Homo sapiens
ALDFAVGEYNK

SEQ ID NO: 153 - protein, 12 aa, Homo sapiens
TIYTPGSTVLYR

SEQ ID NO: 154 - protein, 14 aa, Homo sapiens
IPIEDGSGEVVLSR

SEQ ID NO: 155 - protein, 11 aa, Homo sapiens
THLAPYSDELR

SEQ ID NO: 156 - protein, 9 aa, Homo sapiens
IPTTFENGR

SEQ ID NO: 157 - protein, 16 aa, Homo sapiens
TMLLQPAGSLGSYSYR

SEQ ID NO: 158 - protein, 16 aa, Homo sapiens
APEAQVSVQPNFQQDK

SEQ ID NO: 159 - protein, 17 aa, Homo sapiens
AQGFTEDTIVFLPQTDK

SEQ ID NO: 160 - protein, 15 aa, Homo sapiens
SELEEQLTPVAEETR

SEQ ID NO: 161 - protein, 16 aa, Homo sapiens
TMLLQPAGSLGSYSYR

SEQ ID NO: 162 - protein, 17 aa, Homo sapiens
AQGFTEDTIVFLPQTDK

SEQ ID NO: 163 - protein, 16 aa, Homo sapiens
LVGGPMDASVEEEGVR

SEQ ID NO: 164 - protein, 11 aa, Homo sapiens
ALDFAVGEYNK

SEQ ID NO: 165 - protein, 16 aa, Homo sapiens
TMLLQPAGSLGSYSYR

SEQ ID NO: 166 - protein, 17 aa, Homo sapiens
AQGFTEDTIVFLPQTDK

SEQ ID NO: 167 - protein, 16 aa, Homo sapiens
LVGGPMDASVEEEGVR

SEQ ID NO: 168 - protein, 13 aa, Homo sapiens
GSPAINVAVHVFR

SEQ ID NO: 169 - protein, 9 aa, Homo sapiens
IPTTFENGR

SEQ ID NO: 170 - protein, 15 aa, Homo sapiens
DASGVTFTWTPSSGK

SEQ ID NO: 171 - protein, 9 aa, Homo sapiens
SAVQGPPER

SEQ ID NO: 172 - protein, 12 aa, Homo sapiens
TFTCTAAYPESK

SEQ ID NO: 173 - protein, 10 aa, Homo sapiens
WLQGSQELPR

SEQ ID NO: 174 - protein, 10 aa, Homo sapiens
KGYTQQLAFR

SEQ ID NO: 175 - protein, 16 aa, Homo sapiens
DICEEQVNSLPGSITK

SEQ ID NO: 176 - protein, 14 aa, Homo sapiens
AGDFLEANYMNLQR

SEQ ID NO: 177 - protein, 10 aa, Homo sapiens
DFDFVPPVVR

SEQ ID NO: 178 - protein, 12 aa, Homo sapiens
ASSIIDELFQDR

SEQ ID NO: 179 - protein, 11 aa, Homo sapiens
ELDESLQVAER

SEQ ID NO: 180 - protein, 16 aa, Homo sapiens
LVGGPMDASVEEEGVR

SEQ ID NO: 181 - protein, 20 aa, Homo sapiens
FSSCGGGGGSFGAGGGFGSR

SEQ ID NO: 182 - protein, 10 aa, Homo sapiens
NMQDMVEDYR

SEQ ID NO: 183 - protein, 7 aa, Homo sapiens
QYDSILR

SEQ ID NO: 184 - protein, 13 aa, Homo sapiens
EGLDLQVLEDSGR

SEQ ID NO: 185 - protein, 8 aa, Homo sapiens
QFPTPGIR

SEQ ID NO: 186 - protein, 11 aa, Homo sapiens
LCQDLGPGAFR

SEQ ID NO: 187 - protein, 8 aa, Homo sapiens
FDPSLTQR

SEQ ID NO: 188 - protein, 11 aa, Homo sapiens
SIEVFGQFNGK

SEQ ID NO: 189 - protein, 9 aa, Homo sapiens
DGNTLTYYR

SEQ ID NO: 190 - protein, 13 aa, Homo sapiens
DVVLTTTFVDDIK

SEQ ID NO: 191 - protein, 12 aa, Homo sapiens
AIEDYINEFSVR

SEQ ID NO: 192 - protein, 10 aa, Homo sapiens
WLQGSQELPR

SEQ ID NO: 193 - protein, 11 aa, Homo sapiens
QLYGDTGVLGR

SEQ ID NO: 194 - protein, 15 aa, Homo sapiens
SLPVSDSVLSGFEQR

SEQ ID NO: 195 - protein, 12 aa, Homo sapiens
ASSIIDELFQDR

SEQ ID NO: 196 - protein, 13 aa, Homo sapiens
ILEVVNQIQDEER

SEQ ID NO: 197 - protein, 11 aa, Homo sapiens
LCQDLGPGAFR

SEQ ID NO: 198 - protein, 7 aa, Homo sapiens
YTFELSR

SEQ ID NO: 199 - protein, 15 aa, Homo sapiens
EWVAIESDSVQPVPR

SEQ ID NO: 200 - protein, 11 aa, Homo sapiens
MMAVAADTLQR

SEQ ID NO: 201 - protein, 14 aa, Homo sapiens
GPVLAWINAVSAFR

SEQ ID NO: 202 - protein, 11 aa, Homo sapiens
ALEQDLPVNIK

SEQ ID NO: 203 - protein, 10 aa, Homo sapiens
AIHLDLEEYR

SEQ ID NO: 204 - protein, 9 aa, Homo sapiens
EEILMHLWR

SEQ ID NO: 205 - protein, 8 aa, Homo sapiens
HLEDVFSK

SEQ ID NO: 206 - protein, 11 aa, Homo sapiens
TVFGTEPDMIR

SEQ ID NO: 207 - protein, 8 aa, Homo sapiens
MFQEIVHK

SEQ ID NO: 208 - protein, 8 aa, Homo sapiens
WNYIEGTK

SEQ ID NO: 209 - protein, 12 aa, Homo sapiens
DPTFIPAPIQAK

SEQ ID NO: 210 - protein, 12 aa, Homo sapiens
ALQDQLVLVAAK

SEQ ID NO: 211 - protein, 10 aa, Homo sapiens
LQAILGVPWK

SEQ ID NO: 212 - protein, 17 aa, Homo sapiens
VLSALQAVQGLLVAQGR

SEQ ID NO: 213 - protein, 13 aa, Homo sapiens
SLDFTELDVAAEK

SEQ ID NO: 214 - protein, 9 aa, Homo sapiens
FMQAVTGWK

SEQ ID NO: 215 - protein, 16 aa, Homo sapiens
DTEEEDFHVDQATTVK

SEQ ID NO: 216 - protein, 18 aa, Homo sapiens
VFSNGADLSGVTEEAPLK

SEQ ID NO: 217 - protein, 16 aa, Homo sapiens
APEAQVSVQPNFQQDK

SEQ ID NO: 218 - protein, 9 aa, Homo sapiens
IPTTFENGR

SEQ ID NO: 219 - protein, 12 aa, Homo sapiens
IIMLFTDGGEER

SEQ ID NO: 220 - protein, 10 aa, Homo sapiens
FVVTDGGITR

SEQ ID NO: 221 - protein, 12 aa, Homo sapiens
DPTFIPAPIQAK

SEQ ID NO: 222 - protein, 12 aa, Homo sapiens
ALQDQLVLVAAK

SEQ ID NO: 223 - protein, 13 aa, Homo sapiens
SLDFTELDVAAEK

SEQ ID NO: 224 - protein, 13 aa, Homo sapiens
AETYEGVYQCTAR

SEQ ID NO: 225 - protein, 9 aa, Homo sapiens
QPEYAVVQR

SEQ ID NO: 226 - protein, 13 aa, Homo sapiens
EELVYELNPLDHR

SEQ ID NO: 227 - protein, 13 aa, Homo sapiens
GSFEFPVGDAVSK

SEQ ID NO: 228 - protein, 16 aa, Homo sapiens
TMLLQPAGSLGSYSYR

SEQ ID NO: 229 - protein, 17 aa, Homo sapiens
AQGFTEDTIVFLPQTDK

SEQ ID NO: 230 - protein, 10 aa, Homo sapiens
VGDTLNLNLR

SEQ ID NO: 231 - protein, 14 aa, Homo sapiens
TTNIQGINLLFSSR

SEQ ID NO: 232 - protein, 16 aa, Homo sapiens
GGSTSYGTGSETESPR

SEQ ID NO: 233 - protein, 10 aa, Homo sapiens
QFTSSTSYNR

SEQ ID NO: 234 - protein, 15 aa, Homo sapiens
ESSSHHPGIAEFPSR

SEQ ID NO: 235 - protein, 9 aa, Homo sapiens
LEEQAQQIR

SEQ ID NO: 236 - protein, 16 aa, Homo sapiens
TMLLQPAGSLGSYSYR

SEQ ID NO: 237 - protein, 17 aa, Homo sapiens
AQGFTEDTIVFLPQTDK

SEQ ID NO: 238 - protein, 9 aa, Homo sapiens
YLDGLTAER

SEQ ID NO: 239 - protein, 16 aa, Homo sapiens
TMLLQPAGSLGSYSYR

SEQ ID NO: 240 - protein, 17 aa, Homo sapiens
AQGFTEDTIVFLPQTDK

SEQ ID NO: 241 - protein, 12 aa, Homo sapiens
TSLEDFYLDEER

SEQ ID NO: 242 - protein, 12 aa, Homo sapiens
LNMGITDLQGLR

SEQ ID NO: 243 - protein, 10 aa, Homo sapiens
VGDTLNLNLR

SEQ ID NO: 244 - protein, 12 aa, Homo sapiens
TIYTPGSTVLYR

SEQ ID NO: 245 - protein, 15 aa, Homo sapiens
TVMVNIENPEGIPVK

SEQ ID NO: 246 - protein, 9 aa, Homo sapiens
LEEQAQQIR

SEQ ID NO: 247 - protein, 9 aa, Homo sapiens
LGPLVEQGR

SEQ ID NO: 248 - protein, 12 aa, Homo sapiens
SWFEPLVEDMQR

SEQ ID NO: 249 - protein, 17 aa, Homo sapiens
ELTTEIDNNIEQISSYK

SEQ ID NO: 250 - protein, 12 aa, Homo sapiens
EFTPPVQAAYQK

SEQ ID NO: 251 - protein, 10 aa, Homo sapiens
LLVVYPWTQR

SEQ ID NO: 252 - protein, 13 aa, Homo sapiens
VNVDAVGGEALGR

SEQ ID NO: 253 - protein, 10 aa, Homo sapiens
QMLNIPNQPK

SEQ ID NO: 254 - protein, 10 aa, Homo sapiens
LLDSLPSDTR

SEQ ID NO: 255 - protein, 10 aa, Homo sapiens
FQPTLLTLPR

SEQ ID NO: 256 - protein, 11 aa, Homo sapiens
NFPSPVDAAFR

SEQ ID NO: 257 - protein, 15 aa, Homo sapiens
GECQAEGVLFFQGDR

SEQ ID NO: 258 - protein, 7 aa, Homo sapiens
AVLYNYR

SEQ ID NO: 259 - protein, 16 aa, Homo sapiens
SNLDEDIIAEENIVSR

SEQ ID NO: 260 - protein, 8 aa, Homo sapiens
FQNALLVR

SEQ ID NO: 261 - protein, 13 aa, Homo sapiens
CCAAADPHECYAK

SEQ ID NO: 262 - protein, 14 aa, Homo sapiens
VPQVSTPTLVEVSR

SEQ ID NO: 263 - protein, 11 aa, Homo sapiens
YGLVTYATYPK

SEQ ID NO: 264 - protein, 9 aa, Homo sapiens
LEEQAQQIR

SEQ ID NO: 265 - protein, 9 aa, Homo sapiens
LGPLVEQGR

SEQ ID NO: 266 - protein, 14 aa, Homo sapiens
IPIEDGSGEVVLSR

SEQ ID NO: 267 - protein, 14 aa, Homo sapiens
LAAAVSNFGYDLYR

SEQ ID NO: 268 - protein, 9 aa, Homo sapiens
LGPLVEQGR

SEQ ID NO: 269 - protein, 9 aa, Homo sapiens
LEEQAQQIR

SEQ ID NO: 270 - protein, 19 aa, Homo sapiens
AAPSVTLFPPSSEELQANK

SEQ ID NO: 271 - protein, 9 aa, Homo sapiens
GLQDEDGYR

SEQ ID NO: 272 - protein, 16 aa, Homo sapiens
LVGGPMDASVEEEGVR

SEQ ID NO: 273 - protein, 11 aa, Homo sapiens
ALDFAVGEYNK

SEQ ID NO: 274 - protein, 17 aa, Homo sapiens
AQGFTEDTIVFLPQTDK

SEQ ID NO: 275 - protein, 16 aa, Homo sapiens
TMLLQPAGSLGSYSYR

SEQ ID NO: 276 - protein, 14 aa, Homo sapiens
LAAAVSNFGYDLYR

SEQ ID NO: 277 - protein, 11 aa, Homo sapiens
ELLDTVTAPQK

SEQ ID NO: 278 - protein, 12 aa, Homo sapiens
TYMLAFDVNDEK

SEQ ID NO: 279 - protein, 14 aa, Homo sapiens
EQLGEFYEALDCLR

SEQ ID NO: 280 - protein, 9 aa, Homo sapiens
SDVVYTDWK

SEQ ID NO: 281 - protein, 8 aa, Homo sapiens
TEDTIFLR

SEQ ID NO: 282 - protein, 10 aa, Homo sapiens
WLQGSQELPR

SEQ ID NO: 283 - protein, 8 aa, Homo sapiens
FQNALLVR

SEQ ID NO: 284 - protein, 7 aa, Homo sapiens
VWNYFQR

SEQ ID NO: 285 - protein, 8 aa, Homo sapiens
QPPFTDYR

SEQ ID NO: 286 - protein, 10 aa, Homo sapiens
TEAESWYQTK

SEQ ID NO: 287 - protein, 9 aa, Homo sapiens
EYQELMNVK

SEQ ID NO: 288 - protein, 9 aa, Homo sapiens
FAFQAEVNR

SEQ ID NO: 289 - protein, 16 aa, Homo sapiens
EEEAIQLDGLNASQIR

SEQ ID NO: 290 - protein, 10 aa, Homo sapiens
CSVFYGAPSK

SEQ ID NO: 291 - protein, 9 aa, Homo sapiens
GLQDEDGYR

SEQ ID NO: 292 - protein, 8 aa, Homo sapiens
VEYGFQVK

SEQ ID NO: 293 - protein, 9 aa, Homo sapiens
ITQVLHFTK

SEQ ID NO: 294 - protein, 7 aa, Homo sapiens
FEEILTR

SEQ ID NO: 295 - protein, 13 aa, Homo sapiens
SFLVWVNEEDHLR

SEQ ID NO: 296 - protein, 16 aa, Homo sapiens
VLGAFSDGLAHLDNLK

SEQ ID NO: 297 - protein, 10 aa, Homo sapiens
LLVVYPWTQR

SEQ ID NO: 298 - protein, 13 aa, Homo sapiens
GTFATLSELHCDK

SEQ ID NO: 299 - protein, 12 aa, Homo sapiens
EFTPPVQAAYQK

SEQ ID NO: 300 - protein, 12 aa, Homo sapiens
TSLEDFYLDEER

SEQ ID NO: 301 - protein, 9 aa, Homo sapiens
SSFVAPLEK

SEQ ID NO: 302 - protein, 11 aa, Homo sapiens
LVVEWQLQDDK

SEQ ID NO: 303 - protein, 12 aa, Homo sapiens
EVVADSVWVDVK

SEQ ID NO: 304 - protein, 12 aa, Homo sapiens
EFTPPVQAAYQK

SEQ ID NO: 305 - protein, 12 aa, Homo sapiens
VVAGVANALAHK

SEQ ID NO: 306 - protein, 8 aa, Homo sapiens
VHLTPEEK

SEQ ID NO: 307 - protein, 17 aa, Homo sapiens
AQGFTEDTIVFLPQTDK

SEQ ID NO: 308 - protein, 17 aa, Homo sapiens
AQGFTEDTIVFLPQTDK

SEQ ID NO: 309 - protein, 14 aa, Homo sapiens
VETALEACSLPSSR

SEQ ID NO: 310 - protein, 9 aa, Homo sapiens
LGPLVEQGR

SEQ ID NO: 311 - protein, 9 aa, Homo sapiens
LEEQAQQIR

SEQ ID NO: 312 - protein, 7 aa, Homo sapiens
FACYYPR

SEQ ID NO: 313 - protein, 8 aa, Homo sapiens
QWAGLVEK

SEQ ID NO: 314 - protein, 9 aa, Homo sapiens
LGPLVEQGR

SEQ ID NO: 315 - protein, 9 aa, Homo sapiens
LEEQAQQIR

SEQ ID NO: 316 - protein, 15 aa, Homo sapiens
AATVGSLAGQPLQER

SEQ ID NO: 317 - protein, 9 aa, Homo sapiens
LGPLVEQGR

SEQ ID NO: 318 - protein, 9 aa, Homo sapiens
LEEQAQQIR

SEQ ID NO: 319 - protein, 15 aa, Homo sapiens
AATVGSLAGQPLQER

SEQ ID NO: 320 - protein, 12 aa, Homo sapiens
LNMGITDLQGLR

SEQ ID NO: 321 - protein, 9 aa, Homo sapiens
AEFQDALEK

SEQ ID NO: 322 - protein, 10 aa, Homo sapiens
VGDTLNLNLR

SEQ ID NO: 323 - protein, 13 aa, Homo sapiens
FSNTDYAVGYMLR

SEQ ID NO: 324 - protein, 10 aa, Homo sapiens
LVMGIPTFGR

SEQ ID NO: 325 - protein, 11 aa, Homo sapiens
ELDESLQVAER

SEQ ID NO: 326 - protein, 16 aa, Homo sapiens
EILSVDCSTNNPSQAK

SEQ ID NO: 327 - protein, 10 aa, Homo sapiens
QQTEWQSGQR

SEQ ID NO: 328 - protein, 14 aa, Homo sapiens
VEQAVETEPEPELR

SEQ ID NO: 329 - protein, 15 aa, Homo sapiens
GEVQAMLGQSTEELR

SEQ ID NO: 330 - protein, 15 aa, Homo sapiens
SELEEQLTPVAEETR

SEQ ID NO: 331 - protein, 9 aa, Homo sapiens
SSFVAPLEK

SEQ ID NO: 332 - protein, 12 aa, Homo sapiens
TSLEDFYLDEER

SEQ ID NO: 333 - protein, 7 aa, Homo sapiens
VWNYFQR

SEQ ID NO: 334 - protein, 7 aa, Homo sapiens
WVEELMK

SEQ ID NO: 335 - protein, 9 aa, Homo sapiens
SYPEILTLK

SEQ ID NO: 336 - protein, 9 aa, Homo sapiens
LGPLVEQGR

SEQ ID NO: 337 - protein, 9 aa, Homo sapiens
LEEQAQQIR

SEQ ID NO: 338 - protein, 15 aa, Homo sapiens
AATVGSLAGQPLQER

SEQ ID NO: 339 - protein, 16 aa, Homo sapiens
TMLLQPAGSLGSYSYR

SEQ ID NO: 340 - protein, 17 aa, Homo sapiens
AQGFTEDTIVFLPQTDK

SEQ ID NO: 341 - protein, 7 aa, Homo sapiens
VWNYFQR

SEQ ID NO: 342 - protein, 7 aa, Homo sapiens
WVEELMK

SEQ ID NO: 343 - protein, 8 aa, Homo sapiens
LVAEFDFR

SEQ ID NO: 344 - protein, 9 aa, Homo sapiens
IVQLIQDTR

SEQ ID NO: 345 - protein, 9 aa, Homo sapiens
SIPQVSPVR

SEQ ID NO: 346 - protein, 13 aa, Homo sapiens
GSPAINVAVHVFR

SEQ ID NO: 347 - protein, 12 aa, Homo sapiens
LQSLFDSPDFSK

SEQ ID NO: 348 - protein, 10 aa, Homo sapiens
YGLDSDLSCK

SEQ ID NO: 349 - protein, 9 aa, Homo sapiens
LSYEGEVTK

SEQ ID NO: 350 - protein, 9 aa, Homo sapiens
SSFVAPLEK

SEQ ID NO: 351 - protein, 12 aa, Homo sapiens
TSLEDFYLDEER

SEQ ID NO: 352 - protein, 8 aa, Homo sapiens
ISYEEWAK

SEQ ID NO: 353 - protein, 7 aa, Homo sapiens
VIEYVDR

SEQ ID NO: 354 - protein, 11 aa, Homo sapiens
ALGHLDLSGNR

SEQ ID NO: 355 - protein, 10 aa, Homo sapiens
VAAGAFQGLR

SEQ ID NO: 356 - protein, 8 aa, Homo sapiens
YLFLNGNK

SEQ ID NO: 357 - protein, 12 aa, Homo sapiens
TIYTPGSTVLYR

SEQ ID NO: 358 - protein, 12 aa, Homo sapiens
TSLEDFYLDEER

SEQ ID NO: 359 - protein, 15 aa, Homo sapiens
DGFVQDEGTMFPVGK

SEQ ID NO: 360 - protein, 8 aa, Homo sapiens
VSVFVPPR

SEQ ID NO: 361 - protein, 11 aa, Homo sapiens
LGADMEDVCGR

SEQ ID NO: 362 - protein, 15 aa, Homo sapiens
GEVQAMLGQSTEELR

SEQ ID NO: 363 - protein, 15 aa, Homo sapiens
SELEEQLTPVAEETR

SEQ ID NO: 364 - protein, 11 aa, Homo sapiens
MLQWDDIICVR

SEQ ID NO: 365 - protein, 12 aa, Homo sapiens
LNMGITDLQGLR

SEQ ID NO: 366 - protein, 8 aa, Homo sapiens
GQIVFMNR

SEQ ID NO: 367 - protein, 13 aa, Homo sapiens
EMSGSPASGIPVK

SEQ ID NO: 368 - protein, 12 aa, Homo sapiens
LNMGITDLQGLR

SEQ ID NO: 369 - protein, 8 aa, Homo sapiens
GQIVFMNR

SEQ ID NO: 370 - protein, 13 aa, Homo sapiens
EMSGSPASGIPVK

SEQ ID NO: 371 - protein, 9 aa, Homo sapiens
AEFQDALEK

SEQ ID NO: 372 - protein, 10 aa, Homo sapiens
VGDTLNLNLR

SEQ ID NO: 373 - protein, 12 aa, Homo sapiens
LNDLEEALQQAK

SEQ ID NO: 374 - protein, 15 aa, Homo sapiens
GECQAEGVLFFQGDR

SEQ ID NO: 375 - protein, 9 aa, Homo sapiens
DYFMPCPGR

SEQ ID NO: 376 - protein, 10 aa, Homo sapiens
TGYYFDGISR

SEQ ID NO: 377 - protein, 11 aa, Homo sapiens
CLAFECPENYR

SEQ ID NO: 378 - protein, 16 aa, Homo sapiens
IIEVEEEQEDPYLNDR

SEQ ID NO: 379 - protein, 8 aa, Homo sapiens
FDPSLTQR

SEQ ID NO: 380 - protein, 11 aa, Homo sapiens
LCQDLGPGAFR

SEQ ID NO: 381 - protein, 11 aa, Homo sapiens
QELSEAEQATR

SEQ ID NO: 382 - protein, 12 aa, Homo sapiens
TIYTPGSTVLYR

SEQ ID NO: 383 - protein, 14 aa, Homo sapiens
IPIEDGSGEVVLSR

SEQ ID NO: 384 - protein, 9 aa, Homo sapiens
ATVVYQGER

SEQ ID NO: 385 - protein, 14 aa, Homo sapiens
IPIEDGSGEVVLSR

SEQ ID NO: 386 - protein, 10 aa, Homo sapiens
RPYFPVAVGK

SEQ ID NO: 387 - protein, 11 aa, Homo sapiens
SCDIPVFMNAR

SEQ ID NO: 388 - protein, 16 aa, Homo sapiens
TMLLQPAGSLGSYSYR

SEQ ID NO: 389 - protein, 11 aa, Homo sapiens
LGADMEDVCGR

SEQ ID NO: 390 - protein, 14 aa, Homo sapiens
VEQAVETEPEPELR

SEQ ID NO: 391 - protein, 15 aa, Homo sapiens
GEVQAMLGQSTEELR

SEQ ID NO: 392 - protein, 15 aa, Homo sapiens
SELEEQLTPVAEETR

SEQ ID NO: 393 - protein, 8 aa, Homo sapiens
EPGLQIWR

SEQ ID NO: 394 - protein, 11 aa, Homo sapiens
HVVPNEVVVQR

SEQ ID NO: 395 - protein, 13 aa, Homo sapiens
EQTMSECEAGALR

SEQ ID NO: 396 - protein, 11 aa, Homo sapiens
TLNICEVGTIR

SEQ ID NO: 397 - protein, 8 aa, Homo sapiens
QLEWGLER

SEQ ID NO: 398 - protein, 11 aa, Homo sapiens
HEGSFIQGAEK

SEQ ID NO: 399 - protein, 11 aa, Homo sapiens
IAPANADFAFR

SEQ ID NO: 400 - protein, 11 aa, Homo sapiens
YVMLPVADQEK

SEQ ID NO: 401 - protein, 16 aa, Homo sapiens
SCDLALLETYCATPAK

SEQ ID NO: 402 - protein, 9 aa, Homo sapiens
GIVEECCFR

SEQ ID NO: 403 - protein, 13 aa, Homo sapiens
GLVSWGNIPCGSK

SEQ ID NO: 404 - protein, 10 aa, Homo sapiens
TGESVEFVCK

SEQ ID NO: 405 - protein, 9 aa, Homo sapiens
IDVHLVPDR

SEQ ID NO: 406 - protein, 11 aa, Homo sapiens
ELDESLQVAER

SEQ ID NO: 407 - protein, 9 aa, Homo sapiens
DHAVDLIQK

SEQ ID NO: 408 - protein, 12 aa, Homo sapiens
TEQWSTLPPETK

SEQ ID NO: 409 - protein, 15 aa, Homo sapiens
VLSLAQEQVGGSPEK

SEQ ID NO: 410 - protein, 9 aa, Homo sapiens
QGSFQGGFR

SEQ ID NO: 411 - protein, 12 aa, Homo sapiens
KADGSYAAWLSR

SEQ ID NO: 412 - protein, 13 aa, Homo sapiens
AEMADQAAAWLTR

SEQ ID NO: 413 - protein, 11 aa, Homo sapiens
EIMENYNIALR

SEQ ID NO: 414 - protein, 15 aa, Homo sapiens
GEVQAMLGQSTEELR

SEQ ID NO: 415 - protein, 15 aa, Homo sapiens
KVEQAVETEPEPELR

SEQ ID NO: 416 - protein, 15 aa, Homo sapiens
SELEEQLTPVAEETR

SEQ ID NO: 417 - protein, 14 aa, Homo sapiens
EVDSGNDIYGNPIK

SEQ ID NO: 418 - protein, 9 aa, Homo sapiens
SDGSCAWYR

SEQ ID NO: 419 - protein, 8 aa, Homo sapiens
TGAQELLR

SEQ ID NO: 420 - protein, 11 aa, Homo sapiens
AASGTQNNVLR

SEQ ID NO: 421 - protein, 13 aa, Homo sapiens
EQTMSECEAGALR

SEQ ID NO: 422 - protein, 7 aa, Homo sapiens
YLYEIAR

SEQ ID NO: 423 - protein, 9 aa, Homo sapiens
CCTESLVNR

SEQ ID NO: 424 - protein, 11 aa, Homo sapiens
IETALTSLHQR

SEQ ID NO: 425 - protein, 9 aa, Homo sapiens
LENLEQYSR

SEQ ID NO: 426 - protein, 12 aa, Homo sapiens
VEQATQAIPMER

SEQ ID NO: 427 - protein, 10 aa, Homo sapiens
QMYPELQIAR

SEQ ID NO: 428 - protein, 9 aa, Homo sapiens
ATVNPSAPR

SEQ ID NO: 429 - protein, 8 aa, Homo sapiens
VLDLSCNR

SEQ ID NO: 430 - protein, 13 aa, Homo sapiens
VPPTLEVTQQPVR

SEQ ID NO: 431 - protein, 11 aa, Homo sapiens
IAPANADFAFR

SEQ ID NO: 432 - protein, 11 aa, Homo sapiens
DFYVDENTTVR

SEQ ID NO: 433 - protein, 7 aa, Homo sapiens
IWDVVEK

SEQ ID NO: 434 - protein, 11 aa, Homo sapiens
QPVPGQQMTLK

SEQ ID NO: 435 - protein, 11 aa, Homo sapiens
QELSEAEQATR

SEQ ID NO: 436 - protein, 9 aa, Homo sapiens
GLEVTITAR

SEQ ID NO: 437 - protein, 12 aa, Homo sapiens
TIYTPGSTVLYR

SEQ ID NO: 438 - protein, 14 aa, Homo sapiens
IPIEDGSGEVVLSR

SEQ ID NO: 439 - protein, 10 aa, Homo sapiens
DQDGEILLPR

SEQ ID NO: 440 - protein, 8 aa, Homo sapiens
QELEDLER

SEQ ID NO: 441 - protein, 15 aa, Homo sapiens
IPGIFELGISSQSDR

SEQ ID NO: 442 - protein, 11 aa, Homo sapiens
LPLEYSYGEYR

SEQ ID NO: 443 - protein, 7 aa, Homo sapiens
AVLYNYR

SEQ ID NO: 444 - protein, 16 aa, Homo sapiens
TALASGGVLDASGDYR

SEQ ID NO: 445 - protein, 10 aa, Homo sapiens
WLQGSQELPR

SEQ ID NO: 446 - protein, 11 aa, Homo sapiens
NFPSPVDAAFR

SEQ ID NO: 447 - protein, 8 aa, Homo sapiens
WELCDIPR

SEQ ID NO: 448 - protein, 11 aa, Homo sapiens
HSIFTPETNPR

SEQ ID NO: 449 - protein, 7 aa, Homo sapiens
YEFLNGR

SEQ ID NO: 450 - protein, 20 aa, Homo sapiens
FSSCGGGGGSFGAGGGFGSR

SEQ ID NO: 451 - protein, 9 aa, Homo sapiens
QDGSVDFGR

SEQ ID NO: 452 - protein, 13 aa, Homo sapiens
LESDVSAQMEYCR

SEQ ID NO: 453 - protein, 10 aa, Homo sapiens
EDGGGWWYNR

SEQ ID NO: 454 - protein, 13 aa, Homo sapiens
QGFGNVATNTDGK

SEQ ID NO: 455 - protein, 10 aa, Homo sapiens
EDQYHYLLDR

SEQ ID NO: 456 - protein, 13 aa, Homo sapiens
GFQQLLQELNQPR

SEQ ID NO: 457 - protein, 13 aa, Homo sapiens
TLYLADTFPTNFR

SEQ ID NO: 458 - protein, 8 aa, Homo sapiens
VHLTPEEK

SEQ ID NO: 459 - protein, 13 aa, Homo sapiens
GTFATLSELHCDK

SEQ ID NO: 460 - protein, 16 aa, Homo sapiens
VLGAFSDGLAHLDNLK

SEQ ID NO: 461 - protein, 10 aa, Homo sapiens
LLVVYPWTQR

SEQ ID NO: 462 - protein, 12 aa, Homo sapiens
EFTPPVQAAYQK

SEQ ID NO: 463 - protein, 16 aa, Homo sapiens
LVGGPMDASVEEEGVR

SEQ ID NO: 464 - protein, 10 aa, Homo sapiens
TGDEITYQCR

SEQ ID NO: 465 - protein, 15 aa, Homo sapiens
RAKAELAKETDPLRR

SEQ ID NO: 466 - protein, 14 aa, Homo sapiens
VEQAVETEPEPELR

SEQ ID NO: 467 - protein, 15 aa, Homo sapiens
GEVQAMLGQSTEELR

SEQ ID NO: 468 - protein, 9 aa, Homo sapiens
LEEQAQQIR

SEQ ID NO: 469 - protein, 15 aa, Homo sapiens
SELEEQLTPVAEETR

SEQ ID NO: 470 - protein, 12 aa, Homo sapiens
GPPGPPGGVVVR

SEQ ID NO: 471 - protein, 11 aa, Homo sapiens
GGEILIPCQPR

SEQ ID NO: 472 - protein, 9 aa, Homo sapiens
VEVLAGDLR

SEQ ID NO: 473 - protein, 12 aa, Homo sapiens
FAQLNLAAEDTR

SEQ ID NO: 474 - protein, 9 aa, Homo sapiens
SAVQGPPER

SEQ ID NO: 475 - protein, 10 aa, Homo sapiens
WLQGSQELPR

SEQ ID NO: 476 - protein, 12 aa, Homo sapiens
TFTCTAAYPESK

SEQ ID NO: 477 - protein, 15 aa, Homo sapiens
DASGVTFTWTPSSGK

SEQ ID NO: 478 - protein, 18 aa, Homo sapiens
LTVGAAQVPAQLLVGALR

SEQ ID NO: 479 - protein, 8 aa, Homo sapiens
EPGLQIWR

SEQ ID NO: 480 - protein, 15 aa, Homo sapiens
EVQGFESATFLGYFK

SEQ ID NO: 481 - protein, 11 aa, Homo sapiens
HVVPNEVVVQR

SEQ ID NO: 482 - protein, 17 aa, Homo sapiens
QTQVSVLPEGGETPLFK

SEQ ID NO: 483 - protein, 11 aa, Homo sapiens
VQVTSQEYSAR

SEQ ID NO: 484 - protein, 12 aa, Homo sapiens
GPPGPPGGVVVR

SEQ ID NO: 485 - protein, 12 aa, Homo sapiens
FAQLNLAAEDTR

SEQ ID NO: 486 - protein, 16 aa, Homo sapiens
SYELPDGQVITIGNER

SEQ ID NO: 487 - protein, 10 aa, Homo sapiens
GYSFTTTAER

SEQ ID NO: 488 - protein, 13 aa, Homo sapiens
QEYDESGPSIVHR

SEQ ID NO: 489 - protein, 12 aa, Homo sapiens
GPPGPPGGVVVR

SEQ ID NO: 490 - protein, 14 aa, Homo sapiens
VISDTEADIGSNLR

SEQ ID NO: 491 - protein, 12 aa, Homo sapiens
VTVTPDGTLIIR

SEQ ID NO: 492 - protein, 12 aa, Homo sapiens
FAQLNLAAEDTR

SEQ ID NO: 493 - protein, 11 aa, Homo sapiens
GGEILIPCQPR

SEQ ID NO: 494 - protein, 15 aa, Homo sapiens
GECQAEGVLFFQGDR

SEQ ID NO: 495 - protein, 8 aa, Homo sapiens
RLWWLDLK

SEQ ID NO: 496 - protein, 9 aa, Homo sapiens
DYFMPCPGR

SEQ ID NO: 497 - protein, 11 aa, Homo sapiens
YYCFQGNQFLR

SEQ ID NO: 498 - protein, 13 aa, Homo sapiens
GSPAINVAVHVFR

SEQ ID NO: 499 - protein, 13 aa, Homo sapiens
AADDTWEPFASGK

SEQ ID NO: 500 - protein, 8 aa, Homo sapiens
FDPSLTQR

SEQ ID NO: 501 - protein, 11 aa, Homo sapiens
LCQDLGPGAFR

SEQ ID NO: 502 - protein, 9 aa, Homo sapiens
VLFYVDSEK

SEQ ID NO: 503 - protein, 12 aa, Homo sapiens
TEQWSTLPPETK

SEQ ID NO: 504 - protein, 15 aa, Homo sapiens
VLSLAQEQVGGSPEK

SEQ ID NO: 505 - protein, 9 aa, Homo sapiens
QGSFQGGFR

SEQ ID NO: 506 - protein, 13 aa, Homo sapiens
AEMADQAAAWLTR

SEQ ID NO: 507 - protein, 12 aa, Homo sapiens
ESYNVQLQLPAR

SEQ ID NO: 508 - protein, 9 aa, Homo sapiens
SAVQGPPER

SEQ ID NO: 509 - protein, 10 aa, Homo sapiens
WLQGSQELPR

SEQ ID NO: 510 - protein, 15 aa, Homo sapiens
DASGVTFTWTPSSGK

SEQ ID NO: 511 - protein, 8 aa, Homo sapiens
YFIDFVAR

SEQ ID NO: 512 - protein, 15 aa, Homo sapiens
YNSQNQSNNQFVLYR

SEQ ID NO: 513 - protein, 11 aa, Homo sapiens
TVGSDTFYSFK

SEQ ID NO: 514 - protein, 17 aa, Homo sapiens
QEPSQGTTTFAVTSILR

SEQ ID NO: 515 - protein, 10 aa, Homo sapiens
WLQGSQELPR

SEQ ID NO: 516 - protein, 11 aa, Homo sapiens
EIMENYNIALR

SEQ ID NO: 517 - protein, 12 aa, Homo sapiens
TSLEDFYLDEER

SEQ ID NO: 518 - protein, 10 aa, Homo sapiens
CSVFYGAPSK

SEQ ID NO: 519 - protein, 9 aa, Homo sapiens
GLQDEDGYR

SEQ ID NO: 520 - protein, 8 aa, Homo sapiens
VEYGFQVK

SEQ ID NO: 521 - protein, 9 aa, Homo sapiens
ITQVLHFTK

SEQ ID NO: 522 - protein, 7 aa, Homo sapiens
FACYYPR

SEQ ID NO: 523 - protein, 9 aa, Homo sapiens
VHYTVCIWR

SEQ ID NO: 524 - protein, 14 aa, Homo sapiens
VVIGMDVAASEFFR

SEQ ID NO: 525 - protein, 14 aa, Homo sapiens
VPTANVSVVDLTCR

SEQ ID NO: 526 - protein, 14 aa, Homo sapiens
LISWYDNEFGYSNR

SEQ ID NO: 527 - protein, 9 aa, Homo sapiens
IDVHLVPDR

SEQ ID NO: 528 - protein, 12 aa, Homo sapiens
GPPGPPGGVVVR

SEQ ID NO: 529 - protein, 12 aa, Homo sapiens
FAQLNLAAEDTR

SEQ ID NO: 530 - protein, 12 aa, Homo sapiens
GPPGPVGPPGEK

SEQ ID NO: 531 - protein, 12 aa, Homo sapiens
TIYTPGSTVLYR

SEQ ID NO: 532 - protein, 14 aa, Homo sapiens
IPIEDGSGEVVLSR

SEQ ID NO: 533 - protein, 12 aa, Homo sapiens
TIYTPGSTVLYR

SEQ ID NO: 534 - protein, 14 aa, Homo sapiens
IPIEDGSGEVVLSR

SEQ ID NO: 535 - protein, 12 aa, Homo sapiens
ITWSNPPAQGAR

SEQ ID NO: 536 - protein, 14 aa, Homo sapiens
VGGVQSLGGTGALR

SEQ ID NO: 537 - protein, 8 aa, Homo sapiens
NFGLYNER

SEQ ID NO: 538 - protein, 9 aa, Homo sapiens
HIYLLPSGR

SEQ ID NO: 539 - protein, 8 aa, Homo sapiens
IPSETLNR

SEQ ID NO: 540 - protein, 13 aa, Homo sapiens
QAGLGNHLSGSER

SEQ ID NO: 541 - protein, 9 aa, Homo sapiens
ILGDPEALR

SEQ ID NO: 542 - protein, 10 aa, Homo sapiens
IEIFQTLPVR

SEQ ID NO: 543 - protein, 15 aa, Homo sapiens
MLLELAPTSDNDFGR

SEQ ID NO: 544 - protein, 15 aa, Homo sapiens
GECQAEGVLFFQGDR

SEQ ID NO: 545 - protein, 9 aa, Homo sapiens
DYFMPCPGR

SEQ ID NO: 546 - protein, 11 aa, Homo sapiens
YYCFQGNQFLR

SEQ ID NO: 547 - protein, 15 aa, Homo sapiens
GECQAEGVLFFQGDR

SEQ ID NO: 548 - protein, 11 aa, Homo sapiens
NFPSPVDAAFR

SEQ ID NO: 549 - protein, 8 aa, Homo sapiens
VWVYPPEK

SEQ ID NO: 550 - protein, 16 aa, Homo sapiens
NGVAQEPVHLDSPAIK

SEQ ID NO: 551 - protein, 11 aa, Homo sapiens
ATWSGAVLAGR

SEQ ID NO: 552 - protein, 9 aa, Homo sapiens
CLAPLEGAR

SEQ ID NO: 553 - protein, 12 aa, Homo sapiens
HQFLLTGDTQGR

SEQ ID NO: 554 - protein, 9 aa, Homo sapiens
EGPVLILGR

SEQ ID NO: 555 - protein, 16 aa, Homo sapiens
TMLLQPAGSLGSYSYR

SEQ ID NO: 556 - protein, 16 aa, Homo sapiens
APEAQVSVQPNFQQDK

SEQ ID NO: 557 - protein, 11 aa, Homo sapiens
YGLVTYATYPK

SEQ ID NO: 558 - protein, 13 aa, Homo sapiens
LQNNENNISCVER

SEQ ID NO: 559 - protein, 9 aa, Homo sapiens
ISASAEELR

SEQ ID NO: 560 - protein, 9 aa, Homo sapiens
LAPLAEDVR

SEQ ID NO: 561 - protein, 10 aa, Homo sapiens
ALVQQMEQLR

SEQ ID NO: 562 - protein, 9 aa, Homo sapiens
LEPYADQLR

SEQ ID NO: 563 - protein, 11 aa, Homo sapiens
RVEPYGENFNK

SEQ ID NO: 564 - protein, 16 aa, Homo sapiens
NGVAQEPVHLDSPAIK

SEQ ID NO: 565 - protein, 12 aa, Homo sapiens
HQFLLTGDTQGR

SEQ ID NO: 566 - protein, 11 aa, Homo sapiens
ATWSGAVLAGR

SEQ ID NO: 567 - protein, 14 aa, Homo sapiens
TQSSLVPALTDFVR

SEQ ID NO: 568 - protein, 12 aa, Homo sapiens
LNMGITDLQGLR

SEQ ID NO: 569 - protein, 9 aa, Homo sapiens
SCGLHQLLR

SEQ ID NO: 570 - protein, 10 aa, Homo sapiens
VGDTLNLNLR

SEQ ID NO: 571 - protein, 11 aa, Homo sapiens
ALGHLDLSGNR

SEQ ID NO: 572 - protein, 10 aa, Homo sapiens
VAAGAFQGLR

SEQ ID NO: 573 - protein, 10 aa, Homo sapiens
FVTWIEGVMR

SEQ ID NO: 574 - protein, 7 aa, Homo sapiens
YEFLNGR

SEQ ID NO: 575 - protein, 15 aa, Homo sapiens
VLSLAQEQVGGSPEK

SEQ ID NO: 576 - protein, 9 aa, Homo sapiens
QGSFQGGFR

SEQ ID NO: 577 - protein, 13 aa, Homo sapiens
AEMADQAAAWLTR

SEQ ID NO: 578 - protein, 8 aa, Homo sapiens
EVAGLWIK

SEQ ID NO: 579 - protein, 11 aa, Homo sapiens
TYGLPCHCPFK

SEQ ID NO: 580 - protein, 15 aa, Homo sapiens
GECQAEGVLFFQGDR

SEQ ID NO: 581 - protein, 8 aa, Homo sapiens
VWVYPPEK

SEQ ID NO: 582 - protein, 9 aa, Homo sapiens
DYFMPCPGR

SEQ ID NO: 583 - protein, 11 aa, Homo sapiens
YYCFQGNQFLR

SEQ ID NO: 584 - protein, 13 aa, Homo sapiens
SVLVAAGETATLR

SEQ ID NO: 585 - protein, 12 aa, Homo sapiens
ITWSNPPAQGAR

SEQ ID NO: 586 - protein, 14 aa, Homo sapiens
VGGVQSLGGTGALR

SEQ ID NO: 587 - protein, 14 aa, Homo sapiens
LYTLVLTDPDAPSR

SEQ ID NO: 588 - protein, 16 aa, Homo sapiens
TMLLQPAGSLGSYSYR

SEQ ID NO: 589 - protein, 16 aa, Homo sapiens
APEAQVSVQPNFQQDK

SEQ ID NO: 590 - protein, 12 aa, Homo sapiens
VVEQMCITQYER

SEQ ID NO: 591 - protein, 10 aa, Homo sapiens
TGDEITYQCR

SEQ ID NO: 592 - protein, 16 aa, Homo sapiens
LVGGPMDASVEEEGVR

SEQ ID NO: 593 - protein, 11 aa, Homo sapiens
ALDFAVGEYNK

SEQ ID NO: 594 - protein, 13 aa, Homo sapiens
LPYTASSGLMAPR

SEQ ID NO: 595 - protein, 13 aa, Homo sapiens
GLIDEVNQDFTNR

SEQ ID NO: 596 - protein, 15 aa, Homo sapiens
ESSSHHPGIAEFPSR

SEQ ID NO: 597 - protein, 9 aa, Homo sapiens
WFYIASAFR

SEQ ID NO: 598 - protein, 8 aa, Homo sapiens
TEDTIFLR

SEQ ID NO: 599 - protein, 15 aa, Homo sapiens
YVGGQEHFAHLLILR

SEQ ID NO: 600 - protein, 12 aa, Homo sapiens
TYMLAFDVNDEK

SEQ ID NO: 601 - protein, 15 aa, Homo sapiens
NWGLSVYADKPETTK

SEQ ID NO: 602 - protein, 14 aa, Homo sapiens
EQLGEFYEALDCLR

SEQ ID NO: 603 - protein, 9 aa, Homo sapiens
SDVVYTDWK

SEQ ID NO: 604 - protein, 16 aa, Homo sapiens
TALASGGVLDASGDYR

SEQ ID NO: 605 - protein, 9 aa, Homo sapiens
EPGEFALLR

SEQ ID NO: 606 - protein, 10 aa, Homo sapiens
DQDGEILLPR

SEQ ID NO: 607 - protein, 16 aa, Homo sapiens
LVGGPMDASVEEEGVR

SEQ ID NO: 608 - protein, 13 aa, Homo sapiens
QSLEASLAETEGR

SEQ ID NO: 609 - protein, 13 aa, Homo sapiens
GSPAINVAVHVFR

SEQ ID NO: 610 - protein, 13 aa, Homo sapiens
AADDTWEPFASGK

SEQ ID NO: 611 - protein, 11 aa, Homo sapiens
AEAIGYAYPTR

SEQ ID NO: 612 - protein, 16 aa, Homo sapiens
TMLLQPAGSLGSYSYR

SEQ ID NO: 613 - protein, 9 aa, Homo sapiens
LGPLVEQGR

SEQ ID NO: 614 - protein, 15 aa, Homo sapiens
AATVGSLAGQPLQER

SEQ ID NO: 615 - protein, 9 aa, Homo sapiens
LEEQAQQIR

SEQ ID NO: 616 - protein, 12 aa, Homo sapiens
SWFEPLVEDMQR

SEQ ID NO: 617 - protein, 18 aa, Homo sapiens
EIVLTQSPATLSLSPGER

SEQ ID NO: 618 - protein, 16 aa, Homo sapiens
FSGSGSGTDFTLTISR

SEQ ID NO: 619 - protein, 17 aa, Homo sapiens
VYACEVTHQGLSSPVTK

SEQ ID NO: 620 - protein, 16 aa, Homo sapiens
TMLLQPAGSLGSYSYR

SEQ ID NO: 621 - protein, 17 aa, Homo sapiens
AQGFTEDTIVFLPQTDK

SEQ ID NO: 622 - protein, 12 aa, Homo sapiens
WELLQQVDTTTR

SEQ ID NO: 623 - protein, 11 aa, Homo sapiens
QELSEAEQATR

SEQ ID NO: 624 - protein, 8 aa, Homo sapiens
VVEEQESR

SEQ ID NO: 625 - protein, 9 aa, Homo sapiens
VHYTVCIWR

SEQ ID NO: 626 - protein, 10 aa, Homo sapiens
CSVFYGAPSK

SEQ ID NO: 627 - protein, 7 aa, Homo sapiens
FACYYPR

SEQ ID NO: 628 - protein, 8 aa, Homo sapiens
VEYGFQVK

SEQ ID NO: 629 - protein, 9 aa, Homo sapiens
ITQVLHFTK

SEQ ID NO: 630 - protein, 9 aa, Homo sapiens
GLQDEDGYR

SEQ ID NO: 631 - protein, 8 aa, Homo sapiens
TELLPGDR

SEQ ID NO: 632 - protein, 8 aa, Homo sapiens
DNLAIQTR

SEQ ID NO: 633 - protein, 11 aa, Homo sapiens
INHGILYDEEK

SEQ ID NO: 634 - protein, 11 aa, Homo sapiens
EIMENYNIALR

SEQ ID NO: 635 - protein, 10 aa, Homo sapiens
CEEDEEFTCR

SEQ ID NO: 636 - protein, 8 aa, Homo sapiens
WELCDIPR

SEQ ID NO: 637 - protein, 13 aa, Homo sapiens
CFELQEAGPPDCR

SEQ ID NO: 638 - protein, 9 aa, Homo sapiens
WFYIASAFR

SEQ ID NO: 639 - protein, 8 aa, Homo sapiens
TEDTIFLR

SEQ ID NO: 640 - protein, 15 aa, Homo sapiens
YVGGQEHFAHLLILR

SEQ ID NO: 641 - protein, 12 aa, Homo sapiens
TYMLAFDVNDEK

SEQ ID NO: 642 - protein, 15 aa, Homo sapiens
NWGLSVYADKPETTK

SEQ ID NO: 643 - protein, 14 aa, Homo sapiens
EQLGEFYEALDCLR

SEQ ID NO: 644 - protein, 9 aa, Homo sapiens
SDVVYTDWK

SEQ ID NO: 645 - protein, 8 aa, Homo sapiens
TGAQELLR

SEQ ID NO: 646 - protein, 16 aa, Homo sapiens
TMLLQPAGSLGSYSYR

SEQ ID NO: 647 - protein, 17 aa, Homo sapiens
AQGFTEDTIVFLPQTDK

SEQ ID NO: 648 - protein, 8 aa, Homo sapiens
ELDVLQGR

SEQ ID NO: 649 - protein, 4 aa, Homo sapiens
  MOD_RES (1)..(1): Xaa = Ile or Leu
XXGQ

SEQ ID NO: 650 - protein, 15 aa, Homo sapiens
GILILGQEQDTLGGR

SEQ ID NO: 651 - DNA, 24 bp, Homo sapiens
gagttggacgtcctgcagggtcgt

SEQ ID NO: 652 - DNA, 45 bp, Homo sapiens
gggatccttatcttgggccaggagcaggataccctgggtggccgg

SEQ ID NO: 653 - DNA, 21 bp, Homo sapiens
cgcctcacgctgaagttcctg

SEQ ID NO: 654 - DNA, 23 bp, Homo sapiens
ctggatgaggtggcccctcatgc

SEQ ID NO: 655 - DNA, 22 bp, Homo sapiens
tgttcagccgcttcctgtgcac

SEQ ID NO: 656 - DNA, 22 bp, Homo sapiens
tctagcagtacaatctcgttgg

SEQ ID NO: 657 - protein, 15 aa, Homo sapiens
EWVAIESDSVQPVPR

SEQ ID NO: 658 - protein, 8 aa, Homo sapiens
  MOD_RES (2)..(2): X = Ile or Leu
HXDXEEYR

SEQ ID NO: 659 - protein, 8 aa, Homo sapiens
  MOD_RES (2)..(2): X = Ile or Leu
HXDXEEYR

SEQ ID NO: 660 - DNA, 20 bp, Homo sapiens
  modified_base (10)..(10): n = a ,c ,g, or t
gcctaatggntcccaaactc

SEQ ID NO: 661 - DNA, 22 bp, Homo sapiens
gaggtgaatctgtcagtggatc

SEQ ID NO: 662 - DNA, 22 bp, Homo sapiens
atggaagaggctggctctgttg

SEQ ID NO: 663 - DNA, 22 bp, Homo sapiens
aagagatgggtacctccagagg

SEQ ID NO: 664 - DNA, 42 bp, Homo sapiens
gagtgggtggccatcgagagcgactctgtccagcctgtgcct

SEQ ID NO: 665 - protein, 14 aa, Homo sapiens
EWVAIESDSVQPVP

SEQ ID NO: 666 - DNA, 30 bp, Homo sapiens
gccatccatctagacctagaagaataccgg

SEQ ID NO: 667 - protein, 10 aa, Homo sapiens
AIHLDLEEYR

SEQ ID NO: 668 - DNA, 22 bp, Homo sapiens
acacccaaacatcttggcatcc

SEQ ID NO: 669 - DNA, 22 bp, Homo sapiens
tcagggagtggagatagggaac

SEQ ID NO: 670 - DNA, 22 bp, Homo sapiens
atggaagaggctggctctgttg

SEQ ID NO: 671 - DNA, 22 bp, Homo sapiens
aagagatgggtacctccagagg

SEQ ID NO: 672 - protein, 502 aa, Homo sapiens
RLTLKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVCKGRAKA

SEQ ID NO: 673 - DNA, 1515 bp, Homo sapiens
  misc_feature (1)..(1515): x = a, t, g or c
cgcctcacgctgaagttcctggccgtgctgctggccgcgggcatgctggcgttcctcggtgccgtcatctgcatcatcgccagcgtgcccctggcggccagcccggcgcgggcgctgcccggcggcgccgacaatgcttcggtcgcctcgggcgccgccgcgtccccgggcccgcagcggagcctgagcgcgctgcacggcgcgggcggttcagccgggccccccgcgctgcccggggcacccgcggccagcgcgcacccgctgccgcccgggcccctgttcagccgcttcctgtgcacgccgctggctgctgcctgcccgtcgggggcccagcagggggacgcggcgggcgctgcgccgggcgagcgcgaagagctgctgctgctgcagagcacggccgagcagctgcgccagacggcgctgcagcaggaggcgcgcatccgcgccgaccaggacaccatccgtgagctcaccggcaagctgggccgctgcgagagcggcctgccgcgcggcctccagggcgccgggccccgccgcgacaccatggccgacgggccctgggactcgcctgcgctcattctggagctggaggacgccgtgcgcgccctgcgggaccgcatcgaccgcctggaggagcttccagcccgtgtgaacctctcagctgccccagccccagtctctgctgtgcccaccggcctacactccaagatggaccagctggaggggcagctgctggcccaggtgctggcactggagaaggagcgtgtggccctcagccacagcagccgccggcagaggcaggaagtggaaaaggagttggacgtcctgcagggtcgtgtggctgagctggagcacgggtcctcagcctacagtcctccagatgccttcaagatcagcatccccatccgtaacaactacatgtacgcccgcgtgcggaaggctctgcccgagctctatgcattcaccgcctgcatgtggctgcggtccaggtccagcggcaccggncagggcacccccttctcctactcagtgcccgggcaggccaacgagattgtactgctagaggcgggccatgagcccatggagctgctgatcaacgacaaggtggcccagctgcccctgagcctgaaggacaatggctggcaccacatctgcatcgcctggaccacaagggatggcctatggtctgcctaccaggacggggagctgcagggctccggtgagaacctggctgcctggcaccccatcaagcctcatgggatccttatcttgggccaggagcaggataccctgggtggccggtttgatgccacccaggcctttgtcggtgacattgcccagtttaacctgtgggaccacgccctgacaccagcccaggtcctgggcattgccaactgcactgcgccactgctgggcaacgtccttccctgggaagacaagttggtggaggcctttgggggtgcaacaaaggctgccttcgatgtctgcaaggggagggccaaggcatgaggggcc

SEQ ID NO: 674 - protein, 501 aa, Homo sapiens
  MOD_RES (70)..(70): Xaa = Ile or Leu
MAASLLAVLLLLLLERGMFSSPSPPPALLEKVFQYIDLHQDEFVQTLKEWVAIESDSVQPVPRFRQELFXMMAVAADTLQRLGARVASVDMGPQQLPDGQSLPIPPVILAELGSDPTKGTVCFYGHLDVQPADRGDGWLTDPYVLTEVDGKLYGRGATDNKGPVLAWINAVSAFRALEQDLPVNIKFIIEGMEEAGSVALEELVEKEKDRFFSGVDYIVISDNLWISQRKPAITYGTRGNSYFMVEVKCRDQDFHSGTFGGILHEPMADLVALLGSLVDSSGHILVPGIYDEVVPLTEEEINTYKAIHLDLEEYRNSSRVEKFLFDTKEEILMHLWRYPSLSIHGIEGAFDEPGTKTVIPGRVIGKFSIRLVPHMNVSAVEKQVTRHLEDVFSKRNSSNKMVVSMTLGLHPWIANIDDTQYLAAKRAIRTVFGTEPDMIRDGSTIPIAKMFQEIVHKSVVLIPLGAVDDGEHSQNEKINRWNYIEGTKLFAAFFLEMAQIH

SEQ ID NO: 675 - DNA, 1587 bp, Homo sapiens
  modified_base (1)..(1587): n = a, c, g, or t
gnnnnnnagngntntannannaatgcntttgancatggctgcgtctttgctggctgtgctgctgctgctgctgctggagcgcggcatgttctcctcaccctccccgcccccggcgctgttagagaaagtcttccagtacattgacctncatcaggatgaatttgtgcagacgctgaaggagtgggtggccatcgagagcgactctgtccagcctgtgcctcgcttcagacaagagctcttcanaatgatggccgtggctgcggacacgctgcagcgcctgggggcccgtgtggcctcggtggacatgggtcctcagcagctgcccgatggtcagagtcttccaatacctcccgtcatcctggccgaactggggagcgatcccacgaaaggcaccgtgtgcttctacggccacttggacgtgcagcctgctgaccggggcgatgggtggctcacggacccctatgtgctgacggaggtagacgggaaactttatggacgaggagcgaccgacaacaaaggccctgtcttggcttggatcaatgctgtgagcgccttcagagccctggagcaagatcttcctgtgaatatcaaattcatcattgaggggatggaagaggctggctctgttgccctggaggaacttgtggaaaaagaaaaggaccgattcttctctggtgtggactacattgtaatttcagataacctgtggatcagccaaaggaagccagcaatcacttatggaacccgggggaacagctacttcatggtggaggtgaaatgcagagaccaggattttcactcaggaacctttggtggcatccttcatgaaccaatggctgatctggttgctcttctcggtagcctggtagactcgtctggtcatatcctggtccctggaatctatgatgaagtggttcctcttacagaagaggaaataaatacatacaaagccatccatctagacctagaagaataccggaatagcagccgggttgagaaatttctgttcgatactaaggaggagattctaatgcacctctggaggtacccatctctttctattcatgggatcgagggcgcgtttgatgagcctggaactaaaacagtcatacctggccgagttataggaaaattttcaatccgtctagtccctcacatgaatgtgtctgcggtggaaaaacaggtgacacgacatcttgaagatgtgttctccaaaagaaatagttccaacaagatggttgtttccatgactctaggactacacccgtggattgcaaatattgatgacacccagtatctcgcagcaaaaagagcgatcagaacagtgtttggaacagaaccagatatgatccgggatggatccaccattccaattgccaaaatgttccaggagatcgtccacaagagcgtggtgctaattccgctgggagctgttgatgatggagaacattcgcagaatgagaaaatcaacaggtggaactacatagagggaaccaaattatttgctgcctttttcttagagatggcccagatccattaatcacaagaaccttctagtctgatctgatccactgacagattcacctc

SEQ ID NO: 676 - DNA, 24 bp, Homo sapiens
gagttggacgtcctgcagggtcgt

SEQ ID NO: 677 - DNA, 45 bp, Homo sapiens; identical to SEQ ID NO: 652